(19) United States
(12) Patent Application Publication
(10) Pub. No.: US 2025/0099433 A1
(43) Pub. Date: Mar. 27, 2025

(54) COMPOSITION AND METHOD FOR TREATMENT OF GRAM NEGATIVE BACTERIAL INFECTION

(71) Applicant: The Research Foundation for the State University of New York, Albany, NY (US)

(72) Inventors: Peter J. Tonge, Stony Brook, NY (US); Sneha Basak, Stony Brook, NY (US); Fereidoon Daryaee, Stony Brook, NY (US)

(73) Assignee: The Research Foundation for the State University of New York, Albany, NY (US)

(21) Appl. No.: 18/294,712

(22) PCT Filed: Aug. 4, 2022
(86) PCT No.: PCT/US2022/074510

§ 371 Date: Feb. 2, 2024

Related U.S. Application Data
(60) Provisional application No. 63/367,494, filed on Jul. 1, 2022.
(60) Provisional application No. 63/233,459, filed on Aug. 16, 2021.
(60) Provisional application No. 63/299,483, filed on Jan. 14, 2022.

(51) Int. Cl. A61K 31/4192; A61K 31/5377; C07D 213/64; C07D 249/06; C07D 401/10; C07D 401/14; C07D 405/14; C07D 413/10

(52) U.S. Cl. CPC A61K 31/4192; A61K 31/5377; C07D 213/64; C07D 249/06; C07D 401/10; C07D 401/14; C07D 405/14; C07D 413/10

(57) ABSTRACT

The invention provides a compound having the structure:

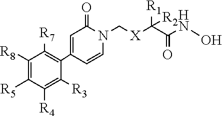

wherein R₁ and R₂ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, -alkyl-OR₁₄, C₁₋₁₀ alkyl, C₂₋₁₀ alkenyl, or C₂₋₁₀ alkynyl; or wherein R₁ and R₂ together formed into a cycloalkyl or cycloheteroalkyl;
wherein R₃ and R₇ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl;
wherein R₄ and R₆ are each independently H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl;
wherein R₅ is —NO₂, —NHR₁₄, —NR₁₄R₁₅, —SR₁₄, —SO₂R₁₄, —(C═O)—NH—R₁₄, —CO₂R₁₄, -alkyl-NR₁₄R₁₅, -alkyl- OR₁₄, C₂₋₁₀ alkenyl, C₂₋₁₀ alkynyl, —(C₀₋₁₀ alkyl)-heterocyclyl,

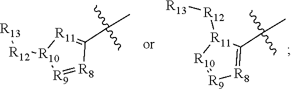

wherein R₈, R₉, R₁₀, and R₁₁ are each independently N or CH;
wherein R₁₂ is C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₃ is halogen, —NO₂, —CN, —NHR₁₄, —NR₁₄R₁₅, —SR₁₄, —SO₂R₁₄, —OR₁₄, —CO₂R₁₄, —CF₃, —NR₁₄R₁₅, —OR₁₄, C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₄ is H, C₂₋₁₀ alkenyl, C₂₋₁₀ alkynyl, —C═O-alkyl-aryl, or alkyl-aryl;
wherein R₁₅ is H, C₁₋₁₀ alkyl, C₂₋₁₀ alkenyl, C₂₋₁₀ alkynyl, —C═O-alkyl-aryl, or alkyl-aryl;
wherein X is CH₂ or NH;
wherein R₃ and R₅ are not both halogen, R₅ and R₇ are not both halogen;
wherein when R₃ or R₇ is halogen, R₄ and R₆ are not alkyl;
wherein when R₅ is —(C₀₋₁₀ alkyl)-heterocyclyl, the heterocyclyl is not tetrahydropyranyl;
wherein when R₃ or R₇ is halogen, R₄ and R₆ are not —OR₁₄;
wherein when R₃ is alkyl or H, R₆ is not —NHR₁₄ and —NR₁₄R₁₅; wherein when R₇ is alkyl or H, R₄ is not —NHR₁₄ and —NR₁₄R₁₅.

This application claims the benefit of U.S. Provisional Application No. 63/229,483, filed Aug. 4, 2021, U.S. Provisional Application No. 63/233,459, filed Aug. 16, 2021 and U.S. Provisional Application No. 63/367,494, filed Jul. 1, 2022, the entire contents of each of which are hereby incorporated by reference into the subject application.

### GOVERNMENT SUPPORT

This invention was made with government support under GM102864 awarded by the National Institutes of Health. The government has certain rights in the invention.

Throughout this application, certain publications are referenced in parentheses. Full citations for these publications may be found immediately preceding the claims. The disclosures of these publications in their entireties are hereby incorporated by reference into this application in order to describe more fully the state of the art to which this invention relates.

### BACKGROUND OF THE INVENTION

Novel therapeutics are urgently needed to treat diseases caused by MDR strains of Pseudomonas aeruginosa and other virulent strains of Gram-negative bacteria, which are a major cause of hospital and community acquired infection. To meet this challenge, the composition and method described herein provides inhibitors of the LpxC enzyme, UDP-3-O—((R)-3-hydroxymyristoyl)-Nacetylglucosamine deacetylase, which catalyzes the first committed step in the biosynthesis of Lipid A, a membrane-associated portion of Lipopolysaccharide (LPS). LPS forms a major component of the outer membrane of Gram-negative bacteria, and is essential for viability and virulence.

The compositions described herein are inhibitors of LpxC with improved safety profiles compared to existing compounds. This is achieved by synthesizing inhibitors that dissociate slowly from LpxC and thus that have extended activity at low drug concentration so that they can be administered at lower doses, resulting in reduced exposure, decreased side effects, increased compliance and reduced drug resistance. These activities are supported by a PK/PD model that integrates drug-target kinetics into predictions of drug activity.

Current estimates indicate that P. aeruginosa is responsible for approximately 10% of all hospital acquired infections, playing a major role in diseases such as hospital-acquired pneumonia, urinary tract infections (UTI) intra-abdominal infections (IAI) and catheter-associated bloodstream infections.

Chronic infection with Pseudomonas is also the primary cause of pulmonary decline and mortality in individuals with cystic fibrosis. Because of the prevalence of drug resistance, Pseudomonas infection is normally treated by combination therapy, such as a β-lactam and aminoglycoside. However, Pseudomonas strains developed increasing rate of multidrug resistant which poses a major barrier to treatment and has led to the use of drugs such as polymyxin that have serious side effects. Compared to infections caused by non-resistant strains, resistant and MDR pathogens increase the average length of hospital stay by about 5 days and 17 days, respectively. The average hospital costs of resistant and MDR pathogens in healthcare-associated infections are about $38,000 and $72,000, respectively. In addition, mean annual per-patient costs following initial P. aeruginosa infection increased by an estimated $18,516 for patients with cystic fibrosis.

Long residence time inhibitors of LpxC to treat serious infections caused by MDR-pathogens with minimized impact on the microbiome are provided. These compositions have extended target engagement to reduce dosing frequency and exposure, resulting in decreased side effects, increased compliance and reduced drug resistance.

The translation of time-dependent drug-target occupancy to extended pharmacological activity at low drug concentration depends on factors such as target vulnerability and the rate of target turnover. The post-antibiotic effect (PAE) caused by inhibitors of bacterial drug targets could be used to assess target vulnerability, and that high levels of target vulnerability coupled with relatively low rates of target re-synthesis resulted in a strong correlation between drug-target residence time and the PAE following compound washout. Although the residence time of inhibitors on UDP-3-O-acyl-N-acetylglucosamine deacetylase (LpxC) in Pseudomonas aeruginosa (paLpxC) result in significant PAE, inhibitors of the equivalent enzyme in Escherichia coli (ecLpxC) do not cause a PAE. Hyperactivity of the fatty acid biosynthesis enzyme FabZ or the inclusion of sub-MIC levels of azithromycin lead to the observation of a PAE for three inhibitors of ecLpxC. FabZ hyperactivity has been shown to stabilize ecLpxC and using mass spectrometry, it was demonstrated that the appearance of a PAE can be directly linked to a 3-fold increase in the stability of ecLpxC. These studies substantiate the importance of target turnover in time-dependent drug activity.

UDP-3-O—(R-3-hydroxymyristoyl)-N-acetylglucosamine deacetylase (LpxC) is a promising drug target in Gram-negative bacteria. There is a correlation between the residence time of inhibitors on the Pseudomonas aeruginosa LpxC (paLpxC) and the post-antibiotic effect (PAE) caused by the inhibitors on the growth of P. aeruginosa. Given that drugs with prolonged activity following compound removal may have advantages in dosing regimens, the structure-kinetic relationship for paLpxC inhibition to rationally modulate the lifetime of inhibitors on the enzyme was explored. Analogs of the pyridone methylsulfone paLpxC inhibitor PF5081090 (1) were synthesized and the kinetics for binding to the enzyme was determined using a fluorescence-based competition assay. Several analogs had longer residence times on paLpxC than 1 (41 min) including PT913, which has a residence time of 124 min. PT913 also has a PAE of 4 h, extending the original correlation observed between residence time and PAE for inhibitors of paLpxC. Collectively, the current application provides a platform for a rational modulation of inhibitor residence time and a development of antibacterial agents which cause a prolonged suppression of bacterial growth.

### SUMMARY OF THE INVENTION

The invention provides a compound having the structure:

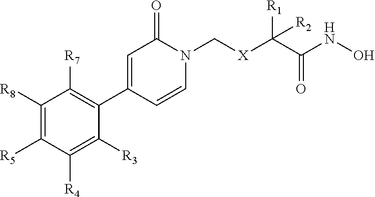

wherein R₁ and R₂ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, -alkyl-OR₁₄, C₁₋₁₀ alkyl, C₂₋₁₀ alkenyl, or C₂₋₁₀ alkynyl; or wherein R₁ and R₂ together formed into a cycloalkyl or cycloheteroalkyl;
wherein R₃ and R₇ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl;
wherein R₄ and R₆ are each independently H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl;
wherein R₅ is —NO₂, —NHR₁₄, —NR₁₄R₁₅, —SR₁₄, —SO₂R₁₄, —(C═O)—NH—R₁₄, —CO₂R₁₄, -alkyl-NR₁₄R₁₅, -alkyl-OR₁₄, C₂₋₁₀ alkenyl, C₂₋₁₀ alkynyl, —(C₀₋₁₀ alkyl)-heterocyclyl,

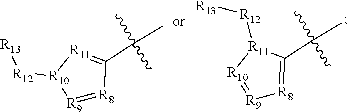

wherein R₈, R₉, R₁₀, and R₁₁ are each independently N or CH;
wherein R₁₂ is C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₃ is halogen, —NO₂, —CN, —NHR₁₄, —NR₁₄R₁₅, —SR₁₄, —SO₂R₁₄, —OR₁₄, —CO₂R₁₄, —CF₃, —NR₁₄R₁₅, —OR₁₄, C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₄ is H, C₂₋₁₀ alkenyl, C₂₋₁₀ alkynyl, —C═O-alkyl-aryl, or alkyl-aryl;
wherein R₁₅ is H, C₁₋₁₀ alkyl, C₂₋₁₀ alkenyl, C₂₋₁₀ alkynyl, —C═O-alkyl-aryl, or alkyl-aryl;
wherein X is CH₂ or NH;
wherein R₃ and R₅ are not both halogen, R₅ and R₇ are not both halogen;
wherein when R₃ or R₇ is halogen, R₄ and R₆ are not alkyl;
wherein when R₅ is —(C₀₋₁₀ alkyl)-heterocyclyl, the heterocyclyl is not tetrahydropyranyl;
wherein when R₃ or R₇ is halogen, R₄ and R₆ are not —OR₁₄;
wherein when R₃ is alkyl or H, R₆ is not —NHR₁₄ and —NR₁₄R₁₅; wherein when R₇ is alkyl or H, R₄ is not —NHR₁₄ and —NR₁₄R₁₅.

The invention provides a compound having the structure:

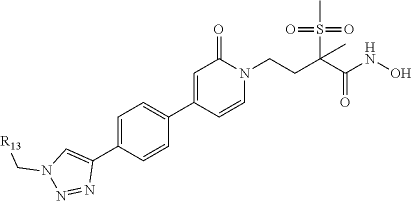

wherein R₁₃ is

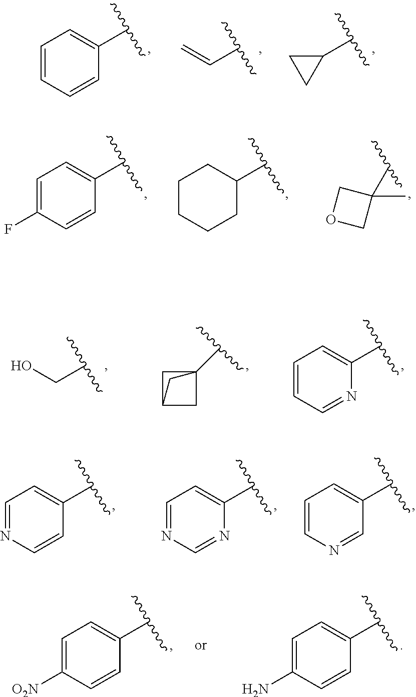

The invention provides a compound having the structure:

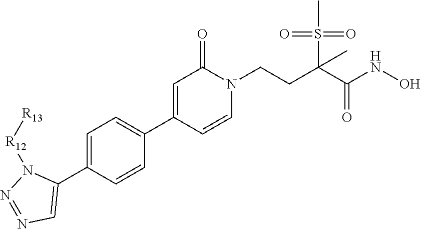

wherein R₁₂ is C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₃ is halogen, —NO₂, —CN, —NHR₁₄, —NR₁₄R₁₅, —SR₁₄, —SO₂R₁₄, —OR₁₄, —CO₂R₁₄, —CF₃, —NR₁₄R₁₅, —OR₁₄, C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₄ is H, C₂₋₁₀ alkenyl, C₂₋₁₀ alkynyl, —C═O-alkyl-aryl, or alkyl-aryl;
wherein R₁₅ is H, C₁₋₁₀ alkyl, C₂₋₁₀ alkenyl, C₂₋₁₀ alkynyl, —C═O-alkyl-aryl, or alkyl-aryl.

The invention provides a compound having the structure:

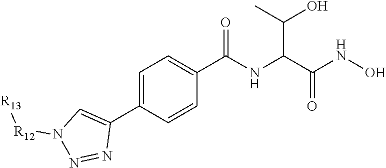

wherein R₁₂ is C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₃ is halogen, —NO₂, —CN, —NHR₁₄, —NR₁₄R₁₅, —SR₁₄, —SO₂R₁₄, —OR₁₄, —CO₂R₁₄, —CF₃, —NR₁₄R₁₅, —OR₁₄, C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₄ is H, C₂₋₁₀ alkenyl, C₂₋₁₀ alkynyl, —C═O-alkyl-aryl, or alkyl-aryl;
wherein R₁₅ is H, C₁₋₁₀ alkyl, C₂₋₁₀ alkenyl, C₂₋₁₀ alkynyl, —C═O-alkyl-aryl, or alkyl-aryl.

The invention provides a compound having the structure:

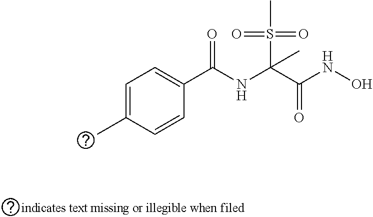 [SMILES: Cc1ccc(C(=O)NC(C)(C(=O)NO)S(C)(=O)=O)cc1]

wherein R₅ is

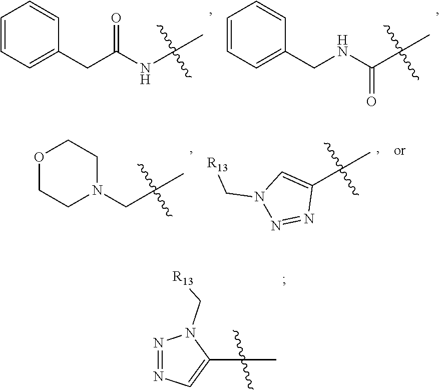

wherein R₁₂ is C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₃ is halogen, —NO₂, —CN, —NHR₁₄, —NR₁₄R₁₅, —SR₁₄, —SO₂R₁₄, —OR₁₄, —CO₂R₁₄, —CF₃, —NR₁₄R₁₅, —OR₁₄, C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₄ is H, C₂₋₁₀ alkenyl, C₂₋₁₀ alkynyl, —C═O-alkyl-aryl, or alkyl-aryl;
wherein R₁₅ is H, C₁₋₁₀ alkyl, C₂₋₁₀ alkenyl, C₂₋₁₀ alkynyl, —C═O-alkyl-aryl, or alkyl-aryl.

The invention provides a compound having the structure:

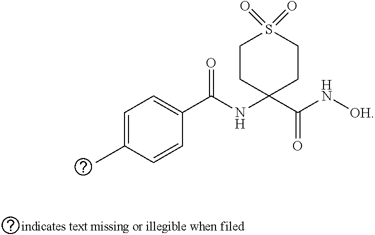

wherein R₅ is —NO₂, —NHR₁₄, —NR₁₄R₁₅, —SR₁₄, —SO₂R₁₄, —(C═O)—NH—R₁₄, —CO₂R₁₄, -alkyl-NR₁₄R₁₅, -alkyl-OR₁₄, C₂₋₁₀ alkenyl, C₂₋₁₀ alkynyl, —(C₀₋₁₀ alkyl)-heterocyclyl,

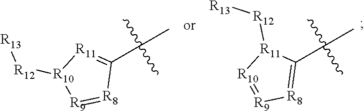

wherein R₈, R₉, R₁₀, and R₁₁ are each independently N or CH;
wherein R₁₂ is C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₃ is halogen, —NO₂, —CN, —NHR₁₄, —NR₁₄R₁₅, —SR₁₄, —SO₂R₁₄, —OR₁₄, —CO₂R₁₄, —CF₃, —NR₁₄R₁₅, —OR₁₄, C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₄ is H, C₂₋₁₀ alkenyl, C₂₋₁₀ alkynyl, —C═O-alkyl-aryl, or alkyl-aryl;
wherein R₁₅ is H, C₁₋₁₀ alkyl, C₂₋₁₀ alkenyl, C₂₋₁₀ alkynyl, —C═O-alkyl-aryl, or alkyl-aryl;
wherein X is CH₂ or NH;
wherein R₃ and R₅ are not both halogen, R₅ and R₇ are not both halogen;
wherein when R₃ or R₇ is halogen, R₄ and R₆ are not alkyl;
wherein when R₅ is —(C₀₋₁₀ alkyl)-heterocyclyl, the heterocyclyl is not tetrahydropyranyl;
wherein when R₃ or R₇ is halogen, R₄ and R₆ are not —OR₁₄;
wherein when R₃ is alkyl or H, R₆ is not —NHR₁₄ and —NR₁₄R₁₅; wherein when R₇ is alkyl or H, R₄ is not —NHR₁₄ and —NR₁₄R₁₅.

The invention provides a pharmaceutical composition comprising a compound having the structure:

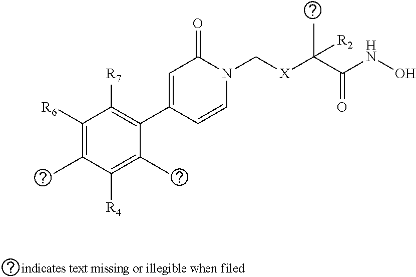

wherein R₁ and R₂ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, -alkyl-OR₁₄, C₁₋₁₀ alkyl, C₂₋₁₀ alkenyl, or C₂₋₁₀ alkynyl; or wherein R₁ and R₂ together formed into a cycloalkyl or cycloheteroalkyl;
wherein R₃ and R₇ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl;
wherein R₄ and R₆ are each independently H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl;
wherein R₅ is —NO₂, —NHR₁₄, —NR₁₄R₁₅, —SR₁₄, —SO₂R₁₄, —(C═O)—NH—R₁₄, —CO₂R₁₄, -alkyl-NR₁₄R₁₅, -alkyl-OR₁₄, C₂₋₁₀ alkenyl, C₂₋₁₀ alkynyl, —(C₀₋₁₀ alkyl)-heterocyclyl,

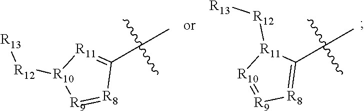

wherein R₈, R₉, R₁₀, and R₁₁ are each independently N or CH;
wherein R₁₂ is C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₃ is halogen, —NO₂, —CN, —NHR₁₄, —NR₁₄R₁₅, —SR₁₄, —SO₂R₁₄, —OR₁₄, —CO₂R₁₄, —CF₃, —NR₁₄R₁₅, —OR₁₄, C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₄ is H, C₂₋₁₀ alkenyl, C₂₋₁₀ alkynyl, —C═O-alkyl-aryl, or alkyl-aryl;
wherein R₁₅ is H, C₁₋₁₀ alkyl, C₂₋₁₀ alkenyl, C₂₋₁₀ alkynyl, —C═O-alkyl-aryl, or alkyl-aryl;
wherein X is CH₂ or NH;
wherein R₃ and R₅ are not both halogen, R₅ and R₇ are not both halogen;
wherein when R₃ or R₇ is halogen, R₄ and R₆ are not alkyl;
wherein when R₅ is —(C₀₋₁₀ alkyl)-heterocyclyl, the heterocyclyl is not tetrahydropyranyl;
wherein when R₃ or R₇ is halogen, R₄ and R₆ are not —OR₁₄;
wherein when R₃ is alkyl or H, R₆ is not —NHR₁₄ and —NR₁₄R₁₅; wherein when R₇ is alkyl or H, R₄ is not —NHR₁₄ and —NR₁₄R₁₅,
with at least one pharmaceutically acceptable excipient, diluent or carrier.

The invention provides a method of inhibiting bacteria growth, comprising contacting the bacteria with an amount of a compound having the structure:

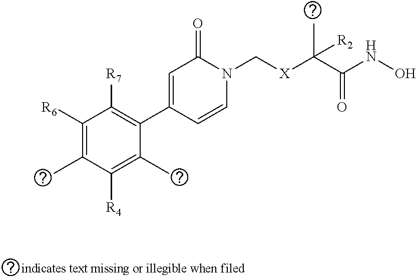

wherein R₁ and R₂ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, -alkyl-OR₁₄, C₁₋₁₀ alkyl, C₂₋₁₀ alkenyl, or C₂₋₁₀ alkynyl; or wherein R₁ and R₂ together formed into a cycloalkyl or cycloheteroalkyl;
wherein R₃ and R₇ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl;
wherein R₄ and R₆ are each independently H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl;
wherein R₅ is —NO₂, —NHR₁₄, —NR₁₄R₁₅, —SR₁₄, —SO₂R₁₄, —(C═O)—NH—R₁₄, —CO₂R₁₄, -alkyl-NR₁₄R₁₅, -alkyl-OR₁₄, C₂₋₁₀ alkenyl, C₂₋₁₀ alkynyl, —(C₀₋₁₀ alkyl)-heterocyclyl,

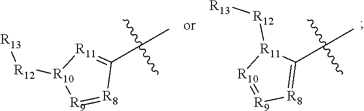

wherein R₈, R₉, R₁₀, and R₁₁ are each independently N or CH;
wherein R₁₂ is C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₃ is halogen, —NO₂, —CN, —NHR₁₄, —NR₁₄R₁₅, —SR₁₄, —SO₂R₁₄, —OR₁₄, —CO₂R₁₄, —CF₃, —NR₁₄R₁₅, —OR₁₄, C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₄ is H, C₂₋₁₀ alkenyl, C₂₋₁₀ alkynyl, —C═O-alkyl-aryl, or alkyl-aryl;
wherein R₁₅ is H, C₁₋₁₀ alkyl, C₂₋₁₀ alkenyl, C₂₋₁₀ alkynyl, —C═O-alkyl-aryl, or alkyl-aryl;
wherein X is CH₂ or NH;
wherein R₃ and R₅ are not both halogen, R₅ and R₇ are not both halogen;
wherein when R₃ or R₇ is halogen, R₄ and R₆ are not alkyl;
wherein when R₅ is —(C₀₋₁₀ alkyl)-heterocyclyl, the heterocyclyl is not tetrahydropyranyl;
wherein when R₃ or R₇ is halogen, R₄ and R₆ are not —OR₁₄;
wherein when R₃ is alkyl or H, R₆ is not —NHR₁₄ and —NR₁₄R₁₅; wherein when R₇ is alkyl or H, R₄ is not —NHR₁₄ and —NR₁₄R₁₅.

The invention provides a method of treating a bacterial infection in a patient, comprising administering to the patient a therapeutically effective amount of a compound having the structure:

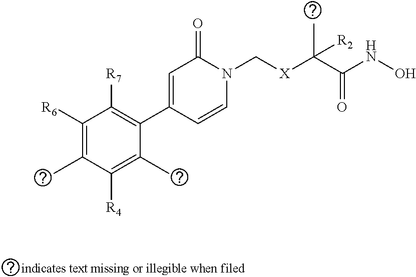

wherein R₁ and R₂ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, -alkyl-OR₁₄, C₁₋₁₀ alkyl, C₂₋₁₀ alkenyl, or C₂₋₁₀ alkynyl; or wherein R₁ and R₂ together formed into a cycloalkyl or cycloheteroalkyl;
wherein R₃ and R₇ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl;
wherein R₄ and R₆ are each independently H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl;
wherein R₅ is —NO₂, —NHR₁₄, —NR₁₄R₁₅, —SR₁₄, —SO₂R₁₄, —(C═O)—NH—R₁₄, —CO₂R₁₄, -alkyl-NR₁₄R₁₅, -alkyl-OR₁₄, C₂₋₁₀ alkenyl, C₂₋₁₀ alkynyl, —(C₀₋₁₀ alkyl)-heterocyclyl,

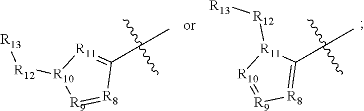

wherein R₈, R₉, R₁₀, and R₁₁ are each independently N or CH;
wherein R₁₂ is C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₃ is halogen, —NO₂, —CN, —NHR₁₄, —NR₁₄R₁₅, —SR₁₄, —SO₂R₁₄, —OR₁₄, —CO₂R₁₄, —CF₃, —NR₁₄R₁₅, —OR₁₄, C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₄ is H, C₂₋₁₀ alkenyl, C₂₋₁₀ alkynyl, —C═O-alkyl-aryl, or alkyl-aryl;
wherein R₁₅ is H, C₁₋₁₀ alkyl, C₂₋₁₀ alkenyl, C₂₋₁₀ alkynyl, —C═O-alkyl-aryl, or alkyl-aryl;
wherein X is CH₂ or NH;
wherein R₃ and R₅ are not both halogen, R₅ and R₇ are not both halogen;
wherein when R₃ or R₇ is halogen, R₄ and R₆ are not alkyl;
wherein when R₅ is —(C₀₋₁₀ alkyl)-heterocyclyl, the heterocyclyl is not tetrahydropyranyl;
wherein when R₃ or R₇ is halogen, R₄ and R₆ are not —OR₁₄;
wherein when R₃ is alkyl or H, R₆ is not —NHR₁₄ and —NR₁₄R₁₅; wherein when R₇ is alkyl or H, R₄ is not —NHR₁₄ and —NR₁₄R₁₅.

### BRIEF DESCRIPTION OF THE FIGURES

FIG. 1. The ecLpxC inhibitors of PT 810, PT 805 and Chiron-90.

FIG. 2. Residence time of the PT 810, PT 805 and Chiron-90.

FIG. 3. Time kill assays for PT805, PT810 and CHIR-090 in (a) wild-type E. coli and (b) FabZ* E. coli. Cultures of wild-type or FabZ* E. coli (10⁶ CFU/mL) in cation-adjusted Mueller-Hinton (CaMH) media were treated with 16×MIC of inhibitor at 37° C. Samples (100 μL) of the cultures were then plated on Muller-Hinton agar plates every hour and CFUs enumerated following incubation of the plates at 37° C. for 16 h. Time kill assay data is shown for PT805, PT810 and CHIR-090 in (a) wild-type E. coli and (b) FabZ* E. coli. Each time point was performed in duplicate or triplicate and the entire experiment was performed in triplicate. The data points in the figure are the mean values from the independent measurements and the error bars represent the standard deviation from the mean. Similar data were observed at lower concentrations of compounds. For instance, 4×MIC of PT805, PT810 and CHIR-090 reduced the starting log CFU/mL of the bacterial culture by greater than 3 logs.

FIG. 4. Post-antibiotic effect of the ecLpxC inhibitors on wild-type and FabZ* E. coli. Cultures of wild-type or FabZ* E. coli (10⁶ CFU/mL) were treated with 1×, 4× or 16×MIC of inhibitor for 1 h followed by 1:1000-fold dilution into fresh cation-adjusted Mueller-Hinton (CaMH) media at 37° C. Samples (100 μL) of the diluted cultures were then plated on Muller-Hinton agar plates every hour and CFUs enumerated following incubation of the plates at 37° C. for 16 h. PAE data for wild-type E. coli are in panels (a) PT805, (c) PT810 and (e) CHIR-090, and for FabZ* E. coli in (b) PT805, (d) PT810 and (f) CHIR-090. The experimental data points are the mean values from triplicate, independent measurements and the error bars represent the standard deviation from the mean.

FIG. 5. Post-antibiotic effect of erythromycin, cefamandole and trimethoprim in wild-type and FabZ* E. coli. Cultures of wild-type or FabZ* E. coli (10⁶ CFU/mL) were treated with 1×, 4× or 16×MIC of each antibiotic for 1 h followed by 1:1000-fold dilution into fresh CaMH media at 37° C. Samples (100 μL) of the diluted cultures were then plated on Muller-Hinton agar plates every hour and CFUs enumerated following incubation of the plates at 37° C. for 16 h. PAE data for wild-type E. coli are in panels (a) Erythromycin, (c) Cefamandole and (e) Trimethoprim, and for FabZ* E. coli in (b) Erythromycin, (d) Cefamandole and (f) Trimethoprim. Each time point was performed in duplicate or triplicate and the entire experiment was performed in triplicate. The data points in the figure are the mean values from the independent measurements, and the error bars represent the standard deviation from the mean.

FIG. 6. Post-antibiotic effect in the presence of sub-MIC azithromycin. Cultures of wild-type E. coli (K12 MG1655) 10⁶ CFU/mL were treated with 0× or 16×MIC of inhibitor for 1 h followed by 1:1000-fold dilution into fresh CaMH media containing 0×, 0.2× or 0.4×MIC azithromycin at 37° C. Samples (100 μL) of the diluted cultures were then plated on Muller-Hinton agar plates every hour and CFUs were enumerated following incubation of the plates at 37° C. for 16 h. PAE data are shown for (a) PT805, (b) PT810 and (c) CHIR-090 and (d) cefamandole. Values in parenthesis are the concentration of azithromycin in the media (0×, 0.2× or 0.4×MIC). The experimental data points are the mean values from triplicate, independent measurements and the error bars represent the standard deviation from the mean.

FIG. 7. Protein turnover by pSILAC mass spectrometry. Protein turnover was quantified in the wild-type strain of E. coli (black), the FabZ* mutant strain (blue), and in the wild-type strain grown in media containing sub-MIC concentration of azithromycin (0.4×MIC) (red). In each case the relative isotopic abundance (RIA) was determined as a function of time after transferring bacterial cultures grown in media containing “heavy” lysine to media containing normal “light” lysine. The RIA was calculated by quantifying the isotopic abundance of peptides generated by trypsin digest using mass spectrometry. ecLpxC (A) was enriched using antibody pull-down with beads coated with the ecLpxC antibody. This method also led to the enrichment of several other proteins that were used as controls including GAPDH (B), 60 kDa chaperonin (C), and malate dehydrogenase (D). Each point represents average of three independent biological replicates where the error bars represent the standard deviation from the mean.

FIG. 8. Protein turnover by pSILAC mass spectrometry Protein turnover was quantified in the wild-type strain of E. coli (black), the FabZ* mutant strain (blue), and in the wild-type strain grown in media containing sub-MIC concentration of azithromycin (0.4×MIC) (red). In each case the relative isotopic abundance (RIA) was determined as a function of time after transferring bacterial cultures grown in media containing “heavy” lysine to media containing normal “light” lysine. The RIA was calculated by quantifying the isotopic abundance of peptides generated by trypsin digest using mass spectrometry. ecLpxC was enriched using antibody pull-down with beads coated with the ecLpxC antibody which led to the enrichment of several other proteins that were used as controls including (a) glutamine synthase, (b) outer membrane protein A, (c) 30S ribosomal protein S16, and (d) 30S ribosomal protein S5. Each point represents the average of three independent biological replicates where the error bars represent the standard deviation from the mean.

FIG. 9. Representative LpxC inhibitors. Biological and microbiological data was taken from the following literature sources: CHIR-090,¹⁵ BB-78485,¹⁶ ACHN-975,¹⁸ L-161,240,¹⁴ PF-5081090,¹⁹ TP0586532,²³ Entasis (compound 19),²² Novartis Compound 1(+/−),²⁰ and Novartis compound 13f.²¹

FIG. 10. Fluorescence spectra of PT855. Fluorescence emission and excitation scans of 200 nM PT855 in buffer (25 mM NaH₂PO₄ pH 8.0 containing 300 mM KCl, 1% DMSO). All measurements were performed with a slit width of 2 nm, and readings were taken at 3 points/sec using a HORIBA QM-8075-11-C fluorimeter.

FIG. 11. Fluorescence competition data for representative LpxC inhibitors. Inhibitors were preincubated with 10 μM paLpxC in 25 mM Na₂PO₄ pH 8.0 buffer containing 300 mM KCl and 2% DMSO (v/v) in a total volume of 50 μL for 18 h at 37° C. For enantiomerically pure compounds, 10 μM of inhibitor was used, whereas 20 μM was used for racemic compounds, based on the rationale that the (R) enantiomer binds preferentially to the enzyme.³⁰ The enzyme-inhibitor complexes were diluted 200-fold into a 200 nM solution of the fluorescent competitor (PT855). The data were analyzed in Kintek assuming that both the inhibitor and competitor follow one-step binding equilibria. Each plot shows the time-dependent change in fluorescence following an addition of the enzyme-inhibitor complex, as well as the change in fluorescence if only enzyme was added to the fluorescent ligand. (a) PT810, (b) PT901, (c) PT902, (d) PT913, (e) PT909, (f) PT916, (g) PT921, (h) PT923, (i) PT925.

FIG. 12. Fluorescence competition data. Inhibitors were preincubated with 10 μM paLpxC in 25 mM NaH₂PO₄ pH 8.0 containing 300 mM KCl, and 2% DMSO (v/v) in a total volume of 50 μL for 18 h at 37° C. For enantiomerically pure compounds, 10 μM of inhibitor was used, whereas 20 μM was used for racemic compounds. The enzyme-inhibitor complexes were diluted 200-fold into a 200 nM solution of the fluorescent competitor (PT855). The data were analyzed in Kintek assuming that both the inhibitor and competitor follow one step binding equilibria. Each plot shows the time-dependent change in fluorescence following addition of the enzyme-inhibitor complex, as well as the change in fluorescence if only enzyme is added to the fluorescent ligand.

FIG. 13. Correlation plot of ΔF values versus Kᵢ. A plot of ΔF and log Kᵢ demonstrates a clear correlation between the change in fluorescence with the Kᵢ value of the inhibitors. The red dotted lines represent linear fit to the data.

FIG. 14. Spin-column assay. A spin-column assay was used to quantify the residence time of inhibitors on paLpxC at 37° C. Following preincubation of enzyme and inhibitor for 16 h, 5 μL of the reaction mixture was rapidly diluted into 2 mL of buffer containing 1 μM of the fluorescent competitor ligand. Samples were then subjected to size exclusion chromatography at different times using a Spin-Trap G-25 column (800×g for 2 min). The eluent was then analyzed using a Biotek Gen5, 3.09 plate reader using □ex 325 nm and □em 420 nm, and the change in fluorescence as a function of time was fit to a first-order rate equation using GraphPad Prism. a) PT805, k=0.010±0.002 min⁻¹, t_{R}=97.3±22.0 min. b) PT901, k=0.14±0.06 min⁻¹, t_{R}=7.3±3.0 min. c) PT907, k=0.007±0.002 min⁻¹, t_{R}=146.5±44.0 min. d) PT920, k=0.013±0.004 min⁻¹, t_{R}=79±24 min. e) PT909, k=0.009±0.002 min⁻¹, t_{R}=106±25 min. f) PF5081090 (1), k=0.012±0.003 min⁻¹, t_{R}=80.9±19.0 min.

FIG. 15. Correlation plot between t_{R} determined at 37° C. using the spin column and fluorescence competition assays. A correlation was observed between the two assays with a slope of 1.6, which indicates that the residence times obtained from the spin column-based assay are consistently longer than the times from competition assay for all the compounds tested. The red line represents linear fit of the data.

FIG. 16. X-ray structure of 1 and PT805 bound to paLpxC.

FIG. 17. Post-antibiotic effect of the paLpxC inhibitors on wild-type P. aeruginosa. Cultures of P. aeruginosa PAO1 (10⁶ CFU/mL) were treated with 4×, 8× or 16× of each antibiotic for 1 h followed by 1:1000-fold dilution into fresh CAMHII media at 37° C. Samples (100 μL) of the diluted cultures were then plated on Muller-Hinton agar plates every hour, and CFUs enumerated following incubation of the plates at 37° C. for 16 h. (a) PT901 (b) PT920 (c) PT805 (d) PT908 (e) PT909 and (f) PT913. The relatively high MIC of PT913 prevented PAE values from being measured for concentrations over 4×MIC. The experimental data points are the mean values from triplicate, independent measurements, and the error bars represent the standard deviation from the mean.

FIG. 18. Time-kill assays. Cultures of Pseudomonas aeruginosa PA01 (10⁶ CFU/mL) were treated with 4×, 8×, and 16×MIC of different LpxC inhibitors in CaMH media at 37° C. Samples (100 μL) of the cultures were then plated on Muller-Hinton agar plates every hour, and CFUs enumerated following incubation of the plates at 37° C. for 16 h. Timekill assay data is shown for PT901, PT908, PT920, PT805, and PT909. The experimental data points are the mean values from triplicate, independent measurements, and the error bars represent the standard deviation from the mean.

FIG. 19. Cytotoxicity of the paLpxC inhibitors. Cytotoxicity was determined using Vero cells (ATCC CCL-81).¹⁴ Cell cultures were grown in DMEM supplemented with 10% fetal bovine serum and aliquoted into a 96-well plate to 2×10⁴ cells/well. After 24 h incubation at 37° C. in 5% CO₂, the media was replaced with serum-free DMEM. Subsequently, the plates were incubated for an additional hour followed by the addition of 0.2 to 200 μM final concentration of inhibitors. An equal volume of the vehicle (DMSO) was added as a control. Plates were the incubated for an additional 24 h at 37° C. in 5% CO₂, and cell viability was assessed by means of an MTT assay (Vybrant MTT Cell Proliferation Assay Kit) by measuring the absorbance of each well at 570 nm. Data are presented in the figure for cells treated with 200 μM of compound.

FIG. 20. Analysis of inhibitor binding kinetics for the paLpxC inhibitors. (a) Correlation between t_{R} and Kᵢ for the paLpxC inhibitors. (b) A 2-D kinetic map in which k_{off} and kₒₙ are plotted on the axes, while the diagonal lines show Kᵢ. In general, compounds containing a triazole have longer residence times (smaller k_{off} values) than compounds that lack a triazole.

FIG. 21. Correlation between Kᵢ and MIC. MIC values were determined using P. aeruginosa strain PAO1 by the microbroth dilution method. MIC Experiments were performed in triplicate, and the reported values are the average of the three independent experiments. Kᵢ values were determined by fitting the fluorescence competition data using Kintek. All kinetic assays were performed at 37° C. in triplicate.

FIG. 22. Correlation of calculated PAE determined at 4×MIC with measured residence time. Both PAE and residence time were measured at 37° C. Compounds a-f (blue circles) are from this work: a) PT901, b) PT920, c) PT805, d) PT908, e) PT909 and f) PT913. Compounds g-k (red circles) are from Walkup et al.: g) 1, h) 3, i) 6, j) 2, and k) 5. Compound i (6) is PF5081090 (1) in the present work. The line is a linear regression to all data points with a slope of 0.023 and R₂ of 0.776.

FIG. 23. Conformations of LpxC Insert II. A) The x-ray structures of paLpxC bound to (a) PT901 (salmon, PDB 3UHM), (b) 1 (cyan, PDB 5UPG), and (c) LPC-058 (slate, PDB 5DRR). B) The X-ray structures of ecLpxC bound to (a) UDP-(3-O—(R-3-hydroxymyristoyl))-glucosamine (green, PDB 4MDT)⁴⁵ and (b) BB-78485 (yellow, PDB 4ISA).

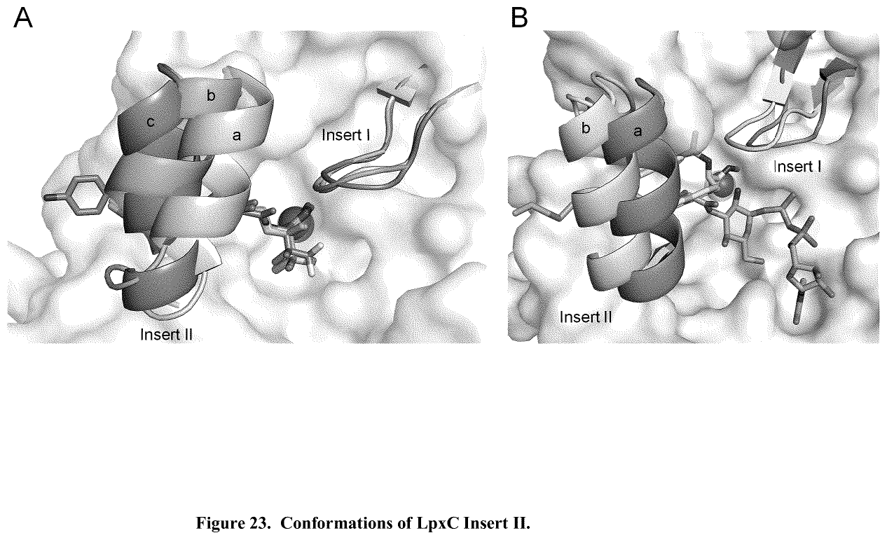

### DETAILED DESCRIPTION OF THE INVENTION

The following detailed description of embodiments of the invention will be made in reference to the accompanying drawings. In describing the invention, explanation about related functions or constructions known in the art are omitted for the sake of clearness in understanding the concept of the invention to avoid obscuring the invention with unnecessary detail.

In some embodiments, the present invention provides a compound having the structure:

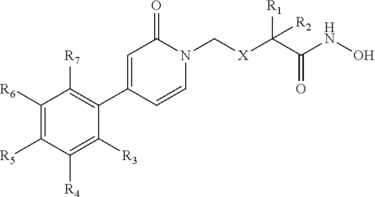

wherein R₁ and R₂ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, -alkyl-OR₁₄, C₁₋₁₀ alkyl, C₂₋₁₀ alkenyl, or C₂₋₁₀ alkynyl; or wherein R₁ and R₂ together formed into a cycloalkyl or cycloheteroalkyl;
wherein R₃ and R₇ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl;
wherein R₄ and R₆ are each independently H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl;
wherein R₅ is —NO₂, —NHR₁₄, —NR₁₄R₁₅, —SR₁₄, —SO₂R₁₄, —(C═O)—NH—R₁₄, —CO₂R₁₄, -alkyl-NR₁₄R₁₅, -alkyl-OR₁₄, C₂₋₁₀ alkenyl, C₂₋₁₀ alkynyl, —(C₀₋₁₀ alkyl)-heterocyclyl,

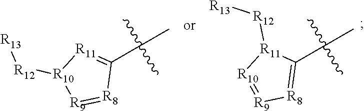

wherein R₈, R₉, R₁₀, and R₁₁ are each independently N or CH;
wherein R₁₂ is C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₃ is halogen, —NO₂, —CN, —NHR₁₄, —NR₁₄R₁₅, —SR₁₄, —SO₂R₁₄, —OR₁₄, —CO₂R₁₄, —CF₃, —NR₁₄R₁₅, —OR₁₄, C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₄ is H, C₂₋₁₀ alkenyl, C₂₋₁₀ alkynyl, —C═O-alkyl-aryl, or alkyl-aryl;
wherein R₁₅ is H, C₁₋₁₀ alkyl, C₂₋₁₀ alkenyl, C₂₋₁₀ alkynyl, —C═O-alkyl-aryl, or alkyl-aryl;
wherein X is CH₂ or NH;
wherein R₃ and R₅ are not both halogen, R₅ and R₇ are not both halogen;
wherein when R₃ or R₇ is halogen, R₄ and R₆ are not alkyl;
wherein when R₅ is —(C₀₋₁₀ alkyl)-heterocyclyl, the heterocyclyl is not tetrahydropyranyl;
wherein when R₃ or R₇ is halogen, R₄ and R₆ are not —OR₁₄;
wherein when R₃ is alkyl or H, R₆ is not —NHR₁₄ and —NR₁₄R₁₅; wherein when R₇ is alkyl or H, R₄ is not —NHR₁₄ and —NR₁₄R₁₅.

In some embodiments, R₁ and R₂ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, -alkyl-OR₁₄, C₁₋₁₀ alkyl, C₂₋₁₀ alkenyl, or C₂₋₁₀ alkynyl; or wherein R₁ and R₂ together formed into a cycloalkyl or cycloheteroalkyl.

In some embodiments, R₃ and R₇ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl.

In some embodiments, R₄ and R₆ are each independently H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl.

In some embodiments, R₅ is —NO₂, —NHR₁₄, —NR₁₄R₁₅, —SR₁₄, —SO₂R₁₄, —(C═O)—NH—R₁₄, —CO₂R₁₄, -alkyl-NR₁₄R₁₅, -alkyl-OR₁₄, C₂₋₁₀ alkenyl, C₂₋₁₀ alkynyl, —(C₀₋₁₀ alkyl)-heterocyclyl,

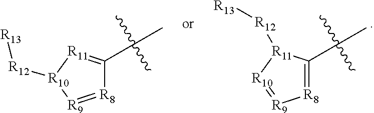

In some embodiments, R₈, R₉, R₁₀, and R₁₁ are each independently N or CH.

In some embodiments, R₁₂ is C₁₋₁₀ alkyl, C₂₋₁₀ alkenyl, C₂₋₁₀ alkynyl, aryl, heteroaryl, cycloalkyl, cycloheteroalky, or heterocyclyl.

In some embodiments, R₁₃ is halogen, —NO₂, —CN, —NHR₁₄, —NR₁₄R₁₅, —SR₁₄, —SO₂R₁₄, —OR₁₄, —CO₂R₁₄, —CF₃, —NR₁₄R₁₅, —OR₁₄, C₁₋₁₀ alkyl, C₂₋₁₀ alkenyl, C₂₋₁₀ alkynyl, aryl, heteroaryl, cycloalkyl, cycloheteroalky, or heterocyclyl.

In some embodiments, R₁₄ is H, C₂₋₁₀ alkenyl, C₂₋₁₀ alkynyl, —C═O-alkyl-aryl, or alkyl-aryl.

In some embodiments, R₁₅ is H, C₁₋₁₀ alkyl, C₂₋₁₀ alkenyl, C₂₋₁₀ alkynyl, —C═O-alkyl-aryl, or alkyl-aryl.

In some embodiments, R₁₅ is H, C₁₋₁₀ alkyl, C₂₋₁₀ alkenyl, C₂₋₁₀ alkynyl, —C═O-alkyl-aryl, or alkyl-aryl.

In some embodiments, X is CH₂ or NH.

In some embodiments, R₃ and R₅ are not both halogen, R₅ and R₇ are not both halogen.

In some embodiments, when R₃ or R₇ is halogen, R₄ and R₆ are not alkyl.

In some embodiments, when R₅ is —(C₀₋₁₀ alkyl)-heterocyclyl, the heterocyclyl is not tetrahydropyranyl,

In some embodiments, R₃ or R₇ is halogen, R₄ and R₆ are not —OR₁₄.

In some embodiments, R₃ is alkyl or H, R₆ is not —NHR₁₄ and —NR₁₄R₁₅; wherein when R₇ is alkyl or H, R₄ is not —NHR₁₄ and —NR₁₄R₁₅.

In some embodiments, R₃ and R₅ are not both halogen, R₅ and R₇ are not both halogen;

In some embodiments, when R₃ or R₇ is halogen, R₄ and R₆ are not alkyl;

In some embodiments, when R₅ is —(C₀₋₁₀ alkyl)-heterocyclyl, the heterocyclyl is not tetrahydropyranyl;

In some embodiments, when R₃ or R₇ is halogen, R₄ and R₆ are not —OR₁₄;

In some embodiments, when R₃ is alkyl or H, R₆ is not —NHR₁₄ and —NR₁₄R₁₅; wherein when R₇ is alkyl or H, R₄ is not —NHR₁₄ and —NR₁₄R₁₅.

In some embodiments, R₃ is H, R₄ and R₆ are each independently H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl.

In some embodiments, R₄ is H, R₃ and R₇ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl.

In some embodiments, R₆ is H, R₃ and R₇ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl.

In some embodiments, R₇ is H, R₄ and R₆ are each independently H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl.

In some embodiments, R₃ is halogen, R₄ and R₆ are each independently H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl.

In some embodiments, R₇ halogen, R₄ and R₆ are each independently H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl.

In some embodiments, R₃ and R₄ are H, R₆ is H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl, and R₇ is H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl.

In some embodiments, R₃ and R₆ are H, R₄ and R₇ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, -alkyl-OR₁₄, C₁₋₁₀ alkyl, C₂₋₁₀ alkenyl or C₂₋₁₀ alkynyl.

In some embodiments, R₃ and R₇ are H, R₄ and R₆ are each independently H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl.

In some embodiments, R₄ and R₆ are H, R₃ and R₇ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl.

In some embodiments, R₄ and R₇ are H, R₃ is H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl, and R₆ is H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl.

In some embodiments, R₆ and R₇ are H, R₃ is H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl, and R₄H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl.

In some embodiments, R₄ and R₆ are each independently H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl.

In some embodiments, R₃, R₄ and R₆ are H, R₇ is H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl.

In some embodiments, R₃, R₄ and R₇ are H, R₆ is H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl.

In some embodiments, R₃, R₆ and R₇ are H, R₄ is H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl.

In some embodiments, R₄, R₆ and R₇ are H, R₃ is H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl.

In some embodiments, halogen is F, Cl, Br and I.

In some embodiments, halogen is F, Cl and Br.

In some embodiments, halogen is F and Cl

In some embodiments, halogen is F.

In some embodiments, halogen is Cl.

In some embodiments, halogen is Br.

In some embodiments, halogen is I.

In some embodiments, R₃ is F, R₄, R₆ and R₇ are H.

In some embodiments, R₇ is F, R₄, R₆ and R₃ are H.

In some embodiments, R₃, R₄, R₆ and R₇ are H.

In some embodiments, R₁ and R₂ are each independently —SO₂R₁₄, —OR₁₄, —CO₂R₁₄, —CF₃, -alkyl-NR₁₄R₁₅, -alkyl-OR₁₄, C₁₋₁₀ alkyl, C₂₋₁₀ alkenyl, or C₂₋₁₀ alkynyl.

In some embodiments, R₁ and R₂ are each independently —SO₂R₁₄ or C₁₋₁₀ alkyl.

In some embodiments, R₁ and R₂ are each independently —SO₂CH₃ or CH₃.

In some embodiments, R₁ and R₂ are each independently H or -alkyl-OR₁₄.

In some embodiments, R₁ and R₂ are each independently H or

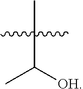 [SMILES: *C(C)O]

In some embodiments, R₁ and R₂ together formed into a cycloheteroalkyl.

In some embodiments, the present invention provides a compound having the structure:

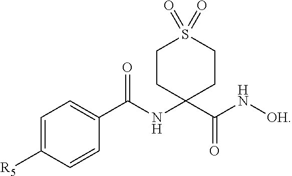

wherein R₅ is —NO₂, —NHR₁₄, —NR₁₄R₁₅, —SR₁₄, —SO₂R₁₄, —(C═O)—NH—R₁₄, —CO₂R₁₄, -alkyl-NR₁₄R₁₅, -alkyl-OR₁₄, C₂₋₁₀ alkenyl, C₂₋₁₀ alkynyl, —(C₀₋₁₀ alkyl)-heterocyclyl;

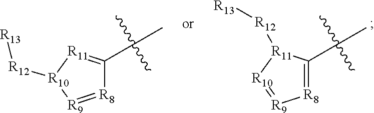

wherein R₁₂ is C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₃ is halogen, —NO₂, —CN, —NHR₁₄, —NR₁₄R₁₅, —SR₁₄, —SO₂R₁₄, —OR₁₄, —CO₂R₁₄, —CF₃, —NR₁₄R₁₅, —OR₁₄, C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₄ is H, C₂₋₁₀ alkenyl, C₂₋₁₀ alkynyl, —C═O-alkyl-aryl, or alkyl-aryl;
wherein R₁₅ is H, C₁₋₁₀ alkyl, C₂₋₁₀ alkenyl, C₂₋₁₀ alkynyl, —C═O-alkyl-aryl, or alkyl-aryl.

In some embodiments, cycloheteroalkyl is thiane-1,1-dioxide.

In some embodiments, R₅ is NO₂, —NHR₁₄, —NR₁₄R₁₅, —SR₁₄, —SO₂R₁₄, —CO₂R₁₄, -alkyl-NR₁₄R₁₅, -alkyl-OR₁₄, C₂₋₁₀ alkenyl, C₂₋₁₀ alkynyl, —(C₀₋₁₀ alkyl)-heterocyclyl,

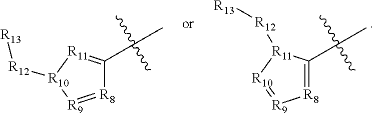

In some embodiments R₅ is —(C₀₋₁₀ alkyl)-heterocyclyl. —(C═O)—NH—R₁₄, —NHR₁₄,

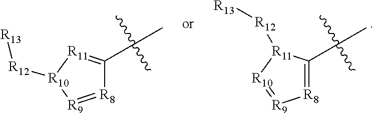

In some embodiments, R₅ is —(C═O)—NH—R₁₄, —NHR₁₄,

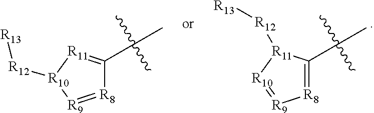

In some embodiments, R₅ is —NHR₁₄,

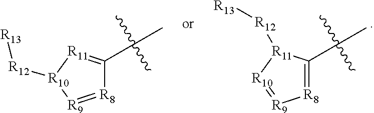

In some embodiments, R₅ is

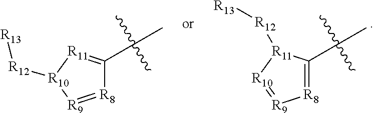

In some embodiments, R₅ is

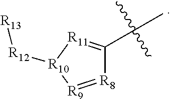

In some embodiments R₅ is

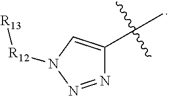

In some embodiments, R₅ is

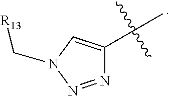

In some embodiments R₅ is

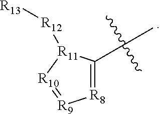

In some embodiments R₅ is

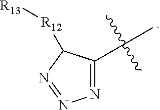

In some embodiments, R₅ is

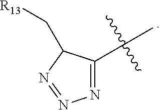

In some embodiments, R₅ is —NHR₁₄, wherein R₁₄ is —C═O-alkyl-aryl.

In some embodiments. R₁₄ is —C═O-methyl-phenyl.

In some embodiments, R₅ is —(C═O)—NH—R₁₄, wherein R₁₄ is alkyl-aryl.

In some embodiments, alkyl-aryl is methyl-phenyl.

In some embodiments, R₅ is —(C₁ alkyl)-heterocyclyl.

In some embodiments, R₅ is

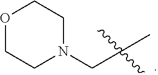 [SMILES: *CN1CCOCC1]

In some embodiments, R₈ is N and R₉, R₁₀, and R₁₁ are CH.

In some embodiments, R₉ is N and R₈, R₁₀, and R₁₁ are CH.

In some embodiments, R₁₀ is N and R₈, R₉, and R₁₁ are CH.

In some embodiments, R₁₁ is N and R₈, R₉, and R₁₀ are CH.

In some embodiments, R₈ and R₉ is N and R₁₀, and R₁₁ are CH.

In some embodiments, R₈ and R₁₀ is N and R₉, and R₁ are CH.

In some embodiments, R₈ and R₁ is N and R₉, and R₁₀ are CH.

In some embodiments, R₉ and R₁₀ is N and R₈, and R₁ are CH.

In some embodiments, R₉ and R₁₀ is N and R₈, and R₁₀ are C.

In some embodiments, R₁₀ and R₁₁ is N and R₈, and R₉ are CH.

In some embodiments, R₈, R₉, and R₁₀ is N and R₁₁ is CH.

In some embodiments, R₈, R₉, and R₁ is N and R₁₀ is CH.

In some embodiments, R₉, R₁₀, and R₁₁ is N and R₈ is CH.

In some embodiments, R₈ is CH and R₉, R₁₀, and R₁₁ are N.

In some embodiments, R₉ is CH and R₈, R₁₀, and R₁₁ are N.

In some embodiments, R₁₀ is CH and R₈, R₉, and R₁₁ are N.

In some embodiments, R₁₁ is CH and R₈, R₉, and R₁₀ are N.

In some embodiments, R₈ and R₉ is CH, and R₁₀, and R₁₁ are N.

In some embodiments, R₈ and R₁₀ is CH and R₉, and R₁₁ are N.

In some embodiments, R₈ and R₁₁ is CH and R₉, and R₁₀ are N.

In some embodiments, R₉ and R₁₀ is CH and R₈, and R₁₁ are N.

In some embodiments, R₉ and R₁₁ is CH and R₈, and R₁₀ are N.

In some embodiments, R₁₀ and R₁₁ is CH and R₈, and R₉ are N.

In some embodiments, R₈, R₉, and R₁₀ is CH and R₁₁ is N.

In some embodiments, R₈, R₉, and R₁ is CH and R₁₀ is N.

In some embodiments, R₉, R₁₀, and R₁₁ is CH and R₈ is N.

In some embodiments, R₈, R₉, and R₁₀ is N and R₁ is CH.

In some embodiments, R₉, R₁₀, and R₁₁ is N and R₈ is CH.

In some embodiments, R₁₂ is C₁₋₁₀ alkyl, C₂₋₁₀ alkenyl or C₂₋₁₀ alkynyl.

In some embodiments, R₁₂ is C₁₋₁₀ alkyl.

In some embodiments, R₁₂ is methyl.

In some embodiments, R₁₃ is halogen, —NO₂, —CN, —NHR₁₄, —NR₁₄R₁₅, —SR₁₄, —SO₂R₁₄, —OR₁₄, —CO₂R₁₄, —CF₃, —NR₁₄R₁₅, —OR₁₄, C₁₋₁₀ alkyl, C₂₋₁₀ alkenyl, C₂₋₁₀ alkynyl, aryl, heteroaryl, cycloalkyl, cycloheteroalky, or heterocyclyl.

In some embodiments, R₁₃ is —NHR₁₄, —NR₁₄R₁₅, —SR₁₄, —SO₂R₁₄, —OR₁₄, —CO₂R₁₄, —CF₃, —NR₁₄R₁₅, —OR₁₄, C₁₋₁₀ alkyl, C₂₋₁₀ alkenyl, C₂₋₁₀ alkynyl, aryl, heteroaryl, cycloalkyl, cycloheteroalky, or heterocyclyl.

In some embodiments, R₁₃ is —OR₁₄, —NHR₁₄, aryl, heteroaryl, cycloalkyl, cycloheteroalky, or heterocyclyl.

In some embodiments, the present invention provides a compound having the structure:

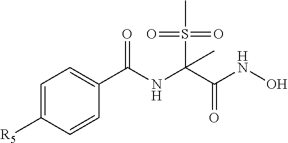

wherein R₅ is

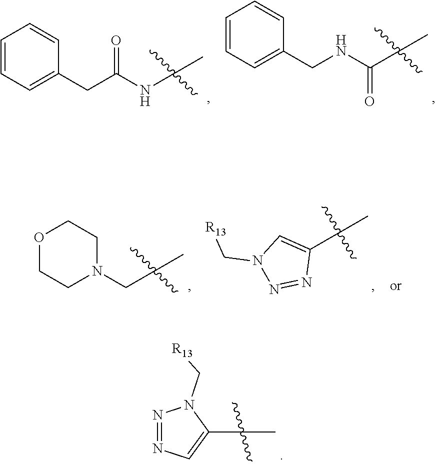

In some embodiments, R₁₃ is

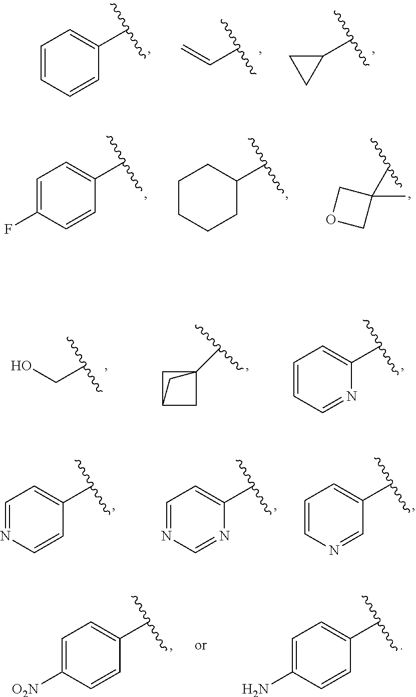

In some embodiments, the present invention provides a compound having the structure:

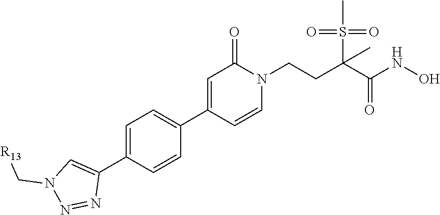

wherein R₁₃ is

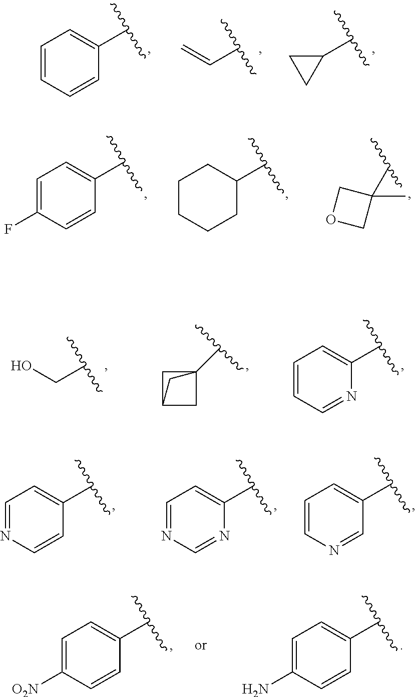

In some embodiments, R₁₃ is phenyl.

In some embodiments, the present invention provides a compound having the structure:

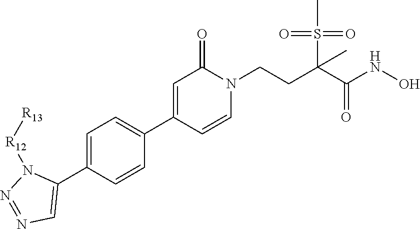

wherein R₁₂ is C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₃ is halogen, —NO₂, —CN, —NHR₁₄, —NR₁₄R₁₅, —SR₁₄, —SO₂R₁₄, —OR₁₄, —CO₂R₁₄, —CF₃, —NR₁₄R₁₅, —OR₁₄, C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₄ is H, C₂₋₁₀ alkenyl, C₂₋₁₀ alkynyl, —C═O-alkyl-aryl, or alkyl-aryl;
wherein R₁₅ is H, C₁₋₁₀ alkyl, C₂₋₁₀ alkenyl, C₂₋₁₀ alkynyl, —C═O-alkyl-aryl, or alkyl-aryl.

In some embodiments, wherein R₁₂ is C₁₋₁₀ alkyl.

In some embodiments, R₁₂ is methyl.

In some embodiments, R₁₃ is aryl.

In some embodiments, R₁₃ is phenyl.

In some embodiments, the present invention provides a compound having the structure:

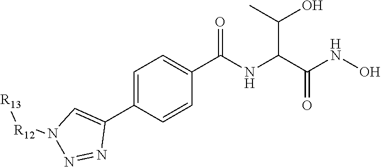

wherein R₁₂ is C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₃ is halogen, —NO₂, —CN, —NHR₁₄, —NR₁₄R₁₅, —SR₁₄, —SO₂R₁₄, —OR₁₄, —CO₂R₁₄, —CF₃, —NR₁₄R₁₅, —OR₁₄, C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₄ is H, C₂₋₁₀ alkenyl, C₂₋₁₀ alkynyl, —C═O-alkyl-aryl, or alkyl-aryl;
wherein R₁₅ is H, C₁₋₁₀ alkyl, C₂₋₁₀ alkenyl, C₂₋₁₀ alkynyl, —C═O-alkyl-aryl, or alkyl-aryl.

In some embodiments, R₁₂ is C₁₋₁₀ alkyl.

In some embodiments, R₁₂ is methyl.

In some embodiments, R₁₃ is aryl.

In some embodiments, R₁₃ is phenyl.

In some embodiments, the present invention provides a compound having the structure:

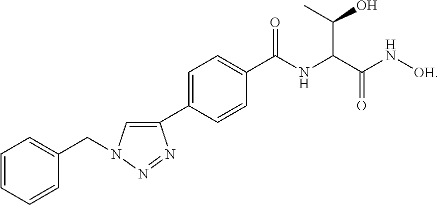 [SMILES: C[C@@H](O)C(NC(=O)c1ccc(-c2cn(Cc3ccccc3)nn2)cc1)C(=O)NO]

In some embodiments, the present invention provides a compound having the structure:

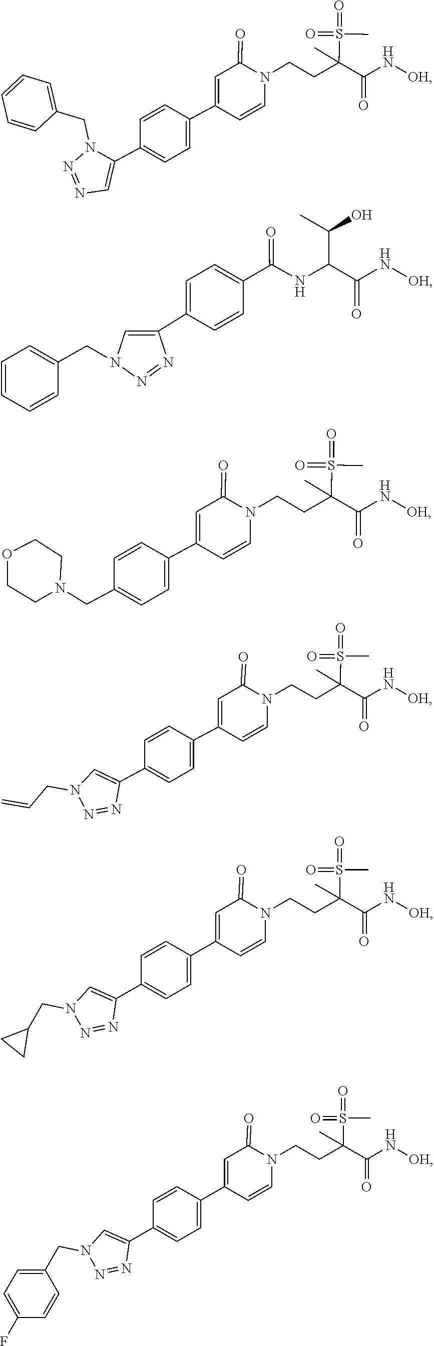

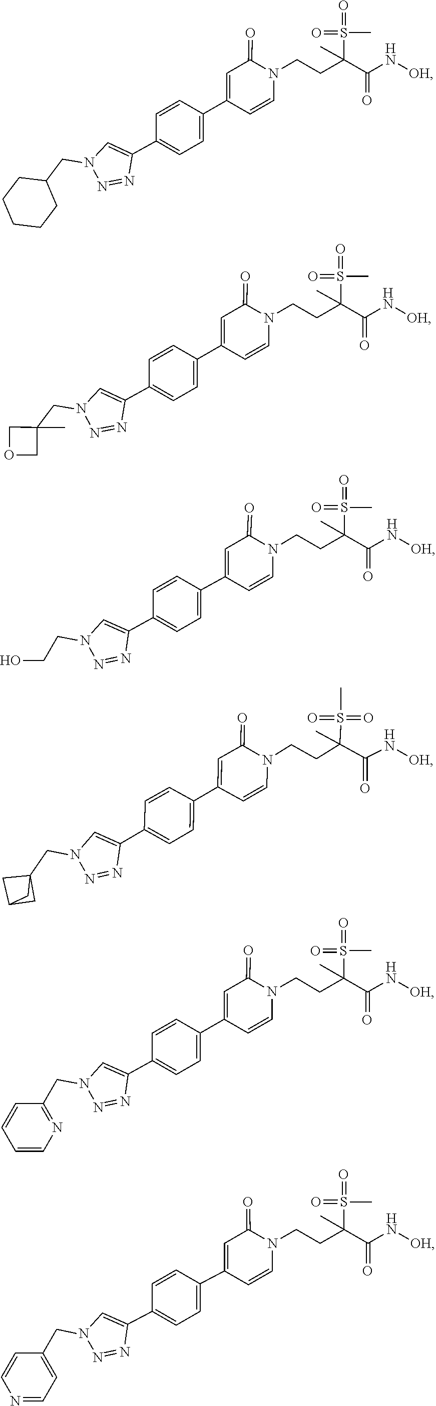 [SMILES: CC(CCn1ccc(-c2ccc(-c3cn(CC4CCCCC4)nn3)cc2)cc1=O)(C(=O)NO)S(C)(=O)=O ; CC1(Cn2cc(-c3ccc(-c4ccn(CCC(C)(C(=O)NO)S(C)(=O)=O)c(=O)c4)cc3)nn2)COC1 ; CC(CCn1ccc(-c2ccc(-c3cn(CCO)nn3)cc2)cc1=O)(C(=O)NO)S(C)(=O)=O ; CC(CCn1ccc(-c2ccc(-c3cn(CC45CC(C4)C5)nn3)cc2)cc1=O)(C(=O)NO)S(C)(=O)=O ; CC(CCn1ccc(-c2ccc(-c3cn(Cc4ccccn4)nn3)cc2)cc1=O)(C(=O)NO)S(C)(=O)=O ; CC(CCn1ccc(-c2ccc(-c3cn(Cc4ccncc4)nn3)cc2)cc1=O)(C(=O)NO)S(C)(=O)=O]

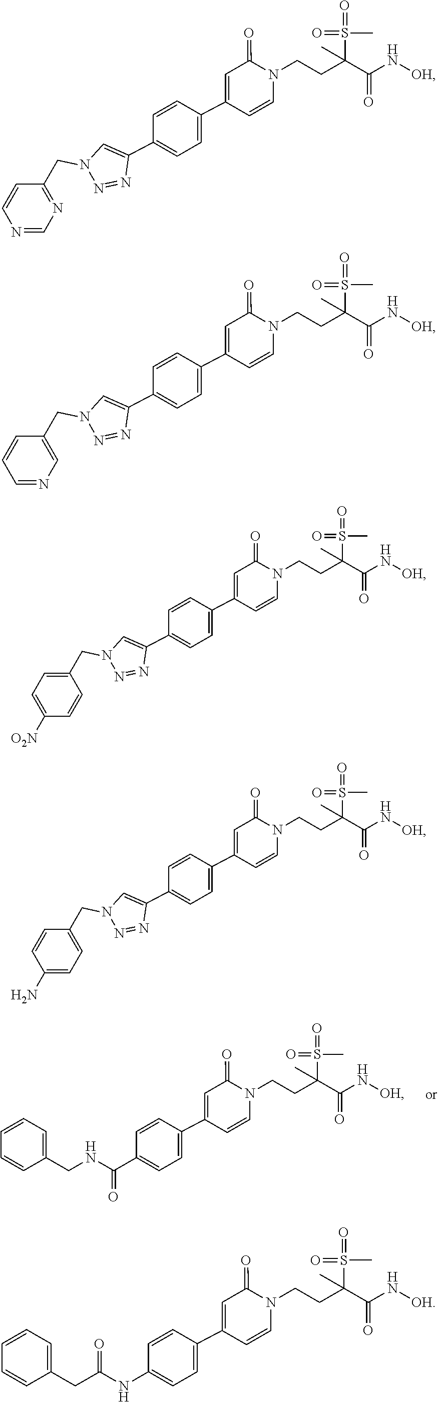

In some embodiments, the present invention provides a pharmaceutical composition comprising a compound having the structure:

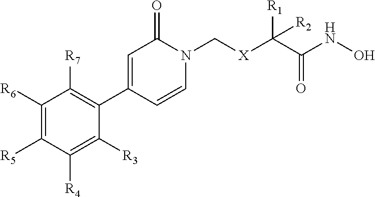

wherein R₁ and R₂ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, -alkyl-OR₁₄, C₁₋₁₀ alkyl, C₂₋₁₀ alkenyl, or C₂₋₁₀ alkynyl; or wherein R₁ and R₂ together formed into a cycloalkyl or cycloheteroalkyl;
wherein R₃ and R₇ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl;
wherein R₄ and R₆ are each independently H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl;
wherein R₅ is —NO₂, —NHR₁₄, —NR₁₄R₁₅, —SR₁₄, —SO₂R₁₄, —(C═O)—NH—R₁₄, —CO₂R₁₄, -alkyl-NR₁₄R₁₅, -alkyl-OR₁₄, C₂₋₁₀ alkenyl, C₂₋₁₀ alkynyl, —(C₀₋₁₀ alkyl)-heterocyclyl,

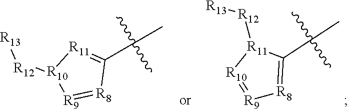

wherein R₈, R₉, R₁₀, and R₁₁ are each independently N or CH;
wherein R₁₂ is C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₃ is halogen, —NO₂, —CN, —NHR₁₄, —NR₁₄R₁₅, —SR₁₄, —SO₂R₁₄, —OR₁₄, —CO₂R₁₄, —CF₃, —NR₁₄R₁₅, —OR₁₄, C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₄ is H, C₂₋₁₀ alkenyl, C₂₋₁₀ alkynyl, —C═O-alkyl-aryl, or alkyl-aryl;
wherein R₁₅ is H, C₁₋₁₀ alkyl, C₂₋₁₀ alkenyl, C₂₋₁₀ alkynyl, —C═O-alkyl-aryl, or alkyl-aryl;
wherein X is CH₂ or NH;
wherein R₃ and R₅ are not both halogen, R₅ and R₇ are not both halogen;
wherein when R₃ or R₇ is halogen, R₄ and R₆ are not alkyl;
wherein when R₅ is —(C₀₋₁₀ alkyl)-heterocyclyl, the heterocyclyl is not tetrahydropyranyl;
wherein when R₃ or R₇ is halogen, R₄ and R₆ are not —OR₁₄;
wherein when R₃ is alkyl or H, R₆ is not —NHR₁₄ and —NR₁₄R₁₅; wherein when R₇ is alkyl or H, R₄ is not —NHR₁₄ and —NR₁₄R₁₅,
with at least one pharmaceutically acceptable excipient, diluent or carrier.

In some embodiments, the present invention provides a method of inhibiting bacteria growth, comprising contacting the bacteria with an amount a compound having the structure:

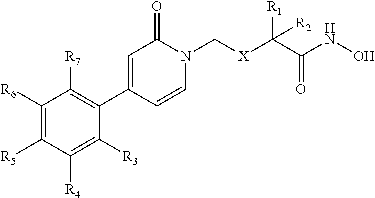

wherein R₁ and R₂ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, -alkyl-OR₁₄, C₁₋₁₀ alkyl, C₂₋₁₀ alkenyl, or C₂₋₁₀ alkynyl; or wherein R₁ and R₂ together formed into a cycloalkyl or cycloheteroalkyl;
wherein R₃ and R₇ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl;
wherein R₄ and R₆ are each independently H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl;
wherein R₅ is —NO₂, —NHR₁₄, —NR₁₄R₁₅, —SR₁₄, —SO₂R₁₄, —(C═O)—NH—R₁₄, —CO₂R₁₄, -alkyl-NR₁₄R₁₅, -alkyl-OR₁₄, C₂₋₁₀ alkenyl, C₂₋₁₀ alkynyl, —(C₀₋₁₀ alkyl)-heterocyclyl,

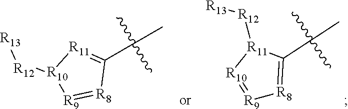

wherein R₈, R₉, R₁₀, and R₁₁ are each independently N or CH;
wherein R₁₂ is C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₃ is halogen, —NO₂, —CN, —NHR₁₄, —NR₁₄R₁₅, —SR₁₄, —SO₂R₁₄, —OR₁₄, —CO₂R₁₄, —CF₃, —NR₁₄R₁₅, —OR₁₄, C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₄ is H, C₂₋₁₀ alkenyl, C₂₋₁₀ alkynyl, —C═O-alkyl-aryl, or alkyl-aryl;
wherein R₁₅ is H, C₁₋₁₀ alkyl, C₂₋₁₀ alkenyl, C₂₋₁₀ alkynyl, —C═O-alkyl-aryl, or alkyl-aryl;
wherein X is CH₂ or NH;
wherein R₃ and R₅ are not both halogen, R₅ and R₇ are not both halogen;
wherein when R₃ or R₇ is halogen, R₄ and R₆ are not alkyl;
wherein when R₅ is —(C₀₋₁₀ alkyl)-heterocyclyl, the heterocyclyl is not tetrahydropyranyl;
wherein when R₃ or R₇ is halogen, R₄ and R₆ are not —OR₁₄;
wherein when R₃ is alkyl or H, R₆ is not —NHR₁₄ and —NR₁₄R₁₅; wherein when R₇ is alkyl or H, R₄ is not —NHR₁₄ and —NR₁₄R₁₅.

In some embodiments, the present invention provides a method of treating a bacterial infection in a patient, wherein the method comprising administering a therapeutically effective amount of a compound having the structure:

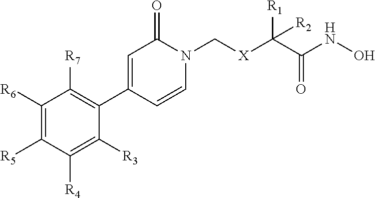

wherein R₁ and R₂ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, -alkyl-OR₁₄, C₁₋₁₀ alkyl, C₂₋₁₀ alkenyl, or C₂₋₁₀ alkynyl; or wherein R₁ and R₂ together formed into a cycloalkyl or cycloheteroalkyl;
wherein R₃ and R₇ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl;
wherein R₄ and R₆ are each independently H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl;
wherein R₅ is —NO₂, —NHR₁₄, —NR₁₄R₁₅, —SR₁₄, —SO₂R₁₄, —(C═O)—NH—R₁₄, —CO₂R₁₄, -alkyl-NR₁₄R₁₅, -alkyl-OR₁₄, C₂₋₁₀ alkenyl, C₂₋₁₀ alkynyl, —(C₀₋₁₀ alkyl)-heterocyclyl,

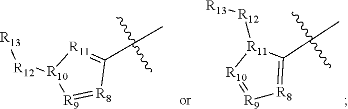

wherein R₈, R₉, R₁₀, and R₁₁ are each independently N or CH;
wherein R₁₂ is C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₃ is halogen, —NO₂, —CN, —NHR₁₄, —NR₁₄R₁₅, —SR₁₄, —SO₂R₁₄, —OR₁₄, —CO₂R₁₄, —CF₃, —NR₁₄R₁₅, —OR₁₄, C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₄ is H, C₂₋₁₀ alkenyl, C₂₋₁₀ alkynyl, —C═O-alkyl-aryl, or alkyl-aryl;
wherein R₁₅ is H, C₁₋₁₀ alkyl, C₂₋₁₀ alkenyl, C₂₋₁₀ alkynyl, —C═O-alkyl-aryl, or alkyl-aryl;
wherein X is CH₂ or NH;
wherein R₃ and R₅ are not both halogen, R₅ and R₇ are not both halogen;
wherein when R₃ or R₇ is halogen, R₄ and R₆ are not alkyl;
wherein when R₅ is —(C₀₋₁₀ alkyl)-heterocyclyl, the heterocyclyl is not tetrahydropyranyl;
wherein when R₃ or R₇ is halogen, R₄ and R₆ are not —OR₁₄;
wherein when R₃ is alkyl or H, R₆ is not —NHR₁₄ and —NR₁₄R₁₅; wherein when R₇ is alkyl or H, R₄ is not —NHR₁₄ and —NR₁₄R₁₅.

In some embodiments, R₃ is H, R₄ and R₆ are each independently H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl.

In some embodiments, R₄ is H, R₃ and R₇ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl.

In some embodiments, R₆ is H, R₃ and R₇ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl.

In some embodiments, R₇ is H, R₄ and R₆ are each independently H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl.

In some embodiments, R₃ is halogen, R₄ and R₆ are each independently H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl.

In some embodiments, R₇ halogen, R₄ and R₆ are each independently H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl.

In some embodiments, R₃ and R₄ are H, R₆ is H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl, and R₇ is H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl.

In some embodiments, R₃ and R₆ are H, R₄ and R₇ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, -alkyl-OR₁₄, C₁₋₁₀ alkyl, C₂₋₁₀ alkenyl or C₂₋₁₀ alkynyl.

In some embodiments, R₃ and R₇ are H, R₄ and R₆ are each independently H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl.

In some embodiments, R₄ and R₆ are H, R₃ and R₇ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl.

In some embodiments, R₄ and R₇ are H, R₃ is H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl, and R₆ is H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl.

In some embodiments, R₆ and R₇ are H, R₃ is H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl, and R₄H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl.

In some embodiments, R₄ and R₆ are each independently H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl.

In some embodiments, R₃, R₄ and R₆ are H, R₇ is H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl.

In some embodiments, R₃, R₄ and R₇ are H, R₆ is H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl.

In some embodiments, R₃, R₆ and R₇ are H, R₄ is H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl.

In some embodiments, R₄, R₆ and R₇ are H, R₃ is H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl.

In some embodiments, halogen is F, Cl, Br and I.

In some embodiments, halogen is F, Cl and Br.

In some embodiments, halogen is F and Cl

In some embodiments, halogen is F.

In some embodiments, halogen is Cl.

In some embodiments, halogen is Br.

In some embodiments, halogen is I.

In some embodiments, R₃ is F, R₄, R₆ and R₇ are H.

In some embodiments, R₇ is F, R₄, R₆ and R₃ are H.

In some embodiments, R₃, R₄, R₆ and R₇ are H.

In some embodiments, R₁ and R₂ are each independently —SO₂R₁₄, —OR₁₄, —CO₂R₁₄, —CF₃, -alkyl-NR₁₄R₁₅, -alkyl-OR₁₄, C₁₋₁₀ alkyl, C₂₋₁₀ alkenyl, or C₂₋₁₀ alkynyl.

In some embodiments, R₁ and R₂ are each independently —SO₂R₁₄ or C₁₋₁₀ alkyl.

In some embodiments, R₁ and R₂ are each independently —SO₂CH₃ or CH₃.

In some embodiments, R₁ and R₂ are each independently H or -alkyl-OR₁₄.

In some embodiments, R₁ and R₂ are each independently H or

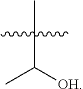 [SMILES: *C(C)O]

In some embodiments, R₁ and R₂ together formed into a cycloheteroalkyl.

In some embodiments, cycloheteroalkyl is thiane-1,1-dioxide.

In some embodiments, R₅ is NO₂, —NHR₁₄, —NR₁₄R₁₅, —SR₁₄, —SO₂R₁₄, —CO₂R₁₄, -alkyl-NR₁₄R₁₅, -alkyl-OR₁₄, C₂₋₁₀ alkenyl, C₂₋₁₀ alkynyl, —(C₀₋₁₀ alkyl)-heterocyclyl,

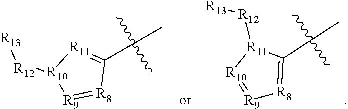

In some embodiments, R₅ is —(C₀₋₁₀ alkyl)-heterocyclyl. —(C═O)—NH—R₁₄, —NHR₁₄,

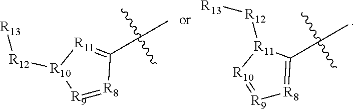

In some embodiments, R₅ is —(C═O)—NH—R₁₄, —NHR₁₄,

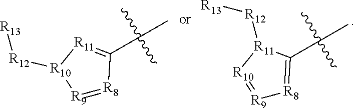

In some embodiments, R₅ is —NHR₁₄,

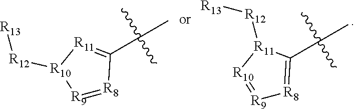

In some embodiments, R₅ is

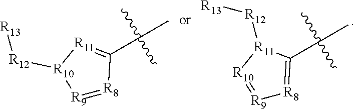

In some embodiments, R₅ is

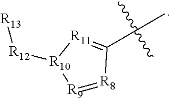

In some embodiments, R₅ is

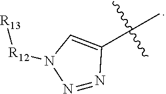

In some embodiments R₅ is

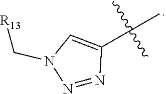

In some embodiments. R₅ is

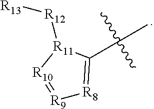

In some embodiments R₅ is

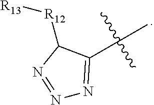

In some embodiments R₅ is

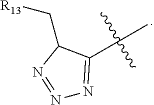

In some embodiments, R₅ is —NHR₁₄, wherein R₁₄ is —C═O-alkyl-aryl.

In some embodiments, R₁₄ is —C═O-methyl-phenyl.

In some embodiments, R₅ is —(C═O)—NH—R₁₄, wherein R₁₄ is alkyl-aryl.

In some embodiments, alkyl-aryl is methyl-phenyl.

In some embodiments, R₅ is —(C₁ alkyl)-heterocyclyl.

In some embodiments, R₅ is

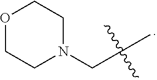 [SMILES: *CN1CCOCC1 ; "."]

In some embodiments, R₈ is N and R₉, R₁₀, and R₁₁ are CH.

In some embodiments, R₉ is N and R₈, R₁₀, and R₁₁ are CH.

In some embodiments, R₁₀ is N and R₈, R₉, and R₁₁ are CH.

In some embodiments, R₁₁ is N and R₈, R₉, and R₁₀ are CH.

In some embodiments, R₈ and R₉ is N and R₁₀, and R₁₁ are CH.

In some embodiments, R₈ and R₁₀ is N and R₉, and R₁ are CH.

In some embodiments, R₈ and R₁ is N and R₉, and R₁₀ are CH.

In some embodiments, R₉ and R₁₀ is N and R₈, and R₁₁ are CH.

In some embodiments, R₉ and R₁ is N and R₈, and R₁₀ are C.

In some embodiments, R₁₀ and R₁ is N and R₈, and R₉ are CH.

In some embodiments, R₈, R₉, and R₁₀ is N and R₁₁ is CH.

In some embodiments, R₈, R₉, and R₁ is N and R₁₀ is CH.

In some embodiments, R₉, R₁₀, and R₁₁ is N and R₈ is CH.

In some embodiments, R₈ is CH and R₉, R₁₀, and R₁₁ are N.

In some embodiments, R₉ is CH and R₈, R₁₀, and R₁₁ are N.

In some embodiments, R₁₀ is CH and R₈, R₉, and R₁₁ are N.

In some embodiments, R₁ is CH and R₈, R₉, and R₁₀ are N.

In some embodiments, R₈ and R₉ is CH, and R₁₀, and R₁₁ are N.

In some embodiments, R₈ and R₁₀ is CH and R₉, and R₁ are N.

In some embodiments, R₈ and R₁ is CH and R₉, and R₁₀ are N.

In some embodiments, R₉ and R₁₀ is CH and R₈, and R₁ are N.

In some embodiments, R₉ and R₁ is CH and R₈, and R₁₀ are N.

In some embodiments, R₁₀ and R₁ is CH and R₈, and R₉ are N.

In some embodiments, R₈, R₉, and R₁₀ is CH and R₁₁ is N.

In some embodiments, R₈, R₉, and R₁ is CH and R₁₀ is N.

In some embodiments, R₉, R₁₀, and R₁₁ is CH and R₈ is N.

In some embodiments, R₈, R₉, and R₁₀ is N and R₁₁ is CH.

In some embodiments, R₉, R₁₀, and R₁₁ is N and R₈ is CH.

In some embodiments, R₁₂ is C₁₋₁₀ alkyl, C₂₋₁₀ alkenyl or C₂₋₁₀ alkynyl.

In some embodiments, R₁₂ is C₁₋₁₀ alkyl.

In some embodiments, is methyl.

In some embodiments, R₁₃ is halogen, —NO₂, —CN, —NHR₁₄, —NR₁₄R₁₅, —SR₁₄, —SO₂R₁₄, —OR₁₄, —CO₂R₁₄, —CF₃, —NR₁₄R₁₅, —OR₁₄, C₁₋₁₀ alkyl, C₂₋₁₀ alkenyl, C₂₋₁₀ alkynyl, aryl, heteroaryl, cycloalkyl, cycloheteroalky, or heterocyclyl.

In some embodiments, R₁₃ is —NHR₁₄, —NR₁₄R₁₅, —SR₁₄, —SO₂R₁₄, —OR₁₄, —CO₂R₁₄, —CF₃, —NR₁₄R₁₅, —OR₁₄, C₁₋₁₀ alkyl, C₂₋₁₀ alkenyl, C₂₋₁₀ alkynyl, aryl, heteroaryl, cycloalkyl, cycloheteroalky, or heterocyclyl.

In some embodiments, R₁₃ is —OR₁₄, —NHR₁₄, aryl, heteroaryl, cycloalkyl, cycloheteroalky, or heterocyclyl.

In some embodiments, R₁₃ is

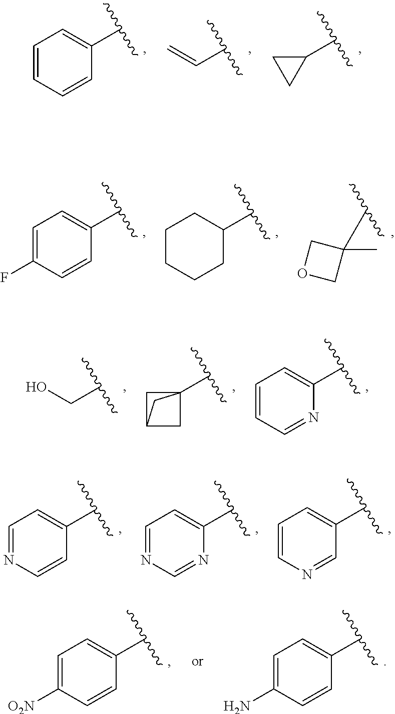

In some embodiments, the present invention provides a method of treating a bacterial infection in a patient, wherein the method comprising administering a therapeutically effective amount of a compound having the structure:

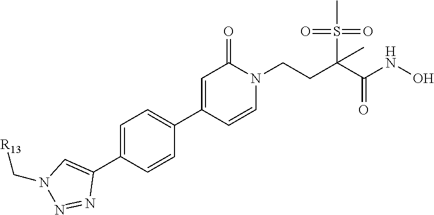

wherein R₁₃ is

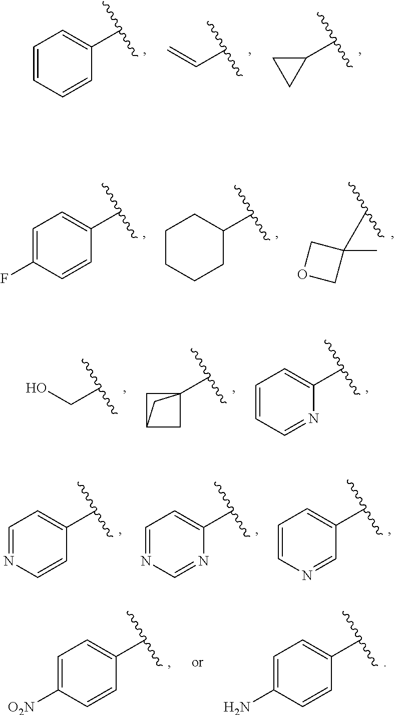

In some embodiments, RR₁₃ is phenyl.

In some embodiments, the present invention provides a method of treating a bacterial infection in a patient, wherein the method comprising administering a therapeutically effective amount of a compound having the structure:

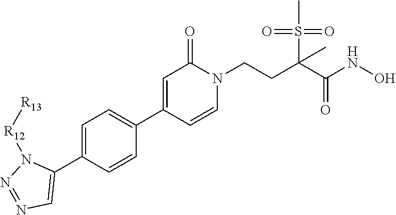

wherein R₁₂ is C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₃ is halogen, —NO₂, —CN, —NHR₁₄, —NR₁₄R₁₅, —SR₁₄, —SO₂R₁₄, —OR₁₄, —CO₂R₁₄, —CF₃, —NR₁₄R₁₅, —OR₁₄, C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₄ is H, C₂₋₁₀ alkenyl, C₂₋₁₀ alkynyl, —C═O-alkyl-aryl, or alkyl-aryl;
wherein R₁₅ is H, C₁₋₁₀ alkyl, C₂₋₁₀ alkenyl, C₂₋₁₀ alkynyl, —C═O-alkyl-aryl, or alkyl-aryl.

In some embodiments, wherein R₁₂ is C₁₋₁₀ alkyl.

In some embodiments, R₁₂ is methyl.

In some embodiments, R₁₃ is aryl.

In some embodiments, R₁₃ is phenyl.

In some embodiments, the present invention provides a method of treating a bacterial infection in a patient, wherein the method comprising administering a therapeutically effective amount of a compound having the structure:

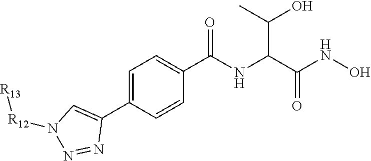

wherein R₁₂ is C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₃ is halogen, —NO₂, —CN, —NHR₁₄, —NR₁₄R₁₅, —SR₁₄, —SO₂R₁₄, —OR₁₄, —CO₂R₁₄, —CF₃, —NR₁₄R₁₅, —OR₁₄, C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₄ is H, C₂₋₁₀ alkenyl, C₂₋₁₀ alkynyl, —C═O-alkyl-aryl, or alkyl-aryl;
wherein R₁₅ is H, C₁₋₁₀ alkyl, C₂₋₁₀ alkenyl, C₂₋₁₀ alkynyl, —C═O-alkyl-aryl, or alkyl-aryl.

In some embodiments, R₁₂ is C₁₋₁₀ alkyl.

In some embodiments, R₁₂ is methyl.

In some embodiments, R₁₃ is aryl.

In some embodiments, R₁₃ is phenyl.

In some embodiments, the present invention provides a method of treating a bacterial infection in a patient, wherein the method comprising administering a therapeutically effective amount of a compound having the structure:

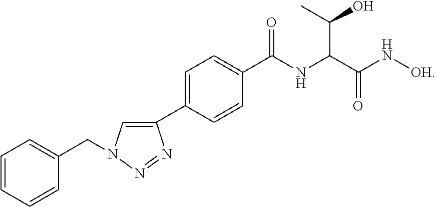 [SMILES: C[C@@H](O)C(NC(=O)c1ccc(-c2cn(Cc3ccccc3)nn2)cc1)C(=O)NO]

In some embodiments, the present invention provides a method of treating a bacterial infection in a patient, wherein the method comprising administering a therapeutically effective amount of a compound having the structure:

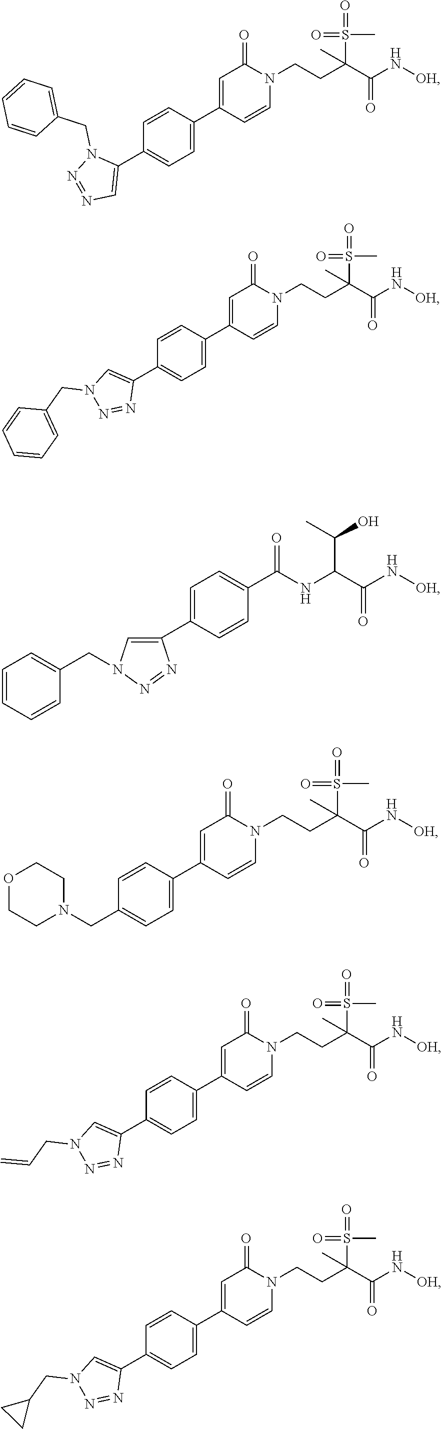 [SMILES: CC(CCn1ccc(-c2ccc(-c3cnnn3Cc3ccccc3)cc2)cc1=O)(C(=O)NO)S(C)(=O)=O ; CC(CCn1ccc(-c2ccc(-c3cn(Cc4ccccc4)nn3)cc2)cc1=O)(C(=O)NO)S(C)(=O)=O ; C[C@@H](O)C(NC(=O)c1ccc(-c2cn(Cc3ccccc3)nn2)cc1)C(=O)NO ; CC(CCn1ccc(-c2ccc(CN3CCOCC3)cc2)cc1=O)(C(=O)NO)S(C)(=O)=O ; C=CCn1cc(-c2ccc(-c3ccn(CCC(C)(C(=O)NO)S(C)(=O)=O)c(=O)c3)cc2)nn1 ; CC(CCn1ccc(-c2ccc(-c3cn(CC4CC4)nn3)cc2)cc1=O)(C(=O)NO)S(C)(=O)=O]

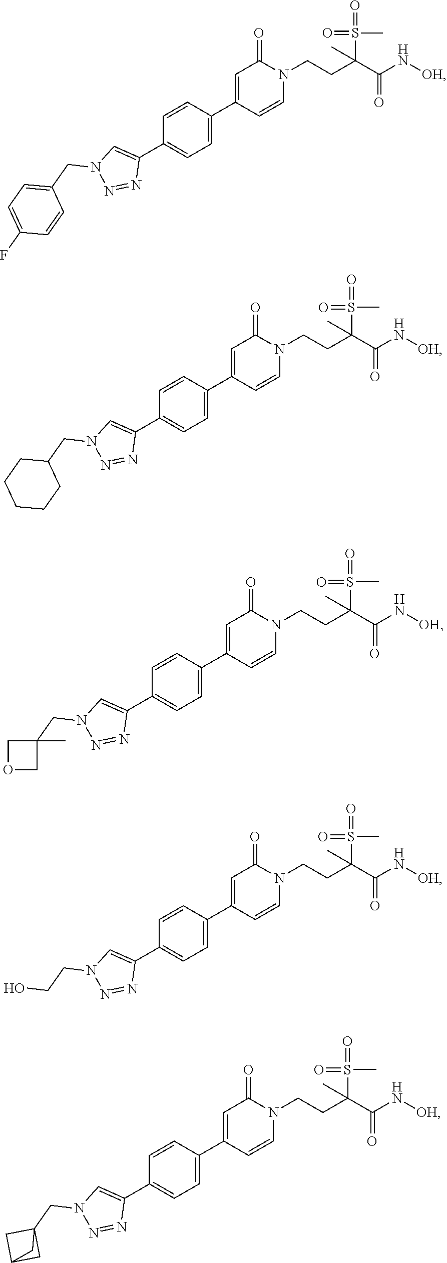 [SMILES: CC(CCn1ccc(-c2ccc(-c3cn(Cc4ccc(F)cc4)nn3)cc2)cc1=O)(C(=O)NO)S(C)(=O)=O ; CC(CCn1ccc(-c2ccc(-c3cn(CC4CCCCC4)nn3)cc2)cc1=O)(C(=O)NO)S(C)(=O)=O ; CC1(Cn2cc(-c3ccc(-c4ccn(CCC(C)(C(=O)NO)S(C)(=O)=O)c(=O)c4)cc3)nn2)COC1 ; CC(CCn1ccc(-c2ccc(-c3cn(CCO)nn3)cc2)cc1=O)(C(=O)NO)S(C)(=O)=O ; CC(CCn1ccc(-c2ccc(-c3cn(CC45CC(C4)C5)nn3)cc2)cc1=O)(C(=O)NO)S(C)(=O)=O]

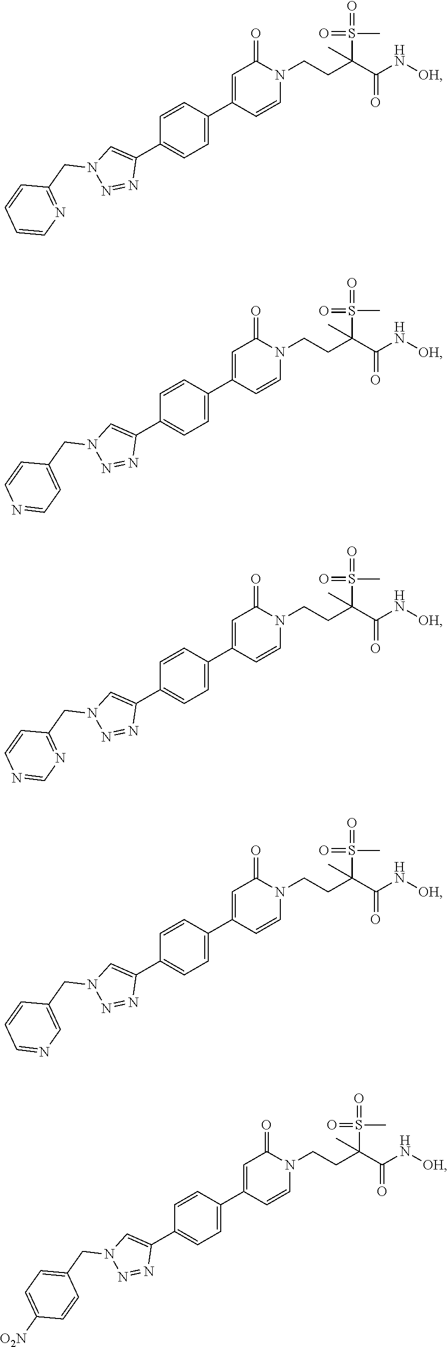 [SMILES: CC(CCn1ccc(-c2ccc(-c3cn(Cc4ccccn4)nn3)cc2)cc1=O)(C(=O)NO)S(C)(=O)=O ; CC(CCn1ccc(-c2ccc(-c3cn(Cc4ccncc4)nn3)cc2)cc1=O)(C(=O)NO)S(C)(=O)=O ; CC(CCn1ccc(-c2ccc(-c3cn(Cc4ccncn4)nn3)cc2)cc1=O)(C(=O)NO)S(C)(=O)=O ; CC(CCn1ccc(-c2ccc(-c3cn(Cc4cccnc4)nn3)cc2)cc1=O)(C(=O)NO)S(C)(=O)=O ; CC(CCn1ccc(-c2ccc(-c3cn(Cc4ccc([N+](=O)[O-])cc4)nn3)cc2)cc1=O)(C(=O)NO)S(C)(=O)=O]

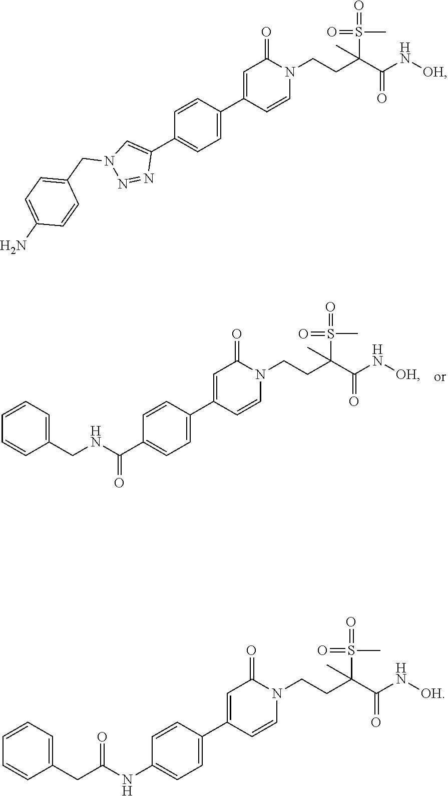 [SMILES: CC(CCn1ccc(-c2ccc(-c3cn(Cc4ccc(N)cc4)nn3)cc2)cc1=O)(C(=O)NO)S(C)(=O)=O ; CC(CCn1ccc(-c2ccc(C(=O)NCc3ccccc3)cc2)cc1=O)(C(=O)NO)S(C)(=O)=O ; CC(CCn1ccc(-c2ccc(NC(=O)Cc3ccccc3)cc2)cc1=O)(C(=O)NO)S(C)(=O)=O]

In some embodiments, the bacterial infection is caused by a Gram-negative bacteria.

In some embodiments, wherein the Gram-negative bacteria is Mannheimia haemotytica, Pasteurella multocida, Histophilus somni, Actinobacillus pleuropneumoniae, Salmonella enteritidis, Salmonella gallinahum, Lawsonia intracellularis, Brachyspira hyodysenteriae, Brachyspira pilosicoli, Acinetobacter baumannii, Acinetobacter spp., Citrobacter spp., Enterobacter aerogenes, Enterobacter cloacae, Escherichia coli, Klebsiella oxytoca, Klebsiella pneumoniae, Serratia marcescens, Stenotrophomonas maltophilia, or Pseudomonas aeruginosa.

In some embodiments, the Gram-negative bacteria is Pseudomonas aeruginosa.

In some embodiments, the Gram-negative bacterial infection is hospital-acquired pneumonia, urinary tract infections (UTI) intra-abdominal infections (IAI), catheter-associated bloodstream infections, respiratory infection, gastrointestinal infection, nosocomial pneumonia, urinary tract infection, bacteremia, sepsis, skin infection, soft-tissue infection, intraabdominal infection, lung infection, endocarditis, diabetic foot infection, osteomyelitis or central nervous system infection.

In some embodiments, the Gram-negative bacterial infection is hospital-acquired pneumonia, urinary tract infections (UTI) intra-abdominal infections (IAI) or catheter-associated bloodstream infections

In some embodiments, the therapeutically effective amount of the compound is administered orally, topically, or by injection.

In some embodiments, the present invention provides a use of a compound to treat bacterial infection caused by a Gram-negative bacteria.

In some embodiments, the Gram-negative bacteria is Mannheimia haemotytica, Pasteurella multocida, Histophilus somni, Actinobacilluspleuropneumoniae, Salmonella enteritidis, Salmonella gallinahum, Lawsonia intracellularis, Brachyspira hyodysenteriae, Brachyspira pilosicoli, Acinetobacter baumannii, Acinetobacter spp., Citrobacter spp., Enterobacter aerogenes, Enterobacter cloacae, Escherichia coli, Klebsiella oxytoca, Klebsiella pneumoniae, Serratia marcescens, Stenotrophomonas maltophilia, and Pseudomonas aeruginosa; wherein the Gram-negative bacterial infection is selected from the group consisting of respiratory infection, gastrointestinal infection, nosocomial pneumonia, urinary tract infection, bacteremia, sepsis, skin infection, soft-tissue infection, intraabdominal infection, lung infection, endocarditis, diabetic foot infection, osteomyelitis or central nervous system infection.

As used herein, “treating”, e.g. of an infection, encompasses inducing prevention, inhibition, regression, or stasis of the disease or a symptom or condition associated with the infection.

As used herein, “alkyl” includes both branched and straight-chain saturated aliphatic hydrocarbon groups having the specified number of carbon atoms and may be unsubstituted or substituted. Thus, C₁-Cₙ as in “C₁-Cₙ alkyl” is defined to include groups having 1, 2, . . . , n−1 or n carbons in a linear or branched arrangement. For example, C₁-C₆, as in “C₁-C₆ alkyl” is defined to include groups having 1, 2, 3, 4, 5, or 6 carbons in a linear or branched arrangement, and specifically includes methyl, ethyl, n-propyl, isopropyl, n-butyl, t-butyl, pentyl, hexyl, and octyl.

As used herein, “alkenyl” refers to a non-aromatic hydrocarbon radical, straight or branched, containing at least 1 carbon to carbon double bond, and up to the maximum possible number of non-aromatic carbon-carbon double bonds may be present, and may be unsubstituted or substituted. For example, “C₂-C₆ alkenyl” means an alkenyl radical having 2, 3, 4, 5, or 6 carbon atoms, and up to 1, 2, 3, 4, or 5 carbon-carbon double bonds respectively. Alkenyl groups include ethenyl, propenyl, butenyl and cyclohexenyl.

The term “alkynyl” refers to a hydrocarbon radical straight or branched, containing at least 1 carbon to carbon triple bond, and up to the maximum possible number of non-aromatic carbon-carbon triple bonds may be present, and may be unsubstituted or substituted. Thus, “C₂-C₆ alkynyl” means an alkynyl radical having 2 or 3 carbon atoms and 1 carbon-carbon triple bond, or having 4 or 5 carbon atoms and up to 2 carbon-carbon triple bonds, or having 6 carbon atoms and up to 3 carbon-carbon triple bonds. Alkynyl groups include ethynyl, propynyl and butynyl.

“Alkylene”, “alkenylene” and “alkynylene” shall mean, respectively, a divalent alkane, alkene and alkyne radical, respectively. It is understood that an alkylene, alkenylene, and alkynylene may be straight or branched. An alkylene, alkenylene, and alkynylene may be unsubstituted or substituted.

As used herein, “aryl” is intended to mean any stable monocyclic, bicyclic or polycyclic carbon ring of up to 10 atoms in each ring, wherein at least one ring is aromatic, and may be unsubstituted or substituted. Examples of such aryl elements include phenyl, p-toluenyl (4-methylphenyl), naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring.

As used herein, the term “polycyclic” refers to unsaturated or partially unsaturated multiple fused ring structures, which may be unsubstituted or substituted.

The term “alkylaryl” refers to alkyl groups as described above wherein one or more bonds to hydrogen contained therein are replaced by a bond to an aryl group as described above. It is understood that an “alkylaryl” group is connected to a core molecule through a bond from the alkyl group and that the aryl group acts as a substituent on the alkyl group. Examples of arylalkyl moieties include, but are not limited to, benzyl (phenylmethyl), p-trifluoromethylbenzyl (4-trifluoromethylphenylmethyl), 1-phenylethyl, 2-phenylethyl, 3-phenylpropyl, 2-phenylpropyl and the like.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include phenyl, pyridine, pyrimidine or pyridizine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “alkylheteroaryl” refers to alkyl groups as described above wherein one or more bonds to hydrogen contained therein are replaced by a bond to an heteroaryl group as described above. It is understood that an “alkylheteroaryl” group is connected to a core molecule through a bond from the alkyl group and that the heteroaryl group acts as a substituent on the alkyl group. Examples of alkylheteroaryl moieties include, but are not limited to, —CH₂—(C₅H₄N), —CH₂—CH₂—(C₅H₄N) and the like.

The term “heterocycle”, “heterocyclyl” or “heterocyclic” refers to a mono- or poly-cyclic ring system which can be saturated or contains one or more degrees of unsaturation and contains one or more heteroatoms. Preferred heteroatoms include N, O, and/or S, including N-oxides, sulfur oxides, and dioxides. Preferably the ring is three to ten-membered and is either saturated or has one or more degrees of unsaturation. The heterocycle may be unsubstituted or substituted, with multiple degrees of substitution being allowed. Such rings may be optionally fused to one or more of another “heterocyclic” ring(s), heteroaryl ring(s), aryl ring(s), or cycloalkyl ring(s). Examples of heterocycles include, but are not limited to, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

The alkyl, alkenyl, alkynyl, aryl, heteroaryl and heterocyclyl substituents may be substituted or unsubstituted, unless specifically defined otherwise.

In the compounds of the present invention, alkyl, alkenyl, alkynyl, aryl, heterocyclyl and heteroaryl groups can be further substituted by replacing one or more hydrogen atoms with alternative non-hydrogen groups. These include, but are not limited to, halo, hydroxy, mercapto, amino, carboxy, cyano and carbamoyl.

As used herein, the term “halogen” refers to F, Cl, Br, and I.

As used herein, “heteroalkyl” includes both branched and straight-chain saturated aliphatic hydrocarbon groups having the specified number of carbon atoms and at least 1 heteroatom within the chain or branch.

As used herein, “heterocycle” or “heterocyclyl” as used herein is intended to mean a 5- to 10-membered nonaromatic ring containing from 1 to 4 heteroatoms selected from the group consisting of O, N and S, and includes bicyclic groups. “Heterocyclyl” therefore includes, but is not limited to the following: imidazolyl, piperazinyl, piperidinyl, pyrrolidinyl, morpholinyl, thiomorpholinyl, tetrahydropyranyl, dihydropiperidinyl, tetrahydrothiophenyl and the like. If the heterocycle contains a nitrogen, it is understood that the corresponding N-oxides thereof are also encompassed by this definition.

As used herein, “cycloalkyl” shall mean cyclic rings of alkanes of three to eight total carbon atoms, or any number within this range (i.e., cyclopropyl, cyclobutyl, cyclopentyl, cyclohexyl, cycloheptyl or cyclooctyl).

As used herein, “monocycle” includes any stable polyatomic carbon ring of up to 10 atoms and may be unsubstituted or substituted. Examples of such non-aromatic monocycle elements include but are not limited to: cyclobutyl, cyclopentyl, cyclohexyl, and cycloheptyl. Examples of such aromatic monocycle elements include but are not limited to: phenyl.

As used herein, “bicycle” includes any stable polyatomic carbon ring of up to 10 atoms that is fused to a polyatomic carbon ring of up to 10 atoms with each ring being independently unsubstituted or substituted. Examples of such non-aromatic bicycle elements include but are not limited to: decahydronaphthalene. Examples of such aromatic bicycle elements include but are not limited to: naphthalene.

The term “ester” is intended to a mean an organic compound containing the R—O—CO—R′ group.

The term “amide” is intended to a mean an organic compound containing the R—CO—NH—R′ or R—CO—N—R′R″ group.

The term “phenyl” is intended to mean an aromatic six membered ring containing six carbons and five hydrogens.

The term “benzyl” is intended to mean a —CH₂R₁ group wherein the R₁ is a phenyl group.

The term “substitution”, “substituted” and “substituent” refers to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and halogens (i.e., F, Cl, Br, and I); alkyl groups, such as methyl, ethyl, n-propyl, isopropryl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

As used herein, the term “excipient” refers to substances that are included in a pharmaceutical dosage form not for their direct therapeutic action, but to aid the manufacturing process, to protect, support or enhance stability, or for bioavailability or patient acceptability.

As used herein, the term “diluent” refers to fillers in pharmaceutical tablets to increase weight and improve content uniformity. Natural diluents include starches, hydrolyzed starches, and partially pregelatinized starches. Diluents provide better tablet properties such as improved cohesion or to promote flow.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure result.

In choosing the compounds of the present invention, one of ordinary skill in the art will recognize that the various substituents, i.e. R¹, R², etc. are to be chosen in conformity with well-known principles of chemical structure connectivity.

The compounds of the present invention include all hydrates, solvates, and complexes of the compounds used by this invention. If a chiral center or another form of an isomeric center is present in a compound of the present invention, all forms of such isomer or isomers, including enantiomers and diastereomers, are intended to be covered herein. Compounds containing a chiral center may be used as a racemic mixture, an enantiomerically enriched mixture, or the racemic mixture may be separated using well-known techniques and an individual enantiomer may be used alone. The compounds described in the present invention are in racemic form or as individual enantiomers. The enantiomers can be separated using known techniques, such as those described in Pure and Applied Chemistry 69, 1469-1474, (1997) IUPAC. In cases in which compounds have unsaturated carbon-carbon double bonds, both the cis (Z) and trans (E) isomers are within the scope of this invention.

The compounds of the subject invention may have spontaneous tautomeric forms. In cases wherein compounds may exist in tautomeric forms, such as keto-enol tautomers, each tautomeric form is contemplated as being included within this invention whether existing in equilibrium or predominantly in one form.

In the compound structures depicted herein, hydrogen atoms are not shown for carbon atoms having less than four bonds to non-hydrogen atoms. However, it is understood that enough hydrogen atoms exist on said carbon atoms to satisfy the octet rule.

This invention also provides isotopic variants of the compounds disclosed herein, including wherein the isotopic atom is ²H and/or wherein the isotopic atom ¹³C. Accordingly, in the compounds provided herein hydrogen can be enriched in the deuterium isotope. It is to be understood that the invention encompasses all such isotopic forms.

It is understood that the structures described in the embodiments of the methods hereinabove can be the same as the structures of the compounds described hereinabove.

It is understood that where a numerical range is recited herein, the present invention contemplates each integer between, and including, the upper and lower limits, unless otherwise stated.

Except where otherwise specified, if the structure of a compound of this invention includes an asymmetric carbon atom, it is understood that the compound occurs as a racemate, racemic mixture, and isolated single enantiomer. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, NY, 1981. For example, the resolution may be carried out by preparative chromatography on a chiral column.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

In the compounds used in the method of the present invention, the substituents may be substituted or unsubstituted, unless specifically defined otherwise.

It is understood that substituents and substitution patterns on the compounds used in the method of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure result.

In choosing the compounds used in the method of the present invention, one of ordinary skill in the art will recognize that the various substituents, i.e. R¹, R², etc. are to be chosen in conformity with well-known principles of chemical structure connectivity.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure result.

In choosing the compounds of the present invention, one of ordinary skill in the art will recognize that the various substituents, i.e. R¹, R², etc. are to be chosen in conformity with well-known principles of chemical structure connectivity.

The various R groups attached to the aromatic rings of the compounds disclosed herein may be added to the rings by standard procedures, for example those set forth in Advanced Organic Chemistry: Part B: Reaction and Synthesis, Francis Carey and Richard Sundberg, (Springer) 5th ed. Edition. (2007), the content of which is hereby incorporated by reference.

The compounds used in the method of the present invention may be prepared by techniques well known in organic synthesis and familiar to a practitioner ordinarily skilled in the art. However, these may not be the only means by which to synthesize or obtain the desired compounds.

The compounds used in the method of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

The compounds used in the method of the present invention may be in a salt form. As used herein, a “salt” is a salt of the instant compounds which has been modified by making acid or base salts of the compounds. In the case of compounds used to treat an infection or disease caused by a pathogen, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkali earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The compounds of the present invention may also form salts with basic amino acids such a lysine, arginine, etc. and with basic sugars such as N-methylglucamine, 2-amino-2-deoxyglucose, etc. and any other physiologically non-toxic basic substance.

As used herein, “administering” an agent may be performed using any of the various methods or delivery systems well known to those skilled in the art. The administering can be performed, for example, orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intrathecally, into a cerebral ventricle, intraventicularly, intratumorally, into cerebral parenchyma or intraparenchchymally.

The compounds used in the method of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds used in the method of the present invention may comprise a single compound or mixtures thereof with additional antitumor agents. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or topically onto a site of disease or lesion, all using dosage forms well known to those of ordinary skill in the pharmaceutical arts.

The compounds used in the method of the present invention can be administered in admixture with suitable pharmaceutical diluents, extenders, excipients, or in carriers such as the novel programmable sustained-release multi-compartmental nanospheres (collectively referred to herein as a pharmaceutically acceptable carrier) suitably selected with respect to the intended form of administration and as consistent with conventional pharmaceutical practices. The unit will be in a form suitable for oral, nasal, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present invention may also be administered in the form of liposome delivery systems, such as small unilamellar vesicles, large unilamellar vesicles, and multilamellar vesicles. Liposomes can be formed from a variety of phospholipids such as lecithin, sphingomyelin, proteolipids, protein-encapsulated vesicles or from cholesterol, stearylamine, or phosphatidylcholines. The compounds may be administered as components of tissue-targeted emulsions.

The compounds used in the method of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar-coated or film-coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, asuitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The compounds used in the method of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials such as solutol and/or ethanol to make them compatible with the type of injection or delivery system chosen.

The compounds and compositions of the present invention can be administered in oral dosage forms as tablets, capsules, pills, powders, granules, elixirs, tinctures, suspensions, syrups, and emulsions.

The compounds may also be administered in intravenous (bolus or infusion), intraperitoneal, subcutaneous, or intramuscular form, or introduced directly, e.g. by topical administration, injection or other methods, to the afflicted area, such as a wound, including ulcers of the skin, all using dosage forms well known to those of ordinary skill in the pharmaceutical arts.

Specific examples of pharmaceutically acceptable carriers and excipients that may be used to formulate oral dosage forms of the present invention are described in U.S. Pat. No. 3,903,297 to Robert, issued Sep. 2, 1975. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

The active ingredient can be administered orally in solid dosage forms, such as capsules, tablets, powders, and chewing gum; or in liquid dosage forms, such as elixirs, syrups, and suspensions, including, but not limited to, mouthwash and toothpaste. It can also be administered parentally, in sterile liquid dosage forms.

Solid dosage forms, such as capsules and tablets, may be enteric-coated to prevent release of the active ingredient compounds before they reach the small intestine. Materials that may be used as enteric coatings include, but are not limited to, sugars, fatty acids, proteinaceous substances such as gelatin, waxes, shellac, cellulose acetate phthalate (CAP), methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), and methyl methacrylate-methacrylic acid copolymers.

The compounds and compositions of the invention can be coated onto stents for temporary or permanent implantation into the cardiovascular system of a subject.

Variations on those general synthetic methods will be readily apparent to those of ordinary skill in the art and are deemed to be within the scope of the present invention.

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

While the invention has been shown and described with reference to certain embodiments of the present invention thereof, it will be understood by those skilled in the art that various changes in from and details may be made therein without departing from the spirit and scope of the present invention and equivalents thereof.

### EXPERIMENTAL DETAILS

### I. The Impact of Target Turn-Over on the Translation of Drug-Target Residence Time to Time-Dependent Antibacterial Activity

Antibacterial drug discovery primarily involves the development of compounds that inhibit targets that are essential for bacteria survival. The identification of these targets can stem from deconvoluting the mode of action of natural product antibacterial agents or of compounds discovered in phenotypic assays, or through ever more sophisticated genetic approaches that alter the amount of target either through knockout or inducible knockdown. However, the difficulty in exploiting essential targets for the development of new antibiotics indicates that essentiality is not sufficient (Payne 2007). In addition to traditional challenges faced by antibacterial drug discovery, such as drug penetration/efflux, the rapid emergence of resistance, and the lack of target conservation across pathogens, the growth conditions used for establishing the importance of a target for bacterial growth may differ from the environment experienced by the bacteria during human infection (Sliver 2011 and Brown 2015). In addition, target vulnerability plays a major role in the context of target druggability and may have a direct impact on the drug levels required for in vivo antibacterial activity (Kaur 2009, Singh 2013 and Krishnamoorthy, 2011).

Target vulnerability quantifies the level of target occupancy required to induce the desired physiological response, such as bacterial cell death (Daryaee, 2020, Basak 2020 and Tonge, 2018). High vulnerability targets are those that require low levels of occupancy to trigger the desired outcome while low vulnerability targets require high levels of occupancy. Target vulnerability is thus an important factor in the determining the drug exposure required for the pharmacodynamic response, and the lower drug levels required for a high vulnerability target is expected to translate into lower, less frequent drug doses and a widening of the therapeutic window. Since antibiotics are often given at high levels for sustained periods of time, approaches that reduce the required drug exposure are likely to improve the success rate of new drug approvals (Walkup, 2015). Most approaches to assessing target vulnerability have focused on examining the phenotypic response to reducing target levels, either by genetic knockdown or selected proteolysis of the target (Ramachandran, 2013 and Wei, 2011). However, reducing enzyme activity by removing the protein from the cell is fundamentally different to inhibiting the enzyme using a small molecule, a difference recognized in the field of targeted protein degradation where the former leads to the event-driven pharmacology and the latter to occupancy-driven pharmacology (Calabrese, 2019).

Post-antibiotic effect (PAE), which is the delay in bacterial growth following compound washout, provides insight into target vulnerability and specifically whether an increase in the residence time of the compound on the target translates to an increase in PAE (Daryaee, 2020 and Basak, 2020). Using this approach, it was demonstrated that the bacterial ribosome and the Pseudomonas aeruginosa LpxC (paLpxC) both exhibit a strong correlation between residence time and PAE, indicating that both are highly vulnerable (Daryaee, 2020 and Wakup, 2015). This application extended the analysis to the LpxC enzyme from Escherichia coli (ecLpxC). LpxC (UDP-3-O-acyl-N-acetylglucosamine deacetylase) catalyzes the first committed step in lipopolysaccharide biosynthesis and is a validated antibacterial target in Gram-negative pathogens (Whittington, 2003 and Barb, 2007). In contrast to paLpxC, inhibitors of ecLpxC show no PAE in Escherichia coli. The half-life of LpxC in P. aeruginosa is 90 min, compared to 9-10 min in E. coli. Genetic tools and mass spectrometry were used to analyze the role of target turnover in the translation of target occupancy to the delay in bacterial regrowth following compound washout (Langklotz, 2011, Ogura, 1999 and Schakermann, 2013). ecLpxC is resynthesized rapidly in wild-type E. coli but that a PAE can be induced if the rate of ecLpxC resynthesis is reduced either by a hyperactive mutant of the fatty acid biosynthesis 3-hydroxyacyl-ACP dehydratase FabZ (FabZ*), or by the addition of sub-MIC levels of a macrolide antibiotic (Ogura, 1999). Therefore, the current application focused on modulation of target vulnerability and the potential role of protein synthesis inhibitors in combination therapy.

### Experiment 1. Post-Antibiotic Effect of LpxC Inhibitors in Wild Type E. coli

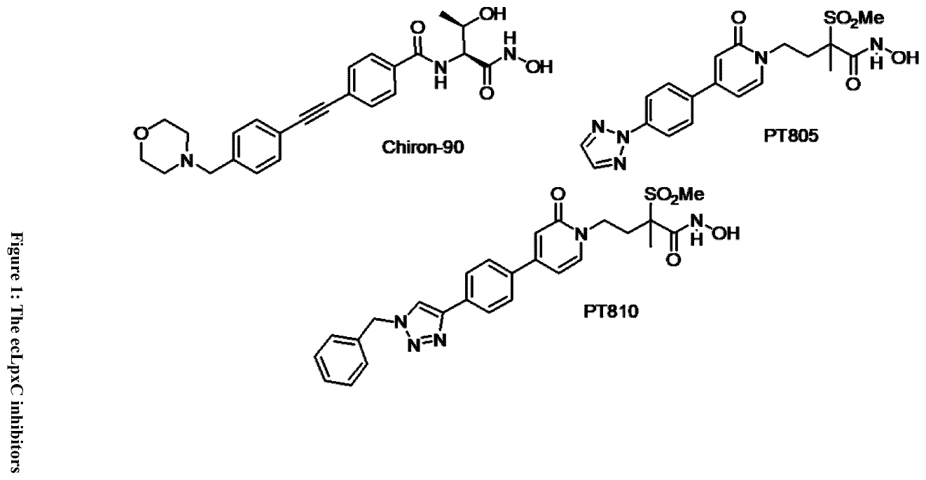

There is a correlation between the residence time of inhibitors on P. aeruginosa LpxC (paLpxC) and the post-antibiotic effect resulting from treatment of P. aeruginosa PA01 (Walkup, 2015). Three LpxC inhibitors were selected for analysis with ecLpxC that included PT805, PT810, and CHIR-090, that binds to ecLpxC through a two-step induced-fit mechanism (FIG. 1). (McClerren, 2012)

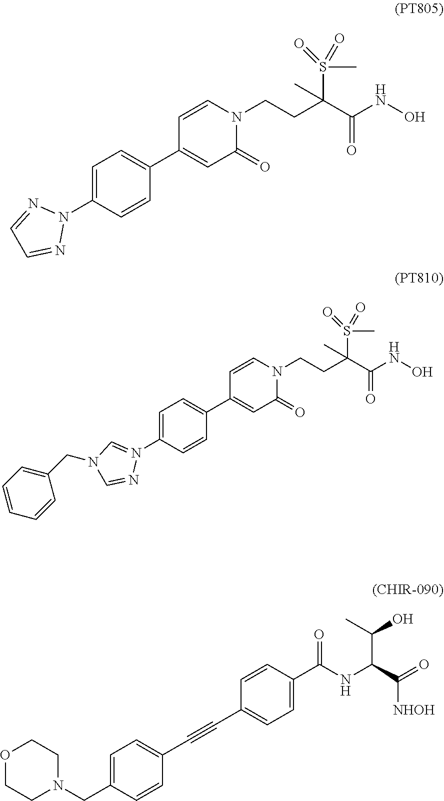

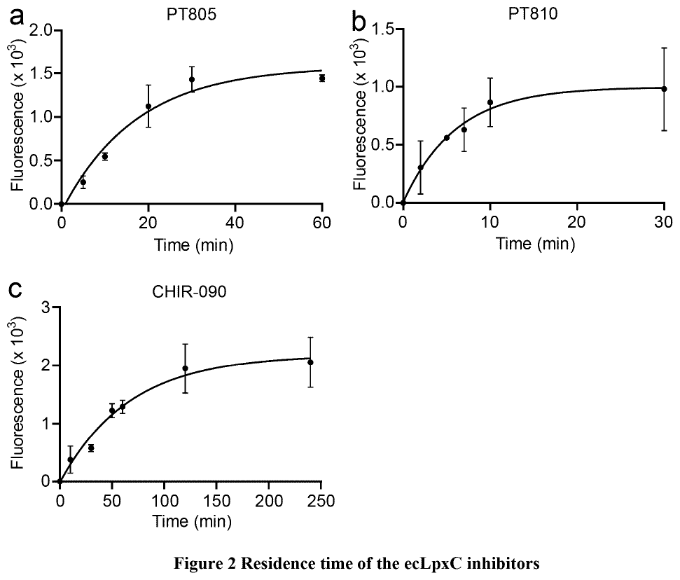

The residence times of the three inhibitors were measured on ecLpxC using a Penefsky column-based method (Penefsky, 1979 and Yu, 2015). Compounds were preincubated with ecLpxC and then mixed with a fluorescent LpxC inhibitor PT900. The enzyme was then rapidly separated from unbound ligand using a SpinTrap G-25 column at different times after adding the fluorescent competitor, and the fluorescence intensity of the eluate was quantified on a BioTek plate reader using λₑₓ 315 nm and λₑₘ 420 nm. This approach gave residence times of 17±7 min, 6±3 min and 66±30 min for PT805, PT810 and CHIR-090 (Table 1, FIG. 2).

TABLE 1
Kinetic and microbiological activity

| | | Wild-type^{a} | | | | | | FabZ*^{a} | | | |
|---|---|---|---|---|---|---|---|---|---|---|---|
| | t_{R} | MIC | PAE (h)^{d} | | | PAE (h) (sub-MIC AZ)^{e} | | MIC | PAE (h)^{d} | | |
| | (min)^{b} | (μM)^{c} | 1x | 4x | 16x | 16x(0.2) | 16x(0.4) | (μM)^{c} | 1x | 4x | 16x |
| PT805 | 17 ± 7 | 0.16 | 0 | 0 | 0 | 1.4 ± 0.1 | 2.4 ± 0.1 | 0.019 | 0.8 ± 0.1 | 1.5 ± 0.1 | 2.1 ± 0.2 |
| PT810 | 6 ± 3 | 0.17 | 0 | 0 | 0 | 1.9 ± 0.8 | 2.6 ± 0.8 | 0.080 | 0.7 ± 0.2 | 0.8 ± 0.2 | 0.9 ± 0.2 |
| CHIR-090 | 66 ± 30 | 0.62 | 0 | 0 | 0 | 2.8 ± 0.3 | 3.7 ± 0.2 | 0.010 | 0.5 ± 0.1 | 1.3 ± 0.3 | 2.4 ± 0.2 |
| Erythromycin | ND | 50 | ND^{f} | 0.7 ± 0.1 | 1.4 ± 0.4 | ND | ND | 6.25 | ND | 0.8 ± 0.2 | 1.5 ± 0.4 |
| Cefamandole | ND | 6.25 | ND | 0 | 0 | 0 | 0 | 1.6 | ND | 0 | 0 |
| Trimethoprim | ND | 2.5 | ND | 0 | 0 | ND | ND | 2.5 | ND | 0 | 0 |

*^{a}Wild-type, E. coli strain (K-12, MG1655); FabZ*, hyperactive FabZ strain (L85P)
^{b}Residence times were determined at 25° C. by monitoring the rate of inhibitor displacement using a fluorescent ligand. The reported values are the average of two independent experiments where the errors represent the standard deviation from the mean.
^{c}MIC values were determined by the microbroth dilution method. Experiments were performed in triplicate and the reported values are the average of the three independent experiments.
^{d}The PAE was calculated using standard procedure, where the time required by the bacteria to recover 1 log after washing out of the inhibitor was compared to the culture having no inhibitor (DMSO) was used as a vehicle.⁷ Experiments were performed in triplicate and the reported values are the average of the three independent experiments with the errors representing the standard deviation from the mean.
^{e}PAE values for wild-type E. coli in media containing sub-MIC concentrations of azithromycin at 37° C. MIC values of azithromycin were first determined for bacteria exposed to 16xMIC of the ecLpxC inhibitor and then diluted into media containing azithromycin of various concentrations. The control MIC was 12 μM whereas the MIC values for bacteria pre-exposed to 16xMIC ecLpxC inhibitor were 3.2 μM, while the MIC following pre-exposure to 16x cefamandole was 6.25 μM. The number in parentheses represents the sub-MIC azithromycin concentration (0.2x or 0.4xMIC) used in the post-exposure phase. Experiments were performed in triplicate and the reported values are the average of the three independent experiments with the errors representing the standard deviation from the mean.
^{f}ND, not determined.

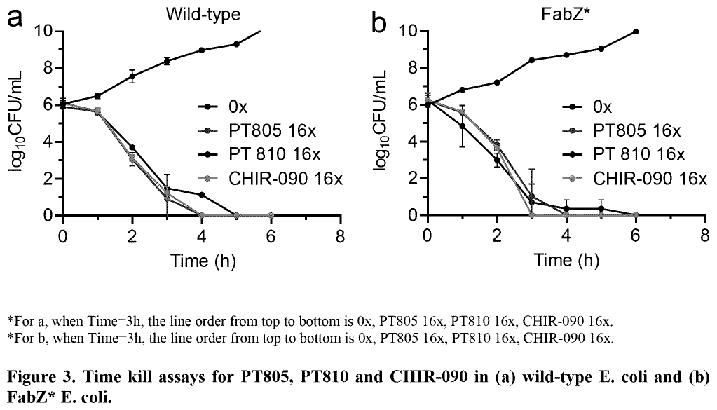

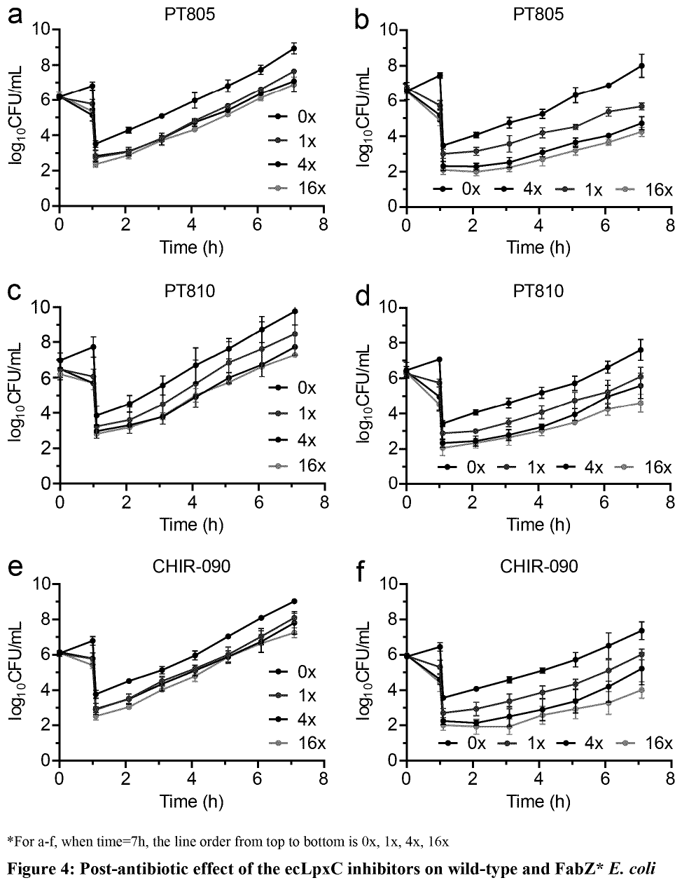

The antimicrobial activity of the compounds was examined. Each ecLpxC inhibitor had potent activity towards a wild-type E. coli strain (K-12 MG1655) with MIC values between 0.2-0.6 μM (Table 1). Time kill assays also revealed that the compounds were bactericidal, reducing the starting log CFU/mL of the bacterial culture by greater than 3 logs (FIG. 3) (Pankey, 2004). The post-antibiotic effect (PAE) of the inhibitors was measured by monitoring the regrowth of bacteria following compound washout. Wild-type E. coli cells were exposed to 1×, 4× and 16×MIC of the inhibitors for 1 h, after which the bacteria were diluted 1:1000 fold into fresh media. Bacterial regrowth was monitored by plating samples as a function of time after wash-out and enumerating CFUs. The PAE was calculated by determining the time required for a 1 log increase in CFU after inhibitor wash-out compared to a culture treated with vehicle (DMSO) (Davoodi, 2020). No PAE could be observed for any of the inhibitors (FIG. 4, Table 1).

Cultures of wild-type or FabZ* E. coli (10⁶ CFU/mL) were treated with 1×, 4× or 16×MIC of inhibitor for 1 h followed by 1:1000-fold dilution into fresh cation-adjusted Mueller-Hinton (CaMH) media at 37° C. Samples (100 μL) of the diluted cultures were then plated on Muller-Hinton agar plates every hour and CFUs enumerated following incubation of the plates at 37° C. for 16 h. PAE data for wild-type E. coli are in panels (a) PT805, (c) PT810 and (e) CHIR-090, and for FabZ* E. coli in (b) PT805, (d) PT810 and (f) CHIR-090. In FIG. 4, the experimental data points are the mean values from triplicate, independent measurements and the error bars represent the standard deviation from the mean.

### Experiment 2. Post-Antibiotic Effect of LpxC Inhibitors in the FabZ E. coli Strain and in the Presence of Macrolides

Based on the hypothesis that the lack of PAE for the ecLpxC inhibitors in wild-type E. coli was due to rapid turnover of the target, PAEs were determined under conditions designed to increase the stability of ecLpxC. This included use of an E. coli strain (FabZ*) containing a hyperactive mutant of the fatty acid biosynthesis O-hydroxyacyl-ACP enzyme (FabZ), and determination of PAEs for wild-type E. coli in the presence of sub-MIC concentrations of macrolides.

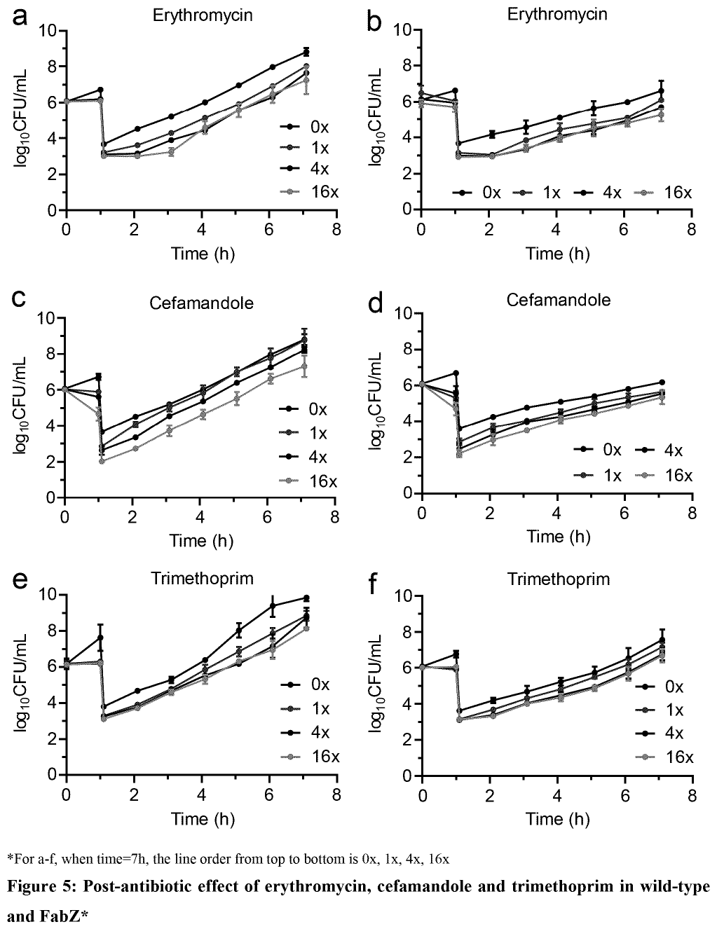

The MIC values were measured for PT805, PT810 and CHIR-090 in the FabZ* strain, which were ˜2-60 fold lower than the corresponding MICs for the wild-type strain, and then determined the PAEs at 1×, 4× and 16×MIC (FIG. 4). In contrast to wild-type E. coli, PAEs of 0.5-2.4 h were observed: for instance, at 4×MIC the PAEs were 1.53±0.13 h, 0.76±0.19 h and 1.34±0.04 for PT805, PT810 and CHIR-090, respectively (Table 1). PAEs of three antibiotics was determined to have different mechanisms of action. In contrast to the ecLpxC inhibitors, no difference in PAE was observed between the wild-type and FabZ* strains for erythromycin, which had a PAE of 0.8±0.05 h in both strains, or cefamandole and trimethoprim which failed to generate a PAE in either strain (FIG. 5, Table 1).

The PAE measurements were repeated for wild-type E. coli in the presence of sub-MIC levels of the protein synthesis inhibitor azithromycin. First, it was determined if azithromycin showed a significant shift in MIC if wild-type E. coli was first exposed to 16×MIC of the inhibitors prior to dilution into media containing azithromycin. In each case the MIC of azithromycin decrease 4-fold from 12 μM to 3.25 μM following pre-treatment with the ecLpxC inhibitors or 2-fold from 12 μM to 6.2 μM following pre-treatment with cefamandole, which was included as a control. The shifted MIC was then used to calculate the sub-MIC concentrations of azithromycin used in the subsequent experiments.

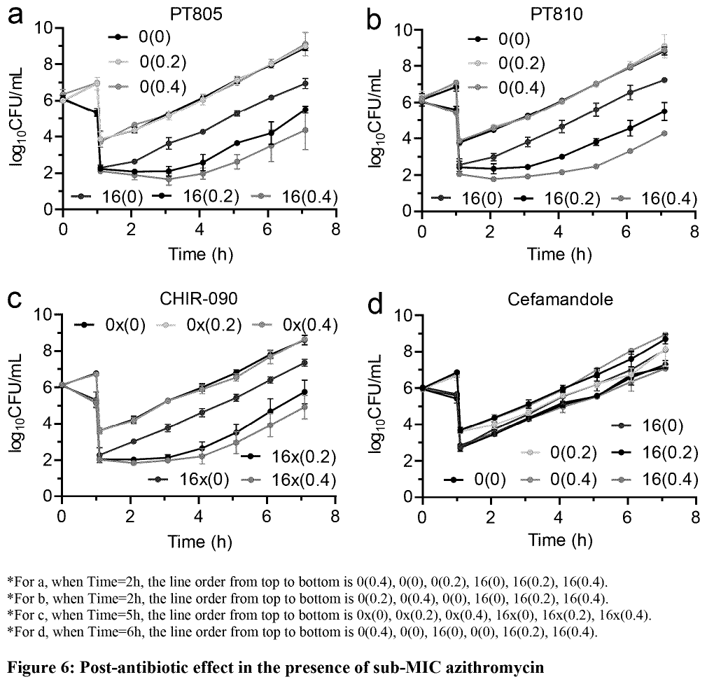

Following exposure of bacteria to 16×MIC of each ecLpxC inhibitor or cefamandole, cultures were diluted 1:1000-fold into media containing 0, 0.2 or 0.4×MIC azithromycin. Whereas no PAE was observed in the absence of macrolides, significant PAEs were observed in the presence of sub-MIC azithromycin for PT805, PT810, and CHIR-090, but not for the control antibiotic cefamandole (FIG. 6, Table 1). For example, the PAE values at 0.4×MIC azithromycin were 2.4±0.1, 2.6±0.8 and 3.7±0.2 h for PT805, PT810 and CHIR-090, respectively (Table 1).

Cultures of wild-type E. coli (K12 MG1655) 10⁶ CFU/mL were treated with 0× or 16×MIC of inhibitor for 1 h followed by 1:1000-fold dilution into fresh CaMH media containing 0×, 0.2× or 0.4×MIC azithromycin at 37° C. Samples (100 μL) of the diluted cultures were then plated on Muller-Hinton agar plates every hour and CFUs were enumerated following incubation of the plates at 37° C. for 16 h. PAE data are shown for (a) PT805, (b) PT810 and (c) CHIR-090 and (d) cefamandole. Values in parenthesis are the concentration of azithromycin in the media (Ox, 0.2× or 0.4×MIC). The experimental data points in FIG. 6. are the mean values from triplicate, independent measurements and the error bars represent the standard deviation from the mean.

### Experiment 3. Rate of ecLpxC Turnover

To quantify protein turnover rates, E. coli cells were metabolically labelled with isotopically-labelled “heavy” lysine ¹³C₆, ¹⁵N₂ (Lys8) and diluted the cells into media containing normal “light” lysine (Lys0). The 0-time point sample taken from purely “heavy” labelled media indicated the starting point of the experiment and also the percent incorporation of the “heavy” label. Subsequently the decay of the heavy label over time was analyzed by a high-resolution mass spectrometry-based method. After harvesting and lysing bacterial cells, ecLpxC was then enriched from the lysate using an antibody-based pull-down method, because it was unable to identify peptides specific to ecLpxC directly from the lysate. The enrichment method increased the coverage, and it was able to identify 20 unique peptides in each of the three biological replicates of which 5 peptides showed lysine incorporation consistently. However, with the enrichment process, other proteins were also present in the enriched sample, which bound non-specifically to the beads (Table 2).

TABLE 2
Proteins Analyzed by pSILAC

| Accession | | Unique peptides | | | PSMs | | | MW | Sum PEP Score | | | RIA Values (H/H + L) |
|---|---|---|---|---|---|---|---|---|---|---|---|---|
| Number | Gene | E1 | E2 | E3 | E1 | E2 | E3 | (kDa) | E1 | E2 | E3 | 0 |
| P0A725 | lpxC | 10 | 19 | 24 | 29 | 207 | 733 | 33.9 | 24.94 | 173.62 | 471.2 | 0.91 ± 0.005 |
| P0A9B2 | gapA | 16 | 31 | 29 | 116 | 268 | 596 | 35.5 | 83.15 | 245.96 | 535.63 | 0.86 ± 0.01 |
| P0A6F5 | groL | 29 | 51 | 68 | 248 | 653 | 2806 | 57.3 | 183.22 | 695.50 | 1786.24 | 0.89 ± 0.004 |
| P61891 | mdh | 13 | 25 | 22 | 57 | 183 | 441 | 32.3 | 60.36 | 203.44 | 277.90 | 0.92 ± 0.006 |
| P0A9C7 | glnA | 19 | 25 | 38 | 69 | 175 | 698 | 51.9 | 64.89 | 211.23 | 667.51 | 0.93 ± 0.01 |
| P0A910 | ompA | 11 | 19 | 21 | 67 | 131 | 305 | 37.2 | 68.46 | 118.50 | 260.52 | 0.94 ± 0.01 |
| P0A7T3 | rpsP | 3 | 7 | 9 | 12 | 99 | 199 | 10.6 | 12.62 | 105.84 | 146.62 | 0.89 ± 0.004 |
| P0A7W3 | rpsA | 8 | 23 | 12 | 21 | 124 | 178 | 17.6 | 28.55 | 127.58 | 161.23 | 0.82 ± 0.05 |

| Accession | RIA Values (H/H + L) | | | | | |
|---|---|---|---|---|---|---|
| Number | 5 | 10 | 20 | 40 | 60 | 80 |
| P0A725 | 0.77 ± 0.08 | 0.69 ± 0.03 | 0.55 ± 0.03 | 0.38 ± 0.006 | 0.27 ± 0.008 | 0.23 ± 0.003 |
| P0A9B2 | 0.79 ± 0.03 | 0.79 ± 0.02 | 0.67 ± 0.03 | 0.59 ± 0.05 | 0.44 ± 0.05 | 0.41 ± 0.05 |
| P0A6F5 | 0.83 ± 0.007 | 0.82 ± 0.03 | 0.67 ± 0.03 | 0.57 ± 0.06 | 0.48 ± 0.06 | 0.42 ± 0.05 |
| P61891 | 0.84 ± 0.04 | 0.80 ± 0.04 | 0.67 ± 0.04 | 0.53 ± 0.04 | 0.44 ± 0.03 | 0.36 ± 0.03 |
| P0A9C7 | 0.87 ± 0.05 | 0.83 ± 0.05 | 0.79 ± 0.04 | 0.70 ± 0.05 | 0.54 ± 0.05 | 0.43 ± 0.05 |
| P0A910 | 0.84 ± 0.03 | 0.83 ± 0.02 | 0.65 ± 0.03 | 0.53 ± 0.05 | 0.52 ± 0.05 | 0.51 ± 0.05 |
| P0A7T3 | 0.77 ± 0.04 | 0.73 ± 0.007 | 0.65 ± 0.02 | 0.62 ± 0.05 | 0.44 ± 0.01 | 0.41 ± 0.01 |
| P0A7W3 | 0.66 ± 0.08 | 0.71 ± 0.04 | 0.56 ± 0.03 | 0.51 ± 0.01 | 0.43 ± 0.02 | 0.34 ± 0.03 |

Unique peptides: Defined as a peptide that exists in only one protein in the proteome of interest.¹
PSMs: Scoring function that assigns a numerical value to the peptide-spectrum (P-S) pair expressing the likelihood that the fragmentation of a peptide with sequence P is recorded in experimental mass spectrum S. PSM score informs on the quality of the spectrum.²
Sum PEP Score: Score that signifies the probability of the observed PSM being incorrect.³ Scores are reported as −10*log₁₀(P), where P is the absolute probability. A probability of 10⁻²⁰ thus becomes a score of 200.
RIA Values (H/H + L): Relative Isotopic Abundance expressed as abundance of heavy labelled peptide (H), divided by the abundance of all (heavy + light) peptide.
Protein Description:
lpxC: UDP-3-O-acyl-N-acetylglucosamine deacetylase OS = Escherichia coli (strain K12)
gapA: Glyceraldehyde-3-phosphate dehydrogenase A OS = Escherichia coli (strain K12)
groL: 60 kDa chaperonin OS = Escherichia coli (strain K12)
mdh: Malate dehydrogenase OS = Escherichia coli (strain K12)
glnA: Glutamine synthetase OS = Escherichia coli (strain K12)
ompA: Outer membrane protein A OS = Escherichia coli (strain K12)
rpsP: 30S ribosomal protein S16 OS = Escherichia coli (strain K12)
rpsA: 30S ribosomal protein S5 OS = Escherichia coli (strain K12)

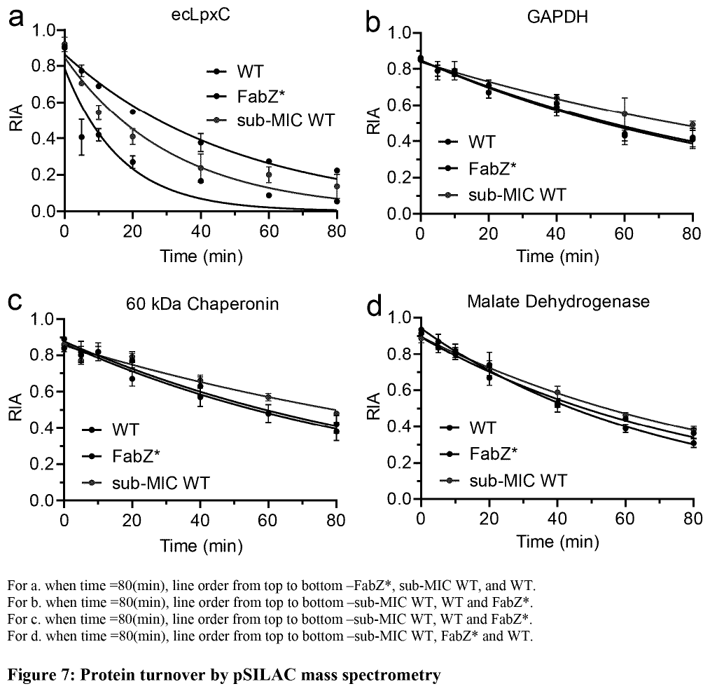

The incorporation of the heavy label in both the wild-type and the mutant strain was ˜90% as determined by Relative Isotopic Abundance (RIA) values (Fröhlich 2013 and Cargile 2004). Time points ranging from 5 to 80 min after transfer to “light” labelled lysine media were measured. For ecLpxC in wild-type E. coli biological replicates showed high reproducibility, although for the wild-type the first 5 min time point showed variability which could be due to the low abundance of the protein (Soufi 2010). The data plotted in FIG. 7 shows a faster decay of the label for ecLpxC in wild-type E. coli as compared to that of the FabZ* mutant giving half-lives of 12 and 35 min, respectively (Table 3).

TABLE 3
Protein turnover rates from pSILAC mass spectrometry

| | Wild-Type^{a} | | | | | |
|---|---|---|---|---|---|---|
| | | | (sub-MIC AZ)^{b} | | FabZ*^{a} | |
| | | Half-life | | Half-life | | Half-life |
| | k (min⁻¹) | (min) | k (min⁻¹) | (min) | k (min⁻¹) | (min) |
| ecLpxC | 0.06 ± 0.01 | 12 ± 2 | 0.03 ± 0.01 | 23 ± 2 | 0.02 ± 0.01 | 35 ± 1 |
| Glyceraldehyde-3- | 0.009 ± 0.001 | 77 ± 6 | 0.007 ± 0.001 | 99 ± 7 | 0.009 ± 0.001 | 71 ± 7 |
| phosphate | | | | | | |
| dehydrogenase | | | | | | |
| 60 kDa Chaperonin | 0.009 ± 0.001 | 73 ± 10 | 0.006 ± 0.001 | 103 ± 18 | 0.009 ± 0.001 | 71 ± 9 |
| Malate dehydrogenase | 0.014 ± 0.005 | 50 ± 1 | 0.011 ± 0.002 | 65 ± 3 | 0.012 ± 0.006 | 57 ± 3 |
| Glutamine synthase | 0.010 ± 0.001 | 69 ± 3 | 0.008 ± 0.001 | 86 ± 11 | 0.009 ± 0.001 | 77 ± 6 |
| Outer membrane | 0.009 ± 0.001 | 77 ± 6 | 0.011 ± 0.001 | 63 ± 8 | 0.011 ± 0.001 | 64 ± 9 |
| protein A | | | | | | |
| 30S ribosomal protein | 0.010 ± 0.001 | 68 ± 5 | 0.008 ± 0.001 | 83 ± 12 | 0.010 ± 0.001 | 68 ± 10 |
| S16 | | | | | | |
| 30S ribosomal protein | 0.010 ± 0.001 | 68 ± 3 | 0.007 ± 0.001 | 93 ± 16 | 0.010 ± 0.001 | 68 ± 10 |
| S5 | | | | | | |

*^{a}Turn-over SILAC experiments were performed in M9 media with wild-type K12 MG1655 E. coli strain and the FabZ hyperactive mutant E. coli strain.
^{b}0.4xMIC azithromycin was included in the media containing normal “light” lysine.
Each experiment was performed in three independent biological replicates with errors representing the standard deviation from the mean.

Protein turnover was quantified in the wild-type strain of E. coli (black), the FabZ* mutant strain (blue), and in the wild-type strain grown in media containing sub-MIC concentration of azithromycin (0.4×MIC) (red). In each case the relative isotopic abundance (RIA) was determined as a function of time after transferring bacterial cultures grown in media containing “heavy” lysine to media containing normal “light” lysine. The RIA was calculated by quantifying the isotopic abundance of peptides generated by trypsin digest using mass spectrometry. ecLpxC (A) was enriched using antibody pull-down with beads coated with the ecLpxC antibody. This method also led to the enrichment of several other proteins that were used as controls including GAPDH (B), 60 kDa chaperonin (C), and malate dehydrogenase (D). Each point in FIG. 7 represents average of three independent biological replicates where the error bars represent the standard deviation from the mean.

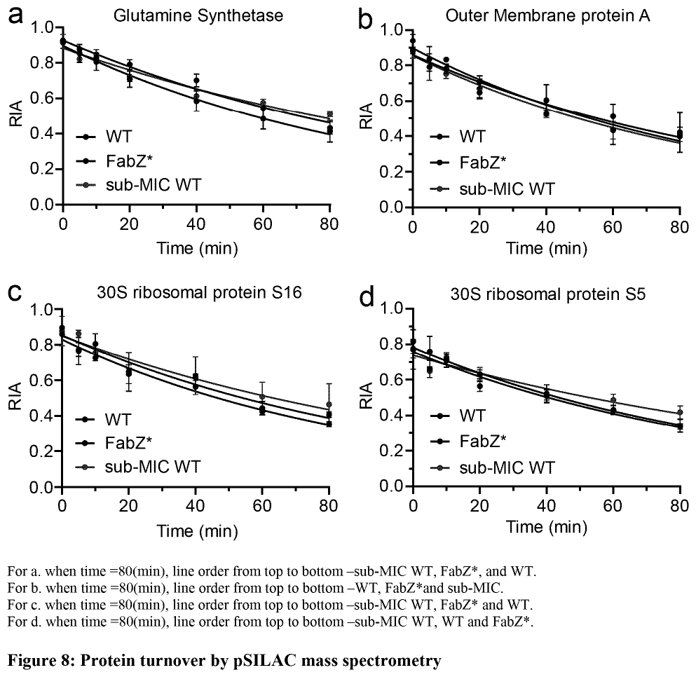

To assess whether the FabZ* mutant strain specifically affects the turn-over of LpxC, advantages of other proteins were taken that were being enriched by the pull-down experiment including several housekeeping proteins that should not be affected by the genetic mutation that specifically targets LpxC turn-over (Soares, 2013). Consistent with expectations, proteins such as GAPDH, 60 kDa chaperonin, malate synthase, glutamine synthase, outer membrane protein A, and the 30S ribosomal proteins S16 and S5, had similar half-lives and degradation constants in both the wild-type and the mutant strains (FIG. 7, FIG. 8, Table 3).

The rate of protein turnover in the presence of sub-MIC concentrations of azithromycin was accessed. pSILAC in the presence of 0.4× (1.25 μM) azithromycin gave a half-life of ecLpxC of 22 min, which was 2-fold higher than the rate in the absence of macrolide. Other proteins detected in the pull-down experiment were similarly affected and showed increased half-lives in the presence of sub-MIC azithromycin (FIG. 7, FIG. 8, Table 3).

### Experimental Details of Synthesis Schemes

### Synthesis of PT900

PT900 (3) was synthesized following the reaction series shown in Scheme S1.⁴

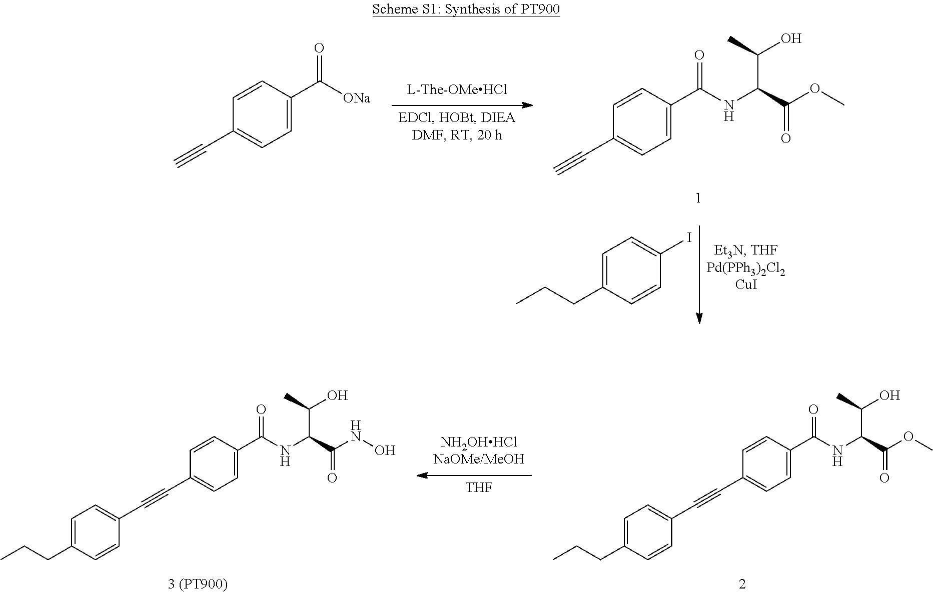

### Methyl (2S,3R)-2-(4-ethynylbenzamido)-3-hydroxybutanoate (1)

Diisopropylethylamine (1.1 mL, 11.9 mmol) was added to a stirred solution of 4-ethynylbenzoic acid (500 mg, 2.97 mmol), L-threonine methyl ester hydrochloride (605.3, 3.6 mmol), EDCI (684.2 mg, 3.6 mmol) and HOBt (442 mg, 3.3 mmol) in anhydrous DMF (15 mL) at 0° C. under N₂. The solution was stirred at 0° C. for 1 h and then at room temperature (rt) for 20 h. The solution was diluted with EtOAc (100 mL) and washed with 1.0 M HCl, saturated NaHCO₃, H₂O, dried over MgSO4, filtered and concentrated in vacuo. The crude material was purified via flash chromatograph to afford the final product methyl (3R)-2-(4-ethynylbenzamido)-3-hydroxybutanoate.

Yield (55%), LCMS: m/z calc for C₁₄H₁₅NO₄ [M+H]⁺: 262.29; found: 262.10.

¹H NMR (700 MHz, CDCl₃) δ ppm 1.30 (d, J=6.45 Hz, 3H) 3.22 (s, 1H) 3.82 (s, 3H) 4.48 (qd, J=6.38, 2.37 Hz, 1H) 4.83 (dd, J=8.82, 2.37 Hz, 1H) 6.89 (d, J=8.60 Hz, 1H) 7.58 (m, J=8.39 Hz, 2H) 7.81 (m, J=8.39 Hz, 2H).

### Methyl (2S,3R)-3-hydroxy-2-(4-((4-propylphenyl)ethynyl)benzamido)butanoate (2)

A solution of methyl (3R)-2-(4-ethynylbenzamido)-3-hydroxybutanoate (0.38 mmol, 100 mg), 1-iodo-4-propylbenzene (0.52 mmol, 0.084 mL) and Et₃N (0.87 mmol, 0.12 mL) in THE (7 mL) was purged with a stream of N₂ for two min and then treated with PdCl₂(PPh₃)₂ (0.013 mmol, 9.4 mg) and CuI (0.024 mmol, 4.6 mg). The reaction mixture was stirred for 40 h, concentrated by rotary evaporation, and purified by flash column chromatography with dichloromethane (DCM)/MeOH.

Yield (63%), LCMS: m/z calc for C₂₃H₂₅NO₄ [M+H]⁺: 380.47; found: 380.20.

¹H NMR (700 MHz, CDCL₃) d ppm 0.97 (t, J=7.31 Hz, 3H) 1.33 (d, J=6.45 Hz, 3H) 1.65-1.70 (m, 2H) 2.60-2.66 (m, 2H) 3.84 (s, 3H) 4.46-4.54 (m, 1H) 4.86 (dd, J=8.71, 2.26 Hz, 1H) 6.90 (d, J=8.60 Hz, 1H) 7.20 (m, J=8.17 Hz, 2H) 7.49 (m, J=8.17 Hz, 2H) 7.60-7.65 (m, 2H) 7.85 (m, J=8.39 Hz, 2H).

### N-((2S,3R)-3-hydroxy-1-(hydroxyamino)-1-oxobutan-2-yl)-4-((4-propylphenyl)ethynyl)benzamide (3)

Sodium methoxide (25 wt % in MeOH, 127 mg, 0.59 mmol) was added to a stirred solution of hydroxylamine hydrochloride (27.5 mg, 0.4 mmol) in anhydrous MeOH 0.5 mL at 0° C. under N₂ atmosphere. After stirring for 20 min, a solution of the methyl ester (50 mg, 0.6 mmol) in 1:1 MeOH/THF (2 mL) was added, and the reaction mixture stirred at 0° C. for 1 h, and then at rt overnight.

The reaction mixture was quenched with 1.0 M HCl, concentrated by rotary evaporator to remove the organic solvents, and diluted with DMSO. Purification by reversed phase HPLC with a C18 column (Luna® 5 μm C18(2) 100 Å 250×10 mm). Chromatography was performed with water as buffer A and acetonitrile as buffer B. Elution was monitored at 260 nm, and the fractions containing the final product was pooled and lyophilization of the collected fraction gave the final product.

Yield (45%) HRMS: m/z calc for C₂₂H₂₄N₂O₄ [M−H]⁻: 379.1663; found: 379.1655.

¹H NMR (700 MHz, DMSO-d₆) δ ppm 0.86 (t, J=7.31 Hz, 3H) 1.05 (d, J=6.45 Hz, 3H) 1.54-1.60 (m, 2H) 2.55 (t, J=7.53 Hz, 2H) 3.98-4.01 (m, 1H) 4.22 (dd, J=8.39, 5.59 Hz, 1H) 4.86 (d, J=6.45 Hz, 1H) 7.23 (m, J=7.96 Hz, 2H) 7.46 (m, J=7.96 Hz, 2H) 7.61 (m, J=8.17 Hz, 2H) 7.91 (m, J=8.17 Hz, 2H) 8.10 (d, J=8.60 Hz, 1H) 8.80-8.84 (m, 1H) 10.64 (s, 1H)

### Synthesis of PT810

PT810 (12) was synthesized using the reaction series shown in Scheme S2 which involved preparation of the Head (8) and Tail (10) portions of the molecule followed by Suzuki Coupling and deprotection.

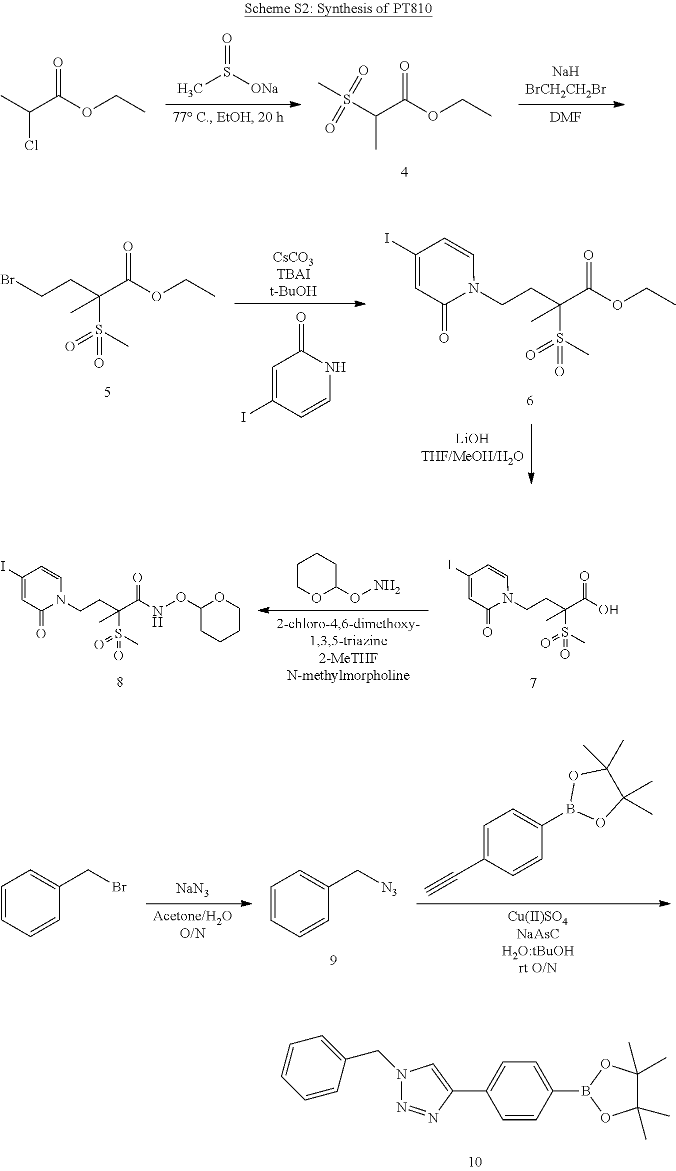

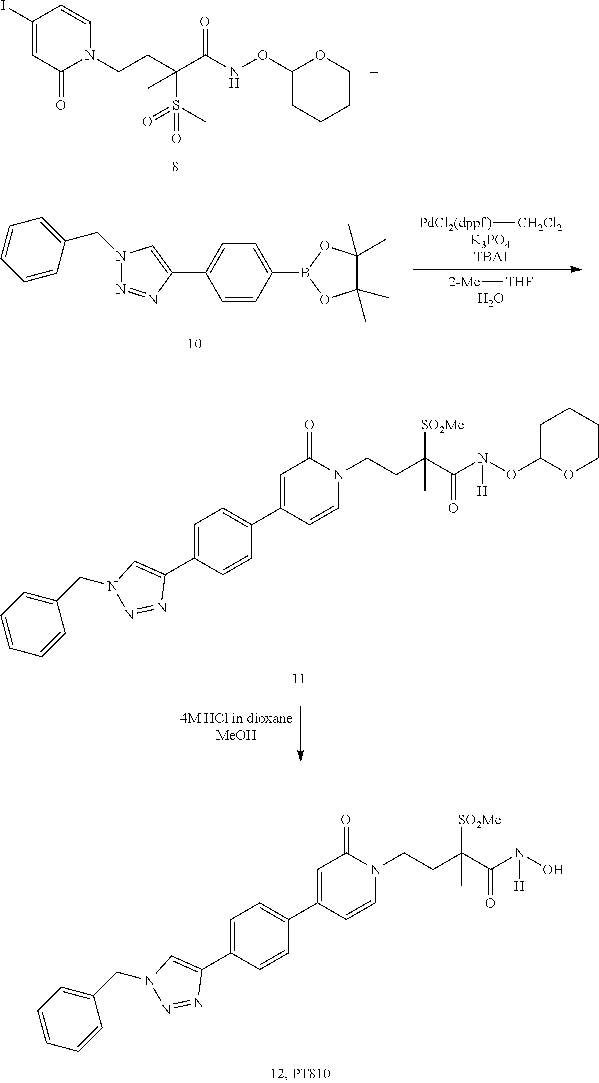

### Synthesis of the Head Group (8)

### Ethyl 2-(methylsulfonyl)propanoate (4)

Sodium methanesulfinate (3.9 g, 38.4 mmol) was added to ethyl 2-chloropropionate (5 g, 892 mmol) in EtOH (16 mL). The reaction mixture was heated to 77° C. and stirred at this temperature for 20 h. The reaction mixture was allowed to cool to rt and the solids were removed by filtration and washed with ethanol. The filtrates were combined and concentrated in vacuo. The crude product was suspended in Et₂O (25 mL), and the solids were removed by filtration. The filtrate was concentrated in vacuo to afford the title compound as a pale-yellow oil.

Yield 67%, LCMS: m/z calc for C₆H₁₂O₄S [M+H]⁺: 181.23; found: 181.10.

¹H NMR (700 MHz, CDCl₃) δ ppm 1.30 (t, J=7.10 Hz, 3H) 1.63 (d, J=7.31 Hz, 3H) 3.03 (s, 3H) 3.88 (q, J=7.17 Hz, 1H) 4.23-4.29 (m, 2H)

### Ethyl 4-bromo-2-methyl-2-(methylsulfonyl)butanoate (5)

Sodium hydride (60% dispersion in mineral oil, 1.076 g, 44.83 mmol) was washed with hexanes (2×10 mL) under nitrogen and then suspended in N,N-dimethylformamide (15 mL). The suspension was treated dropwise with ethyl 2-(methylsulfonyl)propanoate (4, 4.04 g, 22.41 mmol) in DMF (5 mL). The mixture was stirred for 30 min at rt, cooled to 0° C., and treated drop-wise with 1,2-dibromoethane (1.9 mL, 22.41 mmol). The mixture was allowed to warm to rt and stirred overnight, after which the reaction was quenched with saturated NH₄Cl (50 mL) and extracted with Et₂O (4×25 mL). The organic layers were combined and then washed with water:brine (1:1, 4×25 mL), dried with MgSO₄, and concentrated in vacuo. The crude material was purified via silica chromatography with a gradient of 10-20% EtOAc in hexanes to afford the title compound as a pale-yellow oil.

Yield—42%, LCMS: m/z calc for C₈H₁₅BrO₄S [M+H]⁺: 288.18; found: 288.10.

¹H NMR (700 MHz, CDCL₃) δ ppm 1.36 (t, J=7.21 Hz, 3H) 1.67 (s, 3H) 2.57 (ddd, J=14.04, 9.84, 4.73 Hz, 1H) 2.78-2.84 (m, 1H) 3.07 (s, 3H) 3.39 (td, J=10.11, 6.67 Hz, 1H) 3.52 (td, J=10.11, 4.73 Hz, 1H) 4.32 (q, J=7.17 Hz, 2H)

### Ethyl 4-(4-iodo-2-oxopyridin-1(2H)-yl)-2-methyl-2-(methylsulfonyl)butanoate (6)

A solution of ethyl 4-bromo-2-methyl-2-(methylsulfonyl)butanoate (780 mg, 2.72 mmol) in THE (9 mL) was added to a suspension of 4-iodopyridin-2-ol (428.7 mg, 1.94 mmol), CS₂CO₃ (694 mg, 2.13 mmol), and tetra-n-butylammonium iodide (TBAI) (71.6 mg, 0.194 mmol) in t-BuOH (9 mL). The suspension was heated to 35° C. and was stirred at this temperature for 3 days. The reaction mixture was allowed to cool to rt and then filtered. The filter cake was washed with EtOAc and the filtrates were washed with H₂O (100 mL). The aqueous layer was extracted with EtOAc (3×100 mL), and the organic layers were combined, dried with MgSO₄, filtered, and concentrated in vacuo. The crude material was purified via flash chromatography using a gradient of 30-100% EtOAc in n-hexane to afford the title compound.

Yield 82.6%, LCMS: m/z calc for C₁₃H₁₈INO₅S [M+H⁺]: 428.26; found: 428.00.

¹H NMR (400 MHz, CDCL₃) δ ppm 1.35 (t, J=7.09 Hz, 3H) 1.75 (s, 3H) 2.41-2.55 (m, 2H) 3.11 (s, 3H) 3.91-3.99 (m, 1H) 4.16-4.24 (m, 1H) 4.25-4.31 (m, 2H) 6.52 (dd, J=7.09, 1.59 Hz, 1H) 7.00 (d, J=7.21 Hz, 1H) 7.10 (d, J=1.47 Hz, 1H)

### 4-(4-iodo-2-oxopyridin-1(2H)-yl)-2-methyl-2-(methylsulfonyl)butanoic acid (7)

Lithium hydroxide (6 mmol) was added to a solution of the ester (1.0 mmol) in THF:MeOH:H₂O (2:1:1, 1 M) at rt and the mixture was stirred until complete conversion was achieved as monitored by TLC. The pH of the reaction mixture was adjusted to 3 using aq 1 N HCl and the reaction mixture was extracted with EtOAc. The organic layers were combined, dried, filtered, and concentrated in vacuo to afford the desired product.

LCMS: m/z calc for C₁₁H₁₄INO₅S [M−H]⁻: 398.19; found: 398.00.

¹H NMR (700 MHz, DMSO-d₆) δ ppm 1.54 (s, 3H) 2.13 (ddd, J=13.34, 10.33, 4.95 Hz, 1H) 2.40 (ddd, J=13.18, 10.38, 5.92 Hz, 1H) 3.15 (s, 3H) 3.87 (ddd, J=12.69, 10.54, 5.81 Hz, 1H) 3.97-4.03 (m, 1H) 6.60 (dd, J=6.99, 1.83 Hz, 1H) 6.92 (d, J=1.51 Hz, 1H) 7.47 (d, J=7.10 Hz, 1H)

### 4-(4-iodo-2-oxopyridin-1(2H)-yl)-2-methyl-2-(methylsulfonyl)-N-((tetrahydro-2H-pyran-2-yl)oxy)butanamide (8)

N-Methyl morpholine (1.6 eq) was added to a solution of 2-chloro-4,6-dimethoxy-3,5-triazine (1.4 eq) and the carboxylic acid (1.0 eq) in 2-Me-TFH (1 M) and the reaction mixture was stirred for 1 h. O-(Tetrahydro-2H-pyran-2-yl)hydroxylamine (1.6 eq) was added to the reaction mixture which was then stirred overnight at rt. The crude reaction was filtered and the filter pad was washed with DCM. The combined filtrates were concentrated in vacuo, and the crude material was purified via flash chromatography using 0-10% MeOH/DCM on silica to afford the desired product.

Yield 35%, LCMS: m/z calc for C₁₆H₂₃IN₂O₆S [M−H]⁻: 497.32; found: 497.00.

¹H NMR (500 MHz, CDCL₃) δ ppm 1.61-1.65 (m, 3H) 1.71 (m, 3H) 1.81 (dt, J=12.70, 3.72 Hz, 1H) 1.86-1.91 (m, 1H) 1.92-1.97 (m, 1H) 2.31-2.39 (m, 1H) 2.44-2.53 (m, 1H) 3.20-3.22 (m, 3H) 3.60-3.70 (m, 1H) 4.00 (dddd, J=13.85, 11.10, 5.95, 3.13 Hz, 1H) 4.12-4.20 (m, 1H) 4.27-4.34 (m, 1H) 5.14-5.20 (m, 1H) 6.63 (dd, J=7.02, 1.68 Hz, 1H) 7.05 (d, J=7.02 Hz, 1H) 7.18 (d, J=1.68 Hz, 1H) 11.69-11.74 (br.s, 1H)

### Synthesis of the Tail Group (10)

### Conversion of Benzyl Bromide to Benzyl Azide (9)

Benzyl bromide (500 mg, 2.9 mmol) was dissolved in 4:1 acetone:H₂O (10 mL) and sodium azide (361 mg, 5.55 mmol, 1.9 eq.) was added. The reaction was stirred overnight at rt and then extracted with Et2O (3×50 mL) after which the organic phase was washed with H₂O (50 mL) and brine (50 mL). The organic phase was dried with MgSO₄, filtered and solvent was removed in vacuo to yield the desired product as colorless oil (52%).

¹H NMR (500 MHz, CDCL₃) δ ppm 4.37 (s, 2H) 7.32-7.46 (m, 5H)

### 1-Benzyl-4-(4-(4,4,5,5-tetramethyl-1,3,2-dioxaborolan-2-yl)phenyl)-1H-1,2,3-triazole (10)

2-(4-Ethynylphenyl)-4,4,5,5-tetramethyl-1,3,2-dioxaborolane (239.6 mg, 1.05 mmol) and benzyl aside (140 mg, 1.05 mmol) were suspended in 10 mL of a 1:1 H₂O/t-BuOH mixture. Sodium ascorbate (1.05 mmol, 208.2 mg) was added, followed by copper(II) sulfate pentahydrate (262.4 mg, 1.05 mmol).⁵ The heterogeneous mixture was stirred vigorously overnight. The reaction mixture was then extracted with 3×50 mL DCM and water and the organic phase was washed with H₂O (50 mL) and brine (50 mL). The organic phase was dried with MgSO₄, filtered and the solvent was removed in vacuo. The crude was purified by flash chromatography with 0-100% EtOAc/hexane to afford the pure product as white solid.

Yield 77%, LCMS: m/z calc for C₂₁H₂₄BN₃O₂ [M+H]⁺: 362.26; found: 362.20.

¹H NMR (500 MHz, CDCL₃) δ ppm 1.37 (s, 12H) 5.60 (s, 2H) 7.33-7.36 (m, 2H) 7.38-7.44 (m, 3H) 7.72 (s, 1H) 7.81-7.88 (m, 4H)

### Coupling the Head and Tail Groups

### 4-(4-(4-(1-Benzyl-1H-1,2,3-triazol-4-yl)phenyl)-2-oxopyridin-1(2H)-yl)-2-methyl-2-(methylsulfonyl)-N-((tetrahydro-2H-pyran-2-yl)oxy)butanamide (11)

Pd(dppf)Cl₂—CH₂Cl₂ (10 mol %) was added to a mixture of the head group (8, 0.040 mmol, 1 eq), the aryl boronate tail group (10, 0.060 mmol, 1.5 eq), TBAI (0.004 mmol, 0.1 eq) and K₃PO₄ (0.12 mmol, 3 eq) in 2-Me-THF:H₂O (3:1, 2 mL). The suspension was heated to 64° C. and the reaction was stirred at this temperature until shown to be complete by LC-MS. The reaction was allowed to cool to rt, solids were removed via filtration and the filtrate was concentrated in vacuo. The crude material was purified via flash chromatography 0-10% MeOH/DCM on silica to afford the desired product.

Yield 45.4%, LCMS: m/z calc for C₃₁H₃₅N₅O₆S [M+H]⁺: 606.72; found: 606.20.

¹H NMR (500 MHz, CDCL₃) d ppm 1.59-1.67 (m, 3H) 1.73 (d, J=2.44 Hz, 3H) 1.77-1.85 (m, 1H) 1.87-1.94 (m, 1H) 1.94-2.02 (m, 1H) 2.38-2.48 (m, 1H) 2.51-2.61 (m, 1H) 3.22-3.26 (m, 3H) 3.63-3.73 (m, 1H) 4.06-4.14 (m, 1H) 4.18-4.29 (m, 1H) 4.34-4.43 (m, 1H) 5.24 (br. s., 1H) 5.62 (s, 2H) 6.61 (d, J=7.02 Hz, 1H) 6.89 (s, 1H) 7.33-7.37 (m, 2H) 7.39-7.45 (m, 4H) 7.64 (m, J=8.39 Hz, 2H) 7.75 (s, 1H) 7.92 (m, J=8.24 Hz, 2H) 12.25-12.30 (m, 1H)

### 4-(4-(4-(1-Benzyl-1H-1,2,3-triazol-4-yl)phenyl)-2-oxopyridin-1(2H)-yl)-N-hydroxy-2-methyl-2-(methylsulfonyl)butanamide (PT810, 12)

Hydrochloric acid (4 M in 1,4-dioxane) was added to a solution of the tetrahydropyranyl protected-hydroxamate (1.0 eq) in DCM:MeOH (4:1, 1.0 M) at rt and the reaction was stirred until complete conversion was achieved as monitored by TLC. The desired product was purified by reversed phase HPLC with a C18 column. Chromatography was performed with water as buffer A and acetonitrile as buffer B at flow rate of 4 mL/min with an isocratic method of 30% buffer B. Elution was monitored at 260 nm, and the fractions containing the final product was pooled and lyophilization of the collected fraction gave the final product.

Yield 62%, HRMS: m/z calc for C₂₆H₂₇N₅O₅S [M+H]⁺: 522.1806; found: 522.1808.

¹H NMR (700 MHz, DMSO-d₆) δ ppm 1.59 (s, 3H) 2.18 (m, 1H) 2.44 (m, 1H) 3.12 (s, 3H) 3.76 (m, 1H) 4.11-4.15 (m, 1H) 5.67 (s, 2H) 6.72 (dd, J=7.21, 2.04 Hz, 1H) 6.77 (d, J=1.94 Hz, 1H) 7.35-7.42 (m, 5H) 7.78 (d, J=7.10 Hz, 1H) 7.83 (m, J=8.39 Hz, 2H) 7.97 (m, J=8.39 Hz, 2H) 8.77 (s, 1H) 11.18 (s, 1H)

### Synthesis of PT805

PT805 was synthesized as described in Brown, 2012 and Montgomery, 2012. (Scheme S3).

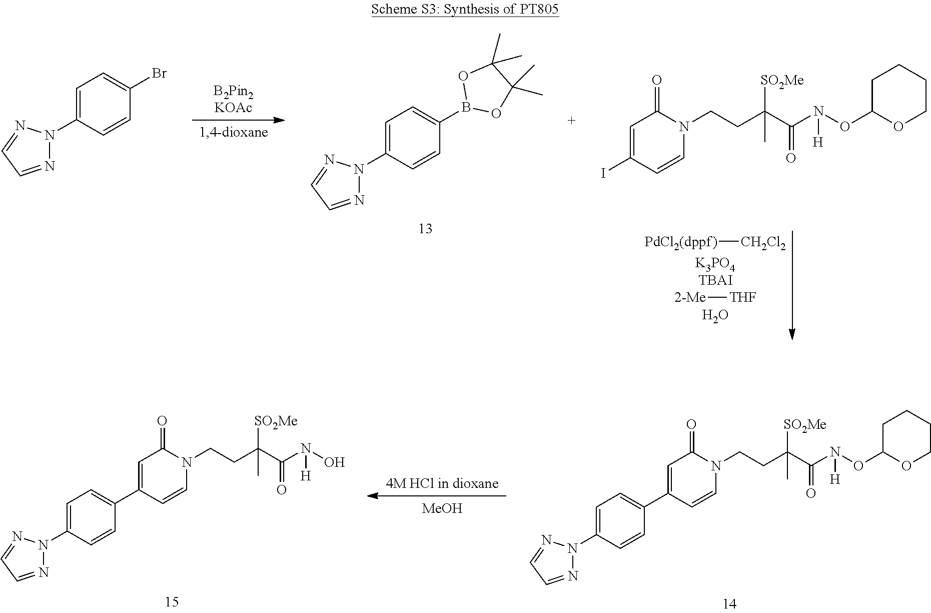

### 2-[4-(4,4,5,5-Tetramethyl-1,3,2-dioxaborolan-2-yl)phenyl]-2H-1,2,3-triazole (13)

Pd(dppf)Cl₂ (106 mg, 0.13 mmol) was added to a mixture of 2-(4-bromophenyl)-2H-1,2,3-triazole (100 mg, 0.45 mmol), 4,4,4′,4′5,5,5′,5′-octamethyl-2,2′-bi-1,3,2-dioxaborolane (134 mg, 0.53 mmol) and potassium acetate (128 mg, 1.3 mmol) in 1,4-dioxane (5 mL). The reaction was heated to 80° C. and stirred at this temperature overnight. The reaction was allowed to cool to rt, diluted with EtOAc (30 mL) and brine (30 mL), filtered through celite after which the organic layer was separated from the filtrate. The aqueous layer was extracted with EtOAc (2×30 mL) and the organics were combined, dried with MgSO₄, filtered and concentrated. The crude material was purified via flash chromatography using EtOAc in hexane (0-50%) to afford the title compound.

Yield-71.0% LCMS: m/z calc for C₁₄H₁₈BN₃O₂ [M+H]⁺: 272.14; found: 272.20.

¹H NMR (500 MHz, CDCL₃) δ ppm 1.39 (s, 12H) 7.85 (s, 2H) 7.95 (m, J=7.93 Hz, 2H) 8.11 (m, J=7.93 Hz, 2H)

### 4-(4-(4-(2H-1,2,3-Triazol-2-yl)phenyl)-2-oxopyridin-1(2H)-yl)-2-methyl-2-(methylsulfonyl)-N-((tetrahydro-2H-pyran-2-yl)oxy)butanamide (14)

Pd(dppf)Cl₂—CH₂Cl₂ (10 mol %) was added to a mixture of the head group (0.040 mmol, 1 eq), the aryl boronate tail group (0.060 mmol, 1.5 eq), TBAI (0.004 mmol, 0.1 eq) and K₃PO₄ (0.12 mmol, 3 eq) in 2-Me-THF:H₂O (3:1, 2 mL). The suspension was heated to 64° C. and the reaction was stirred at this temperature until shown to be complete via LC-MS. The reaction was allowed to cool to rt and solids were removed via filtration. The filtrate was concentrated in vacuo and the crude product was purified via flash chromatography on silica using 0-10% MeOH in DCM to afford the final product.

Yield—49.0%, LCMS: m/z calc for C₂₄H₂₉N₅O₆S [M−H]⁻: 514.58; found: 514.70.

### 4-(4-(4-(2H-1,2,3-Triazol-2-yl)phenyl)-2-oxopyridin-1(2H)-yl)-N-hydroxy-2-methyl-2-(methylsulfonyl)butanamide (15)

Hydrochloric acid (4 M in 1,4-dioxane) was added to a solution of the tetrahydropyranyl protected-hydroxamate (14, 1.0 eq) in DCM:MeOH (4:1, 1.0 M) at rt and the reaction was stirred until complete conversion was achieved as monitored by TLC. The desired product was purified via trituration.

Yield—82%, HRMS: m/z calc for C₁₉H₂₁N₅O₅S [M−H]⁻: 430.1191; found: 430.1182.

¹H NMR (500 MHz, DMSO-d₆) δ ppm 1.58 (s, 3H) 2.15-2.21 (m, 1H) 2.43 (dd, J=11.75, 4.73 Hz, 1H) 3.12 (s, 3H) 3.74-3.81 (m, 1H) 4.11-4.16 (m, 1H) 6.72 (dd, J=7.10, 2.06 Hz, 1H) 6.79 (d, J=2.59 Hz, 1H) 7.80 (d, J=7.17 Hz, 2H) 7.94-7.99 (m, 2H) 8.13 (m, J=8.70 Hz, 2H) 8.19 (s, 1H)

### II. Structure-Kinetic Relationship Studies for the Development of Long Residence Time LPXC Inhibitors

### Experiment 4. Structure-Kinetic Relationship Studies for the Development of Long Residence Time LpxC Inhibitors

In the traditional drug discovery paradigm, thermodynamic parameters such as IC₅₀ and K_{d} values are used extensively to guide compound selection and optimization. However, there is an increasing recognition that drug-target binding kinetics should also be included in drug development, given that the human body is an open thermodynamic system (Swinney, 2004, Copeland, 2006, Vauquelin, 2010, Lu, 2010 and Tonge, 2018). In particular, drugs which dissociate more slowly from their target than the rate of drug elimination may have prolonged activity at low drug concentrations, which would enable dosing frequency to be reduced leading to a widening of the therapeutic window. Given that the rates of formation and breakdown of the drug-target complex may not track with thermodynamic affinity, there is increased emphasis on developing structure-kinetic relationships and understanding the molecular determinants that control the lifetime of the drug-target complex (Tonge, 2018).

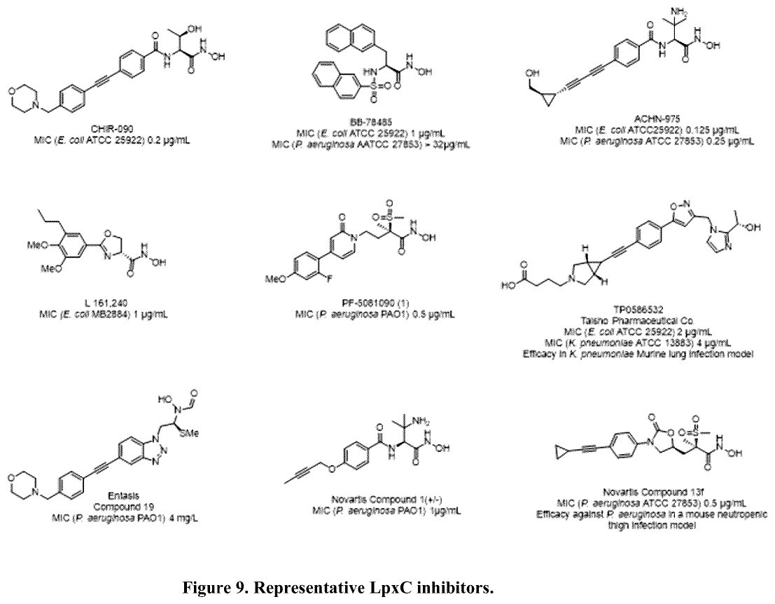

UDP-3-O—(R-3-hydroxymyristoyl)-N-acetylglucosamine deacetylase (LpxC) is a zinc metalloenzyme that catalyzes the first committed step in the biosynthesis of lipid A, a fatty acylated glucosamine disaccharide that anchors lipopolysaccharide (LPS) to the outer membrane of Gram-negative bacteria (Onishi, 1996 and Jackman, 1999). Given that LPS is an essential cell wall component in most Gram-negative pathogens, LpxC is a promising target for antibacterial discovery and many LpxC inhibitors have been developed, the majority of which contain a hydroxamate functional group that chelates the catalytic zinc ion (Celements, 2002, Barb, 2008, Erwin, 2016 and Kalinin, 2017). Some of these compounds are shown in FIG. 9. In addition, several companies including Achaogen, Pfizer, Novartis, Entasis and Taisho Pharma have developed LpxC inhibitors that have in vivo activity in animal models of infection, while the Achaogen compound ACHN-975 was also evaluated in a Phase I clinical trial (Cohen, 0219, Montgomery, 2012, Piizzi, 2017, Lee, 2018, Furuya, 2020). However, despite highly promising in vitro and in vivo activity, no LpxC inhibitor has progressed through clinical trials, and efforts are needed to widen the therapeutic window of LpxC inhibitors. In this regard, compounds with prolonged activity following elimination may be beneficial (Daryaee, 2019).

The inhibition of the LpxC enzyme from Pseudomonas aeruginosa (paLpxC) were studied, and a correlation between residence time and post-antibiotic effect (PAE), which is the delay in bacterial growth following compound washout was demonstrated (Walkup, 2015). The compound series included the pyridone methylsulfone (Compound 1), which has a residence time of 30 min at 37° C. and PAE of 1.26 h at 4×MIC (Walkup, 2015). Compound 1 was evaluated in a mouse model of P. aeruginosa infection, and a PK/PD model that integrates binding kinetics into predictions of in vivo activity was used to accurately predict the efficacy of 1 (Walkup 2015).

Compound 1 lacks the alkyne functionality present in many other LpxC inhibitors that may be linked to safety issues. For example, Pfizer demonstrated that an analog of Compound 1 with an alkyne reacted with glutathione. (Montgomery, 2012) Therefore, this application focused on designing and synthesizing analogs of Compound 1 with longer residence times on paLpxC than Compound 1. The PAE caused by these analogs was evaluated and compounds with residence times up to 124 min and PAE values of 4 h was identified, extending the previous correlation between residence time and PAE. This work lays the foundation for the development of LpxC inhibitors that can be dosed less frequently (Mcclerren, 2005, Walkup, 2015 and Tomaras, 2014)

### Experiment 5. Synthesis of paLpxC Inhibitors

A library of LpxC inhibitors were synthesized in which the head and tail groups were synthesized separately and then coupled via Suzuki coupling to give the final compounds (Jiang, 2019). All the compounds share the methylsulfone head group found in compound 1, except PT903 which is based on L-threonine, and PT920 and PT921 which were previously reported by Novartis and have a cyclic sulfone instead of the methylsulfone (Tables 4 and 5). The methylsulfone head group was synthesized as the racemate as shown in Scheme S1. Briefly, ethyl 2-chloropropionate was refluxed in ethanol with sodium methane sulfinate to synthesize ethyl 2-(methylsulfonyl)propanoate. Alkylation of this compound with 1,2-dibromoethane under basic conditions resulted in the formation of the methyl ester, which was then reacted with 4-iodopyridin-2-ol to provide the corresponding ester. Saponification of the ester resulted in the corresponding acid, which was then protected under standard amide coupling conditions with O-(tetrahydro-2H-pyran-2-yl) hydroxylamine (NH₂OTHP). This common intermediate was then attached to different tail groups using Suzuki coupling, and then deprotected (Scheme 1).

The triazole analog PT805 was synthesized from the appropriate bromophenyl triazole (Scheme S2), while the remainder of the triazole containing tail groups were synthesized by click chemistry from the corresponding boronic acid pinacol ester analogs (PT810 and PT907-PT918, Table 1; PT925, Table 2; Schemes S3, S8-S19, S22). For some of the aliphatic analogs, conversion of the bromine to the corresponding azide followed by a click chemistry reaction was performed in a one-pot reaction. PT901, PT902, and PT904 were synthesized using a Suzuki coupling between the corresponding commercially available boronic esters and the head group followed by HCl deprotection to remove the protecting group (Schemes S4, S5, and S7). Synthesis of PT901 followed the method previously reported by Montgomery et al., and PT903 was synthesized by amide coupling between sodium 4-ethynylbenzoate and L-threonine methyl ester hydrochloride followed by a click chemistry reaction and HCl deprotection (Scheme S6) (Montgomery et al, 2012). PT923 and PT924 were synthesized using Suzuki coupling between the head group and tail groups containing an amide bond (Schemes S20 and S21), while the cyclic sulfone analogs PT920 and PT921 were synthesized as previously described (Fu, 2014).

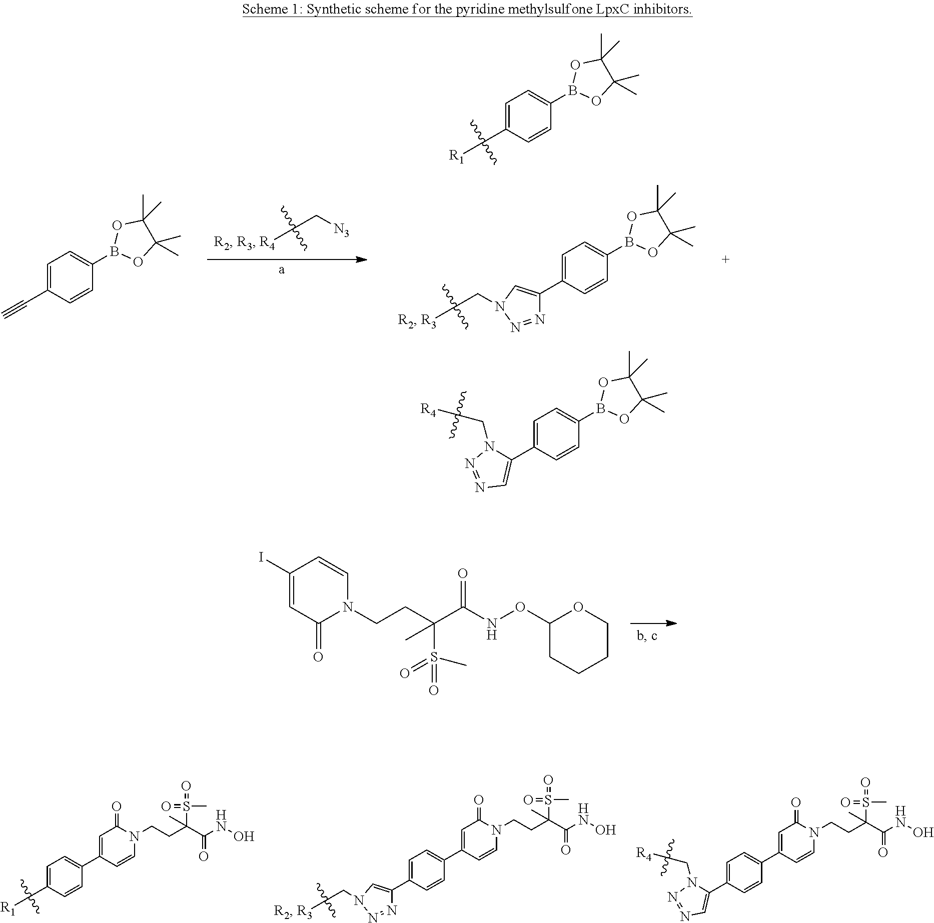

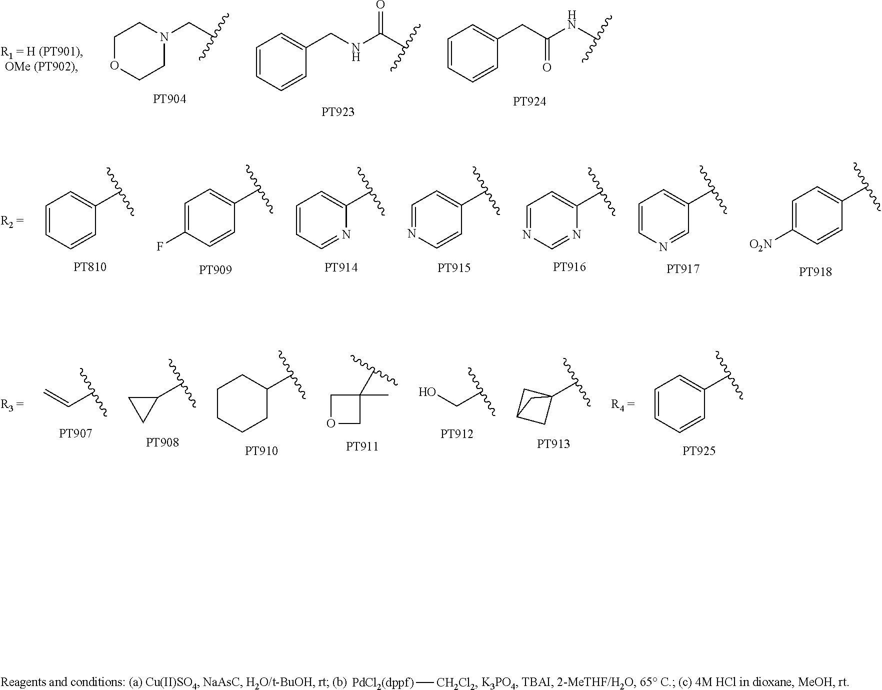

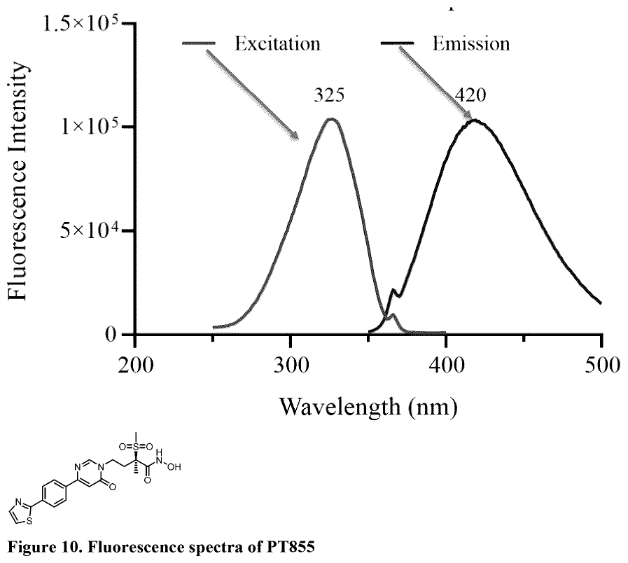

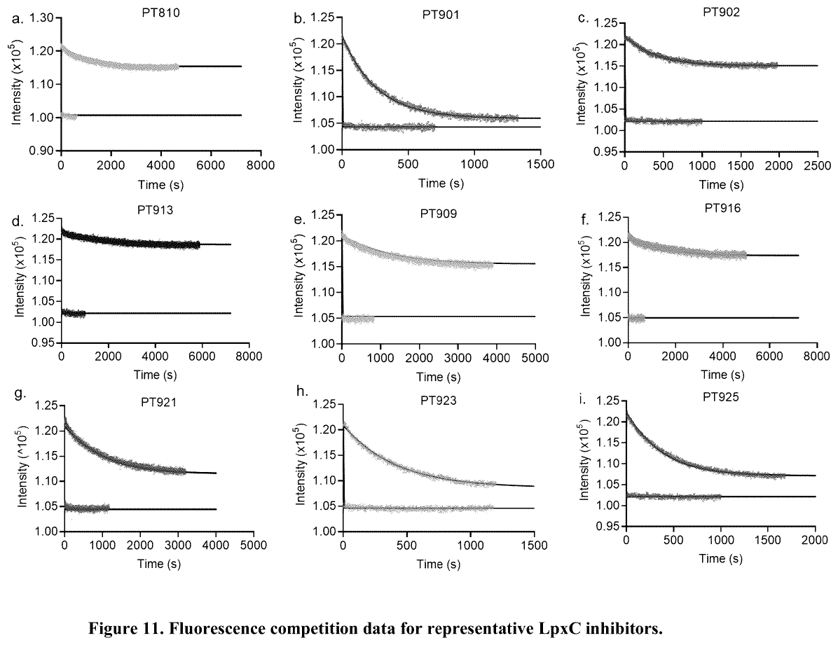

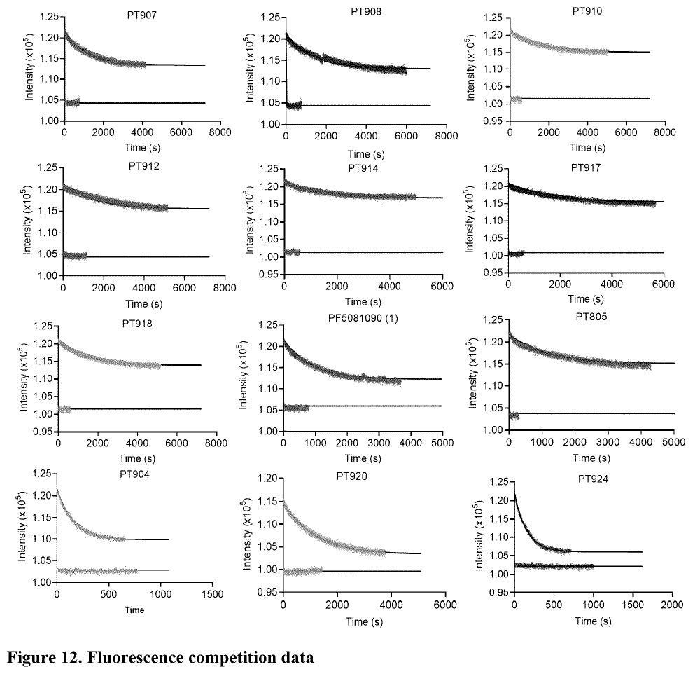

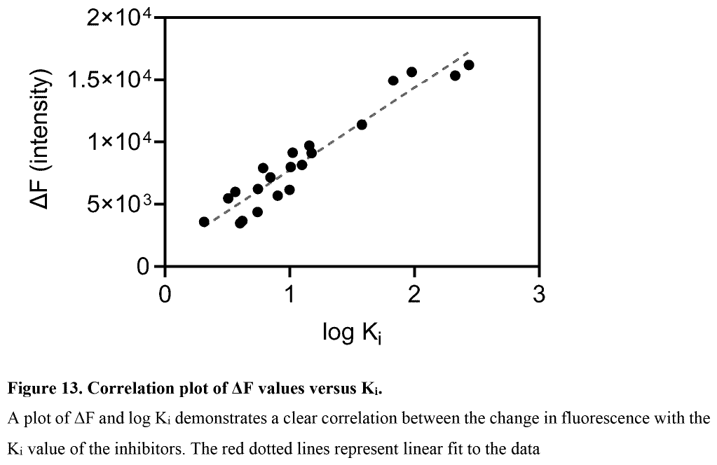

Binding Kinetics. A fluorescence-based competition assay was used to determine the kinetic parameters for binding of the inhibitors to paLpxC. This assay is based on the paLpxC ligand PT855, which fluoresces at 420 nm when excited at 325 nm (FIG. 10), and whose fluorescence is quenched upon binding to paLpxC (Georgi, 2018). Inhibitors (10 μM for enantiomerically pure compounds or 20 μM for racemic mixtures) were incubated with paLpxC (10 μM) for 18 h at 37° C. to ensure complete formation of the final enzyme-inhibitor complex, and then diluted 200-fold into a 200 nM solution of the fluorescent probe (PT855) at 37° C. The rate of dissociation of the enzyme-inhibitor (EI) complex was determined by monitoring the decrease in fluorescence as a function of time as PT855 displaced the inhibitor from the enzyme. Kinetic traces for representative inhibitors with fast, medium, and slow binding kinetics are shown in the FIG. 11, while the remainder are shown in FIG. 12. Compound PT91, which has a short residence time (5.1 min), exhibits a rapid decrease in fluorescence intensity upon dilution of the EI complex into the probe solution. In contrast, long residence time compounds produce a slow decrease in the fluorescence intensity. Binding of both the fluorescent probe and inhibitor were assumed to follow a one-step mechanism, and the overall on (kₒₙ) and off (k_{off}) rates for each inhibitor were extracted by fitting the data in Kintek after constraining kₒₙ and k_{off} values for PT855 (Tables 4 and 5). In each case, the overall change in fluorescence (ΔF) was a function of the relative inhibitor and probe concentrations and their Kᵢ values for paLpxC (FIG. 13). For instance, ΔF for PT901 was ˜98% of the change when free enzyme was added to the solution of the fluorophore since the Kᵢ for PT901 is 213 pM compared to 97 μM for PT855. In contrast, for PT902, ΔF was only 52% of the overall ΔF when free enzyme was added since the Kᵢ value for PT902 is 8-fold smaller than that of PT855.

TABLE 4
Kinetic and microbiological data for the pyridone methylsulfone LpxC inhibitors

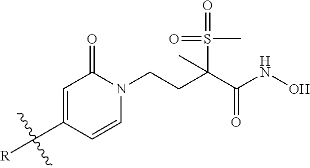

| | | | | | | | t_{R} (min) | |
|---|---|---|---|---|---|---|---|---|
| | | | | | | | Fluorescence | |
| | | | | | | | assay^{d} | |
| | | | MIC | MIC | Kₒₙ | | (Spin | |
| Com- | | | (μM)^{c} | (μM)^{c} | (μM | | column | Kᵢ |
| pound ^{a} | R | clogP^{b} | (PAO1) | (paEP) | ⁻¹s⁻¹)^{d} | K_{off} (s⁻¹)^{d} | assay)^{e} | (pM)^{d} |
| 1 (R) enantiomer | 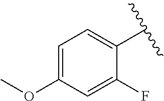 [SMILES: *c1ccc(OC)cc1F] | −0.15 | 0.625 | N.D.^{f} | 25.1 ± 4.1 | 0.00041 ± 0.00004 | 40.5 ± 1.6 (81.0 ± 19.0) | 15 ± 1 |
| PT805 | 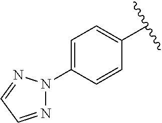 [SMILES: *c1ccc(-n2nccn2)cc1] | −0.57 | 1.56 | 0.4 | 34.6 ± 24.5 | 0.00028 ± 0.00004 | 61.0 ± 9.0 (97.0 ± 22.0) | 10.0 ± 4.1 |
| PT810 | 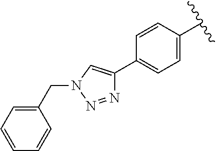 [SMILES: *c1ccc(-c2cn(Cc3ccccc3)nn2)cc1] | 1.59 | 12.5 | 0.78 | 43.8 ± 31.7 | 0.00032 ± 0.00010 | 57.3 ± 18.4 | 10.6 ± 8.9 |
| PT901 | 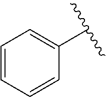 [SMILES: *c1ccccc1] | −0.19 | 3.125 | 0.2 | 18.1 ± 7.9 | 0.0033 ± 0.0001 | 5.1 ± 0.2 (7.3 ± 3.0) | 213.0 ± 111.3 |
| PT902 | 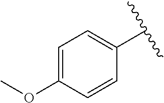 [SMILES: *c1ccc(OC)cc1] | 0.8 | 3.125 | 0.8 | 91.4 ± 17.9 | 0.0011 ± 0.0001 | 15.0 ± 1.5 | 12.6 ± 2.5 |
| PT904 | 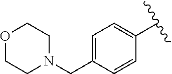 [SMILES: *c1ccc(CN2CCOCC2)cc1] | −1.05 | 50 | N.D.^{f} | 36.4 ± 0.4 | 0.0035 ± 0.0001 | 4.7 ± 0.2 | 96.8 ± 3.0 |
| PT907 | 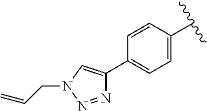 [SMILES: *c1ccc(-c2cn(CC=C)nn2)cc1] | 0.55 | 12.5 | 0.78 | 52.0 ± 29.5 | 0.00022 ± 0.00007 | 82.8 ± 26.0 (146.5 ± 44.0) | 6.1 ± 5.7 |
| PT908 | 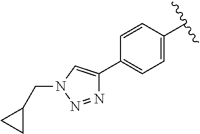 [SMILES: *c1ccc(-c2cn(CC3CC3)nn2)cc1] | 0.58 | 3.125 | 0.4 | 26.4 ± 6.3 | 0.00025 ± 0.00003 | 66.9 ± 8.6 (190.6 ± 92.0) | 10.2 ± 4.3 |
| PT909 | 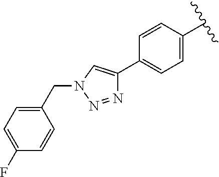 [SMILES: *c1ccc(-c2cn(Cc3ccc(F)cc3)nn2)cc1] | 1.75 | 1.56 | 0.05 | 33.8 ± 13.6 | 0.00025 ± 0.00003 | 68.2 ± 9.6 (106.2 ± 25.0) | 8.0 ± 2.3 |
| PT910 | 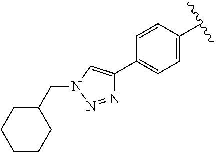 [SMILES: *c1ccc(-c2cn(CC3CCCCC3)nn2)cc1] | 1.83 | 50 | 3.125 | 31.0 ± 3.0 | 0.00017 ± 0.00003 | 100.2 ± 18.5 | 5.5 ± 3.4 |
| PT911 | 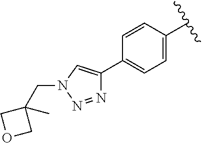 [SMILES: *c1ccc(-c2cn(CC3(C)COC3)nn2)cc1] | 0.13 | 25 | 1.56 | N.D.^{f} | N.D.^{f} | N.D.^{f} | N.D.^{f} |
| PT912 | 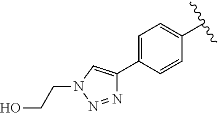 [SMILES: *c1ccc(-c2cn(CCO)nn2)cc1] | −0.66 | >50 | >12.5 | 59.6 ± 22.9 | 0.00018 ± 0.00002 | 92.3 ± 11.2 | 3.7 ± 2.3 |
| PT913 | 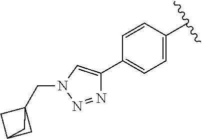 [SMILES: *c1ccc(-c2cn(CC34CC(C3)C4)nn2)cc1] | 0.95 | 12.5 | 0.78 | 85.2 ± 32.5 | 0.00015 ± 0.00005 | 123.8 ± 37.2 | 2.1 ± 1.6 |
| PT914 | 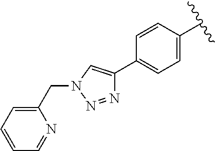 [SMILES: *c1ccc(-c2cn(Cc3ccccn3)nn2)cc1] | 0.68 | 6.25 | 0.2 | 37.2 ± 23.0 | 0.00016 ± 0.00006 | 118.5 ± 44.0 | 5.5 ± 3.4 |
| PT915 | 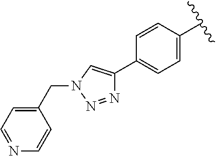 [SMILES: *c1ccc(-c2cn(Cc3ccncc3)nn2)cc1] | 0.25 | 50 | 6.25 | N.D.^{e} | N.D.^{e} | N.D.^{e} | N.D.^{e} |
| PT916 | 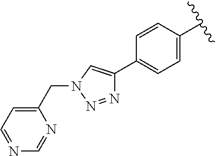 [SMILES: *c1ccc(-c2cn(Cc3ccncn3)nn2)cc1] | 0.08 | 50 | 3.125 | 44.3 ± 14.5 | 0.00017 ± 0.00003 | 102.7 ± 20.2 | 4.2 ± 2.2 |
| PT917 | 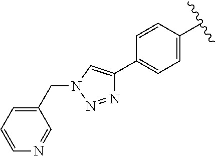 [SMILES: *c1ccc(-c2cn(Cc3cccnc3)nn2)cc1] | 0.35 | N.D.^{e} | N.D.^{e} | 42.8 ± 1.3 | 0.00014 ± 0.00001 | 121.3 ± 4.4 | 3.2 ± 0.2 |
| PT918 | 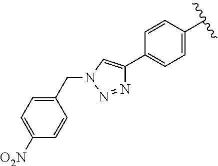 [SMILES: *c1ccc(-c2cn(Cc3ccc([N+](=O)[O-])cc3)nn2)cc1] | 1.62 | 6.25 | 0.1 | 30.7 ± 0.1 | 0.00021 ± 0.00002 | 78.5 ± 8.4 | 7.0 ± 1.0 |
| PT919 | 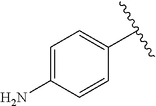 [SMILES: *c1ccc(N)cc1] | 0.79 | 12.5 | 0.78 | 64.27 ± 59.76 | 0.000143 ± 0.00003 | 121.15 ± 30 | 4 ± 3 |
| PT923 | 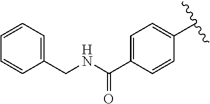 [SMILES: *c1ccc(C(=O)NCc2ccccc2)cc1] | 0.69 | 25 | 3.125 | 23.8 ± 18.8 | 0.0021 ± 0.0003 | 8.0 ± 1.2 | 274.3 ± 76.6 |
| PT924 | 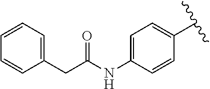 [SMILES: *c1ccc(NC(=O)Cc2ccccc2)cc1] | 0.56 | 50 | 3.125 | 44.8 ± 5.4 | 0.0043 ± 0.0009 | 4.0 ± 1.0 | 95.4 ± 16.2 |
| PT925 | 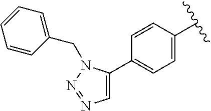 [SMILES: *c1ccc(-c2cnnn2Cc2ccccc2)cc1] | 1.22 | 50 | 12.5 | 27.8 ± 3.2 | 0.00187 ± 0.00018 | 9.0 ± 0.9 | 67.7 ± 10.2 |

^{a} Compound 1 (PF5081090) is the (R) enantiomer. All other compounds were synthesized as racemates.
^{b}clogP values were determined using ChemDraw.
^{c}MIC values were determined against P. aeruginosa PAO1 and an efflux-pump mutant strain (ΔMexABCDXY) (paEP) by the microbroth dilution method. Experiments were performed in triplicate, and the reported values are the average of the three independent measurements.
^{d}kₒₙ, k_{off}, Kᵢ values were determined in triplicate at 37° C. using the fluorescence competition method. t_{R} = 1/k_{off}.
^{e}Residence time (t_{R}) was determined at 37° C. in triplicate by monitoring the direct displacement of inhibitors using the spin column assay.
^{f}N.D., not determined.

TABLE 5
Kinetic and microbiological data for the non pyridone sulfone analogs

| | | | | | | | t_{R} (min) | |
|---|---|---|---|---|---|---|---|---|
| | | | | | | | Fluorescence | |
| | | | | | | | assay^{c} | |
| | | | MIC | MIC | Kₒₙ | | (Spin | |
| | | | (μM)^{b} | (μM)^{b} | (μM | | column | Kᵢ |
| Number | Structure | clogP^{a} | (PAO1) | (paEP) | ⁻¹s⁻¹)^{c} | K_{off} (s⁻¹)^{c} | assay)^{d} | (pM)^{c} |
| PT903 | 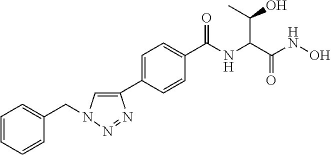 [SMILES: C[C@@H](O)C(NC(=O)c1ccc(-c2cn(Cc3ccccc3)nn2)cc1)C(=O)NO] | 1.69 | >50 | >12.5 | No binding was observed | | | |
| PT920 | 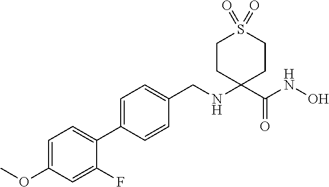 [SMILES: COc1ccc(-c2ccc(CNC3(C(=O)NO)CCS(=O)(=O)CC3)cc2)c(F)c1] | 1.06 | 0.4 | 0.03 | 16.6 ± 8.5 | 0.00053 ± 0.00007 | 32.0 ± 4.8 (79.0 ± 24.0) | 37.9 ± 24.0 |
| PT921 | 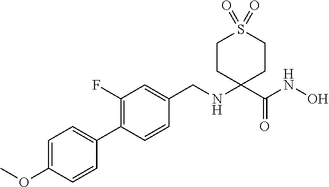 [SMILES: COc1ccc(-c2ccc(CNC3(C(=O)NO)CCS(=O)(=O)CC3)cc2F)cc1] | 1.06 | 0.8 | 0.1 | 34.5 ± 6.3 | 0.00049 ± 0.00001 | 34.3 ± 1.0 | 14.4 ± 2.5 |

^{a}clogP values were determined using ChemDraw.
^{b}MIC values were determined against P. aeruginosa PAO1 and an efflux-pump mutant strain (ΔMexABCDXY) (paEP) by the microbroth dilution method. Experiments were performed in triplicate, and the reported values are the average of the three independent measurements.
^{c}kₒₙ, k_{off}, Kᵢ values were determined in triplicate at 37° C. using the fluorescence competition method. t_{R} = 1/k_{off}.
^{d}Residence time (t_{R}) was determined at 37° C. in triplicate by monitoring the direct displacement of inhibitors using the spin column assay.
^{e}N.D., not determined

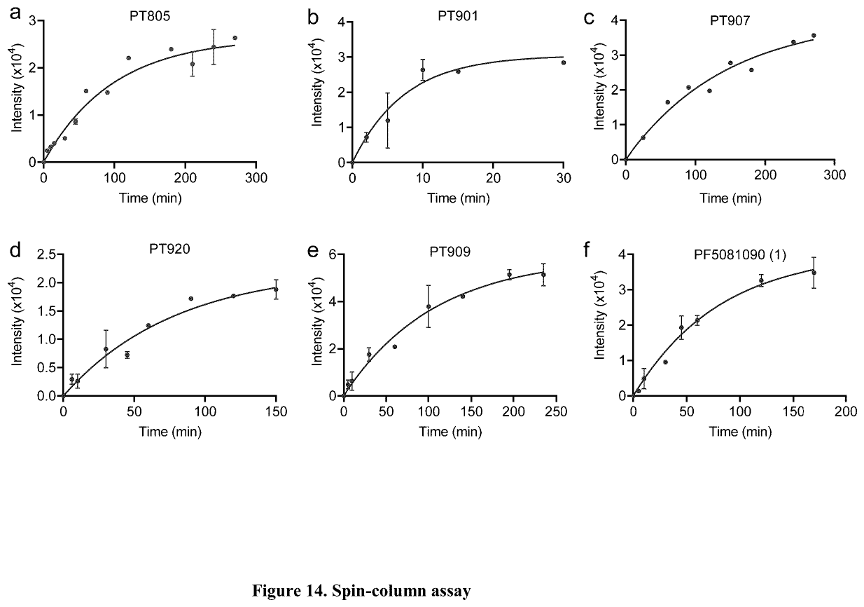

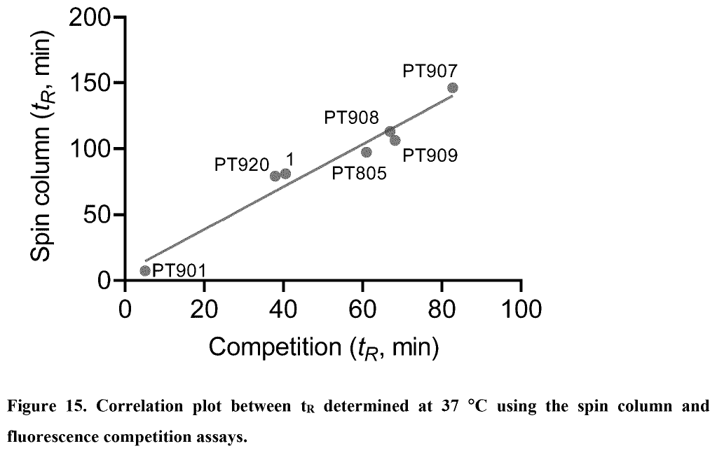

A Penefsky column-based method was used to separately measure k_{off} for each inhibitor (Basu, 2012). Preincubated solutions of enzyme (10 μM) and inhibitor (10 or 20 μM) were diluted 200-fold into a solution of PT855 (1 μM) at 37° C. Aliquots of this solution were withdrawn at various times and subjected to centrifugal gel filtration chromatography using PD SpinTrap™ G-25 columns. The fluorescence of the eluate was quantified, and the data were fit to a single exponential equation to obtain k_{off} (FIG. 14, Tables 4 and 5). The residence times obtained from the spin column assay followed the same trend as those determined using the fluorescence displacement kinetic assay (FIG. 15), although the values from the spin column assay were ˜1.6-fold greater. A similar observation was made for inhibitors of InhA, an enoyl acyl reductase enzyme involved in fatty acid biosynthesis pathway (Yu, 2015).

### Experiment 6 Structural Biology

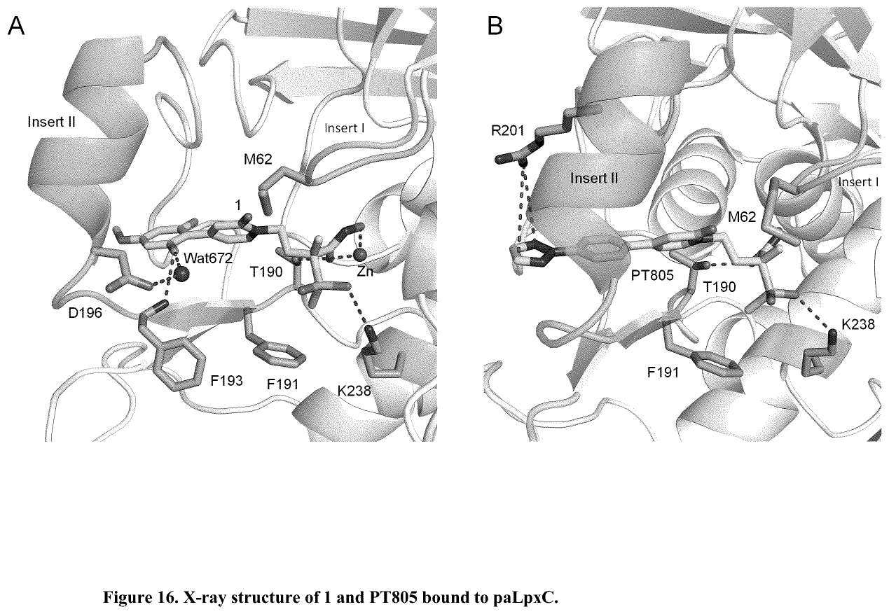

X-ray crystallography was used to determine the structure of paLpxC in complex with inhibitors Compound 1 and PT805. As observed previously in the structure of similar compounds bound to paLpxC, both Compound 1 and PT805 chelate the active site Zn²⁺ through the hydroxamate, which also forms a hydrogen bonding interaction with T190 (FIG. 16). The methyl group positioned at the quaternary center makes a van der Waals contact with M62, while the methyl on the sulfone forms a hydrophobic interaction with F191. In addition, an oxygen atom in the sulfone moiety engages in hydrogen bonding interactions with K238. Although PT805 was synthesized as the racemate, only the (R) configuration of the methylsulfone is present in the structure, consistent with the observation that the (R) enantiomer binds preferentially to the enzyme (Brown, 2012). The phenyl pyridone tail occupies the hydrophobic tunnel of the active site that normally accommodates the C₁₀-C₁₆ aliphatic chain of the substrate (Erwin, 2016). Compound 1 has an additional interaction with F193 and a water molecule (Wat672) through the fluorine substituent, while the triazole ring of PT805 has an additional π-cationic interaction with R201.

As shown in FIG. 16, the structures of (A) Compound 1 (PF5081090) (PDB 5UPG) and (B) PT805 (PDB 6CAX) bound to paLpxC were solved by molecular replacement. Two conformations of Compound 1 are present in the structure, in which the terminal phenyl ring is flipped so that the fluorine occupies two positions (green atoms). In one conformation, the fluorine makes contacts with the side chain amino group of F193 and a water molecule (Wat672), which is also bound to D196. PT805 lacks an ortho-fluoro substituent on the terminal phenyl ring and instead has a triazole ring which is within 6 Å of R201. This is consistent with the presence of a π-cation interaction between enzyme and inhibitor. The head groups of both inhibitors make similar contacts with the enzyme. The methyl group on the chiral center forms a van der Waals interaction with M62, while the methylsulfone interacts with F191 and K238. The hydroxamate is hydrogen bonded to T190 and also chelates the active site zinc ion. Insert I and Insert II are also shown, which are thought to be important for controlling the kinetics of inhibition, since inhibitors with a range of residence times result in enzyme-inhibitor structures in which these structural features occupy different positions.

### Experiment 7 Microbiology and Cytotoxicity

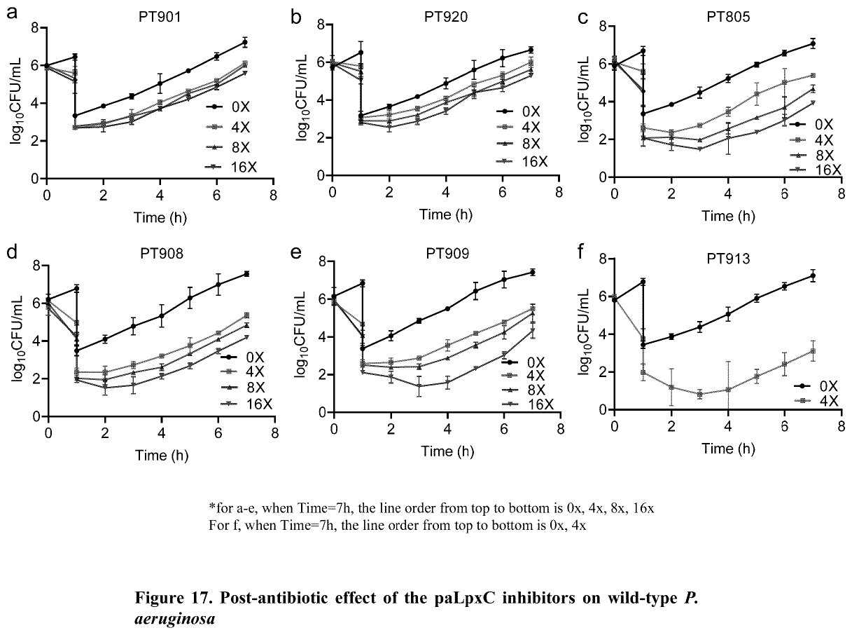

The MIC values for the LpxC inhibitors against a wild-type strain (PAO1) and efflux pump mutant (ΔMexABCDXY) strain of P. aeruginosa (Tables 4 and 5) were measured. The MIC values for all the inhibitors were lower against the efflux pump mutant compared to the wild-type strain, indicating that these compounds are relatively good substrates for efflux pumps. Six paLpxC inhibitors were selected with residence times ranging from 5 min to 125 min and determined the post-antibiotic effect (PAL) caused by the compounds against PAO1 (FIG. 17). Bacterial cultures were exposed to 4×, 8× and 16×MIC of each inhibitor for 1 h at 37° C. followed by 1000-fold dilution into fresh CAMHII media and plating of 100 μL aliquots of the diluted cultures on Muller-Hinton agar plates. After incubating the plates overnight at 37° C., CFUs were enumerated, and the PAE values were calculated using a standard method where the time for the bacteria to recover 1 log post drug washout compared to the control was determined (FIG. 17, Table 6). All the inhibitors exhibited PAEs, and the values increased with increasing concentration of the drugs and as a function of residence time. For instance, the PAL caused by PT901, which is a short residence time compound (t_{R} 5 min), was 0.7 h, whereas the long residence time compound PT913 (t_{R} 124 min) exhibited a much longer PAL of 4 h.

TABLE 6
Residence time and PAE values of the paLpxC inhibitors

| | | MIC | | | |
|---|---|---|---|---|---|
| | | (PAO1)^{a, c} | PAE (h)^{d} | | |
| Number | t_{R} (min)^{b} | (μM) | 4x | 8x | 16x |
| PT805 | 61.0 ± 9.0 | 1.56 | 1.4 ± 0.1 | 2.2 ± 0.3 | 3.4 ± 0.2 |
| PT901 | 5.0 ± 0.2 | 3.125 | 0.7 ± 0.1 | 1.1 ± 0.1 | 1.3 ± 0.1 |
| PT908 | 67.0 ± 8.6 | 3.125 | 1.6 ± 0.1 | 2.2 ± 0.1 | 3.1 ± 0.1 |
| PT909 | 68.2 ± 9.6 | 1.56 | 1.7 ± 0.1 | 2.5 ± 0.1 | 3.5 ± 0.2 |
| PT913 | 124.0 ± 37.2 | 12.5 | 4.0 ± 0.2 | N.D.^{f} | N.D.^{f} |
| PT920 | 32.0 ± 4.8 | 0.4 | 1.0 ± 0.1 | 1.3 ± 0.2 | 1.7 ± 0.2 |

^{a}Wild-type Pseudomonas aeruginosa strain (PAO1).
^{b}Residence times were determined at 37° C. by monitoring the rate of inhibitor displacement using a fluorescent ligand. The reported values are the average of three independent experiments, where the errors represent the standard deviation from the mean.
^{c}MIC values were determined by the microbroth dilution method. Experiments were performed in triplicate, and the reported values are the average of the three independent experiments.
^{d}The PAE was calculated using a standard procedure, in which the time required by the bacteria to recover 1 log CFU after washing out the inhibitor was compared to cultures treated with vehicle (DMSO).³⁴ Experiments were performed in triplicate, and the reported values are the average of the three independent experiments with the errors representing the standard deviation from the mean.
^{e}PAE values for P. aeruginosa were determined at 37° C.
^{f}N.D., not determined.

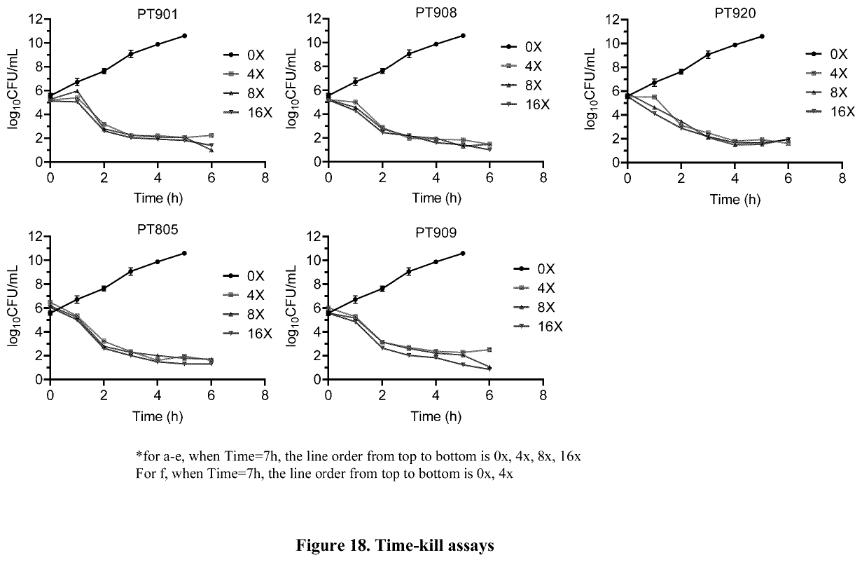

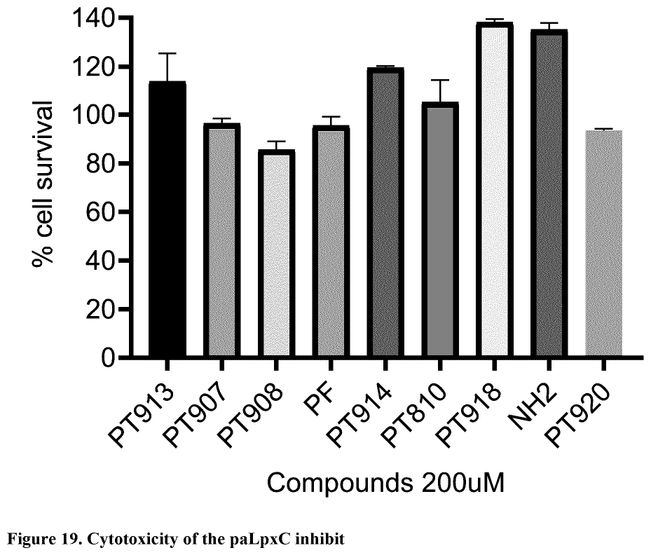

Time-kill assays were performed by exposing cultures of wild-type P. aeruginosa PAO1 (10⁶ CFU/mL) in CAMH media at 37° C. to 4×, 8× and 16×MIC of inhibitor. Samples (100 μL) were taken every hour and plated on Muller-Hinton agar followed by enumeration of CFUs after incubating the plates at 37° C. for 16 h (Basu, 2021). PT901, PT908, PT920, PT805, and PT909 all reduced the starting log CFU/mL of the bacterial culture by more than 3 logs, indicating that they are bactericidal (FIG. 18). The cytotoxicity of the compounds against Vero cells was accessed using an MTT assay, in which cell viability was measured by reading the absorbance of each well at 570 nm (Si, 2019) All the compounds exhibited>80% cell viability at the highest concentration of inhibitor tested (200 μM) (FIG. 19).

### Experimental Detail on Synthesis Scheme

### Methods

Compounds. PF5081090 (1) was purchased from Sigma Aldrich (Saint Louis, Missouri), and PT855 was a gift from Chronus Pharmaceuticals (Stony brook, NY). Synthesis of PT805, P1901, PT902, PT920, and PT921 have been described previously (Montgomery, 2012 and Fu, 2014)

Bacterial Strains. Wild-type P. aeruginosa strain (PAO1) was obtained from ATCC (BAA 1744). The efflux-pump deficient P. aeruginosa strain (ΔMexABCDXY), N150, was derived from PAO1. (Masuda, 2000)

Cloning, Expression, and Purification of paLpxC (used for the enzymatic assays). Genomic DNA was extracted from the wild type PAO1 strain (Promega Inc, Illinois, Chicago) and the lpxC gene (residues 1-299) was amplified from genomic DNA with an annealing temperature of 51.2° C. using the following primers:

Forward
5′GGAATTCCATATGATCAAACAACGCATCATCCGGGCTA3′
Reverse
5′ATGAATTCCTACACTGCCGCCGCCGGGCGCATA3′

The amplified gene was then digested with Nde1 and EcoR1 restriction enzymes and ligated into a pET-24a vector digested with the same restriction enzymes. The correct construct was confirmed by DNA sequencing and protein expression was performed at 18° C. using E. coli B121 (DE3) cells in 2XYT medium containing 50 μg/mL of kanamycin. Following overnight incubation 18° C., bacterial cells from 1 L cultures were harvested by centrifugation at 5000 rpm (4° C.) for 20 min, and the cell pellet was stored at −20° C. Cell pellets were resuspended in 40 mL of lysis buffer (10 mM NaH₂PO₄ buffer pH 7.0, containing 10 mM MgCl₂ and 0.1 mM ZnCl₂) and lysed by sonication. The cell debris was removed by ultracentrifugation (40,000×g, Ti45 rotor) for 1 h at 4° C.), and paLpxC was purified using a previously published method (Mochalkin, 2008). Protein was precipitated from the supernatant using (NH₄)₂SO₄ (50% saturation), and the precipitate was recovered by centrifugation at 40,000×g for 1 h at 4° C. The protein pellet was resuspended in 10-20 mL of buffer A (10 mM NaH₂PO₄ pH 7.0) and desalted using a HiPrep 26/10 desalting column (GE). The eluate was then loaded onto a HiTrap™ Q FF (14.7×29.8 mm) anion-exchange column (GE) which was washed with 100 mL of buffer A containing 10-20 mM of NaCl. Chromatography was performed using a gradient of 10-500 mM NaCl, and fractions containing paLpxC (150-200 mM NaCl) were pooled and concentrated to a volume of 10 mL using a 10 kDa MWCO concentrator (Amicon, Miami, Florida). The concentrated protein solution was loaded onto a HiLoad 16/600 Superdex-200 column equilibrated with 25 mM HEPES buffer pH 7.0 containing 2 mM DTT and 50 mM NaCl. Fractions containing paLpxC were analyzed for purity using SDS-PAGE, flash-frozen with liquid N₂, and stored at −80° C.

Cloning, Expression and Purification of His-tagged paLpxC (used for crystallization) paLpxC (CID12015, SSGCID ID PsaeA.00166.a.DG15, corresponding to UniProt P47205 residues 1-304) was codon-engineered for E. coli expression in-silico using GeneComposer™ and further optimized to balance GC content, exclude cryptic Shine Dalgarno sequences and exclude BamHI and HindIII restriction sites. The resulting gene insert nucleotide sequence was flanked with 5′ GGATCC (BamHI) and 3′ TGATAAGCTT (HindIII) restriction sites such that two stop codons followed the last codon of the Open Reading Frame (ORF). The final gene insert was then synthesized by ATUM (formerly DNA 2.0) and delivered in a shuttle vector. The gene insert was then digested with BamHI and HindIII and subcloned to BamHI/HindIII-digested vector pEMB54, which is an ampicillin resistant, arabinose-inducible vector with pMB1 origin of replication and 6×His-Smt3 under the PBAD (araBp) promoter, including a multiple cloning site containing BamHI and HindIII sites following the Smt3 sequence. Genes cloned into pEMB54 via BamHI/HindIII are fused in-frame with 6×His-Smt3 leaving a serine linker following the C-terminal double glycine of Smt3. Following digestion, the vector and insert fragments were gel-purified, the insert ligated into the vector and the ligation transformed into competent E. coli TOP10 cells. One transformant was miniprepped and submitted for Sanger DNA sequencing of the ORF and flanking regions, which was subsequently confirmed to be 100% correct. Cells were grown in large-scale quantities in Terrific Broth media supplemented with 50 g/ml ampicillin. Protein expression was induced with 0.1% arabinose and 0.1 mM ZnCl₂ when the OD₆₀₀ reached values between 0.5 and 0.7. Following growth overnight at 25° C., cells were harvested by centrifugation at 7,500×g for 20 min at 4° C. and stored at −80° C.

Cell pellets were resuspended in 25 mM Tris pH 8.0 buffer containing 300 mM NaCl, 5% glycerol, 0.02% CHAPS, 5 mM imidazole, 1 mM tris(2-carboxyethyl) phosphine (TCEP), 50 mM Arg, 100 mg lysozyme, 250 U benzonase, and supplemented with EDTA-free protease inhibitor cocktail (Sigma-Aldrich). Cells were lysed by sonication using 5 cycles in pulsed mode with 1 min rest on ice between cycles. Each cycle consisted of thirty 2 second (s)-pulses at 70% amplitude and cooling for 2 s between pulses. The lysate was subsequently centrifuged at 42,000×g for 35 min at 4° C. to remove unbroken cells and cell debris. The supernatant was then filtered through 0.2 m filters and loaded onto a HiTrap™ Ni column previously equilibrated with equilibration buffer (EB) consisting of 25 mM Tris pH 8.0, 200 mM NaCl, 5 mM imidazole, 50 mM Arg, 1 mM TCEP and 0.25% glycerol. After collection of the flow through, the column was washed with 10 column volumes of EB. paLpxC was finally eluted using a linear gradient of imidazole between 5 and 500 mM in EB. Fractions containing paLpxC were combined using a 3.5 kDa cut-off dialysis cassette and cleaved with Ulp1 for 4 h and then dialyzed overnight at 4° C. in 25 mM Tris pH 8 buffer containing 200 mM NaCl and 1 mM TCEP. The cleaved protein was loaded onto a HiTrap™ Ni column, and the same buffers were used for washing (10 column volumes) and elution (12 column volumes). Fractions containing paLpxC were pooled, concentrated 10-fold using a 10 kDa Vivaspin PES Turbo ultrafiltration unit (4000 RCF, 10 min). The protein was further purified by size exclusion chromatography (SEC) using a HiPrep™ 26/60 Sephacryl™ S-100 HR column (GE Healthcare, Illinois, Chicago) previously equilibrated with 25 mM HEPES pH 7.5 buffer, containing 150 mM NaCl and 2 mM TCEP. Fractions containing paLpxC were pooled, concentrated as described above, flash-frozen using liquid N₂, and stored at −80° C. All purification steps were conducted at 4° C. using an AKTA fast protein liquid chromatography (FPLC) system (GE Healthcare). Protein purity was assessed by SDS-PAGE.

Crystallization and Structure Determination. Automated, high-throughput crystallization experiments were performed with ˜5 mg/mL paLpxC that had been diluted with 25 mM HEPES pH 8.0 buffer containing 150 mM NaCl and 2 mM TCEP and then incubated for 2 h with 1 mM ZnCl₂ and 1 mM inhibitor. The optimized crystallization conditions involved paLpxC mixed in a 1:1 ratio with a Molecular Dimensions Wizard Precipitant Synergy 2 (MD15-PS—B) E1 precipitant solution composed of 100 mM Hepes pH 7.7 buffer containing 50 mM CaCl₂), 4% (w/v) propanol, 25% (w/v) PEG 3350. Crystallization was performed with sitting drop vapor diffusion at 20° C. using the seeding technique, and crystals grew in 7-15 days reaching sizes between 50 and 100 μm in their longest dimension. Crystals of the paLpxC-inhibitor complexes were cryo-protected using 20% ethylene glycol in the crystallization buffer.

Data sets were collected in-house with a Rigaku FR-E+ detector at a wavelength of 1.54 Å or at beamline 21-ID-F at the Advanced Photon Source (APS), with a RAYONIX MX-300 detector at a wavelength of 0.98 Å, under a stream of nitrogen (100 K). Data were indexed and integrated with XDS/XSCALE, and the structure was solved by molecular replacement with MOLREP as implemented in MoRDa using the structure of paLpxC (PDB 3UHM) as the search model. Iterative rounds of manual rebuilding and refinement were performed using COOT, and PHENIX, and structures were examined, validated, and improved using MolProbity (Kabsch, 2010, Emsley, 2010, Adams, 2010 and Chen, 2010). The final structures of paLpxC in complex with 1 (PF5081090) and PT805 were solved at a resolution of 1.7 Å and 1.25 Å, respectively. The crystallographic data collection and structure refinement statistics are summarized in Table S1.

Inhibitor Binding Kinetics Determined using a Fluorescence Competition Assay. The kinetic parameters for each inhibitor were determined using a competition assay based on the fluorescent paLpxC inhibitor PT855 (λₑₓ 325 nm, λₑₘ 420 nm), whose fluorescence is quenched upon binding to paLpxC. Enzyme-inhibitor (EI) complexes were formed by incubating 10 μM paLpxC with 10 μM (enantiomerically pure) or 20 μM (racemic) inhibitor in 50 μL of 25 mM NaH₂PO₄ buffer pH 8.0 containing 300 mM KCl and 2% DMSO (v/v) at 37° C. for 18 h to ensure complete formation of the EI complex. Subsequently, the enzyme-inhibitor complex was diluted 200-fold into 1 mL of a 200 nM solution of PT855, and the change in fluorescence intensity was monitored as a function of time at 37° C. A control experiment in which the enzyme was incubated with DMSO was used to determine the maximum change in fluorescence caused by the addition of 50 nM enzyme to the solution of PT855.

Kinetic parameters were extracted by fitting the change in fluorescence to a model that assumes there are two simultaneous one-step binding equilibria shown in Scheme 2, where E is paLpxC, I is the non-fluorescent inhibitor, F is the fluorescent competitor PT855, and kₒₙ(F), kₒₙ(I), k_{off}(F), and k_{off}(I) are the on and off rates for formation and breakdown of the respective EF and EI complexes.

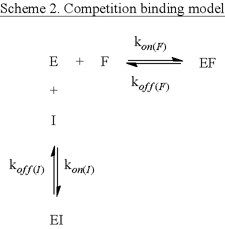

KinTek Explorer was used to fit the time-dependent change in fluorescence (fₜ) to the model shown in Scheme 2 using Equation 1, where f₁ and f₂ are the scaling factors that relate the fluorescence intensity of PT855 and the E-PT855 complex to the concentrations of these species at time t ([F]ₜ and [EF]ₜ, respectively) (Johnson, 2009).

$${f}_{t} = {f}_{1} * {[ F ]}_{t} + {f}_{2} * {[ E F ]}_{t} \qquad 1$$

To estimate kₒₙ and k_{off} of PT855, the experiment was first performed with 1, which the kinetic and thermodynamic parameters have been previously reported (Walkup, 2015). The parameters for 1 were fixed, and the fitting of the model in KinTek provided the on and off rates for binding of PT855 to paLpxC. The kinetic parameters of the competitor were then locked and used to determine the on and off rates of each inhibitor whilst also keeping f₁ and f₂ constant. Subsequently, the thermodynamic equilibrium constant Kᵢ and inhibitor residence time t_{R} were calculated using the relationships Kᵢ=k_{off}/kₒₙ and t_{R}=1/k_{off}, respectively.

Inhibitor Residence Time Measurements using a Spin Column Assay. The residence time of the drug-target complex was measured using a Penefsky column-based direct displacement method as described previously for ecLpxC.¹¹ Briefly, 50 μL of a solution containing 10 μM of paLpxC and 10 μM of enantiomerically pure inhibitor or 20 μM of racemic inhibitor in 25 mM NaH₂PO₄ buffer pH 8.0 containing 300 mM KCl and 0.1 mg/mL BSA was incubated for 18 h at 37° C. to ensure complete formation of the final enzyme-inhibitor complex (EI). Subsequently, 10 μL of the incubation mixture was diluted into 2 mL of the same buffer containing 1 μM of PT855, and 100 μL of this mixture was then aliquoted at different time points and loaded onto a PD SpinTrap™ G-25 column (Cytiva) that had been equilibrated with buffer prior to sample loading. The columns were then centrifuged in a swinging bucket rotor (Eppendorf 5810R, 15-amp version) at 800*g for 2 min, and 100 μL of the flow-through was dispensed into a 96 well plate for measuring the fluorescence intensity in a plate reader using λₑₓ 325 nm and λₑₘ 420 nm. The fluorescence intensities corresponding to loading diluted EI without the fluorescent competitor and loading the competitor alone were measured separately to determine the dynamic range and endpoint of the assay. The large excess of competitor over [EI] eliminated the rebinding of the inhibitor to the enzyme so that the rate of formation of the E-PT855 directly gives the off rate of the inhibitor from the enzyme. Rates were calculated by fitting the change in fluorescence intensity over time to a one-phase association equation in GraphPad Prism.

Minimum Inhibitory Concentration (MIC) Measurements. MIC values were determined in triplicate against wild-type (PAO1) and efflux pump mutant strains of P. aeruginosa using the microbroth dilution assay following the Clinical and Laboratory Standard Institute guidelines.¹² Overnight bacterial cultures were used to inoculate fresh cation-adjusted Mueller-Hinton (CAMH) medium and allowed to grow to the mid-log phase (OD₆₀₀ 0.6) at 37° C. in an orbital shaker. A final inoculum concentration of 10⁶ CFU/mL per well was added to medium in transparent 96-well plates containing 2-fold dilutions of inhibitors to give final concentrations ranging from 0.05 μM to 50 μM. The MIC was defined as the minimum concentration of inhibitor at which no visible growth could be detected after 24 h of incubation at 37° C.

Post-antibiotic Effect (PAE). PAEs were determined using P. aeruginosa PAO1. Overnight bacterial cultures were used to inoculate fresh cation-adjusted Mueller-Hinton II medium (CAMH-II) and allowed to grow to the mid-log phase (OD₆₀₀ 0.6) at 37° C. in an orbital shaker. Subsequently, the culture was diluted 100-fold into fresh medium containing 0×, 4×, 8×, 16×MIC of the compound and shaken for 1 h at 37° C. The compound was washed out by diluting the culture 1000-fold into fresh CAMH-II medium, and the regrowth of bacteria was monitored by taking 100 μL aliquots each hour and plating serial dilutions on Muller-Hinton agar plates. CFUs were enumerated after overnight incubation of the plates at 37° C. The PAE was calculated as the time required for the antibiotic-treated cell population to increase 1 log₁₀ CFU minus the time needed for the control population to increase by 1 log₁₀ CFU.¹³ Experiments were performed in triplicate, and the reported values are the average of the three independent experiments with the errors representing the standard deviation from the mean.

Time-Kill Assays. Cultures of P. aeruginosa PAO1 were prepared in CAMH-II medium and grown to mid-log phase (OD₆₀₀ 0.6) in a shaker at 37° C. Cultures were then diluted 100-fold into fresh medium containing 0, 4×, 8×, or 16×MIC of the inhibitors or DMSO vehicle. Cultures were shaken at 37° C. for 6 h, and 100 μL aliquots were taken at each 1 h time interval and plated in serial dilutions in a Mueller-Hinton agar plate. Kill curves were obtained by counting the colonies after overnight incubation of the plates at 37° C. Bactericidal activity was defined as a reduction in CFUs of 3 log₁₀ CFU/mL within the first 3 h (Basu, 2021).

Cytotoxicity. Compound cytotoxicity was determined using Vero cells (ATCC CCL-81) (Si, 2019). Cell cultures were grown in DMEM supplemented with 10% fetal bovine serum and aliquoted into a 96-well plate to 2×10⁴ cells/well. After 24 h incubation at 37° C. in 5% CO₂, the medium was replaced with serum-free DMEM. Subsequently, the plates were incubated for an additional hour followed by the addition of 0.2 to 200 μM final concentration of inhibitors. An equal volume of the vehicle (DMSO) was added as a control. Following incubation for 24 h at 37° C. in 5% CO₂, cell viability was assessed by means of an MTT assay (Vybrant MTT Cell Proliferation Assay Kit). The absorbance of each well was measured at 570 nm, and the data were used to determine compound cytotoxicity.

TABLE 6
Crystallographic data collection and refinement statistics

| | LpxC with inhibitor 1 | | LpxC with inhibitor | |
|---|---|---|---|---|
| | (PF5081090) | | PT805 | |
| Data collection statistics | | | | |
| Radiation source | Home source | | APS 21-ID-F | |
| Wavelength (Å) | 1.54 | | 0.98 | |
| Detector | Rigaku FR-E+ | | RAYONIX MX300HE | |
| Temperature (K) | 100 | | 100 | |
| Space group | P1 | | P1 | |
| Unit cell dimensions | 35.84 47.40 48.15 | | 35.76 47.47 48.23 | |
| abc (Å) | (111.27 109.12 98.12) | | (111.26 109.17 97.9) | |
| Resolution range (Å) | 1.70-40.91 | (1.70-1.74) | 50-1.25 | (1.28-1.25) |
| 1/δ(I) | 18.42 | (2.38) | 8.59 | (2.18) |
| Multiplicity | 7.24 | (3.28) | 2.64 | (2.66) |
| Completeness (%) | 97.7 | (88.6) | 94.1 | (91.4) |
| CC_{1/2} (%) | 99.8 | (73.3) | 99.6 | (72.6) |
| R_{merge} (%) | 8.4 | (53.9) | 7.3 | (48.0) |

| Total number reflections | 208,431 | 184,550 |
|---|---|---|
| No. of unique reflections | 28,780 | 69,777 |
| Data refinement statistics | | |
| Number of reflections | 28,779 | 69,774 |
| used in refinement | | |
| Number of free reflections used in | 2,043 | 1,951 |
| refinement | | |
| R_{work}/R_{free} (%) | 14.7/18.6 | 16.0/17.1 |
| Wilson B-factor (Å²) | 9.93 | 8.36 |
| Solvent content (%)/Matthews | 40.05/2.05 | 40.05/2.05 |
| coefficient (Å³/Dalton) | | |
| Overall B factor (Å²) | 13.34 | 14.09 |
| RMS deviations from ideal values | | |
| Bonds lengths (Å) | 0.006 | 0.012 |
| Bond angles (°) | 0.848 | 1.38 |
| Ramachandran plot statistics (%) | | |
| Favored | 97.32 | 97.32 |
| Outliers | 0 | 0 |
| Molprobity score/percentile | 1.18/99^{th} | 1.28/99^{th} |
| PDB | 5UPG | 6CAX |

Values in parentheses are for the highest-resolution shell.

### Synthesis of LpxC Inhibitors

### Synthesis of the Head Group

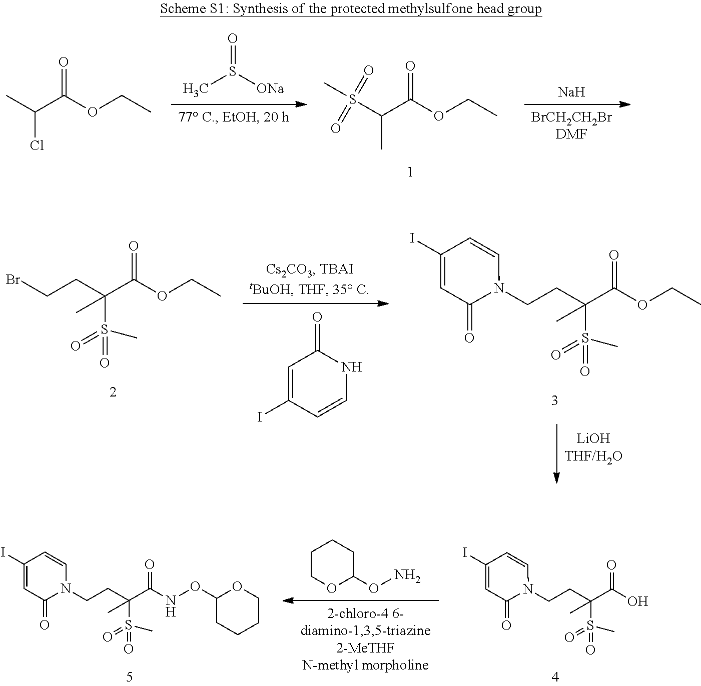

### Ethyl 2-(methylsulfonyl)propanoate (1)

Sodium methanesulfinate (3.9 g, 38.4 mmol) was added to ethyl 2-chloropropionate (5 g, 892 mmol) in EtOH (16 mL). The reaction mixture was heated to 77° C. and stirred at this temperature for 20 h. The reaction mixture was allowed to cool to rt, and the solids were removed by filtration and washed with ethanol. The filtrates were combined and concentrated in vacuo. The crude product was suspended in Et₂O (25 mL), and the solids were removed by filtration. The filtrate was concentrated in vacuo to afford the title compound as a pale-yellow oil. Yield 67%

LCMS: m/z calc for C₆H₁₃O₄S [M+H]⁺: 181.23; found: 181.10.

¹H NMR (700 MHz, CDCl₃) δ ppm 1.30 (t, J=7.10 Hz, 3H), 1.63 (d, J=7.31 Hz, 3H), 3.03 (s, 3H), 3.88 (q, J=7.17 Hz, 1H), 4.23-4.29 (m, 2H).

### Ethyl 4-bromo-2-methyl-2-(methylsulfonyl)butanoate (2)

Sodium hydride (60% dispersion in mineral oil, 1.076 g, 44.83 mmol) was washed with hexanes (2×10 mL) under nitrogen and then suspended in N,N-dimethylformamide (15 mL). The suspension was treated dropwise with ethyl 2-(methylsulfonyl)propanoate (4, 4.04 g, 22.41 mmol) in DMF (5 mL). The mixture was stirred for 30 min at rt, cooled to 0° C., and treated drop-wise with 1,2-dibromoethane (1.9 mL, 22.41 mmol). The mixture was allowed to warm to rt and stirred overnight, after which the reaction was quenched with saturated NH₄Cl (50 mL) and extracted with Et₂O (4×25 mL). The organic layers were combined and then washed with water:brine (1:1, 4×25 mL), dried with MgSO₄, and concentrated in vacuo. The crude material was purified via silica chromatography with a gradient of 10-20% EtOAc in hexanes to afford the title compound as a pale-yellow oil. Yield 42%

LCMS: m/z calc for C₈H₁₆BrO₄S [M+H]⁺: 288.18; found: 288.10.

¹H NMR (700 MHz, CDCL₃) δ ppm 1.36 (t, J=7.21 Hz, 3H), 1.67 (s, 3H), 2.57 (ddd, J=14.04, 9.84, 4.73 Hz, 1H), 2.78-2.84 (m, 1H), 3.07 (s, 3H), 3.39 (td, J=10.11, 6.67 Hz, 1H), 3.52 (td, J=10.11, 4.73 Hz, 1H), 4.32 (q, J=7.17 Hz, 2H).

### Ethyl 4-(4-iodo-2-oxopyridin-1(2H)-yl)-2-methyl-2-(methylsulfonyl)butanoate (3)

A solution of ethyl 4-bromo-2-methyl-2-(methylsulfonyl)butanoate (780 mg, 2.72 mmol) in THE (9 mL) was added to a suspension of 4-iodopyridin-2-ol (428.7 mg, 1.94 mmol), Cs₂CO₃ (694 mg, 2.13 mmol), and tetra-n-butylammonium iodide (TBAI) (71.6 mg, 0.194 mmol) in t-BuOH (9 mL). The suspension was heated to 35° C. and stirred for 3 days. The reaction mixture was allowed to cool to rt and then filtered. The filter cake was washed with EtOAc, and the filtrates were washed with H₂O (100 mL). The aqueous layer was extracted with EtOAc (3×100 mL), and the organic layers were combined, dried with MgSO₄, filtered, and concentrated in vacuo. The crude material was purified via flash chromatography using a gradient of 30-100% EtOAc in hexane to afford the title compound.

Yield 82%.

LCMS: m/z calc for C₁₃H₁₉INO₅S [M+H]⁺: 428.26; found: 428.00.

¹H NMR (400 MHz, CDCl₃) δ ppm 1.35 (t, J=7.09 Hz, 3H), 1.75 (s, 3H), 2.41-2.55 (m, 2H), 3.11 (s, 3H), 3.91-3.99 (m, 1H), 4.16-4.24 (m, 1H), 4.25-4.31 (m, 2H), 6.52 (dd, J=7.09, 1.59 Hz, 1H), 7.00 (d, J=7.21 Hz, 1H), 7.10 (d, J=1.47 Hz, 1H).

### 4-(4-iodo-2-oxopyridin-1(2H)-yl)-2-methyl-2-(methylsulfonyl)butanoic acid (4)

Lithium hydroxide (6 mmol) was added to a solution of the ester (1.0 mmol) in THF:MeOH:H₂O (2:1:1, 1 M) at rt, and the mixture was stirred until complete conversion was achieved as monitored by TLC. The pH of the reaction mixture was adjusted to 3 using aq 1 N HCl and the reaction mixture was extracted with EtOAc. The organic layers were combined, dried, filtered, and concentrated in vacuo to afford the desired product without further purification.

LCMS: m/z calc for C₁₁H₁₃INO₅S [M−H]⁻: 398.19; found: 398.00.

¹H NMR (700 MHz, DMSO-d₆) δ ppm 1.54 (s, 3H), 2.13 (ddd, J=13.34, 10.33, 4.95 Hz, 1H), 2.40 (ddd, J=13.18, 10.38, 5.92 Hz, 1H), 3.15 (s, 3H), 3.87 (ddd, J=12.69, 10.54, 5.81 Hz, 1H), 3.97-4.03 (m, 1H), 6.60 (dd, J=6.99, 1.83 Hz, 1H), 6.92 (d, J=1.51 Hz, 1H), 7.47 (d, J=7.10 Hz, 1H).

### 4-(4-iodo-2-oxopyridin-1(2H)-yl)-2-methyl-2-(methylsulfonyl)-N-((tetrahydro-2H-pyran-2-yl)oxy)butanamide (5)

N-Methyl morpholine (1.6 eq) was added to a solution of 2-chloro-4,6-diamino-1,3,5-triazine (1.4 eq) and the carboxylic acid (1.0 eq) in 2-methyl THE (1 M), and the reaction mixture was stirred for 1 h. O-(Tetrahydro-2H-pyran-2-yl)hydroxylamine (1.6 eq) was added to the reaction mixture which was then stirred overnight at rt. The crude reaction was filtered and the filter pad was washed with DCM. The combined filtrates were concentrated in vacuo, and the crude material was purified via flash chromatography using 0-10% MeOH/DCM on silica to afford the desired product. Yield 35%.

LCMS: m/z calc for C₁₆H₂₂IN₂O₆S [M−H]⁻: 497.32; found: 497.00.

¹H NMR (500 MHz, CDCl₃) δ ppm 1.61-1.65 (m, 3H), 1.71 (m, 3H), 1.81 (dt, J=12.70, 3.72 Hz, 1H), 1.86-1.91 (m, 1H), 1.92-1.97 (m, 1H), 2.31-2.39 (m, 1H), 2.44-2.53 (m, 1H), 3.20-3.22 (m, 3H), 3.60-3.70 (m, 1H), 4.00 (dddd, J=13.85, 11.10, 5.95, 3.13 Hz, 1H), 4.12-4.20 (m, 1H), 4.27-4.34 (m, 1H), 5.14-5.20 (m, 1H), 6.63 (dd, J=7.02, 1.68 Hz, 1H), 7.05 (d, J=7.02 Hz, 1H), 7.18 (d, J=1.68 Hz, 1H), 11.69-11.74 (br.s, 1H).

### Synthesis of the Tail Group (Described in Method A and B)

### Method A

### A1. Conversion of Aryl Bromide to Aryl Azide

Aryl bromide (1 eq) was dissolved in 4:1 acetone: H₂O (10 mL), and sodium azide (1.9 eq) was added. The reaction was stirred overnight at room temperature (rt) and then extracted with Et2O (3×50 mL) after which the organic phase was washed with H₂O (50 mL) and brine (50 mL). The organic phase was dried with MgSO₄ and filtered, and the solvent was removed in vacuo to yield the desired product without further purification.

### A2. Conversion of Aryl Azide to Aryl Boronic Pinacol Ester

2-(4-Ethynylphenyl)-4,4,5,5-tetramethyl-1,3,2-dioxaborolane (1.05 mmol) and benzyl azide (1.05 mmol) were suspended in 10 mL of a 1:1 H2O:t-BuOH mixture. Sodium ascorbate (1.05 mmol) was added followed by copper (II) sulfate pentahydrate (1.05 mmol).5 The heterogeneous mixture was stirred vigorously overnight. The reaction mixture was then extracted with 3×50 mL DCM and water, and the organic phase was washed with H₂O (50 mL) and brine (50 mL). The organic phase was dried with MgSO₄ and filtered, and the solvent was removed in vacuo. The crude material was purified by flash chromatography with 0-100% EtOAc/hexanes to afford the pure product as white solid.

### Method B

B. Alkyl bromide (1 eq) was dissolved in DMSO (10 mL), and sodium azide (1.9 eq) was added. The reaction was stirred overnight at rt, and the next day, 2-(4-Ethynylphenyl)-4,4,5,5-tetramethyl-1,3,2-dioxaborolane, 1:1 H2O:t-BuOH, sodium ascorbate, and copper (II) sulfate pentahydrate were added to the reaction mixture. The heterogeneous mixture was stirred vigorously overnight. The reaction mixture was then extracted with 3×50 mL DCM and water, and the organic phase was washed with H2O (50 mL) and brine (50 mL). The organic phase was dried with MgSO₄ and filtered, and the solvent was removed in vacuo. The crude material was purified with flash chromatography.

### Suzuki Coupling

Pd(dppf)Cl2-CH2Cl2 (10 mol %) was added to a mixture of the head group (8, 0.040 mmol, 1 eq), the aryl boronate tail group (10, 0.060 mmol, 1.5 eq), TBAI (0.004 mmol, 0.1 eq), and K3PO4 (0.12 mmol, 3 eq) in 2-methyl-THF:H₂O (3:1, 2 mL). The suspension was heated to 64° C., and the reaction was stirred at this temperature until complete by LC-MS. The reaction was allowed to cool to rt, solids were removed via filtration, and the filtrate was concentrated in vacuo. The crude material was purified via flash chromatography 0-10% MeOH/DCM on silica to afford the desired product.

### HCl Deprotection

Hydrochloric acid (4 M in 1,4-dioxane) was added to a solution of the tetrahydropyranyl protected-hydroxamate (1.0 eq) in dichloromethane: methanol (4:1, 1.0 M) at rt, and the reaction was stirred until complete conversion was achieved as monitored by TLC. The desired product was purified by reversed phase HPLC with a C18 column. Chromatography was performed with water as solvent A and acetonitrile as solvent B at a flow rate of 4 mL/min with an isocratic method of 30% solvent B. Elution was monitored at 260 nm, and the fractions containing the final product were pooled. Lyophilization of the collected fractions gave the final product.

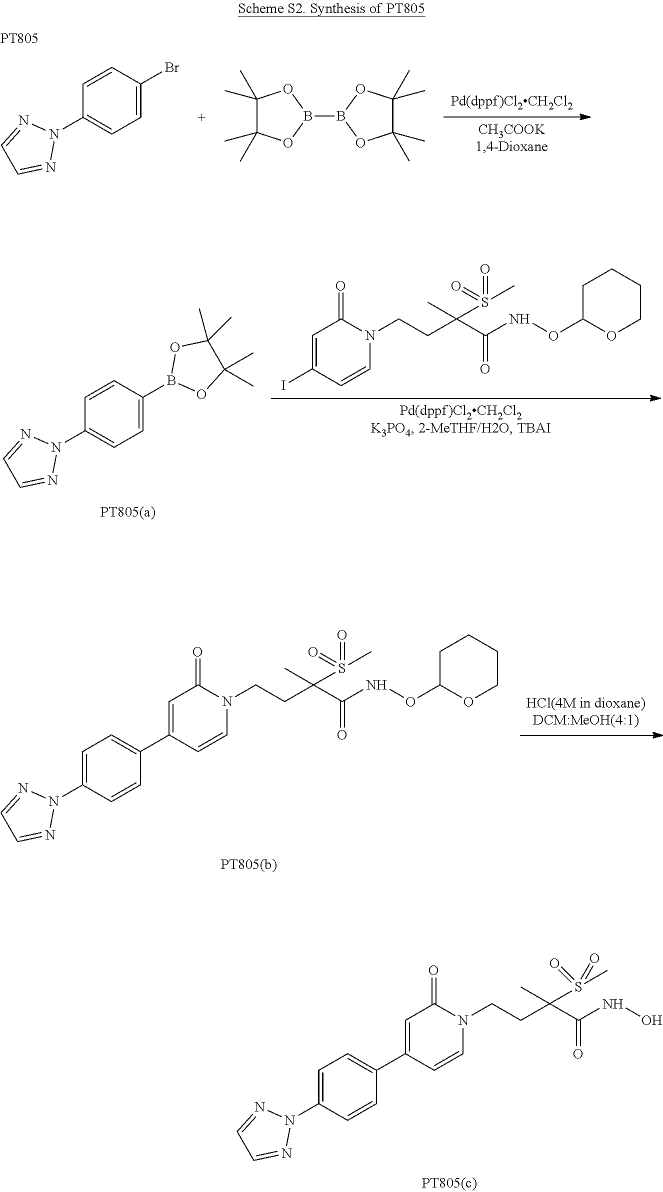

PT85(a)-2-[4-(4,4,5,5-Tetramethyl-1,3,2-dioxaborolan-2-yl)phenyl]-2H-1,2,3-triazole (Yield-71%) ¹H NMR (500 MHz, Chloroform-d) δ ppm 1.39 (s, 12H) 7.85 (s, 2H) 7.95 (m, 2H) 8.11 (m, 2H)

LCMS: m/z calc for C14H18BN3O2 [M+H]⁺: 272.14; found: 272.20.

PT805(b)—4-(4-(4-(2H-1,2,3-Triazol-2-yl)phenyl)-2-oxopyridin-1(2H)-yl)-2-methyl-2-(methylsulfonyl)-N-((tetrahydro-2H-pyran-2-yl)oxy)butanamide (Yield 49%)

LCMS: m/z calc for C24H29N5O6S [M−H]⁻: 514.58; found: 514.70.

PT805(c)—4-(4-(4-(2H-1,2,3-Triazol-2-yl)phenyl)-2-oxopyridin-1(2H)-yl)-N-hydroxy-2-methyl-2-(methylsulfonyl)butanamide (Yield 85%)

¹H NMR (500 MHz, DMSO-d₆) δ ppm 1.58 (s, 3H), 2.15-2.21 (m, 1H), 2.43 (dd, J=11.75, 4.73 Hz, 1H), 3.12 (s, 3H), 3.74-3.81 (m, 1H), 4.11-4.16 (m, 1H), 6.72 (dd, J=7.10, 2.06 Hz, 1H), 6.79 (d, J=2.59 Hz, 1H), 7.80 (d, J=7.17 Hz, 2H), 7.94-7.99 (m, 2H), 8.13 (m, 2H), 8.19 (s, 1H).

HRMS: m/z calc for C19H21N5O5S [M−H]⁻: 430.1191; found: 430.1182.

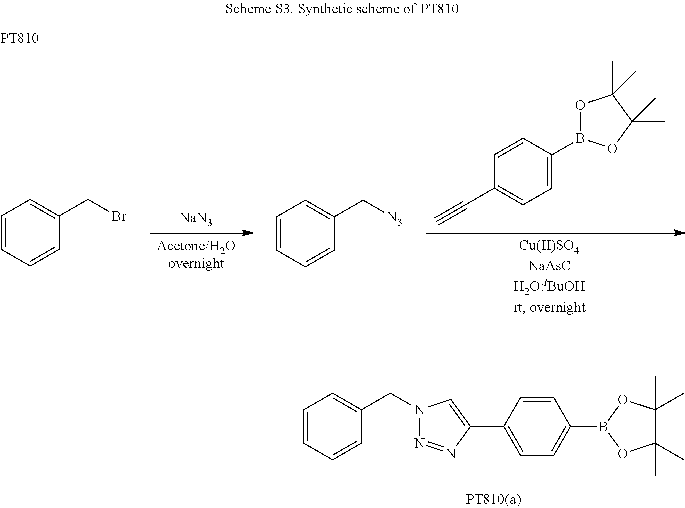

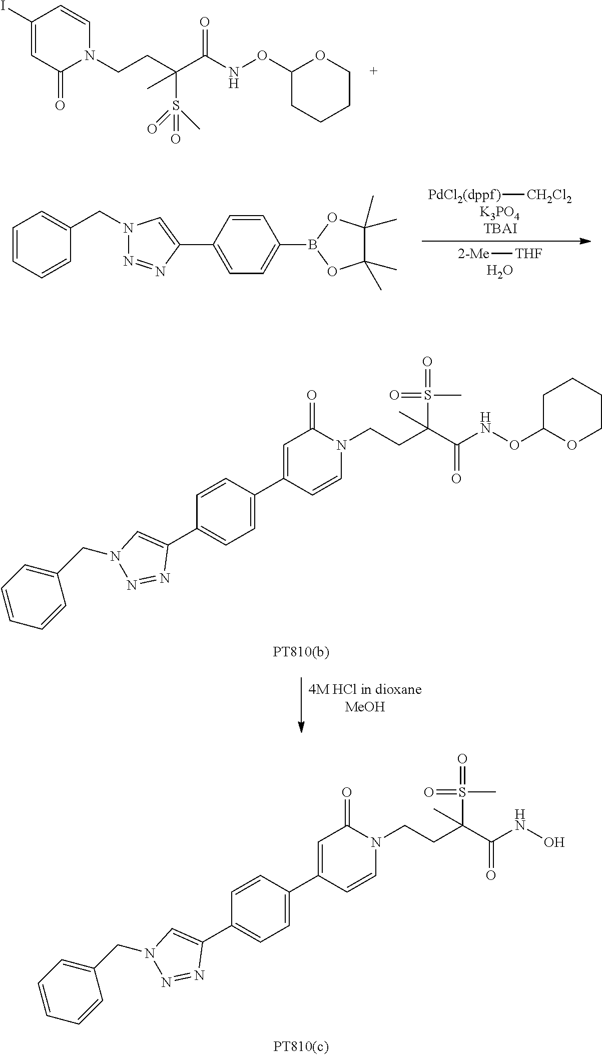

### PT810(a) was Synthesized Using Click Chemistry

PT810(a)—1-benzyl-4-(4-(4,4,5,5-tetramethyl-1,3,2-dioxaborolan-2-yl)phenyl)-1H-1,2,3-triazole (Yield 77%)¹H NMR (500 MHz, Chloroform-d) δ ppm 1.37 (s, 12H) 5.60 (s, 2H) 7.33-7.36 (m, 2H) 7.38-7.44 (m, 3H) 7.72 (s, 1H) 7.81-7.88 (m, 4H)

LCMS: m/z calc for C₂₁H₂₅BN₃O₂ [M+H]⁺: 362.20; found: 362.20.

### PT810(b) was Synthesized Using Suzuki Coupling

PT810(b)—4-(4-(4-(1-benzyl-1H-1,2,3-triazol-4-yl)phenyl)-2-oxopyridin-1(2H)-yl)-2-methyl-2-(methylsulfonyl)-N-((tetrahydro-2H-pyran-2-yl)oxy)butanamide (Yield 45%)¹H NMR (500 MHz, Chloroform-d) δ ppm 1.59-1.67 (m, 3H), 1.73 (d, J=2.44 Hz, 3H), 1.77-1.85 (m, 1H), 1.87-1.94 (m, 1H) 1.94-2.02 (m, 1H) 2.38-2.48 (m, 1H) 2.51-2.61 (m, 1H) 3.22-3.26 (m, 3H), 3.63-3.73 (m, 1H), 4.06-4.14 (m, 1H), 4.18-4.29 (m, 1H), 4.34-4.43 (m, 1H), 5.24 (br. s., 1H), 5.62 (s, 2H), 6.61 (d, J=7.02 Hz, 1H), 6.89 (s, 1H), 7.33-7.37 (m, 2H), 7.39-7.45 (m, 4H), 7.64 (m, 2H), 7.75 (s, 1H), 7.92 (m, 2H), 11.18 (br. s., 1H).

LCMS: m/z calc for C₃₁H₃₄N₅O₆S [M−H]⁻: 604.22; found: 604.20.

### PT810(c) was Synthesized Using HCl Deprotection

PT810(c)—4-(4-(4-(1-benzyl-1H-1,2,3-triazol-4-yl)phenyl)-2-oxopyridin-1(2H)-yl)-N-hydroxy-2-methyl-2-(methylsulfonyl)butanamide (Yield 62%)¹H NMR (700 MHz, DMSO-d₆) δ ppm 1.59 (s, 3H), 2.18 (ddd, J=12.91, 11.62, 5.16 Hz, 1H), 2.44 (td, J=12.21, 4.84 Hz, 2H), 3.12 (s, 3H), 3.76 (td, J=12.05, 4.73 Hz, 1H), 4.11-4.15 (m, 2H), 5.67 (s, 2H), 6.72 (dd, J=7.21, 2.04 Hz, 1H), 6.77 (d, J=1.94 Hz, 1H), 7.35-7.42 (m, 5H), 7.78 (d, J=7.10 Hz, 1H), 7.83 (m, 2H), 7.97 (m, 2H), 8.77 (s, 1H).

¹³C NMR (176 MHz, DMSO-d₆) δ 163.95, 162.05, 150.76, 146.44, 139.56, 136.41, 136.22, 132.30, 129.30, 128.69, 128.42, 128.31, 127.79, 127.04, 126.13, 122.64, 116.06, 115.45, 105.03, 68.52, 53.57, 44.94, 37.25, 31.60, 15.27.

HRMS: m/z calc for C₂₆H₂₈N₅O₅S [M+H]⁺: 522.1806; found: 522.1808.

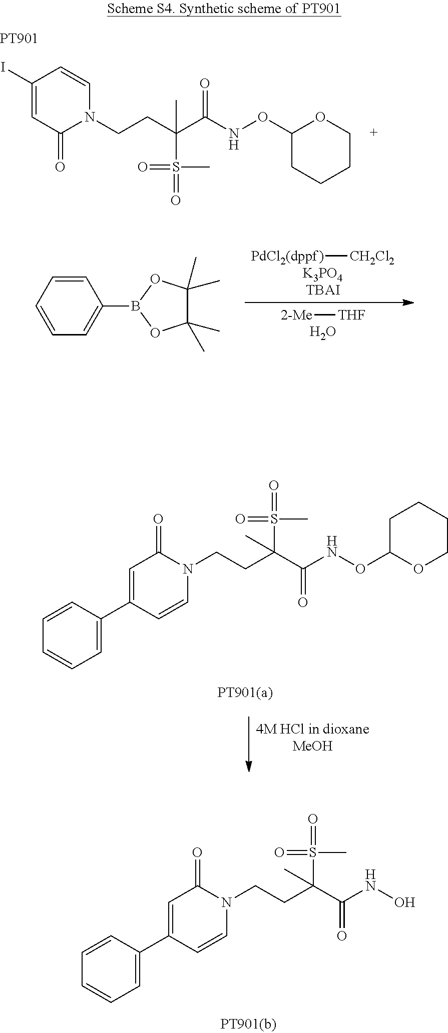

### PT901(a) was Synthesized Using Suzuki Coupling

PT901(a)—2-methyl-2-(methylsulfonyl)-4-(2-oxo-4-phenylpyridin-1(2H)-yl)-N-((tetrahydro-2H-pyran-2-yl)oxy)butanamide(a) (Yield 88%)

PT901(a)—¹H NMR (500 MHz, Chloroform-d) δ ppm 1.59-1.65 (m, 3H), 1.74 (s, 3H), 1.79-1.85 (m, 1H), 1.88-2.00 (m, 2H), 2.40-2.48 (m, 1H), 2.52-2.63 (m, 1H), 3.24 (s, 3H), 3.64-3.73 (m, 1H), 4.07-4.15 (m, 1H), 4.19-4.28 (m, 1H), 4.39 (ddd, J=13.43, 11.52, 3.89 Hz, 1H), 5.19-5.25 (m, 1H), 6.59 (dd, J=7.02, 1.83 Hz, 1H), 6.87 (d, J=1.68 Hz, 1H), 7.41-7.45 (m, 1H), 7.46-7.52 (m, 3H), 7.59 (dd, J=7.55, 2.06 Hz, 2H).

LCMS: m/z calc for C₂₂H₂₇N₂O₆S_{[M-H]}⁻: 447.16; found: 447.10.

### PT901(b) was Synthesized Using HCl Deprotection

PT901(b)—N-hydroxy-2-methyl-2-(methylsulfonyl)-4-(2-oxo-4-phenylpyridin-1(2H)-yl)butanamide (Yield 74%)

PT901(b)—¹H NMR (500 MHz, Methanol-d₄) δ ppm 1.73 (s, 3H), 2.38-2.44 (m, 1H), 2.58-2.63 (m, 1H), 3.13 (s, 3H), 3.92-3.99 (m, 1H), 4.32 (ddd, J=12.78, 11.02, 5.19 Hz, 1H), 6.78 (dd, J=7.02, 1.83 Hz, 1H), 6.80-6.82 (m, 1H), 7.48-7.52 (m, 3H), 7.69 (dd, J=7.78, 1.53 Hz, 2H), 7.73 (d, J=7.02 Hz, 1H).

¹³C NMR (176 MHz, Methanol-d₄) δ 163.45, 163.42, 153.45, 138.35, 136.82, 129.57, 128.84, 126.53, 115.17, 107.01, 68.44, 45.58, 35.94, 30.98, 14.06.

HRMS: m/z calc for C₁₇H₂₁N₂O₅S [M+H]⁺: 365.1166; found: 365.1166.

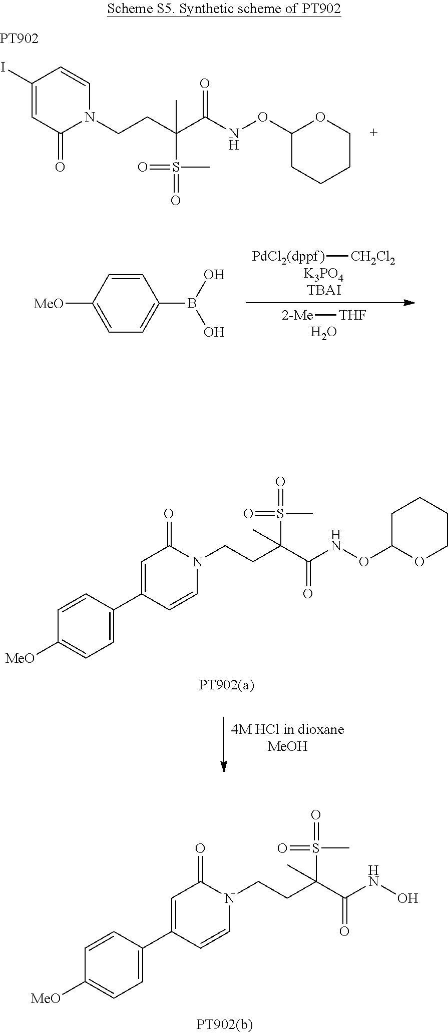

### PT901(a) was Synthesized Using Suzuki Coupling

PT902(a)—4-(4-(4-methoxyphenyl)-2-oxopyridin-1(2H)-yl)-2-methyl-2-(methylsulfonyl)-N-((tetrahydro-2H-pyran-2-yl)oxy)butanamide (Yield 66%)

PT902(a)—¹H NMR (500 MHz, Chloroform-d) δ ppm 1.58-1.69 (m, 3H), 1.72 (s, 3H), 1.78-1.83 (m, 1H), 1.88-1.95 (m, 2H), 2.39-2.46 (m, 1H), 2.53 (ddd, J=14.61, 11.56, 5.87 Hz, 1H), 3.18-3.26 (m, 3H), 3.61-3.72 (m, 1H), 3.87 (s, 3H), 4.04-4.12 (m, 1H), 4.18-4.26 (m, 1H), 4.31-4.39 (m, 1H), 5.23 (br. s., 1H), 6.56 (dd, J=7.02, 1.83 Hz, 1H), 6.79-6.83 (m, 1H), 6.99 (m, J=8.85 Hz, 2H), 7.39 (d, J=7.02 Hz, 1H), 7.55 (m, 2H).

LCMS: m/z calc for C₂₃H₂₉N₂O₇S [M−H]⁻: 477.17; found: 477.20.

### PT902(b) was Synthesized Using HCl Deprotection

PT902(b)—N-hydroxy-4-(4-(4-methoxyphenyl)-2-oxopyridin-1(2H)-yl)-2-methyl-2-(methylsulfonyl)butanamide (Yield 56%)

PT902(b)—¹H NMR (500 MHz, Methanol-d₄) δ ppm 1.72 (s, 3H), 2.40 (ddd, J=13.39, 11.25, 5.11 Hz, 1H), 2.55-2.63 (m, 1H), 3.14 (s, 3H), 3.86 (s, 3H), 3.93 (ddd, J=12.63, 11.41, 4.96 Hz, 1H), 4.26-4.34 (m, 1H), 6.73-6.79 (m, 2H), 7.04 (d, J=8.85 Hz, 2H), 7.64-7.70 (m, 3H).

HRMS: m/z calc for C₁₈H₂₃N₂O₆S [M+H]⁺: 395.1271; found: 395.1274.

¹³C NMR (176 MHz, MeOD) δ 164.89, 163.38, 161.61, 153.32, 138.33, 128.54, 128.00, 114.32, 113.37, 68.49, 54.55, 45.71, 35.98, 31.01, 14.06.

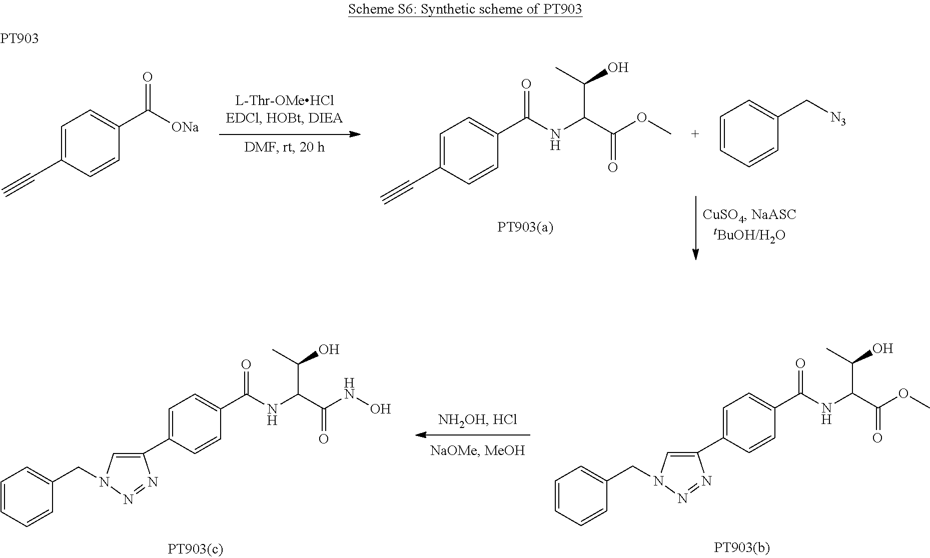

PT903(a)—Methyl (2S,3R)-2-(4-ethynylbenzamido)-3-hydroxybutanoate (Yield 55%)

Diisopropylethylamine (1.1 mL, 11.9 mmol) was added to a stirred solution of 4-ethynylbenzoic acid (500 mg, 2.97 mmol), L-threonine methyl ester hydrochloride (605.3, 3.6 mmol), EDCI (684.2 mg, 3.6 mmol), and HOBt (442 mg, 3.3 mmol) in anhydrous DMF (15 mL) at 0° C. under N₂. The solution was stirred at 0° C. for 1 h and then at rt for 20 h. The solution was diluted with EtOAc (100 mL) and washed with 1.0 M HCl, saturated NaHCO₃, H₂O, dried over MgSO₄, filtered, and concentrated in vacuo. The crude material was purified via flash chromatography to afford the final product methyl (3R)-2-(4-ethynylbenzamido)-3-hydroxybutanoate.

LCMS: m/z calc for C₁₄H₁₆NO₄ [M+H]⁺: 262.29; found: 262.10.

PT903(a)—¹H NMR (700 MHz, Chloroform-d) δ ppm 1.30 (d, J=6.45 Hz, 3H), 3.22 (s, 1H), 3.82 (s, 3H), 4.48 (qd, J=6.38, 2.37 Hz, 1H), 4.83 (dd, J=8.82, 2.37 Hz, 1H), 6.89 (d, J=8.60 Hz, 1H), 7.58 (m, 2H), 7.81 (m, 2H).

LCMS: m/z calc for C₁₄H₁₆NO₄ [M+H]⁺: 262.11; found: 262.10.

### PT903(b) was Synthesized Using Click Chemistry

PT903(b)—methyl(3R)-2-(4-(1-benzyl-1H-1,2,3-triazol-4-yl)benzamido)-3-hydroxybutanoate (Yield 81%)

PT903(b)—¹H NMR (400 MHz, Chloroform-d) δ ppm 1.33 (d, J=6.48 Hz, 3H), 3.83 (s, 3H), 4.49 (qd, J=6.38, 2.26 Hz, 1H), 4.86 (dd, J=8.68, 2.32 Hz, 1H), 5.62 (s, 2H), 6.94 (d, J=8.68 Hz, 1H), 7.32-7.38 (m, 2H), 7.39-7.47 (m, 3H), 7.76 (s, 1H), 7.91 (s, 4H).

LCMS: m/z calc for C₂₁H₂₃N₄O₄ [M+H]⁺: 395.17; found: 395.10.

PT903(c)—4-(1-benzyl-1H-1,2,3-triazol-4-yl)-N-((3R)-3-hydroxy-1-(hydroxyamino)-1-oxobutan-2-yl)benzamide Sodium methoxide (25 wt % in MeOH, 127 mg, 0.59 mmol) was added to a stirred solution of hydroxylamine hydrochloride (27.5 mg, 0.4 mmol) in anhydrous MeOH (0.5 mL) at 0° C. under N₂ atmosphere. After stirring for 20 min, a solution of the methyl ester (50 mg, 0.6 mmol) in 1:1 MeOH:THF (2 mL) was added, and the reaction mixture was stirred at 0° C. for 1 h and then at rt overnight. The reaction mixture was quenched with 1.0 M HCl, concentrated by rotary evaporator to remove the organic solvents, and diluted with DMSO. Purification was performed by reversed phase HPLC with a C18 column (Luna® 5 μm C18(2) 100 Å 250×10 mm). Chromatography was performed with water as solvent A and acetonitrile as solvent B. Elution was monitored at 260 nm, and the fractions containing the final product were pooled. Lyophilization of the collected fractions gave the final product. (Yield 39%)

PT903(c)—¹H NMR (400 MHz, DMSO-d₆) δ ppm 1.10 (d, J=6.36 Hz, 3H), 3.98-4.07 (m, 1H), 4.29 (dd, J=8.38, 5.44 Hz, 1H), 4.90 (d, J=6.24 Hz, 1H), 5.67 (s, 2H), 7.33-7.44 (m, 5H), 7.93-8.03 (m, 4H), 8.05 (d, J=8.44 Hz, 1H), 8.76 (s, 1H), 8.85 (s, 1H), 10.68 (s, 1H).

¹³C NMR (176 MHz, DMSO-d₆) δ 167.55, 166.41, 146.40, 136.37, 133.85, 133.71, 129.32, 128.71, 128.69, 128.45, 125.26, 122.88, 66.93, 58.47, 53.59, 20.84.

HRMS: m/z calc for C₂₀H₂₂N₅O₄ [M+H]⁺: 396.1599; found: 396.1667.

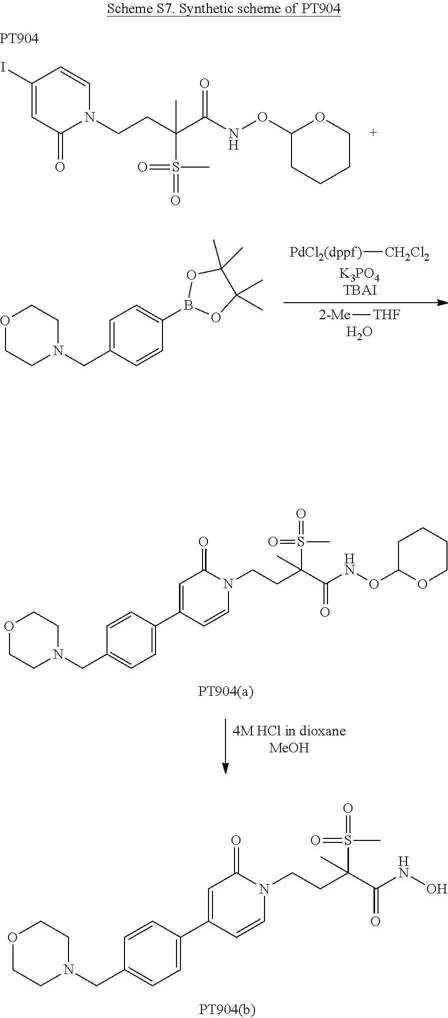

### PT904(a) was Synthesized Using Suzuki Coupling

PT904(a)—2-methyl-2-(methylsulfonyl)-4-(4-(4-(morpholinomethyl)phenyl)-2-oxopyridin-1(2H)-yl)-N-((tetrahydro-2H-pyran-2-yl-oxy)butanamide (Yield 57%)

PT904(a)—¹H NMR (700 MHz, Chloroform-d) δ ppm 1.64 (br. s., 2H), 1.67 (br. s., 1H), 1.73 (br. s., 3H), 1.81 (d, J=12.69 Hz, 1H), 1.91 (d, J=10.11 Hz, 1H), 1.96 (d, J=14.41 Hz, 1H), 2.45 (d, J=11.19 Hz, 1H), 2.49 (br. s., 4H), 2.53-2.60 (m, 1H), 3.24 (br. s., 3H), 3.56 (s, 2H), 3.65 (d, J=11.19 Hz, 1H), 3.74 (br. s., 4H), 4.07-4.14 (m, 1H), 4.19-4.27 (m, 1H), 4.34-4.41 (m, 1H), 5.24 (br. s., 1H), 6.58 (d, J=6.67 Hz, 1H), 6.86 (br. s., 1H) 7.42 (d, J=7.10 Hz, 1H), 7.45 (m, 2H), 7.55 (m, 2H).

LCMS: m/z calc for C₂₇H₃₆N₃O₇S [M−H]⁻: 546.23; found: 546.20.

### PT904(b) was Synthesized Using HCl Deprotection

PT904(b)—N-hydroxy-2-methyl-2-(methylsulfonyl)-4-(4-(4-(morpholinomethyl)phenyl)-2-oxopyridin-1(2H)-yl)butanamide (Yield 46%)

HRMS: m/z calc for C₂₂H₃₀N₃O₆S [M+H]⁺: 462.1700; found: 464.1843.

¹H NMR (700 MHz, Methanol-d₄) δ ppm 1.74 (s, 3H), 2.38-2.43 (m, 1H), 2.60-2.64 (m, 1H), 3.13 (s, 3H), 3.25-3.30 (m, 2H), 3.43 (d, J=12.48 Hz, 2H), 3.76 (t, J=12.80 Hz, 2H), 3.96-4.00 (m, 1H), 4.09 (d, J=15.70 Hz, 2H), 4.34 (td, J=11.89, 5.06 Hz, 1H), 4.46 (s, 2H), 6.80 (d, J=6.88 Hz, 1H), 6.85 (s, 1H), 7.69 (d, J=7.96 Hz, 2H), 7.79 (d, J=7.10 Hz, 1H), 7.86 (d, J=7.96 Hz, 2H).

¹³C NMR (176 MHz, Methanol-d₄) δ 164.88, 163.34, 152.20, 138.80, 132.61, 131.96, 129.87, 127.49, 115.67, 106.82, 68.48, 66.73, 65.55, 63.52, 60.15, 51.53, 45.61, 35.95, 31.05, 13.93.

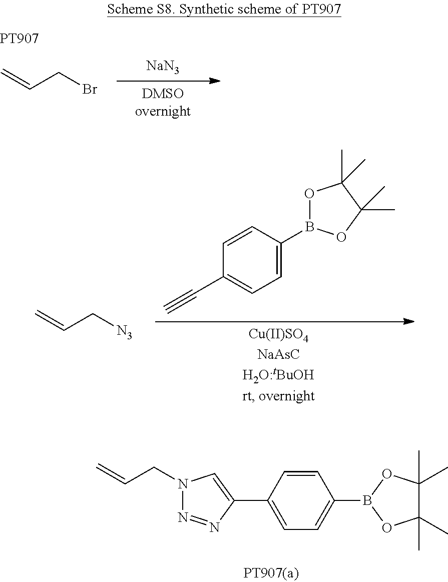

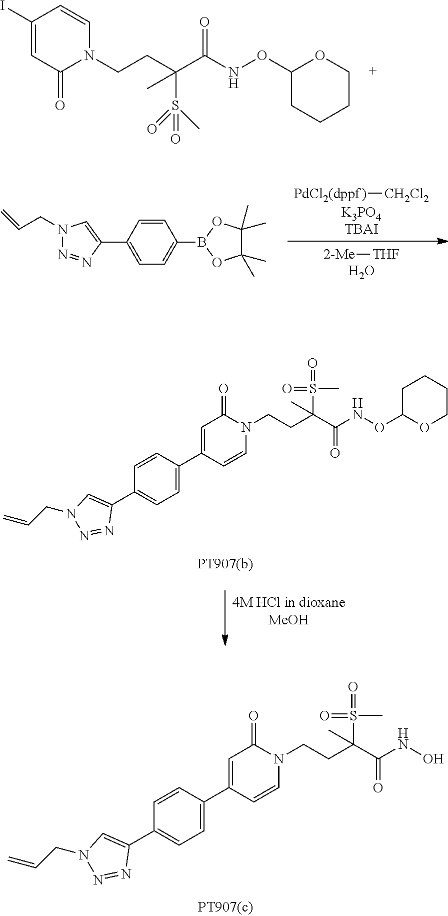

### PT907(a) was Synthesized Using Click Chemistry

PT907(a)—1-allyl-4-(4-(4,4,5,5-tetramethyl-1,3,2-dioxaborolan-2-yl)phenyl)-1H-1,2,3-triazole (Yield 87%)

PT907(a)—¹H NMR (700 MHz, Chloroform-d) δ ppm 1.37 (s, 12H), 5.04 (d, J=6.24 Hz, 2H), 5.35-5.42 (m, 2H), 6.08 (ddt, J=16.89, 10.43, 6.24, 6.24 Hz, 1H), 7.82 (s, 1H), 7.86 (q, J=8.17 Hz, 4H).

LCMS: m/z calc for C₁₇H₂₃BN₃O₂ [M+H]⁺: 312.19; found: 312.20.

### PT907(b) was Synthesized Using Suzuki Coupling

PT907(b)—4-(4-(4-(1-allyl-1H-1,2,3-triazol-4-yl)phenyl)-2-oxopyridin-1(2H)-yl)-2-methyl-2-(methylsulfonyl)-N-((tetrahydro-2H-pyran-2-yl)oxy)butanamide (Yield 72%)

PT907(b)—¹H NMR (400 MHz, DMSO-d₆) δ ppm 1.55 (br. s., 3H), 1.65-1.77 (m, 3H), 2.18-2.27 (m, 1H), 3.11 (d, J=6.97 Hz, 3H), 3.17 (d, J=12.84 Hz, 1H), 3.30 (s, 1H), 3.54 (d, J=11.00 Hz, 1H), 3.72-3.85 (m, 1H), 4.03 (s, 1H), 4.99 (br. s., 1H), 5.10 (d, J=5.75 Hz, 2H), 5.22-5.35 (m, 2H), 6.06-6.18 (m, 1H), 6.73 (d, J=6.97 Hz, 1H), 6.78 (s, 1H), 7.77 (dd, J=11.13, 7.09 Hz, 1H), 7.84 (m, 2H), 7.98 (m, 2H), 8.67 (s, 1H).

LCMS: m/z calc for C₂₇H₃₂N₅O₆S [M−H]⁻: 554.21; found: 554.20.

### PT907(c) was Synthesized Using HCl Deprotection

PT907(c)—4-(4-(4-(1-allyl-1H-1,2,3-triazol-4-yl)phenyl)-2-oxopyridin-1(2H)-yl)-N-hydroxy-2-methyl-2-(methylsulfonyl)butanamide (Yield 72%)

PT907(c)—¹H NMR (700 MHz, DMSO-d₆) δ ppm 1.59 (s, 3H), 2.17-2.21 (m, 1H), 2.42-2.47 (m, 1H), 3.12 (s, 3H), 3.75-3.79 (m, 1H), 4.11-4.15 (m, 1H), 5.06-5.11 (m, 2H), 5.26 (d, J=15.70 Hz, 1H), 5.33 (dd, J=10.33, 1.29 Hz, 1H), 6.10-6.15 (m, 1H), 6.73 (dd, J=7.10, 1.94 Hz, 1H), 6.78 (d, J=1.72 Hz, 1H), 7.78 (d, J=7.10 Hz, 1H), 7.83-7.87 (m, 2H), 7.97-8.01 (m, 2H), 8.68 (s, 1H), 11.18 ((br. s., 1H).

¹³C NMR (176 MHz, DMSO-d₆) δ 163.96, 162.06, 150.79, 146.24, 139.56, 136.20, 133.07, 132.38, 127.80, 127.78, 126.12, 122.48, 119.48, 115.45, 105.04, 68.52, 57.97, 52.41, 44.94, 37.14, 31.60, 14.56.

HRMS: m/z calc for C₂₂H₂₆N₅O₅S [M+H]⁺: 472.1649; found: 472.1645.

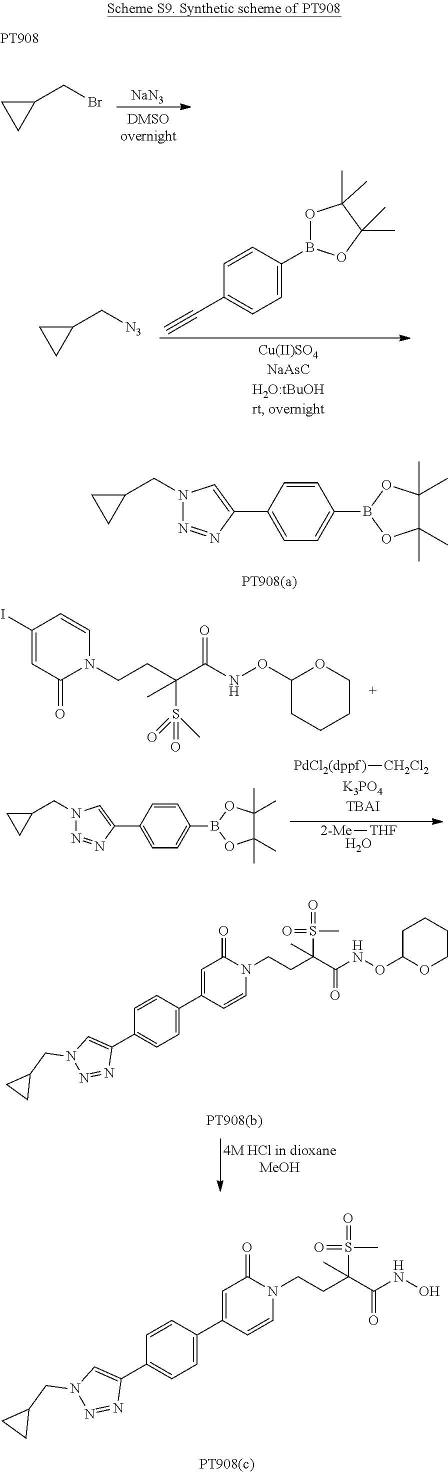

### PT908(a) was Synthesized Using Click Chemistry

PT908(a)—1-(cyclopropylmethyl)-4-(4-(4,4,5,5-tetramethyl-1,3,2-dioxaborolan-2-yl)phenyl)-1H-1,2,3-triazole (Yield 87%)

PT908(a)—¹H NMR (700 MHz, Chloroform-d) δ ppm 0.48-0.53 (m, 2H) 0.74-0.79 (m, 2H) 1.27 (s, 1H) 1.39 (s, 12H) 4.30 (d, J=7.31 Hz, 2H) 7.83-7.91 (m, 4H) 7.94 (s, 1H)

LCMS: m/z calc for C₁₈H₂₅BN₃O₂ [M+H]: 326.21; found: 326.20.

### PT908(b) was Synthesized Using Suzuki Coupling

PT908(b)—4-(4-(4-(1-(cyclopropylmethyl)-1H-1,2,3-triazol-4-yl)phenyl)-2-oxopyridin-1(2H)-yl)-2-methyl-2-(methylsulfonyl)-N-((tetrahydro-2H-pyran-2-yl)oxy)butanamide (Yield 550%)

PT908(b)—¹H NMR (700 MHz, Chloroform-d) δ ppm 0.78 (d, J=7.31 Hz, 2H), 1.08 (t, J=6.78 Hz, 2H), 1.26-1.31 (m, 1H), 1.38-1.40 (m, 1H), 1.52 (dd, J=12.91, 6.24 Hz, 2H), 1.75 (br. s., 3H), 1.79-1.84 (m, 1H), 1.90-1.95 (m, 1H), 1.95-2.02 (m, 2H), 2.43-2.48 (m, 1H), 2.54-2.60 (m, 1H), 3.22-3.29 (m, 3H), 3.67 (br. s., 1H), 3.72 (br. s., 1H), 4.10-4.14 (m, 1H), 4.32 (d, J=7.31 Hz, 2H), 4.39-4.44 (m, 1H), 5.26 (br. s., 1H), 6.64 (d, J=6.45 Hz, 1H), 6.92 (br. s., 1H), 7.45 (d, J=6.88 Hz. 1H), 7.68 (d, J=7.96 Hz, 2H. 7.95-8.02 (m, 3H).

LCMS: m/z calc for C₂₈H₃₄N₅O₆S [M−H]⁻: 568.22; found: 568.20.

### PT908(c) was Synthesized Using HCl Deprotection

PT908(c)—4-(4-(4-(1-(cyclopropylmethyl)-1H-1,2,3-triazol-4-yl)phenyl)-2-oxopyridin-1(2H)-yl)-N-hydroxy-2-methyl-2-(methylsulfonyl)butanamide (Yield 63%)

PT908(c)—¹H NMR (500 MHz, Methanol-d₄) δ ppm 0.54 (q, J=4.88 Hz, 2H), 0.70-0.75 (m, 2H), 1.31 (br. s., 1H), 1.40-1.46 (m, 1H), 1.74 (s, 3H), 2.39-2.45 (m, 1H), 2.59-2.65 (m, 1H), 3.14 (s, 3H), 3.97 (td, J=11.94, 4.96 Hz, 1H), 4.35 (s, 3H), 6.83 (dd, J=7.02, 1.83 Hz, 1H), 6.86-6.89 (m, 1H), 7.76 (d, J=7.02 Hz, 1H), 7.81 (m, 2H), 7.99 (m, 2H), 8.50 (s, 1H).

¹³C NMR (176 MHz, DMSO-d₆) δ 163.95, 162.06, 150.80, 146.08, 139.55, 136.10, 132.53, 127.80, 126.08, 122.08, 115.42, 105.05, 68.52, 54.49, 44.94, 37.14, 31.60, 14.56.

HRMS: m/z calc for C₂₃H₂₈N₅O₅S [M+H]⁺: 486.1806; found: 486.1807.

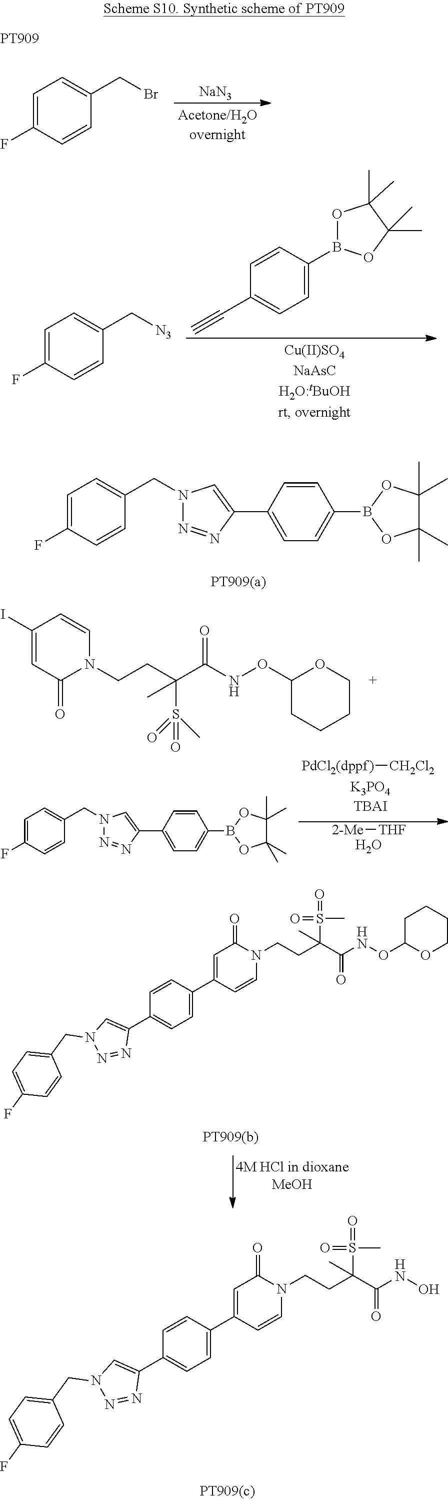

### PT909(a) was Synthesized Using Click Chemistry

PT909(a)—1-(4-fluorobenzyl)-4-(4-(4,4,5,5-tetramethyl-1,3,2-dioxaborolan-2-yl)phenyl)-1H-1,2,3-triazole (Yield 59%)

PT909(a)—¹H NMR (700 MHz, Chloroform-d) δ ppm 1.37 (s, 12H) 5.57 (s, 2H) 7.11 (t, J=8.50 Hz, 2H) 7.34 (dd, J=8.50, 5.27 Hz, 2H) 7.75 (br. s., 1H) 7.81-7.90 (m, 4H) LCMS: m/z calc for C₂₁H₂₄BFN₃O₂ [M+H]⁺: 380.20; found: 380.10.

### PT909(b) was Synthesized Using Suzuki Coupling

PT909(b)—4-(4-(4-(1-(4-fluorobenzyl)-1H-1,2,3-triazol-4-yl)phenyl)-2-oxopyridin-1(2H)-yl)-2-methyl-2-(methylsulfonyl)-N-((tetrahydro-2H-pyran-2-yl)oxy)butanamide (Yield 47%)

PT909(b)—¹H NMR (700 MHz, Chloroform-d) δ ppm 1.62-1.70 (m, 3H), 1.73-1.74 (m, 3H), 1.78-1.85 (m, 1H), 1.90-2.02 (m, 2H), 2.40-2.48 (m, 1H), 2.52-2.59 (m, 1H), 3.23-3.27 (m, 3H), 3.65 (d, J=11.40 Hz, 1H), 4.07-4.14 (m, 1H), 4.20-4.29 (m, 1H), 4.38-4.43 (m, 1H), 5.25 (br. s., 1H), 5.60 (s, 2H), 6.61 (dd, J=6.99, 1.61 Hz, 1H), 6.87-6.93 (m, 1H), 7.12 (t, J=8.50 Hz, 2H), 7.35 (dd, J=8.60, 5.16 Hz, 2H), 7.44 (d, J=7.10 Hz, 1H), 7.65 (m, 2H) 7.74 (s, 1H), 7.93 (m, 2H).

LCMS: m/z calc for C₃₁H₃₃FN₅O₆S [M−H]⁻: 622.21; found: 622.20.

### PT909(c) was Synthesized Using HCl Deprotection

PT909(c)—4-(4-(4-(1-(4-fluorobenzyl)-1H-1,2,3-triazol-4-yl)phenyl)-2-oxopyridin-1(2H)-yl)-N-hydroxy-2-methyl-2-(methylsulfonyl)butanamide (Yield 62%)

PT909(c)—¹H NMR (700 MHz, DMSO-d₆) δ ppm 1.58 (s, 3H), 2.15-2.20 (m, 1H), 2.40-2.45 (m, 1H), 3.11 (s, 3H), 3.75 (dd, J=11.94, 7.21 Hz, 1H), 4.10-4.14 (m, 1H), 5.66 (s, 2H), 6.71 (dd, J=7.10, 1.94 Hz, 1H), 6.76 (d, J=1.72 Hz, 1H), 7.24 (t, J=8.82 Hz, 2H), 7.44 (dd, J=8.60, 5.59 Hz, 2H), 7.77 (d, J=7.10 Hz, 1H), 7.83 (m, 2H), 7.95 (m, 2H), 8.74 (s, 1H), 11.17 (br. s., 1H).

¹³C NMR (176 MHz, DMSO-d₆) δ 163.95, 162.05, 150.75, 146.46, 143.80, 139.56, 136.23, 132.63, 132.27, 130.82, 130.77, 129.54, 127.83, 127.80, 126.18, 126.13, 124.47, 123.06, 122.56, 116.21, 116.09, 115.45, 105.02, 68.52, 52.76, 52.66, 44.94, 37.12, 31.59, 14.55.

HRMS: m/z calc for C₂₆H₂₅FN₅O₅S [M−H]: 540.1711; found: 540.1711.

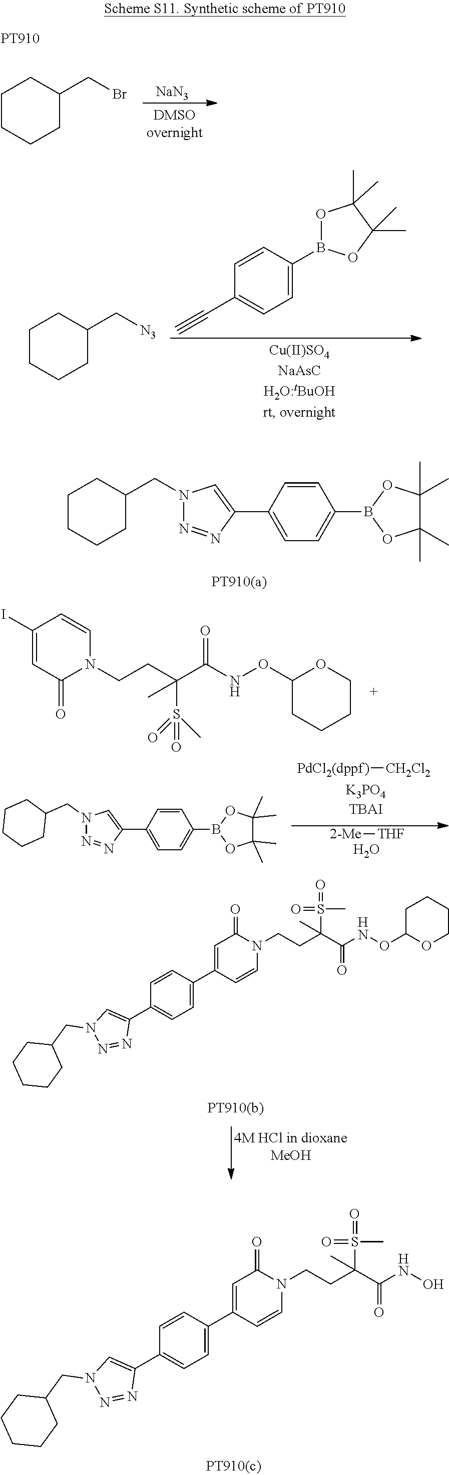

### PT910(a) was Synthesized Using Click Chemistry

PT910(a)—1-(cyclohexylmethyl)-4-(4-(4,4,5,5-tetramethyl-1,3,2-dioxaborolan-2-yl)phenyl)-1H-1,2,3-triazole (Yield 67%)

PT910(a)—¹H NMR (500 MHz, Chloroform-d) δ ppm 1.01-1.11 (m, 2H), 1.16-1.32 (m, 3H), 1.64-1.71 (m, 3H), 1.74-1.82 (m, 2H), 1.96 (dtt, J=14.75, 7.41, 7.41, 3.68, 3.68 Hz, 1H), 4.25 (d, J=7.17 Hz, 2H), 7.80 (br. s., 1H), 7.88 (s, 4H).

LCMS: m/z calc for C₂₁H₃₁BN₃O₂ [M+H]⁺: 368.25; found: 368.20.

### PT910(b) was Synthesized Using Suzuki Coupling

PT910(b)—4-(4-(4-(1-(cyclohexylmethyl)-1H-1,2,3-triazol-4-yl)phenyl)-2-oxopyridin-1(2H)-yl)-2-methyl-2-(methylsulfonyl)-N-((tetrahydro-2H-pyran-2-yl)oxy)butanamide (Yield 73%)

LCMS: m/z calc for C₃₁H₄₀N₅O₆S [M−H]⁻: 611.28; found: 611.20.

### PT910(c) was Synthesized Using HCl Deprotection

PT910(c)—4-(4-(4-(1-(cyclohexylmethyl)-1H-1,2,3-triazol-4-yl)phenyl)-2-oxopyridin-1(2H)-yl)-N-hydroxy-2-methyl-2-(methylsulfonyl)butanamide (Yield 81%)

¹H NMR (500 MHz, DMSO-d₆) δ ppm 0.97-1.05 (m, 2H), 1.13-1.32 (m, 6H), 1.59 (s, 3H) 1.66-1.72 (m, 2H), 1.84-1.92 (m, 2H), 1.97-2.04 (m, 1H), 2.17-2.23 (m, 1H), 3.12 (s, 3H) 3.77 (td, J=12.05, 4.58 Hz, 2H), 4.14 (td, J=11.86, 4.96 Hz, 2H), 4.27 (d, J=7.17 Hz, 2H), 6.72 (dd, J=7.02, 1.83 Hz, 1H), 6.76-6.81 (m, 1H), 7.78 (d, J=7.17 Hz, 1H), 7.84 (m, 2H), 7.96 (m, 2H), 8.67 (s, 1H), 9.27 (s, 1H), 11.18 (br. s., 1H).

¹³C NMR (176 MHz, DMSO-d₆) δ 163.96, 162.06, 150.79, 145.94, 139.56, 136.10, 132.50, 127.79, 126.06, 122.79, 115.42, 105.03, 68.52, 55.88, 44.94, 38.56, 37.13, 31.59, 30.27, 26.20, 25.54, 14.55.

HRMS: m/z calc for C₂₆H₃₄N₅O₅S [M+H]⁺: 528.2275; found: 528.2272.

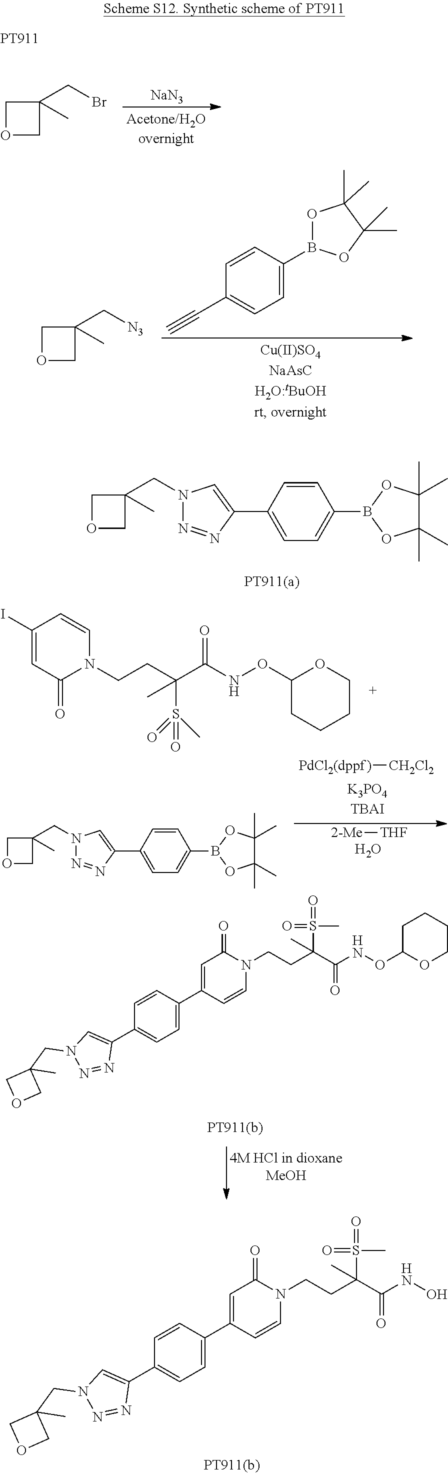

### PT911(a) was Synthesized Using Click Chemistry

PT911(a)—1-((3-methyloxetan-3-yl)methyl)-4-(4-(4,4,5,5-tetramethyl-1,3,2-dioxaborolan-2-yl)phenyl)-1H-1,2,3-triazole (Yield 46%)

PT911(a)—¹H NMR (700 MHz, Chloroform-d) δ ppm 1.33 (s, 3H), 1.37 (s, 12H), 4.65 (s, 2H), 4.72 (d, J=6.24 Hz, 2H), 7.78 (s, 1H), 7.84-7.89 (m, 4H).

LCMS: m/z calc for C₁₉H₂₇BN₃O₃ [M+H]⁺: 356.22; found: 356.20.

### PT911(b) was Synthesized Using Suzuki Chemistry

PT911(b)-2-methyl-4-(4-(4-(1-((3-methyloxetan-3-yl)methyl)-1H-1,2,3-triazol-4-yl)phenyl)-2-oxopyridin-1(2H)-yl)-2-(methylsulfonyl)-N-((tetrahydro-2H-pyran-2-yl)oxy)butanamide (Yield 74%)

PT911(b)—¹H NMR (700 MHz, Chloroform-d) δ ppm 1.36 (s, 3H), 1.64-1.71 (m, 3H), 1.75 (s, 3H), 1.80-1.84 (m, 1H), 1.90-1.94 (m, 1H), 1.97 (d, J=15.27 Hz, 1H), 2.42-2.47 (m, 1H), 2.55-2.60 (m, 1H), 3.23-3.27 (m, 3H), 3.67 (br. s., 1H), 3.72 (d, J=11.40 Hz, 1H), 4.09-4.14 (m, 1H), 4.22-4.27 (m, 1H), 4.39-4.44 (m, 1H), 4.51 (d, J=6.24 Hz, 2H), 4.69 (s, 2H), 4.75 (d, J=6.24 Hz, 2H), 5.25 (br. s., 1H), 6.64 (d, J=7.10 Hz, 1H), 6.93 (br. s., 1H), 7.46 (d, J=7.10 Hz, 1H), 7.68 (m, 2H), 7.83 (s, 1H), 7.97 (m, 2H).

LCMS: m/z calc for C₂₉H₃₆N₅O₇S [M−H]⁻: 598.23; found: 598.20.

### PT911(c) was Synthesized Using HCl Deprotection

PT911(c)—N-hydroxy-2-methyl-4-(4-(4-(1-((3-methyloxetan-3-yl)methyl)-1H-1,2,3-triazol-4-yl)phenyl)-2-oxopyridin-1(2H)-yl)-2-(methylsulfonyl)butanamide (Yield 52%)

PT911(c)—¹H NMR (700 MHz, DMSO-d₆) δ ppm 0.89 (s, 3H), 1.59 (s, 3H), 2.17-2.22 (m, 1H), 2.39-2.44 (m, 1H), 3.12 (s, 3H), 3.59 (d, J=11.19 Hz, 2H), 3.66 (d, J=11.19 Hz, 2H), 3.75-3.78 (m, 1H), 4.14 (dd, J=11.94, 4.84 Hz, 1H), 4.43 (d, J=2.15 Hz, 2H), 6.73 (dd, J=7.10, 1.94 Hz, 1H), 6.78 (d, J=1.94 Hz, 1H), 7.79 (d, J=7.10 Hz, 1H), 7.85 (m, 2H), 7.98 (m, 2H), 8.63 (s, 1H), 11.18 (br. s., 1H).

HRMS: m/z calc for C₂₄H₂₈N₅O₆S [M−H]⁻: 522.1554; found: 552.1683.

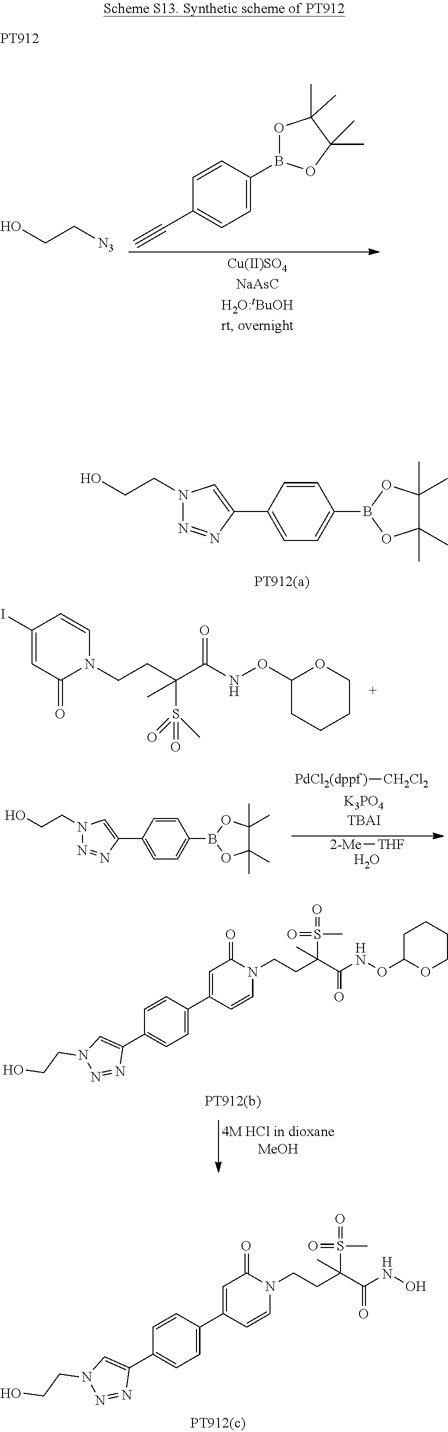

### PT912(a) was Synthesized Using Click Chemistry

PT912(a)—2-(4-(4-(4,4,5,5-tetramethyl-1,3,2-dioxaborolan-2-yl)phenyl)-1H-1,2,3-triazol-1-yl)ethan-1-ol (Yield 83%)

PT912(a)—¹H NMR (700 MHz, Chloroform-d) δ ppm (m, 2H) 7.82 (m, J=8.17 Hz, 2H) 7.86 (m, J=8.17 Hz, 2H) 7.92 (s, 1H)

LCMS: m/z calc for C₁₆H₂₃BN₃O₃ [M+H]⁺: 316.19; found: 316.10.

### PT912(b) was Synthesized Using Suzuki Coupling

PT912(b)—4-(4-(4-(1-(2-hydroxyethyl)-1H-1,2,3-triazol-4-yl)phenyl)-2-oxopyridin-1(2H)-yl)-2-methyl-2-(methylsulfonyl)-N-((tetrahydro-2H-pyran-2-yl)oxy)butanamide (Yield 55%)

PT912(b)—¹H NMR (700 MHz, Chloroform-d) δ ppm 1.62-1.66 (m, 2H), 1.72 (s, 3H), 1.79-1.86 (m, 1H), 1.86-2.00 (m, 3H), 2.41 (t, J=10.65 Hz, 1H), 2.50-2.58 (m, 1H), 3.19-3.26 (m, 3H), 3.63-3.73 (m, 1H), 4.02-4.08 (m, 1H), 4.16 (br. s., 2H), 4.18-4.27 (m, 1H), 4.30 (t, J=12.05 Hz, 1H), 4.58 (br. s., 2H), 5.23 (br. s., 1H), 6.57 (d, J=5.81 Hz, 1H), 6.83 (br. s., 1H), 7.41 (br. s., 1H), 7.58 (m, 2H), 7.87 (m, 2H), 8.01 (br. s., 1H).

LCMS: m/z calc for C₂₆H₃₂N₅O₇S [M−H]⁻: 558.20; found: 558.20.

### PT912(c) was Synthesized Using HCl Deprotection

PT912(c)—N-hydroxy-4-(4-(4-(1-(2-hydroxyethyl)-1H-1,2,3-triazol-4-yl)phenyl)-2-oxopyridin-1(2H)-yl)-2-methyl-2-(methylsulfonyl)butanamide (Yield 87%)

PT912(c)—¹H NMR (700 MHz, DMSO-d₆) δ ppm 1.59 (s, 3H), 2.19 (dd, J=12.15, 7.21 Hz, 1H), 2.45 (dd, J=11.40, 7.96 Hz, 1H), 3.12 (s, 3H), 3.77 (d, J=4.52 Hz, 1H), 3.84 (d, J=5.38 Hz, 2H), 4.13 (d, J=4.95 Hz, 1H), 4.46 (t, J=5.38 Hz, 2H), 5.10 (t, J=5.16 Hz, 1H), 6.73 (dd, J=7.10, 2.15 Hz, 1H), 6.78 (d, J=1.94 Hz, 1H), 7.78 (d, J=7.10 Hz, 1H), 7.84 (m, 2H), 7.98 (m, 2H), 8.66 (s, 1H), 11.18 (br. s, 1H).

¹³C NMR (176 MHz, DMSO-d₆) δ 163.96, 162.06, 150.81, 145.87, 139.54, 136.05, 132.58, 127.80, 126.04, 122.92, 115.41, 105.04, 68.52, 60.25, 52.96, 44.94, 37.13, 31.59, 25.42, 14.57.

HRMS: m/z calc for C₂₁H₂₆N₅O₆S [M+H]⁺: 476.1598; found: 476.1602.

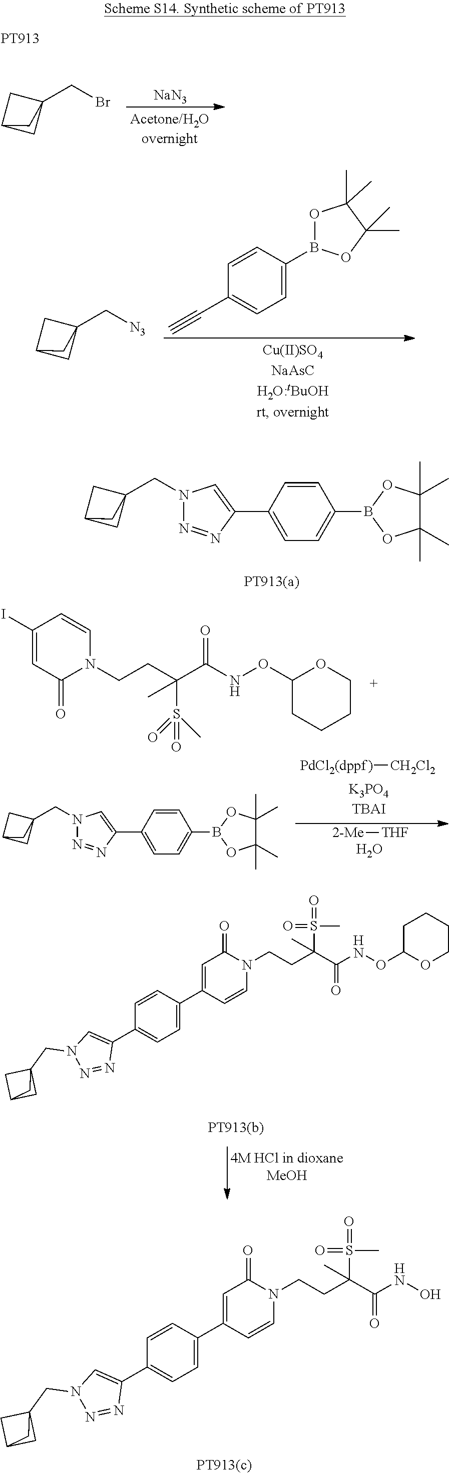

### PT913(a) was Synthesized Using Click Chemistry

PT913(a)—1-(bicyclo[1.1.1]pentan-1-ylmethyl)-4-(4-(4,4,5,5-tetramethyl-1,3,2-dioxaborolan-2-yl)phenyl)-1H-1,2,3-triazole (Yield 60%)

PT913(a)—¹H NMR (700 MHz, Chloroform-d) δ ppm 1.37 (s, 12H) 1.80 (s, 6H) 4.45 (s, 2H) 7.76 (br. s., 1H) 7.85-7.89 (m, 4H)

LCMS: m/z calc for C20H27BN₃O2 [M+H]⁺: 352.22; found: 352.20.

### PT913(b) was Synthesized Using Suzuki Coupling

PT913(b)—4-(4-(4-(1-(bicyclo[1.1.1]pentan-1-ylmethyl)-1H-1,2,3-triazol-4-yl)phenyl)-2-oxopyridin-1(2H)-yl)-2-methyl-2-(methylsulfonyl)-N-((tetrahydro-2H-pyran-2-yl)oxy)butanamide (Yield 79%)

PT913(b)—¹H NMR (500 MHz, Chloroform-d) δ ppm 1.02 (t, J=6.71 Hz, 1H), 1.24 (s, 6H), 1.57-1.68 (m, 3H), 1.73 (br. s., 3H), 1.85-2.00 (m, 3H), 2.43 (t, J=11.06 Hz, 1H), 2.52-2.57 (m, 1H), 3.17-3.27 (m, 3H), 3.62-3.72 (m, 1H), 4.09 (br. s., 1H), 4.18-4.27 (m, 1H), 4.36 (d, J=10.68 Hz, 1H), 4.47 (s, 2H), 5.23 (br. s., 1H), 6.61 (d, J=5.34 Hz, 1H), 6.89 (s, 1H), 7.40-7.50 (m, 1H), 7.65 (m, 2H), 7.80 (s, 1H), 7.96 (m, 2H).

LCMS: m/z calc for C₃₀H₃₈N₅O₆S [M+H]⁺: 594.24; found: 594.20.

### PT913(c) was Synthesized Using HCL Deprotection

PT913(c)—4-(4-(4-(1-(bicyclo[1.1.1]pentan-1-ylmethyl)-1H-1,2,3-triazol-4-yl)phenyl)-2-oxopyridin-1(2H)-yl)-N-hydroxy-2-methyl-2-(methylsulfonyl)butanamide (Yield 83%)

PT913(c)—¹H NMR (700 MHz, DMSO-d₆) δ ppm 1.29-1.34 (m, 1H), 1.57-1.60 (m, 3H), 1.73 (s, 6H), 2.19 (td, J=12.26, 5.16 Hz, 1H), 2.42-2.47 (m, 1H), 3.12 (s, 3H), 3.77 (td, J=12.05, 4.73 Hz, 1H), 4.14 (td, J=11.94, 5.16 Hz, 1H), 4.50 (s, 2H), 6.73 (dd, J=7.10, 1.94 Hz, 1H), 6.78 (d, J=1.94 Hz, 1H), 7.78 (d, J=7.10 Hz, 1H), 7.84 (m, 2H), 7.99 (m, 2H), 8.62 (s, 1H), 11.18 (br. s., 1H).

¹³C NMR (176 MHz, DMSO-d₆) δ 163.96, 162.06, 150.80, 146.05, 139.56, 136.14, 132.46, 127.78, 126.12, 122.36, 115.43, 105.04, 68.52, 57.97, 51.82, 49.85, 44.94, 43.55, 37.13, 31.61, 27.56, 23.52, 19.69, 14.55.

HRMS: m/z calc for C₂₅H₃₀N₅O₅S [M+H]⁺: 512.1962; found: 512.1961.

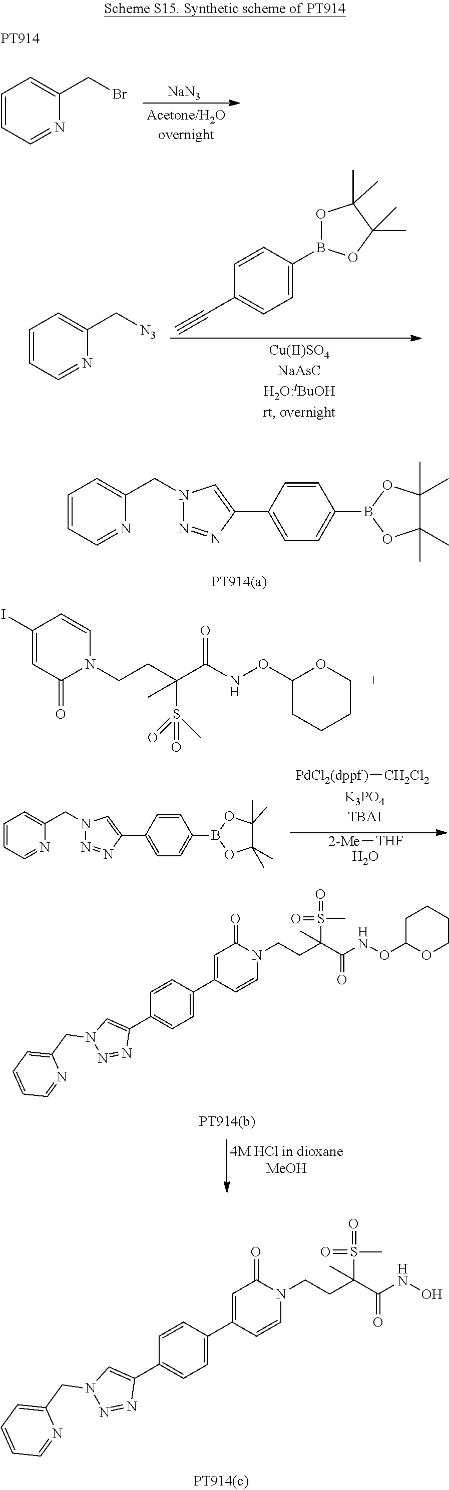

### PT914(a) was Synthesized Using Click Chemistry

PT914(a)—2-((4-(4-(4,4,5,5-tetramethyl-1,3,2-dioxaborolan-2-yl)phenyl)-1H-1,2,3-triazol-1-yl)methyl)pyridine

PT914(a)—¹H NMR (700 MHz, Chloroform-d) δ ppm 1.38 (s, 12H) 5.74 (s, 2H) 7.28-7.29 (m, 1H) 7.30-7.33 (m, 1H) 7.74 (t, J=7.74 Hz, 1H) 7.85-7.89 (m, 4H) 8.01 (s, 1H) 8.64 (d, J=4.52 Hz, 1H)

LCMS: m/z calc for C₂₀H₂₃BN₄O₂ [M+H]⁺: 363.20; found: 363.20.

### PT914(b) was Synthesized Using Suzuki Coupling

PT914(b)-2-methyl-2-(methylsulfonyl)-4-(2-oxo-4-(4-(1-(pyridin-2-ylmethyl)-1H-1,2,3-triazol-4-yl)phenyl)pyridin-1(2H)-yl)-N-((tetrahydro-2H-pyran-2-yl)oxy)butanamide (Yield 73%)

PT914(b)—¹H NMR (700 MHz, Chloroform-d) δ ppm 1.63-1.70 (m, 3H), 1.74 (s, 3H), 1.80-1.84 (m, 1H), 1.90-2.00 (m, 2H), 2.41-2.48 (m, 1H), 2.53-2.61 (m, 1H), 3.23-3.28 (m, 3H), 3.65 (d, J=11.40 Hz, 1H), 4.08-4.14 (m, 1H), 4.20-4.28 (m, 1H), 4.36-4.44 (m, 1H), 5.25 (br. s., 1H), 5.76 (s, 2H), 6.62 (dd, J=6.88, 1.72 Hz, 1H), 6.87-6.92 (m, 1H), 7.31-7.38 (m, 2H), 7.44 (d, J=7.10 Hz, 1H), 7.66 (m, 2H), 7.74-7.79 (m, 1H), 7.96 (m, 2H), 8.06 (s, 1H), 8.65 (d, J=4.09 Hz, 1H).

LCMS: m/z calc for C₃₀H₃₃N₆O₆S [M−H]⁻: 605.22; found: 605.20.

### PT914(c) was Synthesized Using HCl Deprotection

PT914(c)—N-hydroxy-2-methyl-2-(methylsulfonyl)-4-(2-oxo-4-(4-(1-(pyridin-2-ylmethyl)-1H-1,2,3-triazol-4-yl)phenyl)pyridin-1(2H)-yl)butanamide (Yield 78%)

PT914(c)—¹H NMR (700 MHz, DMSO-d₆) δ ppm 1.59 (s, 3H), 2.17-2.21 (m, 1H), 2.44 (dd, J=11.94, 7.42 Hz, 1H), 3.12 (s, 3H), 3.76 (dd, J=11.94, 7.21 Hz, 1H), 4.11-4.15 (m, 1H), 5.79 (s, 2H), 6.73 (dd, J=7.10, 1.94 Hz, 1H), 6.78 (d, J=1.94 Hz, 1H), 7.36-7.40 (m, 2H), 7.78 (d, J=7.31 Hz, 1H), 7.83-7.88 (m, 3H), 7.99 (d, J=8.39 Hz, 2H), 8.57 (d, J=4.09 Hz, 1H), 8.78 (s, 1H), 11.17 (br. s., 1H).

¹³C NMR (176 MHz, DMSO-d₆) δ 163.95, 162.05, 155.34, 150.77, 149.96, 146.26, 139.56, 137.91, 136.20, 132.33, 127.80, 126.13, 123.82, 123.37, 122.75, 115.45, 105.03, 68.52, 55.09, 44.94, 37.13, 31.60, 14.56.

HRMS: m/z calc for C₂₅H₂₇N₆O₅S [M+H]⁺: 523.1758; found: 523.1754.

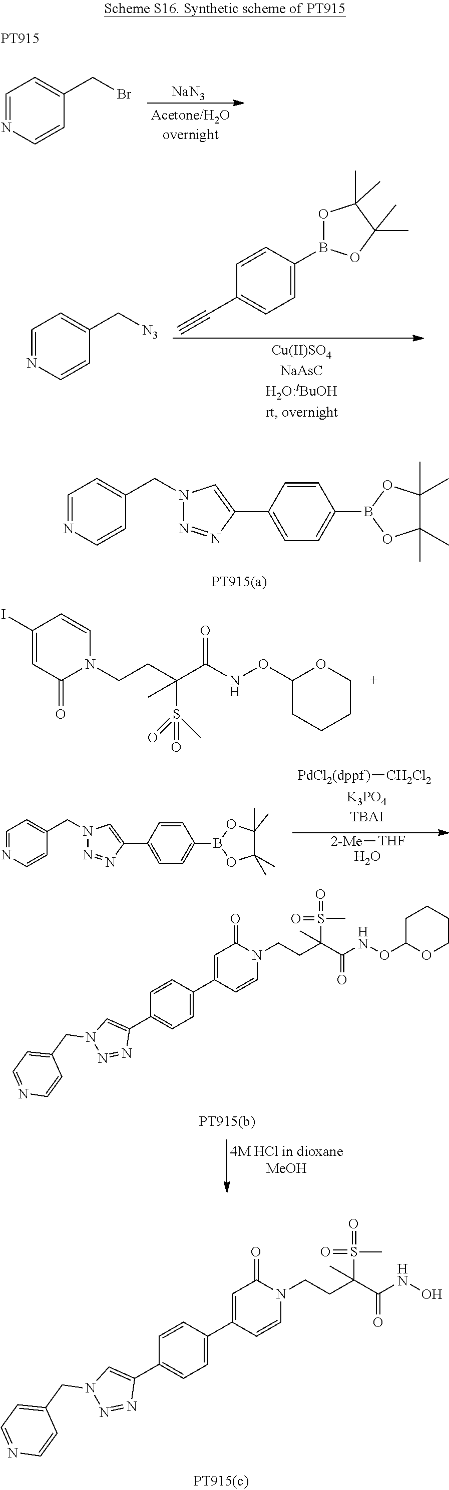

### PT915(a) was Synthesized Using Click Chemistry

PT915(a)—4-((4-(4-(4,4,5,5-tetramethyl-1,3,2-dioxaborolan-2-yl)phenyl)-1H-1,2,3-triazol-1-yl)methyl)pyridine (Yield 75%)

PT915 (a)—¹H NMR (700 MHz, Chloroform-d) δ ppm 1.36 (br. s., 12H) 5.65 (br. s., 2H) 7.22 (br. s., 2H) 7.79-7.91 (m, 5H) 8.66 (br. s., 2H)

LCMS: m/z calc for C₂₀H₂₄BN₄O₂ [M+H]⁺: 363.20; found: 363.20.

### PT915(b) was Synthesized Using Suzuki Coupling

PT915(b)-2-methyl-2-(methylsulfonyl)-4-(2-oxo-4-(4-(1-(pyridin-4-ylmethyl)-1H-1,2,3-triazol-4-yl)phenyl)pyridin-1(2H)-yl)-N-((tetrahydro-2H-pyran-2-yl)oxy)butanamide (Yield 54%)

PT917(b)—¹H NMR (400 MHz, DMSO-d₆) δ ppm 2.15-2.28 (m, 1H), 2.40-2.48 (m, 1H), 3.02-3.20 (m, 3H), 3.54 (d, J=11.13 Hz, 1H), 3.72-3.87 (m, 1H), 4.02-4.19 (m, 2H), 4.99 (br. s., 1H), 5.77 (s, 2H), 6.73 (d, J=6.97 Hz, 1H), 6.78 (s, 1H), 7.28 (d, J=5.26 Hz, 2H), 7.74-7.81 (m, 1H), 7.85 (m, 2H), 7.98 (m, J=8.19 Hz, 2H), 8.59 (d, J=5.62 Hz, 2H), 8.80 (s, 1H).

LCMS: m/z calc for C₃₀H₃₃N₆O₆S [M−H]⁻: 605.22; found: 605.20.

### PT915(c) was Synthesized Using HCl Deprotection

PT915(c)—N-hydroxy-2-methyl-2-(methylsulfonyl)-4-(2-oxo-4-(4-(1-(pyridin-4-ylmethyl)-1H-1,2,3-triazol-4-yl)phenyl)pyridin-1(2H)-yl)butanamide (Yield 68%)

PT915(c)—¹H NMR (500 MHz, DMSO-d₆) δ ppm 1.58 (s, 3H), 2.16-2.20 (m, 1H), 2.44 (dd, J=11.37, 8.01 Hz, 1H), 3.11 (s, 3H), 3.77 (dd, J=11.83, 4.50 Hz, 1H), 4.13 (dd, J=11.75, 6.87 Hz, 1H), 6.00 (s, 2H), 6.72 (dd, J=7.17, 1.98 Hz, 1H), 6.78 (d, J=1.83 Hz, 1H), 7.69 (d, J=5.04 Hz, 2H), 7.78 (d, J=7.02 Hz, 1H), 7.86 (m, 2H), 7.98 (m, 2H), 8.84 (s, 3H), 11.18 (br. s., 1H).

¹³C NMR (176 MHz, Methanol-d₄) δ 164.93, 163.40, 156.16, 152.54, 147.39, 142.47, 138.52, 136.77, 131.52, 130.94, 128.36, 127.26, 127.24, 126.81, 125.98, 125.92, 125.33, 122.60, 115.36, 115.08, 106.72, 68.44, 51.89, 48.12, 45.60, 35.91, 31.01, 13.99.

HRMS: m/z calc for C₂₅H₂₇N₆O₅S [M+H]⁺: 523.1758; found: 523.1754.

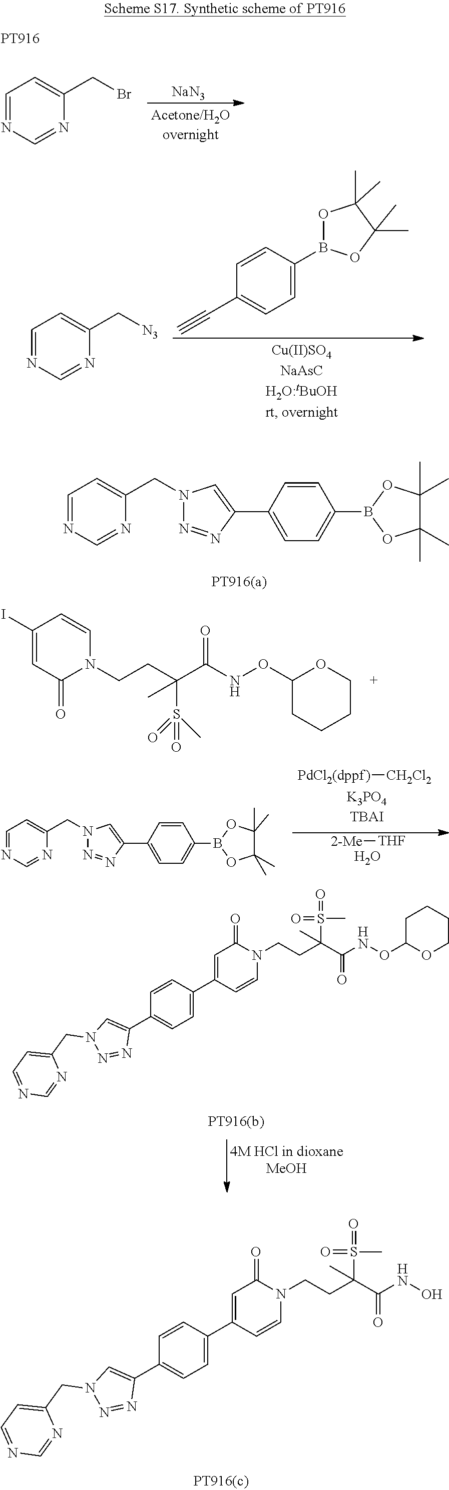

### PT916(a) was Synthesized Using Click Chemistry

PT916(a)—4-((4-(4-(4,4,5,5-tetramethyl-1,3,2-dioxaborolan-2-yl)phenyl)-1H-1,2,3-triazol-1-yl)methyl)pyrimidine (Yield 80%)

PT916(a)—¹H NMR (700 MHz, Chloroform-d) δ ppm 1.37 (s, 12H) 5.71 (s, 2H) 7.16 (d, J=5.16 Hz, 1H) 7.84-7.90 (m, 4H) 8.00 (s, 1H) 8.76 (d, J=4.95 Hz, 1H) 9.24 (s, 1H)

LCMS: m/z calc for C₂₀H₂₄BN₄O₂ [M+H]⁺: 364.20; found: 364.20.

### PT916(b) was Synthesized Using Suzuki Coupling

PT916(b)-2-methyl-2-(methylsulfonyl)-4-(2-oxo-4-(4-(1-(pyrimidin-4-ylmethyl)-1H-1,2,3-triazol-4-yl)phenyl)pyridin-1(2H)-yl)-N-((tetrahydro-2H-pyran-2-yl)oxy)butanamide (Yield 63%)

PT916(b)—¹H NMR (700 MHz, DMSO-d₆) δ ppm 1.54 (br. s., 3H), 1.60 (d, J=8.39 Hz, 3H), 1.70 (d, J=12.05 Hz, 3H), 2.20-2.25 (m, 1H), 2.43-2.49 (m, 1H), 3.11 (d, J=12.26 Hz, 3H), 3.54 (d, J=11.19 Hz, 1H), 3.74-3.85 (m, 1H), 4.05-4.19 (m, 2H), 4.99 (br. s., 1H), 5.88 (s, 2H), 6.74 (d, J=7.10 Hz, 1H), 6.79 (s, 1H), 7.40-7.43 (m, 1H), 7.75-7.80 (m, 1H), 7.83-7.89 (m, 3H), 8.00 (d, J=7.96 Hz, 2H), 8.81 (s, 1H), 8.85 (d, J=5.38 Hz, 1H), 9.18 (s, 1H).

LCMS: m/z calc for C₂₉H₃₂N₇O₆S [M−H]⁻: 364.20; found: 364.200.

### PT916(c) was Synthesized Using HCl Deprotection

PT916(c)—N-hydroxy-2-methyl-2-(methylsulfonyl)-4-(2-oxo-4-(4-(1-(pyrimidin-4-ylmethyl)-1H-1,2,3-triazol-4-yl)phenyl)pyridin-1(2H)-yl)butanamide (Yield 75%)

PT916(c)—¹H NMR (500 MHz, DMSO-d₆) δ ppm 1.57-1.61 (m, 3H), 1.99-2.02 (m, 1H), 2.19 (d, J=4.88 Hz, 1H), 3.12 (s, 3H), 3.76-3.79 (m, 1H), 4.13-4.16 (m, 1H), 5.88 (s, 2H), 6.69-6.76 (m, 2H), 6.78 (d, J=1.83 Hz, 1H), 7.42 (d, J=5.34 Hz, 1H), 7.79 (d, J=7.17 Hz, 2H), 7.83-7.88 (m, 2H), 7.97-8.02 (m, 2H), 8.81 (s, 1H), 8.85 (d, J=5.19 Hz, 1H), 9.16-9.20 (m, 1H), 11.18 (br. s, 1H).

HRMS: m/z calc for C₂₄H₂₅N₇O₅S [M+H]⁺: 524.1711; found: 514.1714.

¹³C NMR (176 MHz, DMSO-d₆) δ 163.93, 161.83, 159.01, 158.62, 150.76, 146.38, 139.58, 136.29, 132.21, 127.83, 126.15, 123.82, 119.92, 115.48, 105.03, 68.52, 53.95, 44.94, 37.14, 31.60, 14.56.

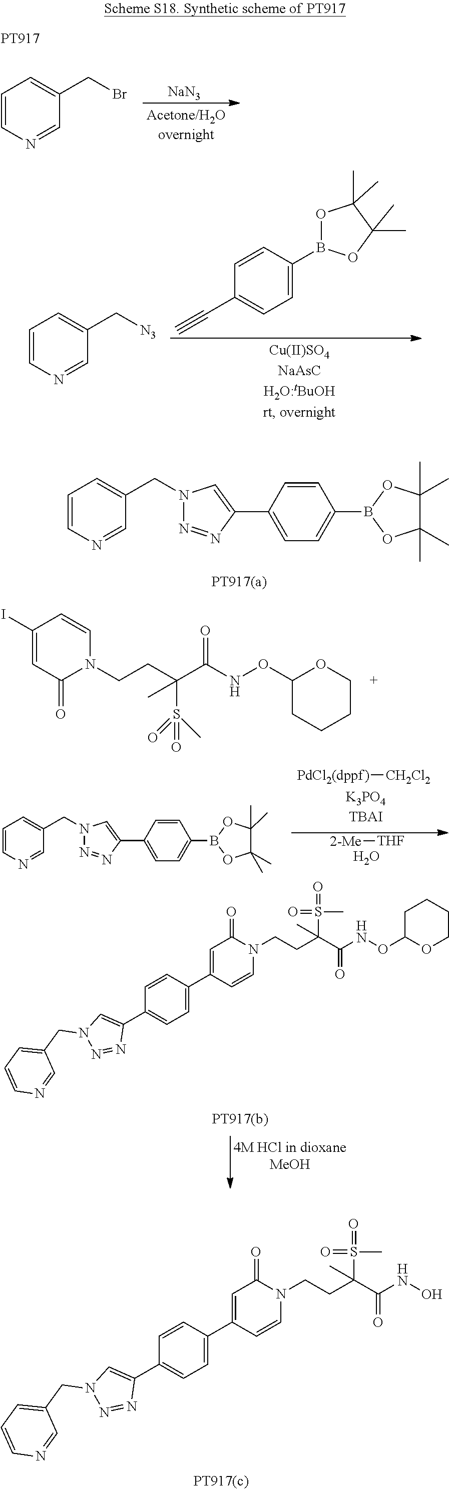

### PT917(a) was Synthesized Using Click Chemistry

PT917(a)—3-((4-(4-(4,4,5,5-tetramethyl-1,3,2-dioxaborolan-2-yl)phenyl)-1H-1,2,3-triazol-1-yl)methyl)pyridine (Yield 64%)

PT917(a)—¹H NMR (400 MHz, Chloroform-d) δ ppm 1.37 (s, 12H), 5.63 (s, 2H), 7.35 (dd, J=7.70, 4.89 Hz, 1H), 7.66 (d, J=7.82 Hz, 1H), 7.77 (s, 1H), 7.82-7.89 (m, 4H), 8.64-8.71 (m, 2H).

LCMS: m/z calc for C₂₀H₂₄BN₄O₂ [M+H]⁺: 363.20; found: 363.20.

### PT917(b) was Synthesized Using Suzuki Coupling

PT917(b)-2-methyl-2-(methylsulfonyl)-4-(2-oxo-4-(4-(1-(pyridin-3-ylmethyl)-1H-1,2,3-triazol-4-yl)phenyl)pyridin-1(2H)-yl)-N-((tetrahydro-2H-pyran-2-yl)oxy)butanamide (Yield 61%)

PT917(b)—¹H NMR (700 MHz, Chloroform-d) δ ppm 1.60-1.65 (m, 2H), 1.65-1.70 (m, 1H), 1.73 (br. s., 3H), 1.78-1.83 (m, 1H), 1.90-1.99 (m, 2H), 2.40-2.46 (m, 1H), 2.52-2.60 (m, 1H), 3.24 (d, J=5.81 Hz, 3H), 3.65 (d, J=11.40 Hz, 1H), 4.07-4.12 (m, 1H), 4.19-4.27 (m, 1H), 4.35-4.41 (m, 1H), 5.24 (br. s., 1H), 5.69 (br. s., 2H), 6.60 (d, J=6.67 Hz, 1H), 6.88 (s, 1H), 7.43 (d, J=6.88 Hz, 1H), 7.65 (m, 2H), 7.73 (d, J=7.10 Hz, 1H), 7.84 (br. s., 1H), 7.93 (m, 2H).

LCMS: m/z calc for C₃₀H₃₃N₆O₆S [M−H]⁻: 605.22; found: 605.20.

### PT917(c) was Synthesized HCl Deprotection

PT917(c)—N-hydroxy-2-methyl-2-(methylsulfonyl)-4-(2-oxo-4-(4-(1-(pyridin-3-ylmethyl)-1H-1,2,3-triazol-4-yl)phenyl)pyridin-1(2H)-yl)butanamide (Yield 82%)

PT917(c)—¹H NMR (500 MHz, DMSO-d₆) δ ppm 1.59 (s, 3H), 2.18 (dd, J=11.67, 7.55 Hz, 1H), 2.44 (dd, J=11.83, 7.55 Hz, 1H), 3.12 (s, 3H), 3.77 (dd, J=12.13, 7.10 Hz, 1H), 4.13 (dd, J=11.75, 6.71 Hz, 1H), 5.87 (s, 2H), 6.72 (dd, J=7.10, 1.91 Hz, 1H), 6.78 (d, J=1.83 Hz, 1H), 7.79 (d, J=7.02 Hz, 1H), 7.80-7.87 (m, 3H), 7.96 (d, J=8.39 Hz, 2H), 8.24 (d, J=7.63 Hz, 1H), 8.79 (d, J=4.88 Hz, 1H), 8.82 (s, 1H), 8.90 (s, 1H), 11.18 (br. s., 1H).

¹³C NMR (176 MHz, DMSO-d₆) δ 163.90, 162.04, 150.71, 150.39, 146.55, 143.69, 139.61, 136.37, 134.90, 132.07, 127.87, 127.04, 126.18, 123.11, 115.47, 105.03, 68.51, 63.74, 52.80, 50.27, 44.95, 37.14, 31.59, 14.57.

HRMS: m/z calc for C₂₅H₂₇N₆O₅S [M+H]⁺: 523.1758; found: 523.1755.

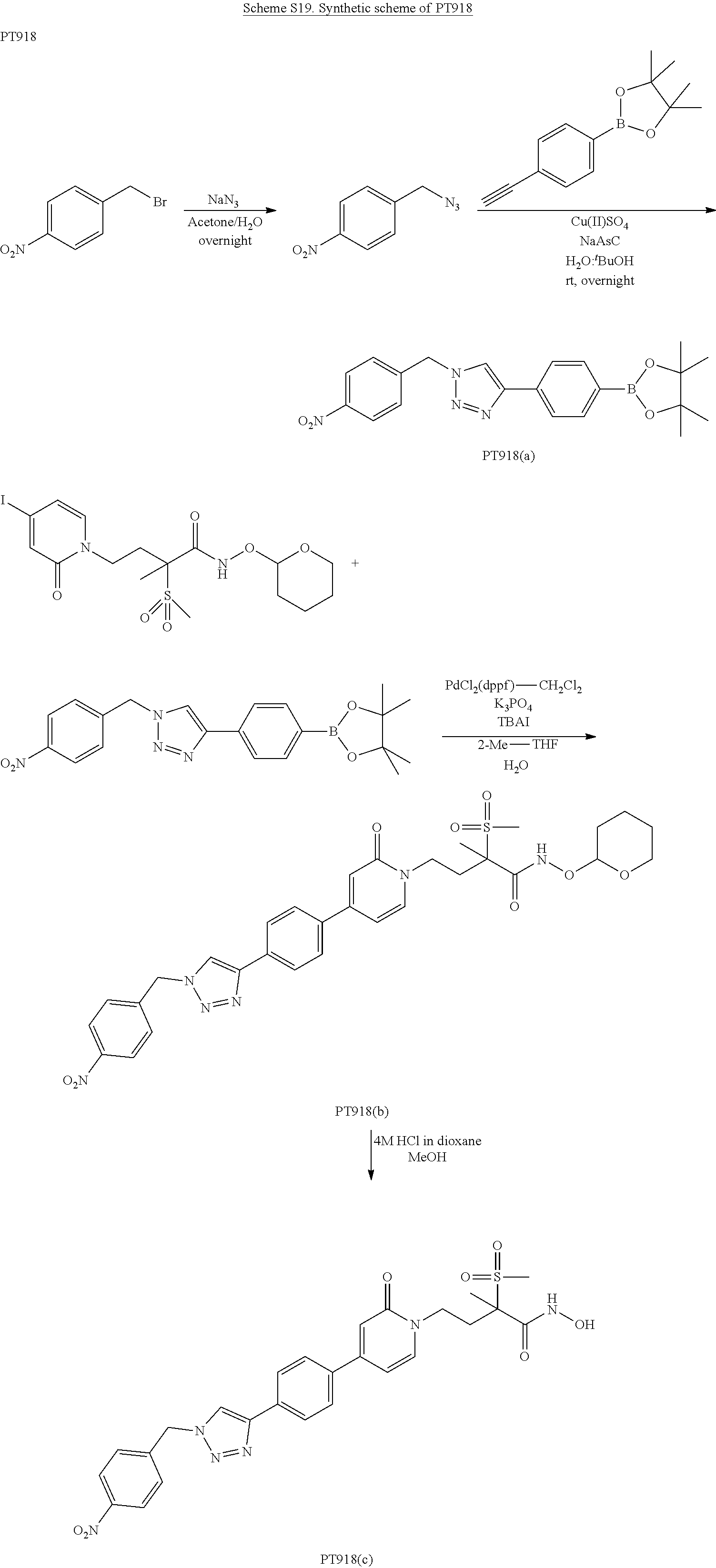

### PT918(a) was Synthesized Using Click Chemistry

PT918(a)—1-(4-nitrobenzyl)-4-(4-(4,4,5,5-tetramethyl-1,3,2-dioxaborolan-2-yl)phenyl)-1H-1,2,3-triazole (Yield 76%)

PT918(a)—¹H NMR (700 MHz, Chloroform-d) δ ppm 1.36 (s, 12H), 5.71 (s, 2H) 7.47 (m, J=8.82 Hz, 2H) 7.79 (s, 1H) 7.82-7.88 (m, 4H) 8.26 (m, J=8.82 Hz, 2H)

LCMS: m/z calc for C₂₁H₂₄BN₄O₄ [M+H]⁺: 380.20; found: 380.10.

### PT918(b) was Synthesized Using Suzuki Coupling

PT918(b)-2-methyl-2-(methylsulfonyl)-4-(4-(4-(1-(4-nitrobenzyl)-1H-1,2,3-triazol-4-yl)phenyl)-2-oxopyridin-1(2H)-yl)-N-((tetrahydro-2H-pyran-2-yl)oxy)butanamide (Yield 57%)

PT918(b)—¹H NMR (700 MHz, Chloroform-d) δ ppm 1.61-1.66 (m, 2H), 1.73 (s, 3H), 1.80-1.85 (m, 1H), 1.85-1.97 (m, 3H), 2.37-2.46 (m, 1H), 2.52-2.59 (m, 1H), 3.21-3.26 (m, 3H), 3.64 (d, J=11.40 Hz, 1H), 4.06-4.13 (m, 1H), 4.18-4.27 (m, 1H), 4.32-4.40 (m, 1H), 5.23 (br. s., 1H), 5.76 (s, 2H), 6.60-6.65 (m, 1H), 6.87-6.90 (m, 1H), 7.44 (d, J=7.10 Hz, 1H), 7.50 (d, J=8.60 Hz, 2H), 7.65 (d, J=8.39 Hz, 2H), 7.89 (s, 1H), 7.94 (d, J=8.17 Hz, 2H), 8.23-8.30 (m, 2H).

LCMS: m/z calc for C₃₁H₃₃N₆O₈S [M−H]⁻: 649.21; found: 649.20.

### PT918(c) was Synthesized Using HCl Deprotection

PT918(c)—N-hydroxy-2-methyl-2-(methylsulfonyl)-4-(4-(4-(1-(4-nitrobenzyl)-1H-1,2,3-triazol-4-yl)phenyl)-2-oxopyridin-1(2H)-yl)butanamide (Yield 64%)

PT918(c)—¹H NMR (500 MHz, Methanol-d₄) δ ppm 1.74 (s, 3H), 2.40-2.44 (m, 1H), 2.61-2.66 (m, 1H), 3.13 (s, 3H), 3.97-4.01 (m, 1H), 4.32-4.37 (m, 1H), 5.85 (s, 2H), 6.84-6.90 (m, 2H), 7.62 (m, 2H), 7.77-7.83 (m, 3H), 7.99 (m, 2H), 8.30 (d, J=8.70 Hz, 2H), 8.54 (s, 1H).

¹³C NMR (176 MHz, DMSO-d₆) δ 163.95, 162.07, 150.75, 147.76, 146.56, 143.78, 139.57, 136.33, 132.13, 129.54, 127.83, 126.19, 124.47, 123.06, 115.47, 105.06, 68.52, 52.66, 44.95, 37.12, 31.59, 14.55.

HRMS: m/z calc for C₂₆H₂₇N₆O₇S [M+H]⁺: 567.16564; found: 567.1649.

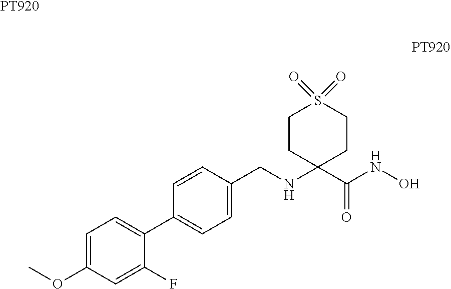 [SMILES: "PT920" ; COc1ccc(-c2ccc(CNC3(C(=O)NO)CCS(=O)(=O)CC3)cc2)c(F)c1]

### 4-(((2′-fluoro-4′-methoxy-[1,1′-biphenyl]-4-yl)methyl)amino)-N-hydroxytetrahydro-2H-thiopyran-4-carboxamide 1,1-dioxide

PT920-¹H NMR (700 MHz, Methanol-d4) δ ppm 2.50 (br. s., 4H), 2.87 (br. s., 2H), 3.14-3.27 (m, 3H), 3.84 (s, 3H), 4.11 (br. s., 2H), 6.76-6.89 (m, 2H), 7.40 (t, J=8.80 Hz, 1H), 7.54 (d, J=7.04 Hz, 2H), 7.57-7.63 (m, 2H).

HRMS: m/z calc for C₂₀H₂₄FN₂O₅S [M+H]⁺: 423.1384; found: 423.1386.

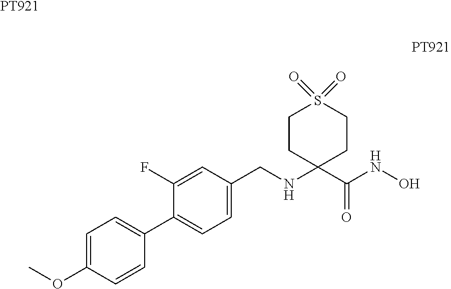 [SMILES: "PT921" ; COc1ccc(-c2ccc(CNC3(C(=O)NO)CCS(=O)(=O)CC3)cc2F)cc1]

### 4-(((2-fluoro-4′-methoxy-[1,1′-biphenyl]-4-yl)methyl)amino)-N-hydroxytetrahydro-2H-thiopyran-4-carboxamide 1,1-dioxide

PT921-¹H NMR (700 MHz, DMSO-d6) δ ppm 2.19-2.40 (m, 3H), 3.05 (br. s., 2H), 3.27-3.41 (m, 3H), 3.78-3.83 (m, 5H), 7.26-7.49 (m, 5H, 6.99-7.08 (m, 2H).

HRMS: m/z calc for C₂₀H₂₄FN₂O₅S [M+H]⁺: 423.1384; found: 423.1384.

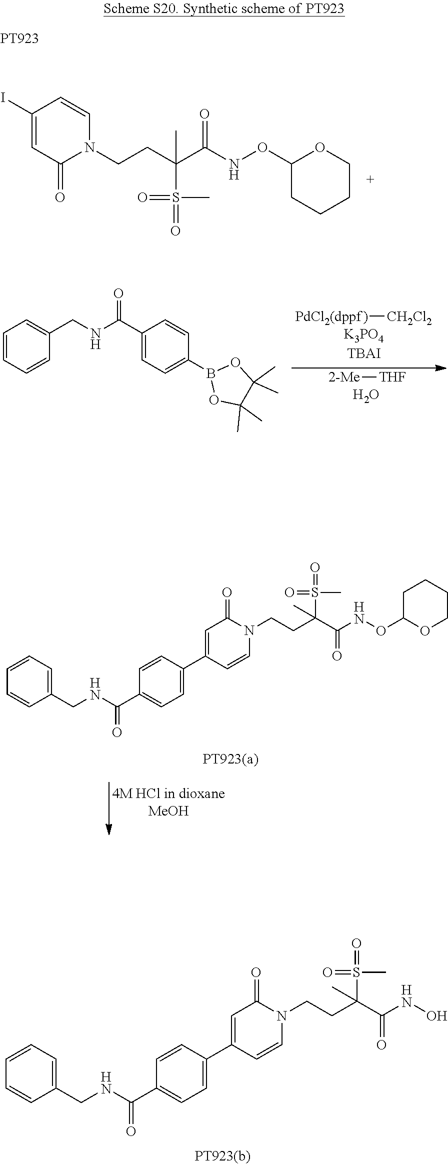

### PT923(a) was Synthesized Using Suzuki Coupling

PT923(a)—N-benzyl-4-(1-(3-methyl-3-(methylsulfonyl)-4-oxo-4-(((tetrahydro-2H-pyran-2-yl)oxy)amino)butyl)-2-oxo-1,2-dihydropyridin-4-yl)benzamide (Yield 89%)

PT923(a)—¹H NMR (500 MHz, Chloroform-d) δ ppm 1.59-1.66 (m, 3H), 1.70 (s, 3H), 1.76-1.82 (m, 1H), 1.85-1.96 (m, 2H), 2.35-2.43 (m, 1H), 2.48-2.57 (m, 1H), 3.18 (d, J=3.97 Hz, 3H), 3.61-3.69 (m, 1H), 4.15-4.26 (m, 1H), 4.28-4.36 (m, 1H), 4.66 (d, J=5.65 Hz, 2H), 5.18 (d, J=19.38 Hz, 1H), 6.55 (d, J=7.02 Hz, 1H), 6.82 (s, 1H), 6.94 (t, J=4.88 Hz, 1H), 7.33-7.39 (m, 4H), 7.44 (d, J=7.02 Hz, 1H), 7.60 (m, 2H), 7.92 (m, 2H).

LCMS: m/z calc for C₃₀H₃₄N₃O₇S [M−H]⁻: 580.21; found: 580.20.

### PT923(b) was Synthesized Using HCl Deprotection

PT923(b)—N-benzyl-4-(1-(4-(hydroxyamino)-3-methyl-3-(methylsulfonyl)-4-oxobutyl)-2-oxo-1,2-dihydropyridin-4-yl)benzamide (Yield 77%)

PT923(b)—¹H NMR (500 MHz, Methanol-d₄) δ ppm 1.73 (s, 3H), 2.40 (ddd, J=13.35, 11.06, 5.19 Hz, 1H), 2.62 (ddd, J=13.35, 10.91, 5.19 Hz, 1H), 3.13 (s, 3H), 3.97 (td, J=11.98, 5.04 Hz, 1H), 4.33 (ddd, J=12.70, 11.02, 5.11 Hz, 1H), 4.62 (s, 2H), 6.80 (dd, J=7.10, 1.75 Hz, 1H), 6.85-6.89 (m, 1H), 7.28 (d, J=7.17 Hz, 1H), 7.33-7.41 (m, 4H), 7.77 (d, J=7.17 Hz, 1H), 7.81 (m, 2H), 7.99 (m, 2H).

¹³C NMR (176 MHz, Methanol-d₄) δ 167.89, 164.89, 163.12, 152.62, 139.73, 138.70, 135.49, 128.17, 127.79, 127.18, 126.84, 115.44, 107.38, 68.41, 45.82, 43.21, 35.88, 31.08, 13.85.

HRMS: m/z calc for C₂₅H₂₈N₃O₆S [M+H]⁺: 498.1693; found: 498.1688.

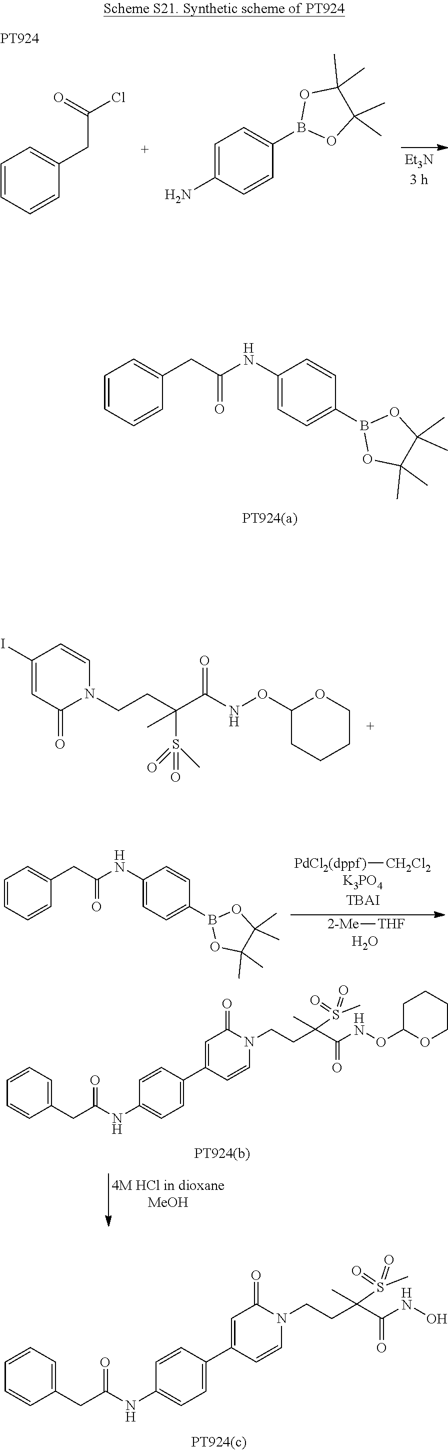

### PT924(a)—2-Phenyl-N-(4-(4,4,5,5-Tetramethyl-1,3,2-Dioxaborolan-2-Yl)Phenyl)Acetamide

The boronate ester (500 mg, 1.0 eq) was dissolved in DCM (10 mL). To the solution, triethylamine (0.6 mL, 1.5 eq) and phenylacetyl chloride (0.33 ml, 1.1 eq) were added, and the solution was stirred for 3 h. The solution was then washed with 1(N) HCl, 5% NaHCO₃, and brine. The organic layer was dried over MgSO₄, filtered, and evaporated to a white solid, which was crystalized from ethyl acetate to give the desired product. (Yield 81%)

PT924(a)—¹H NMR (700 MHz, Chloroform-d) δ ppm 1.35 (s, 12H), 3.77 (s, 2H), 7.10 (br. s., 1H), 7.35-7.40 (m, 3H), 7.41-7.47 (m, 4H), 7.74 (d, J=8.39 Hz, 2H).

LCMS: m/z calc for C₂₀H₂₅BNO₃ [M+H]⁺: 338.19; found: 338.20.

### PT924(b) was Synthesized Using Suzuki Coupling

PT924(b)—N-hydroxy-2-methyl-2-(methylsulfonyl)-4-(2-oxo-4-(4-(2-phenylacetamido)phenyl)pyridin-)—N-((tetrahydro-2H-pyran-2-yl)oxy)butanamide (Yield 38%)

LCMS: m/z calc for C₂₀H₂₃BNO₃ [M−H]⁻: 580.22; found: 580.21.

### PT924(c) was Synthesized Using HCL Deprotection

PT924(c)—N-hydroxy-2-methyl-2-(methylsulfonyl)-4-(2-oxo-4-(4-(2-phenylacetamido)phenyl)pyridin-1(2H)-yl)butanamide (Yield 53%)

PT924(c)—¹H NMR (500 MHz, Methanol-d₄) δ ppm 1.72 (s, 3H), 2.38-2.42 (m, 1H), 2.58-2.61 (m, 1H), 3.13 (s, 3H), 3.73 (s, 2H), 3.94-3.97 (m, 1H), 4.30-4.34 (m, 1H), 6.77-6.82 (m, 2H), 7.28 (d, J=7.02 Hz, 1H), 7.33-7.40 (m, 4H), 7.66-7.76 (m, 5H).

¹³C NMR (176 MHz, Methanol-d₄) δ 171.21, 164.90, 163.45, 152.78, 140.38, 138.35, 135.24, 131.92, 128.78, 128.25, 127.16, 126.65, 120.06, 119.97, 114.21, 106.96, 68.48, 58.09, 45.61, 43.36, 35.97, 30.98, 14.06.

HRMS: m/z calc for C₂₅H₂₈N₃O₆S [M+H]⁺: 498.1693; found: 498.1691.

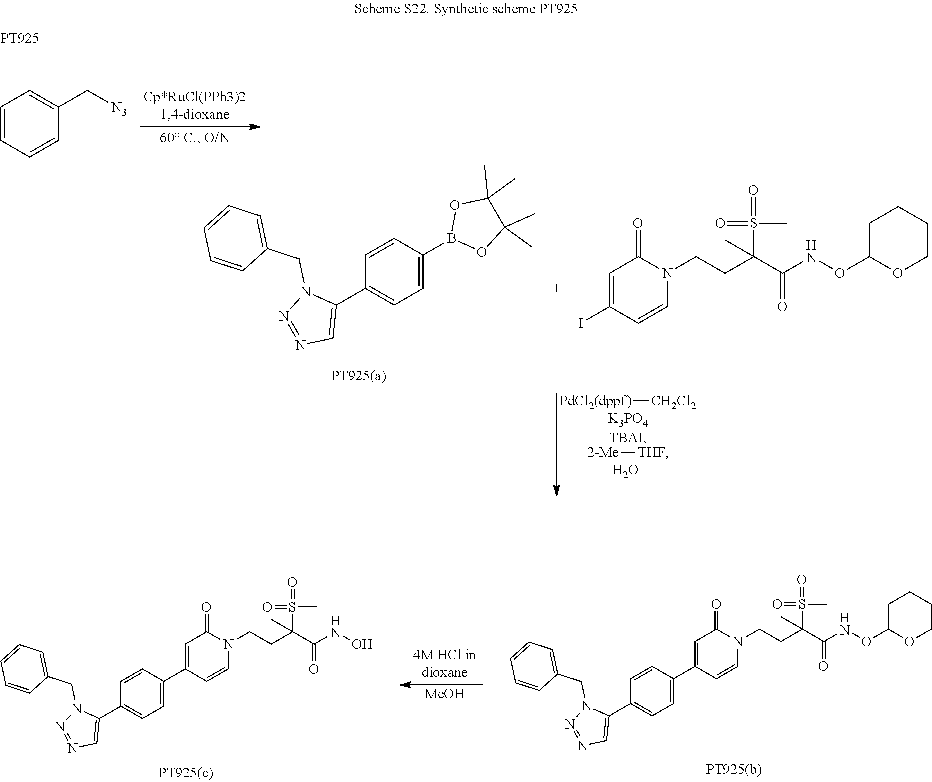

### PT925(a)—1-benzyl-5-(4-(4,4,5,5-tetramethyl-1,3,2-dioxaborolan-2-yl)phenyl)-1H-1,2,3-triazole

A solution of (azidomethyl)benzene (1 eq) and 2-(4-ethynylphenyl)-4,4,5,5-tetramethyl-1,2,3-dioxaborolane (1 eq) in 5 mL 1,4-dioxane was added to Cp*RuCl(PPh₃)₂ (0.02 eq) dissolved in 25 mL of 1,4-dioxane. The vial was purged with nitrogen, sealed, and heated in oil bath at 60° C. for 12 h, at which point TLC and LC-MS analysis indicated complete conversion of the starting materials. The mixture was purified using flash chromatography with hexanes/ethyl acetate. (Yield 46%)

PT925(a)—¹H NMR (700 MHz, Chloroform-d) δ ppm 1.38 (s, 12H), 5.57 (s, 2H), 7.09-7.14 (m, 2H), 7.29-7.33 (m, 4H), 7.79 (s, 1H), 7.87 (d, J=8.17 Hz, 2H).

LCMS: m/z calc for C₂₁H₂₅BN₃O₂ [M+H]⁺: 362.21; found: 362.20.

### PT925(b) was Synthesized Suzuki Coupling

PT925(b)—4-(4-(4-(1-benzyl-1H-1,2,3-triazol-5-yl)phenyl)-2-oxopyridin-1(2H)-yl)-2-methyl-2-(methylsulfonyl)-N-((tetrahydro-2H-pyran-2-yl)oxy)butanamide (Yield 54%)

PT925(b)—¹H NMR (700 MHz, Chloroform-d) δ ppm 1.61-1.65 (m, 2H), 1.70-1.72 (m, 1H), 1.73-1.75 (m, 3H), 1.79-1.84 (m, 1H), 1.88-1.94 (m, 1H), 1.95-2.01 (m, 1H), 2.41-2.47 (m, 1H), 2.53-2.60 (m, 1H), 3.24-3.26 (m, 3H), 3.65 (d, J=11.40 Hz, 1H), 4.12 (td, J=12.48, 6.24 Hz, 1H), 4.19-4.26 (m, 1H), 4.37-4.43 (m, 1H), 5.24 (br. s., 1H), 5.62 (s, 2H), 6.57 (d, J=8.17 Hz, 1H), 6.88 (s, 1H), 7.12 (d, J=5.59 Hz, 2H), 7.31-7.35 (m, 3H), 7.38 (m, 2H), 7.47 (d, J=6.88 Hz, 1H), 7.63 (m, 2H), 7.82 (s, 1H).

LCMS: m/z calc for C₃₁H₃₄N₅O₆S [M−H]⁻: 604.22; found: 604.20.

### PT925(c) was Synthesized HCl Deprotection

PT925(c) 4-(4-(4-(1-benzyl-1H-1,2,3-triazol-5-yl)phenyl)-2-oxopyridin-1(2H)-yl)-2-methyl-2-(methylsulfonyl)butanamide (Yield 69%)

¹H NMR (500 MHz, DMSO-d₆) δ ppm 1.58 (s, 3H), 2.18 (td, J=12.21, 5.04 Hz, 1H), 2.39-2.49 (m, 1H), 3.33 (s, 3H), 3.77 (td, J=11.94, 4.81 Hz, 1H), 4.13 (td, J=11.86, 4.96 Hz, 1H), 5.75 (s, 2H), 6.71 (d, J=7.17 Hz, 1H), 6.78 (s, 1H), 7.04 (d, J=7.17 Hz, 2H), 7.25-7.34 (m, 3H), 7.60 (m, 2H), 7.79 (d, J=7.02 Hz, 1H), 7.85 (m, 2H), 8.02-8.08 (m, 1H), 9.26 (s, 1H), 11.16 (br. s., 1H).

¹³C NMR (176 MHz, Methanol-d₄) δ 163.34, 152.12, 138.67, 137.83, 135.50, 132.95, 129.28, 128.52, 127.88, 127.23, 126.74, 115.61, 106.69, 68.46, 51.76, 45.60, 35.93, 31.04, 13.96.

HRMS: m/z calc for C₂₆H₂₈N₅O₅S [M+H]⁺: 522.1806; found: 522.1808.

### Purity Determination by HPLC

Compound purity was determined by HPLC, with solvent A: 0.1% TFA/H2O, B: 0.1% TFA/MeOH, gradient: 15%-95% MeOH over 9 min at a flowrate 0.5 mL/min. Wavelength: 299 nm. The low-res LC/MS analysis was done on Agilent 6110 Single Quadrupole equipped with an Agilent 1200 HPLC system. Column: InfinityLab Poroshell 120 EC-C₁₈ (3×50 mm 2.7 um).

### HPLC Method

A: 0.1% TFA/H₂O, B: 0.1% TFA/MeOH

| Time [min] | A [%] | B [%] |
|---|---|---|
| 0.00 | 85.0 | 15.0 |
| 0.70 | 85.0 | 15.0 |
| 6.00 | 5.0 | 95.0 |
| 7.00 | 5.0 | 95.0 |
| 7.10 | 85.0 | 15.0 |
| 9.00 | 85.0 | 15.0 |

### DISCUSSION

Drug selectivity has both thermodynamic and kinetic components enabling compound selection and optimization to be guided by both the affinity for the target as well as the rate constants for formation and breakdown of the drug-target complex (Georgi, 2018 and Sykes, 2012). To delineate the factors that underly the implementation of kinetic selectivity, it was identified that target vulnerability and target turnover are critical factors in modulating the translation of extended target occupancy to prolonged drug activity (Davvodi, 2020 and Basak, 2020). In antibacterial space, time-dependent drug activity can be assessed using the post-antibiotic effect (PAE), which is the delay in bacterial growth following compound washout (Bundtzen, 1981). While several mechanisms can be responsible for the PAE, there are correlations between drug-target residence time and PAE demonstrating that the ribosome and the LpxC enzyme from P. aeruginosa were highly vulnerable targets in contrast to, for example, the penicillin binding proteins (PBPs) from several bacterial species (Daryaee, 2016 and Tuomanen, 1986). The currently application explored the vulnerability of LpxC in E. coli and demonstrate that no PAE can be observed of inhibitors of E. coli LpxC. Using a strategy to stabilize ecLpxC, the lack of a PAE in E. coli with the rapid turnover of ecLpxC was now directly link.

Three ecLpxC inhibitors were chosen for analysis with residence times on the enzyme ranging from 6-66 min at 25° C. In contrast to inhibitors of paLpxC in P. aeruginosa, no PAE was observed for the ecLpxC inhibitors in a wild-type strain of E. coli (walkup, 2015). In both cases the LpxC inhibitors are cidal, and in P. aeruginosa even paLpxC inhibitors with short residence time gave a PAE of ˜1 h (walkup, 2015). Cognizant of studies which showed that ecLpxC was much less stable than the corresponding enzyme in P. aeruginosa, it was hypothesized that the lack of PAE in E. coli might be due to rapid target turnover, which was proposed to be the reason why β-lactam antibiotics do not generate a PAE in E. coli despite being covalent inhibitors of PBPs. (Davoodi, 2020)

The stability and intracellular levels of ecLpxC are controlled by the essential AAA protease FtsH (Fuhrer, 2006). Using pulse chase labelling with ³⁵S, Ogura et al. determined the half-life of ecLpxC to be 5.8 min at 37° C. and demonstrated that LpxC stability was dramatically increased (ti/2>60 min) in an E. coli strain with a mutation that resulted in hyperactivity of the fatty acid biosynthesis enzyme b-hydroxyacyl-ACP dehydrase FabZ (L85P, FabZ*) (Ogura, 1999). Both LPS and fatty acid biosynthesis use a common precursor, R-3-hydroxymyristoyl-ACP, and the increase in stability of ecLpxC in the FabZ* strain is thought to compensate for hyperactivity of FabZ and ensure that fatty acid and LPS biosynthesis remains balanced (Ogura, 1999). Subsequently, the susceptibility of LpxC to FtsH degradation in different bacterial species were analyzed and concluded that the C-terminus of LpxC serves as a degradation signal for FtsH. By monitoring the degradation of LpxC following inhibition of protein synthesis, ecLpxC and paLpxC were found to have half-lives of 4 min and 93 min, respectively, and that overexpression of FabZ was found to increase the half-life of ecLpxC to 69 min (langklotz, 2011 and Schakermann, 2013).

The currently application revealed that whereas the ecLpxC inhibitors do not give a PAE in wild-type E. coli, PAE values of 0.9-2.4 h were observed at 16×MIC in the FabZ* strain. This effect appeared to be LpxC-specific since the control antibiotics erythromycin (macrolide), cefamandole (β-lactam) and trimethoprim (folate biosynthesis) had the same PAE values in both wild-type and FabZ* strains (Table 1). Consistent with previous findings, neither cefamandole nor trimethoprim showed a PAE with the wild-type strain, a result that was reproduced in the FabZ* strain. Similarly, erythromycin gave a PAE of 0.7-1.4 h (4× and 16×MIC) in both strains, again consistent with previous reports (Bundtzen, 2981, Minguez, 1993 and Odenholt-Tornqvist, 1995)

pSILAC was used to quantify ecLpxC turnover and measured half-lives of 12 min and 35 min in wild-type and FabZ* E. coli, respectively (Table 2), in general agreement with previous studies (Ogura, 1999 and Thomanek, 2018). The ecLpxC antibody pull-down method also resulted in the enrichment of several other proteins that served as controls for the pSILAC experiments and revealed that hyperactivity of FabZ only affected the stability of ecLpxC. While the control proteins all had significantly longer half-lives than ecLpxC in wild-type E. coli to begin with (12 compared to 50-80 min), the results support a specific effect on LpxC stability.

To further explore the relationship between protein stability and PAE, PAE values and the rate of protein turnover were measured in the presence of sub-MIC concentrations of azithromycin (0.2-0.4×MIC). Sub-MIC concentrations of the macrolides azithromycin and erythromycin are known to reduce the rate of protein synthesis, generally without affecting viability, it seems that the presence of sub-MIC macrolide in the PAE phase would reduce the rate of ecLpxC biosynthesis, and or of the protease FtsH that regulates levels or ecLpxC, thereby resulting in an increase in protein stability. (Odenholt-Tornqvist, 1995, Champney, 1999 and Tateda, 2000). Indeed, whereas no PAE was observed for wild-type E. coli in the absence of macrolide, introduction of even 0.2×MIC azithromycin resulted in a robust (1.4-2.8 h) PAE for the ecLpxC inhibitors. To validate if sub-MIC macrolide had indeed increased the stability of ecLpxC, pSILAC experiments were preformed which revealed a 2-fold increase in the half-life of ecLpxC (t_{1/2} 12 to 23 min) in the presence of 0.4×MIC azithromycin. Consistent with a general effect on protein synthesis, the sub-MIC macrolide also resulted in an increase in the half-lives of the control proteins (1.2-1.4-fold) with the exception of OmpA which showed a slight decrease in stability (Table 3).

Although many potent inhibitors of LpxC have been developed, there have been none that progressed through clinical trials. ACHN-975 entered clinical trials but was found to have dose-limiting cardiovascular toxicity (Cohen, 2019). A subsequent medicinal chemistry study was unsuccessful at identifying which structural features might be contributing to the toxicity, but demonstrated that the hydroxamate was not responsible for the adverse effects. The residence time of inhibitors on paLpxC was shown to be correlated with the delay in recovery of bacterial growth following treatment of P. aeruginosa (PAO1) with the compounds and subsequent washout (PAE) (Walkup, 2015). Since an increase in PAE will likely reduce the frequency of dosing, strategies that increase the residence time of inhibitors on paLpxC may increase the therapeutic window. Consequently, the objective of the current application was to determine the molecular basis that controls the residence time of inhibitors on paLpxC.

Compound 1 (PF5081090) was chosen as the lead for the structure-kinetic relationship studies. This compound lacks the alkyne moiety which is present in many LpxC inhibitors and may be a metabolic liability (Montgomery, 2012). Compound 1 has potent biochemical and microbiological activity (Kᵢ* paLpxC 20 μM, MIC PAO1 0.6 μM), a residence time on paLpxC of 30 min at 37° C., and causes a PAE of 1.26 h at 4×MIC against PAO1 (Walkup, 2015). In addition, Compound 1 also has in vivo antibacterial activity in animal models of P. aeruginosa and Klebsiella pneumonia infection, and a PK/PD model was developed that successfully predicted the in vivo activity of Compound 1 in a model of P. aeruginosa infection (Tomaras, 2014).

LpxC inhibitors consist of three components including a bidentate chelating ligand, primarily a hydroxamic acid which chelates the catalytic Zn²⁺, a polar head group which partially occupies the substrate sugar-binding pocket, and a hydrophobic tail which occupies the hydrophobic tunnel that normally accommodates the C₁₀-C₁₆ aliphatic chain of the substrate (Erwin, 2016). Previous SAR studies of biphenyl methylsulfone hydroxamate analogs have demonstrated that the 4-position of the terminal aryl ring is tolerant to a broad range of substituents (Brown, 2012) A library of compounds with a variety of substituents at the para-position of the terminal benzene ring of compound 1 was designed. This included a series of compounds generated by click chemistry reactions and several analogs of 1 previously reported by Pfizer, for which residence time data was not available. Apart from 1, which was commercially available as the (R) enantiomer, all the pyridone methylsulfones were prepared as the racemates in which it was assumed that only the (R) enantiomer bound to the enzyme (Si, 2019). Two compounds reported by Novartis were also analyzed, in which the methylsulfone of 1 had been replaced by a cyclic sulfone.

Analysis of the binding kinetics data in Table 4 revealed that the triazole substituent is generally well tolerated with most analogs and produced residences times that are longer than that of 1. PT913, PT914, and PT917, bearing bicyclopentane or pyridine groups on the triazole, have the longest residence times of ˜120 min, which are ˜3-fold longer than 1. In addition, PT805, which has a triazole ring connected directly via the central nitrogen to the phenyl ring, has a residence time that is 1.5-fold longer than 1. However, the regiochemistry of the triazole is important since PT925 with a benzyl group on N1 has a residence time of 9 min compared to the regioisomer with the benzyl group at N3, which has a residence time of 57 min (PT810). In contrast, analogs of 1 either lacking substituents on the phenyl ring (PT901) or only possessing the methoxy group (PT902) had shorter residence times than 1 (5 and 15 min compared to 40 min) (Table 5). Analogs with substituents connected directly to the phenyl ring such as PT904 (morpholine), PT923 (N-benzylacetamide), and PT924 (N-methyl-2-phenylacetamide) have short residence times (4-8 min). We also explored the impact of altering the head group, although these compounds also had a phenyl ring in place of the central pyridone found in 1 (Table 5). While no binding was observed for PT903, which has the threonine group found in CHIR-090, the cyclic sulfone group was well tolerated with PT920 and PT921 having residence times of ˜30 min.

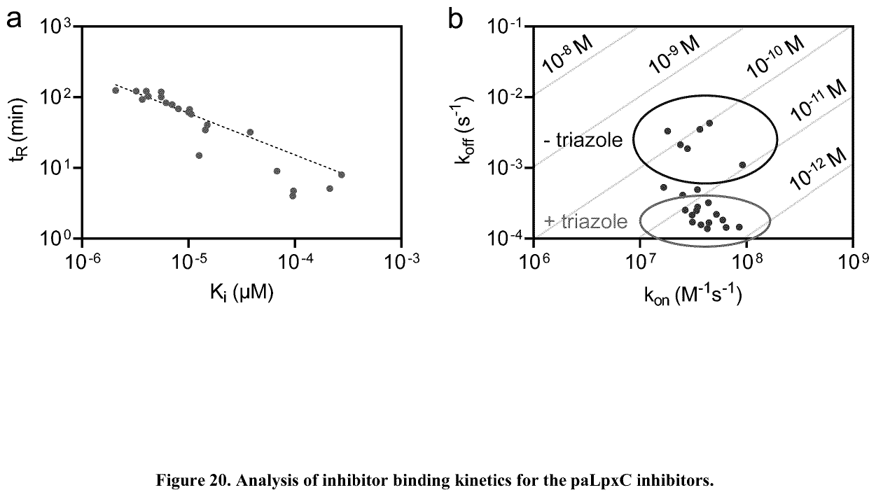

The changes in residence time for the paLpxC inhibitors correlated with an increase in thermodynamic affinity, indicating that ground state stabilization was the principal factor in modulating off rate (walkup, 2015). This trend was also observed in the current set of compounds, in which an increase in residence time was generally correlated with a decrease in Kᵢ (FIG. 20). Further information about the relationship between kₒₙ, k_{off}, and Kᵢ can be visualized using a using a 2-D kinetic map (FIG. 20) (Schneider, 2013, Schoop 2015 and Spagnuolo, 2017). In general, the triazole based compounds have longer residence times and better potency (smaller Kᵢ values) than the non-triazole-based compounds.

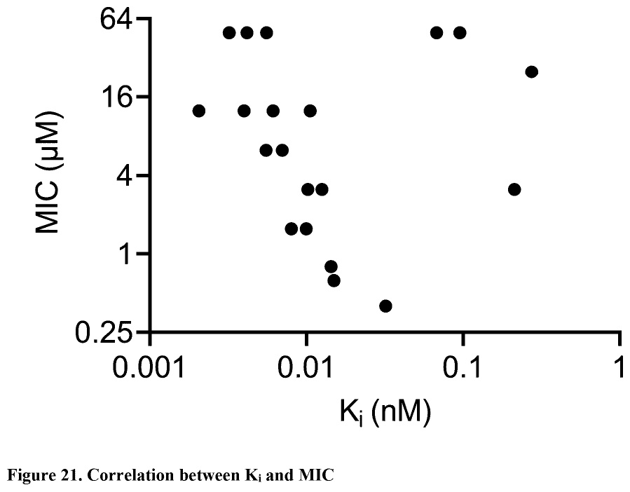

The cellular activity of the paLpxC inhibitors were characterized. All compounds tested for cytotoxicity showed greater than 80% viability against Vero cells up to a 200 μM concentration. In addition, the compounds displayed bactericidal activity against PAO1 (FIG. 18), and the MIC value for each paLpxC inhibitor was lower for the ΔMexABCDXY strain of P. aeruginosa compared to PAO1, indicating that efflux plays a significant role in their antimicrobial activity (Tables 4 and 5). Compound 1 has an MIC of 0.625 μM against PAO1, which drops to 0.018 μM for the pump mutant (Walkup, 2015). The triazole analog PT805 and the cyclic sulfones PT920 and PT921, previously reported by Pfizer and Novartis, respectively, have comparably robust activities (Cohen 2019). Similarly, the p-fluorophenyl triazole analog PT909 reported here has MIC values of 1.56 and 0.05 μM against PAO1 and the pump mutant, respectively. No correlation was observed between MIC and Kᵢ (FIG. 21), which could be because the Kᵢ values measured here are for the overall inhibition of the enzyme and include both the initial fast and slow steps in the two-step formation of the final enzyme-inhibitor complex(Daryaee, 2019). In addition, guidelines on antibacterial compound development indicate that low or even negative clogP values are desirable, and we note that Compound 1 has a clogP of −0.15 and an MIC of 0.625 M which is lower than all the triazole analogs. During our SAR investigation, we substituted the benzene ring of PT810 (clogP 1.59) with substituents such as bicyclopentane (PT913, clogP 0.95), 4-pyridine (PT915, clogP 0.25) and pyrimidine (PT916, clogP 0.08) to generate compounds with improved clogP (O'Shea, 2008 and Richter, 2017). However, there is no observation of a correlation between cLogP and MIC, although the MIC for the 2-pyridine analog PT914 (clogP 0.68) was 2-fold lower than that for PT810.

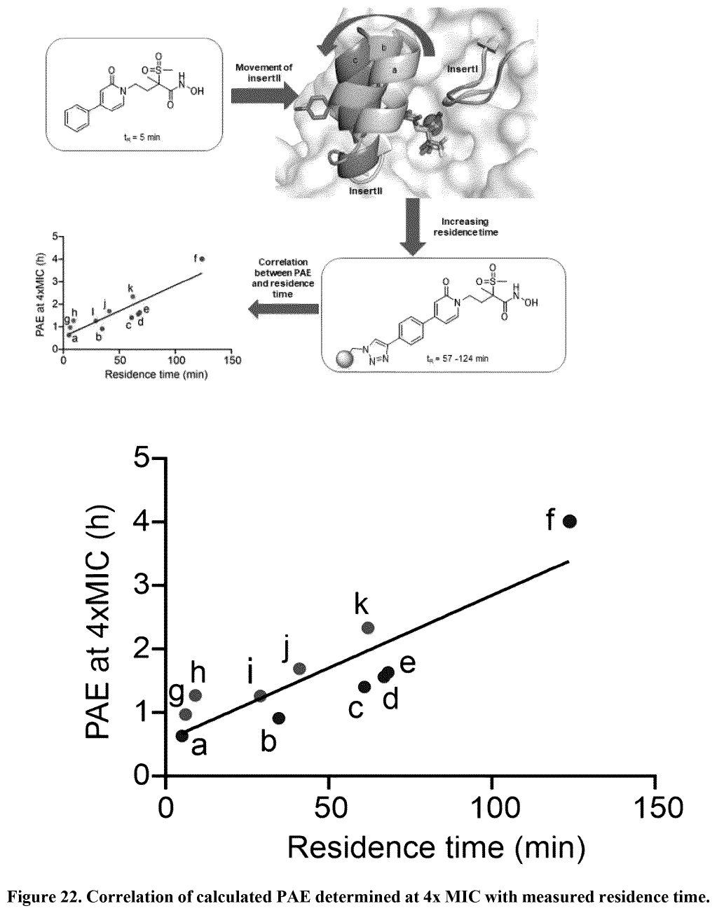

Time-dependent antibacterial activity was evaluated by determining the PAEs for several paLpxC inhibitors, which had a range of residence times of up to 124 min (PT913) (Davoodi, 2020). As observed previously for inhibitors of LpxC and FabI, the PAE of each compound increased with drug concentration due to increased levels of target engagement (Walkup, 2015 and Daryaee, 2016). In addition, although no correlation was observed between MIC and PAE, a positive correlation was found between residence time and PAE (Table 6). In FIG. 22, the residence time and PAE values were plotted for the paLpxC inhibitors together with data previously reported by Walkup et al., giving a linear correlation with a slope of 0.023 (Walkup, 2015). The data support the fundamental premise that further increases in residence time should have a beneficial impact on time-dependent antibacterial activity.

To gain insight into the molecular factors that modulate residence time, the x-ray structures of paLpxC was overlaid in complex with inhibitors of varying binding kinetics (FIG. 7). A principal difference in these structures is the position of the Insert II helix (195-204 in paLpxC) that comprises part of the hydrophobic substrate tunnel and which interacts with the tail of the inhibitors. In FIG. 7, the structures of paLpxC was overlaid bound to the pyridone methylsulfone inhibitors PT901 (PDB 3UHM) and 1 (PDB 5UPG), as well as the diacetylene inhibitor LPC-058 (PDB 5DRR) (Lee, 2016). PT901 is a short residence time inhibitor, and the increase in residence time for Compound 1 (41 min) is accompanied by a movement of Insert II towards the inhibitor. A similar movement is observed in the structure of PT805 bound to paLpxC (PDB 6cax), which has a residence time of 61 min (not shown). The paLpxC:LPC-058 structure shows the largest movement of Insert II. Although the kinetics for inhibition of paLpxC by LPC-058 have not been reported, this inhibitor has a residence time of ˜12 h on LpxC from E. coli. Analysis of other structures in the protein data bank reveals that Insert II generally lies between the limits set by PT901 and LPC-058. Specific interactions between Insert II and the inhibitor that are thought to be important for stabilizing the complexes and potentially modulating binding kinetics include R201, which forms a π-cation interaction with the triazole of PT805.

The observed movement of Insert II is in line with previous observations (Lee, 2011). Based on the structure of CHIR-090 bound to LpxC from Aquifex aeolicus (aaLpxC), Barb et al. proposed that interactions of the inhibitor with Insert II were involved in the two-step slow-onset inhibition of the enzyme by CHIR-090, which has a residence time of >24 h on the enzyme (McClerren, 2005). Movement of Insert II has also been linked to changes in inhibitor potency. For instance, Surivet et al. speculated that the increase in affinity of isoindolin-1-one 10 compared to oxazol-2-one (R)-8, was due to closer contact of Insert II with the inhibitor (Surivet, 2020). In addition, it was noted that Insert II adopted a closed conformation when the potent inhibitor BB-78485 was bound to the enzyme but was in an open conformation when the hydrophobic tunnel was occupied by the substrate fatty acyl group (Mochalkin, 2008).

Although a structure of paLpxC bound to product is not available, in FIG. 7, the structures of ecLpxC bound to BB-78485 (PDB 4ISA) and UDP-(3-O—(R-3-hydroxymyristoyl))-glucosamine (PDB 4MDT) was overlaid, which emphasizes the movement of the Insert II helix from an open to a closed conformation (Lee, 2016). In previously study on InhA, the enoyl-ACP reductase from Mycobacterium tuberculosis, the time-dependent inhibition of this enzyme was attributed to a movement of helix-6 from an open to a closed conformation (Li, 2014 and Lai, 2015). The open conformation bound substrate and rapid-reversible inhibitors, while the slow-onset inhibition of the enzyme resulted from a closed conformation of helix-6, in which amino acid side chains from this helix occupied part of the active site. A similar model can be proposed for LpxC. In the structure of BB-78485 bound to paLpxC, interactions that likely stabilize the closed conformation include 1197, which forms a van der Waals contact with V216, and R201, which is hydrogen bonded to the backbone C═O of G209. As noted previously, R201 also interacts with more extended inhibitors than BB-78485. For instance, it forms a π-cation interaction with the triazole of PT805 (FIG. 16). The importance of this contact is supported by the short residence time of PT904, in which the triazole in PT805 is replaced by a morpholine group (Table 5). The π-cation interaction is likely recapitulated in the complex with PT810, which has a residence time of 57 min, but not in the complexes of paLpxC with PT923 and PT924, which are two open-chain amide analogs of PT810 (Table 4). Lastly, studies with the triazole containing inhibitors PT903, in which the pyridone methylsulfone is replaced by a phenyl-threonine group (Table 5), and PT925, a regioisomer of PT810, show that precise positioning of the triazole is important for productive interactions with the enzyme.

Finally, it is important to note that other factors such as lipophilicity and MW may also play a role in modulating residence time (Pan, 2013). An analysis of time-dependent enzyme inhibitors revealed that compounds with clogP>5 were more likely to have a long residence time (Miller, 2012). While similar conclusions have been drawn from studies in other targets, an increase in lipophilicity is also often associated with a reduction in solubility which can be a concern in drug development. Miller, 2012, Lu, 2018 and Tresadern, 2011) Analyzing the library of the triazole based LpxC inhibitors described here, it was indemnified that compounds with long residence time but with reduced clogP or potentially improved solubility. For example, comparison of PT810 (clogP 1.59, t_{R} 57 min) with PT913 (clogP 0.95, t_{R} 124 min) or PT917 (clogP 0.35, t_{R} 121.3 min) shows the residence time could be improved without increasing the lipophilicity of the compounds.

### CONCLUSION

The impact of molecular approaches that stabilize LpxC in E. coli highlight the central role that protein turnover plays in controlling the translation of sustained target occupancy to prolonged drug activity. Using the PAE as a metric for time-dependent antibacterial activity, it was shown that rapid target turnover masks the effect of drug-target residence time. A reduction in the rate of target turnover restores the coupling between target occupancy and PAE, with only a relatively small (2-3-fold) change in stability needed to generate a robust PAE. At a wider level, the data reinforce that covalent inhibitor discovery should focus on targets that turnover slowly and that approaches to increase target stability, for example using transcription/translation inhibitors, are likely to result in a significant widening of the therapeutic window derived from kinetic selectivity (Aeschlimann, 1999).

Analogs of the paLpxC inhibitor PF-5081090 (1) were synthesized to build a structure-kinetic relationship for time-dependent enzyme inhibition. A subset of the analogs was generated using click chemistry, which had longer residence times on paLpxC than Compound 1. Structural data support a model in which the Insert II helix moves from an open conformation found in complexes of the enzyme with substrate or short residence time compounds to a closed conformation for long residence time inhibitors. Analogous to studies with the enoyl-ACP reductase InhA, the current application proposed that the open to closed movement of the Insert II helix is responsible for the slow step on the binding reaction coordinate. Microbiological studies show that the increase in paLpxC residence time translates to a lengthening of the PAE after compound washout. Given that drugs with extended activity following clearance can be dosed less frequently, this application provides a foundation for the development of LpxC inhibitors with an improved therapeutic window.

### REFERENCE

1. Payne, D. J., Gwynn, M. N., Holmes, D. J. and Pompliano, D. L. (2007) Drugs for bad bugs: confronting the challenges of antibacterial discovery. Nat Rev Drug Discov 6, 29-40. DOI: 10.1038/nrd2201.
2. Silver, L. L. (2011) Challenges of antibacterial discovery. Clin Microbiol Rev 24, 71-109. DOI: 10.1128/CMR.00030-10.
3. Tommasi, R., Brown, D. G., Walkup, G. K., Manchester, J. I. and Miller, A. A. (2015) ESKAPEing the labyrinth of antibacterial discovery. Nat Rev Drug Discov 14, 529-542. DOI: 10.1038/nrd4572.
4. Kaur, P., Agarwal, S. and Datta, S. (2009) Delineating bacteriostatic and bactericidal targets in mycobacteria using IPTG inducible antisense expression. PLoS One 4, e5923. DOI: 10.1371/journal.pone.0005923.
5. Ramachandran, V., Singh, R., Yang, X., Tunduguru, R., Mohapatra, S., Khandelwal, S., Patel, S. and Datta, S. (2013) Genetic and chemical knockdown: a complementary strategy for evaluating an anti-infective target. Adv Appl Bioinform Chem 6, 1-13. DOI: 10.2147/AABC.539198.
6. Wei, J. R., Krishnamoorthy, V., Murphy, K., Kim, J. H., Schnappinger, D., Alber, T., Sassetti, C. M., Rhee, K. Y. and Rubin, E. J. (2011) Depletion of antibiotic targets has widely varying effects on growth. Proc Natl Acad Sci USA 108, 4176-81. DOI: 10.1073/pnas.1018301108.
7. Davoodi, S., Daryaee, F., Chang, A., Walker, S. G. and Tonge, P. J. (2020) Correlating Drug-Target Residence Time and Post-antibiotic Effect: Insight into Target Vulnerability. ACS Infect Dis 6, 629-636. DOI: 10.1021/acsinfecdis.9b00484.
8. Basak, S. and Tonge, P. J., Drug Target Residence Time and the Pharmacodynamics of Antibacterial Agents. In Med Chem Rev, Bronson, J. J., Ed. Medicinal Chemistry Division of the American Chemical Society: Washington, D C., 2020; Vol. 55, pp 367-380.
9. Tonge, P. J. (2018) Drug-Target Kinetics in Drug Discovery. ACS Chem Neurosci 9, 29-39. DOI: 10.1021/acschemneuro.7b00185.
10. Walkup, G. K., You, Z., Ross, P. L., Allen, E. K., Daryaee, F., Hale, M. R., O'Donnell, J., Ehmann, D. E., Schuck, V. J., Buurman, E. T., Choy, A. L., Hajec, L., Murphy-Benenato, K., Marone, V., Patey, S. A., Grosser, L. A., Johnstone, M., Walker, S. G., Tonge, P. J. and Fisher, S. L. (2015) Translating slow-binding inhibition kinetics into cellular and in vivo effects. Nat Chem Biol 11, 416-23. DOI: 10.1038/nchembio.1796.
11. Schapira, M., Calabrese, M. F., Bullock, A. N. and Crews, C. M. (2019) Targeted protein degradation: expanding the toolbox. Nat Rev Drug Discov 18, 949-963. DOI: 10.1038/s41573-019-0047-y.
12. Whittington, D. A., Rusche, K. M., Shin, H., Fierke, C. A. and Christianson, D. W. (2003) Crystal structure of LpxC, a zinc-dependent deacetylase essential for endotoxin biosynthesis. Proc Natl Acad Sci USA 100, 8146-50. DOI: 10.1073/pnas.1432990100.
13. Barb, A. W., McClerren, A. L., Snehelatha, K., Reynolds, C. M., Zhou, P. and Raetz, C. R. (2007) Inhibition of lipid A biosynthesis as the primary mechanism of CHIR-090 antibiotic activity in Escherichia coli. Biochemistry 46, 3793-802. DOI: 10.1021/bi6025165.
14. Langklotz, S., Schakermann, M. and Narberhaus, F. (2011) Control of lipopolysaccharide biosynthesis by FtsH-mediated proteolysis of LpxC is conserved in enterobacteria but not in all gram-negative bacteria. J Bacteriol 193, 1090-7. DOI: 10.1128/JB.01043-10.
15. Ogura, T., Inoue, K., Tatsuta, T., Suzaki, T., Karata, K., Young, K., Su, L. H., Fierke, C. A., Jackman, J. E., Raetz, C. R., Coleman, J., Tomoyasu, T. and Matsuzawa, H. (1999) Balanced biosynthesis of major membrane components through regulated degradation of the committed enzyme of lipid A biosynthesis by the AAA protease FtsH (HflB) in Escherichia coli. Mol Microbiol 31, 833-44. DOI: 10.1046/j.1365-2958.1999.01221.x.
16. Schakermann, M., Langklotz, S. and Narberhaus, F. (2013) FtsH-mediated coordination of lipopolysaccharide biosynthesis in Escherichia coli correlates with the growth rate and the alarmone (p)ppGpp. J Bacteriol 195, 1912-9. DOI: 10.1128/JB.02134-12.
17. Thomanek, N., Arends, J., Lindemann, C., Barkovits, K., Meyer, H. E., Marcus, K. and Narberhaus, F. (2018) Intricate Crosstalk Between Lipopolysaccharide, Phospholipid and Fatty Acid Metabolism in Escherichia coli Modulates Proteolysis of LpxC. Front Microbiol 9, 3285. DOI: 10.3389/fmicb.2018.03285.
18. Montgomery, J. I., Brown, M. F., Reilly, U., Price, L. M., Abramite, J. A., Arcari, J., Barham, R., Che, Y., Chen, J. M., Chung, S. W., Collantes, E. M., Desbonnet, C., Doroski, M., Doty, J., Engtrakul, J. J., Harris, T. M., Huband, M., Knafels, J. D., Leach, K. L., Liu, S., Marfat, A., McAllister, L., McElroy, E., Menard, C. A., Mitton-Fry, M., Mullins, L., Noe, M. C., O'Donnell, J., Oliver, R., Penzien, J., Plummer, M., Shanmugasundaram, V., Thoma, C., Tomaras, A. P., Uccello, D. P., Vaz, A. and Wishka, D. G. (2012) Pyridone methylsulfone hydroxamate LpxC inhibitors for the treatment of serious gram-negative infections. J Med Chem 55, 1662-70. DOI: 10.1021/jm2014875.
19. McClerren, A. L., Endsley, S., Bowman, J. L., Andersen, N. H., Guan, Z., Rudolph, J. and Raetz, C. R. (2005) A slow, tight-binding inhibitor of the zinc-dependent deacetylase LpxC of lipid A biosynthesis with antibiotic activity comparable to ciprofloxacin. Biochemistry 44, 16574-83. DOI: 10.1021/bi0518186.
20. Penefsky, H. S. (1979) A centrifuged-column procedure for the measurement of ligand binding by beef heart F1. Methods Enzymol 56, 527-30. DOI: 10.1016/0076-6879(79)56050-9.
21. Yu, W., Neckles, C., Chang, A., Bommineni, G. R., Spagnuolo, L., Zhang, Z., Liu, N., Lai, C., Truglio, J. and Tonge, P. J. (2015) A [(32)P]NAD(+)-based method to identify and quantitate long residence time enoyl-acyl carrier protein reductase inhibitors. Anal Biochem 474, 40-9. DOI: 10.1016/j.ab.2014.12.022.
22. Pankey, G. A. and Sabath, L. D. (2004) Clinical relevance of bacteriostatic versus bactericidal mechanisms of action in the treatment of Gram-positive bacterial infections. Clin Infect Dis 38, 864-70. DOI: 10.1086/381972.
23. Keilhauer, E. C., Hein, M. Y. and Mann, M. (2015) Accurate protein complex retrieval by affinity enrichment mass spectrometry (AE-MS) rather than affinity purification mass spectrometry (AP-MS). Mol Cell Proteomics 14, 120-35. DOI: 10.1074/mcp.M114.041012.
24. Fröhlich, F., Christiano, R. and Walther, T. C. (2013) Native SILAC: Metabolic Labeling of Proteins in Prototroph Microorganisms Based on Lysine Synthesis Regulation. Mol Cell Proteomics 12, 1995. DOI: 10.1074/mcp.M112.025742.
25. Cargile, B. J., Bundy, J. L., Grunden, A. M. and Stephenson, J. L. (2004) Synthesis/degradation ratio mass spectrometry for measuring relative dynamic protein turnover. Anal Chem 76, 86-97. DOI: 10.1021/ac034841a.
26. Soufi, B., Kumar, C., Gnad, F., Mann, M., Mijakovic, I. and Macek, B. (2010) Stable Isotope Labeling by Amino Acids in Cell Culture (SILAC) Applied to Quantitative Proteomics of Bacillus subtilis. J Proteome Res 9, 3638-3646. DOI: 10.1021/pr100150w.
27. Soares, N. C., Spät, P., Krug, K. and Macek, B. (2013) Global Dynamics of the Escherichia coli Proteome and Phosphoproteome During Growth in Minimal Medium. J Proteome Res 12, 2611-2621. DOI: 10.1021/pr3011843.
28. Copeland, R. A., Pompliano, D. L. and Meek, T. D. (2006) Drug-target residence time and its implications for lead optimization. Nat Rev Drug Discov 5, 730-9. DOI: 10.1038/nrd2082.
29. Georgi, V., Schiele, F., Berger, B. T., Steffen, A., Marin Zapata, P. A., Briem, H., Menz, S., Preusse, C., Vasta, J. D., Robers, M. B., Brands, M., Knapp, S. and Fernandez-Montalvan, A. (2018) Binding Kinetics Survey of the Drugged Kinome. J Am Chem Soc 140, 15774-15782. DOT: 10.1021/jacs.8b08048.
30. Sykes, D. A., Dowling, M. R., Leighton-Davies, J., Kent, T. C., Fawcett, L., Renard, E., Trifilieff, A. and Charlton, S. J. (2012) The Influence of receptor kinetics on the onset and duration of action and the therapeutic index of NVA237 and tiotropium. J Pharmacol Exp Ther 343, 520-8. DOI: 10.1124/jpet.112.194456.
31. Daryaee, F. and Tonge, P. J. (2019) Pharmacokinetic-pharmacodynamic models that incorporate drug-target binding kinetics. Curr Opin Chem Biol 50, 120-127. DOI: 10.1016/j.cbpa.2019.03.008.
32. Berger, B. T., Amaral, M., Kokh, D. B., Nunes-Alves, A., Musil, D., Heinrich, T., Schroder, M., Neil, R., Wang, J., Navratilova, I., Bomke, J., Elkins, J. M., Muller, S., Frech, M., Wade, R. C. and Knapp, S. (2021) Structure-kinetic relationship reveals the mechanism of selectivity of FAK inhibitors over PYK2. Cell Chem Biol 28, 686-698 e7. DOI: 10.1016/j.chembiol.2021.01.003.
33. Singh, J., Petter, R. C., Baillie, T. A. and Whitty, A. (2011) The resurgence of covalent drugs. Nat Rev Drug Discov 10, 307-17. DOI: 10.1038/nrd3410.
34. Bundtzen, R. W., Gerber, A. U., Cohn, D. L. and Craig, W. A. (1981) Postantibiotic suppression of bacterial growth. Rev Infect Dis 3, 28-37. DOI: 10.1093/clinids/3.1.28.
35. Daryaee, F., Chang, A., Schiebel, J., Lu, Y., Zhang, Z., Kapilashrami, K., Walker, S. G., Kisker, C., Sotriffer, C. A., Fisher, S. L. and Tonge, P. J. (2016) Correlating Drug-Target Kinetics and In vivo Pharmacodynamics: Long Residence Time Inhibitors of the FabI Enoyl-ACP Reductase. Chem Sci 7, 5945-5954. DOI: 10.1039/C6SC01000H.
36. Tuomanen, E. (1986) Newly made enzymes determine ongoing cell wall synthesis and the antibacterial effects of cell wall synthesis inhibitors. J Bacteriol 167, 535-43. DOI: 10.1128/jb.167.2.535-543.1986.
37. Fuhrer, F., Langklotz, S. and Narberhaus, F. (2006) The C-terminal end of LpxC is required for degradation by the FtsH protease. Mol Microbiol 59, 1025-36. DOI: 10.1111/j.1365-2958.2005.04994.x.
38. Minguez, F., Ramos, C., Loscos, A., Chiu, M. L. and Prieto, J. (1993) In vitro killing kinetics and postantibiotic effect of josamycin and other macrolides on several bacteria. Chemotherapy 39, 163-8. DOI: 10.1159/000239121.
39. Odenholt-Tornqvist, I., Lowdin, E. and Cars, O. (1995) Postantibiotic effects and postantibiotic sub-MIC effects of roxithromycin, clarithromycin, and azithromycin on respiratory tract pathogens. Antimicrob Agents Chemother 39, 221-6. DOI: 10.1128/aac.39.1.221.
40. Champney, W. S. and Tober, C. L. (1999) Molecular investigation of the postantibiotic effects of clarithromycin and erythromycin on Staphylococcus aureus cells. Antimicrob Agents Chemother 43, 1324-8. DOI: 10.1128/AAC.43.6.1324.
41. Tateda, K., Ishii, Y., Matsumoto, T., Kobayashi, T., Miyazaki, S. and Yamaguchi, K. (2000) Potential of macrolide antibiotics to inhibit protein synthesis of Pseudomonas aeruginosa: suppression of virulence factors and stress response. J Infect Chemother 6, 1-7. DOI: 10.1007/s101560050042.
42. Jensen, K. F. (1993) The Escherichia coli K-12 “wild types” W3110 and MG1655 have an rph frameshift mutation that leads to pyrimidine starvation due to low pyrE expression levels. J Bacteriol 175, 3401-7. DOI: 10.1128/jb.175.11.3401-3407.1993.
43. Clements, J. M., Coignard, F., Johnson, I., Chandler, S., Palan, S., Waller, A., Wijkmans, J. and Hunter, M. G. (2002) Antibacterial activities and characterization of novel inhibitors of LpxC. Antimicrob Agents Chemother 46, 1793-9. DOI: 10.1128/AAC.46.6.1793-1799.2002.
44. CLSI, Methods for Dilution Antimicrobial Susceptibility Tests for Bacteria That Grow Aerobically. In CLSI Standard M07, 11 ed.; Clinical and Laboratory Standards Institute: Wayne, PA, 2018.
45. Soufi, B. and Macek, B. (2014) Stable isotope labeling by amino acids applied to bacterial cell culture. Methods Mol Biol 1188, 9-22. DOI: 10.1007/978-1-4939-1142-4_2.
46. Pratt, J. M., Petty, J., Riba-Garcia, I., Robertson, D. H., Gaskell, S. J., Oliver, S. G. and Beynon, R. J. (2002) Dynamics of protein turnover, a missing dimension in proteomics. Mol Cell Proteomics 1, 579-91. DOI: 10.1074/mcp.m200046-mcp200.
47. Heo, S., Diering, G. H., Na, C. H., Nirujogi, R. S., Bachman, J. L., Pandey, A. and Huganir, R. L. (2018) Identification of long-lived synaptic proteins by proteomic analysis of synaptosome protein turnover. Proc Natl Acad Sci USA 115, E3827. DOI: 10.1073/pnas.1720956115.
48. Swinney, D. C. (2004) Biochemical mechanisms of drug action: what does it take for success?Nat Rev Drug Discov, 3, 801-8. DOI: 10.1038/nrdl500.
49. Copeland, R. A.; Pompliano, D. L.; Meek, T. D. (2006) Drug-target residence time and its implications for lead optimization Nat Rev Drug Discov, 5, 730-9. DOI: 10.1038/nrd2082.
50. Vauquelin, G.; Charlton, S. J. (2010) Long-lasting target binding and rebinding as mechanisms to prolong in vivo drug action Br J Pharmacol, 161, 488-508. DOI: 10.1111/j.1476-5381.2010.00936.x.
51. Copeland, R. A. (2016) The drug-target residence time model: a 10-year retrospective Nat Rev Drug Discov, 15, 87-95. DOI: 10.1038/nrd.2015.18.
52. Lu, H.; Tonge, P. J. (2010) Drug-target residence time: critical information for lead optimization Curr Opin Chem Biol, 14, 467-74. DOI: 10.1016/j.cbpa.2010.06.176.
53. Tonge, P. J. (2018) Drug-Target Kinetics in Drug Discovery ACS Chem Neurosci, 9, 29-39. DOI: 10.1021/acschemneuro.7b00185.
54. Onishi, H. R.; Pelak, B. A.; Gerckens, L. S.; Silver, L. L.; Kahan, F. M.; Chen, M. H.; Patchett, A. A.; Galloway, S. M.; Hyland, S. A.; Anderson, M. S.; Raetz, C. R. (1996) Antibacterial agents that inhibit lipid A biosynthesis Science, 274, 980-2. DOI: 10.1126/science.274.5289.980.
55. Jackman, J. E.; Raetz, C. R.; Fierke, C. A. (1999) UDP-3-O—(R-3-hydroxymyristoyl)-N-acetylglucosamine deacetylase of Escherichia coli is a zinc metalloenzyme Biochemistry, 38, 1902-11. DOI: 10.1021/bi982339s.
56. Barb, A. W.; Jiang, L.; Raetz, C. R.; Zhou, P. (2007) Structure of the deacetylase LpxC bound to the antibiotic CHIR-090: Time-dependent inhibition and specificity in ligand binding Proc Natl Acad Sci USA, 104, 18433-8. DOI: 10.1073/pnas.0709412104.
57. Clements, J. M.; Coignard, F.; Johnson, I.; Chandler, S.; Palan, S.; Waller, A.; Wijkmans, J.; Hunter, M. G. (2002) Antibacterial activities and characterization of novel inhibitors of LpxC Antimicrob Agents Chemother, 46, 1793-9. DOI: 10.1128/AAC.46.6.1793-1799.2002.
58. Barb, A. W.; Zhou, P. (2008) Mechanism and inhibition of LpxC: an essential zinc-dependent deacetylase of bacterial lipid A synthesis Curr Pharm Biotechnol, 9, 9-15. DOI: 10.2174/138920108783497668.
59. Erwin, A. L. (2016) Antibacterial Drug Discovery Targeting the Lipopolysaccharide Biosynthetic Enzyme LpxC Cold Spring Harb Perspect Med, 6. DOI: 10.1101/cshperspect.a025304.
60. Kalinin, D. V.; Holl, R. (2017) LpxC inhibitors: a patent review (2010-2016) Expert Opin Ther Pat, 27, 1227-1250. DOI: 10.1080/13543776.2017.1360282.
61. Chen, M. H.; Steiner, M. G.; de Laszlo, S. E.; Patchett, A. A.; Anderson, M. S.; Hyland, S. A.; Onishi, H. R.; Silver, L. L.; Raetz, C. R. (1999) Carbohydroxamido-oxazolidines: antibacterial agents that target lipid A biosynthesis Bioorg Med Chem Lett, 9, 313-8. DOI: 10.1016/s0960-894× (98)00749-5.
62. McClerren, A. L.; Endsley, S.; Bowman, J. L.; Andersen, N. H.; Guan, Z.; Rudolph, J.; Raetz, C. R. (2005) A slow, tight-binding inhibitor of the zinc-dependent deacetylase LpxC of lipid A biosynthesis with antibiotic activity comparable to ciprofloxacin Biochemistry, 44, 16574-83. DOI: 10.1021/bi0518186.
63. Mochalkin, I.; Knafels, J. D.; Lightle, S. (2008) Crystal structure of LpxC from Pseudomonas aeruginosa complexed with the potent BB-78485 inhibitor Protein Sci, 17, 450-7. DOI: 10.1110/ps.073324108.
64. Krause, K. M.; Haglund, C. M.; Hebner, C.; Serio, A. W.; Lee, G.; Nieto, V.; Cohen, F.; Kane, T. R.; Machajewski, T. D.; Hildebrandt, D.; Pillar, C.; Thwaites, M.; Hall, D.; Miesel, L.; Hackel, M.; Burek, A.; Andrews, L. D.; Armstrong, E.; Swem, L.; Jubb, A.; Cirz, R. T. (2019) Potent LpxC Inhibitors with In Vitro Activity against Multidrug-Resistant Pseudomonas aeruginosa Antimicrob Agents Chemother, 63. DOI: 10.1128/AAC.00977-19.
65. Cohen, F.; Aggen, J. B.; Andrews, L. D.; Assar, Z.; Boggs, J.; Choi, T.; Dozzo, P.; Easterday, A. N.; Haglund, C. M.; Hildebrandt, D. J.; Holt, M. C.; Joly, K.; Jubb, A.; Kamal, Z.; Kane, T. R.; Konradi, A. W.; Krause, K. M.; Linsell, M. S.; Machajewski, T. D.; Miroshnikova, O.; Moser, H. E.; Nieto, V.; Phan, T.; Plato, C.; Serio, A. W.; Seroogy, J.; Shakhmin, A.; Stein, A. J.; Sun, A. D.; Sviridov, S.; Wang, Z.; Wlasichuk, K.; Yang, W.; Zhou, X.; Zhu, H.; Cirz, R. T. (2019) Optimization of LpxC Inhibitors for Antibacterial Activity and Cardiovascular Safety ChemMedChem, 14, 1560-1572. DOI: 10.1002/cmdc.201900287.
66. Montgomery, J. I.; Brown, M. F.; Reilly, U.; Price, L. M.; Abramite, J. A.; Arcari, J.; Barham, R.; Che, Y.; Chen, J. M.; Chung, S. W.; Collantes, E. M.; Desbonnet, C.; Doroski, M.; Doty, J.; Engtrakul, J. J.; Harris, T. M.; Huband, M.; Knafels, J. D.; Leach, K. L.; Liu, S.; Marfat, A.; McAllister, L.; McElroy, E.; Menard, C. A.; Mitton-Fry, M.; Mullins, L.; Noe, M. C.; O'Donnell, J.; Oliver, R.; Penzien, J.; Plummer, M.; Shanmugasundaram, V.; Thoma, C.; Tomaras, A. P.; Uccello, D. P.; Vaz, A.; Wishka, D. G. (2012) Pyridone methylsulfone hydroxamate LpxC inhibitors for the treatment of serious gram-negative infections J Med Chem, 55, 1662-70. DOI: 10.1021/jm2014875.
67. Piizzi, G.; Parker, D. T.; Peng, Y.; Dobler, M.; Patnaik, A.; Wattanasin, S.; Liu, E.; Lenoir, F.; Nunez, J.; Kerrigan, J.; McKenney, D.; Osborne, C.; Yu, D.; Lanieri, L.; Bojkovic, J.; Dzink-Fox, J.; Lilly, M. D.; Sprague, E. R.; Lu, Y.; Wang, H.; Ranjitkar, S.; Xie, L.; Wang, B.; Glick, M.; Hamann, L. G.; Tommasi, R.; Yang, X.; Dean, C. R. (2017) Design, Synthesis, and Properties of a Potent Inhibitor of Pseudomonas aeruginosa Deacetylase LpxC J Med Chem, 60, 5002-5014. DOI: 10.1021/acs.jmedchem.7b00377.
68. Lee, P. S.; Lapointe, G.; Madera, A. M.; Simmons, R. L.; Xu, W.; Yifru, A.; Tjandra, M.; Karur, S.; Rico, A.; Thompson, K.; Bojkovic, J.; Xie, L.; Uehara, K.; Liu, A.; Shu, W.; Bellamacina, C.; McKenney, D.; Morris, L.; Tonn, G. R.; Osborne, C.; Benton, B. M.; McDowell, L.; Fu, J.; Sweeney, Z. K. (2018) Application of Virtual Screening to the Identification of New LpxC Inhibitor Chemotypes, Oxazolidinone and Isoxazoline J Med Chem, 61, 9360-9370. DOI: 10.1021/acs.jmedchem.8b01287.
69. Furuya, T.; Shapiro, A. B.; Comita-Prevoir, J.; Kuenstner, E. J.; Zhang, J.; Ribe, S. D.; Chen, A.; Hines, D.; Moussa, S. H.; Carter, N. M.; Sylvester, M. A.; Romero, J. A. C.; Vega, C. V.; Sacco, M. D.; Chen, Y.; O'Donnell, J. P.; Durand-Reville, T. F.; Miller, A. A.; Tommasi, R. A. (2020) N-Hydroxyformamide LpxC inhibitors, their in vivo efficacy in a mouse Escherichia coli infection model, and their safety in a rat hemodynamic assay Bioorg Med Chem, 28, 115826. DOI: 10.1016/j.bmc.2020.115826.
70. Ushiyama, F.; Takashima, H.; Matsuda, Y.; Ogata, Y.; Sasamoto, N.; Kurimoto-Tsuruta, R.; Ueki, K.; Tanaka-Yamamoto, N.; Endo, M.; Mima, M.; Fujita, K.; Takata, I.; Tsuji, S.; Yamashita, H.; Okumura, H.; Otake, K.; Sugiyama, H. (2021) Lead optimization of 2-hydroxymethyl imidazoles as non-hydroxamate LpxC inhibitors: Discovery of TP0586532 Bioorg Med Chem, 30, 115964. DOI: 10.1016/j.bmc.2020.115964.
71. Daryaee, F.; Tonge, P. J. (2019) Pharmacokinetic-pharmacodynamic models that incorporate drug-target binding kinetics Curr Opin Chem Biol, 50, 120-127. DOI: 10.1016/j.cbpa.2019.03.008.
72. Walkup, G. K.; You, Z.; Ross, P. L.; Allen, E. K.; Daryaee, F.; Hale, M. R.; O'Donnell, J.; Ehmann, D. E.; Schuck, V. J.; Buurman, E. T.; Choy, A. L.; Hajec, L.; Murphy-Benenato, K.; Marone, V.; Patey, S. A.; Grosser, L. A.; Johnstone, M.; Walker, S. G.; Tonge, P. J.; Fisher, S. L. (2015) Translating slow-binding inhibition kinetics into cellular and in vivo effects Nat Chem Biol, 11, 416-23. DOI: 10.1038/nchembio.1796.
73. Tomaras, A. P.; McPherson, C. J.; Kuhn, M.; Carifa, A.; Mullins, L.; George, D.; Desbonnet, C.; Eidem, T. M.; Montgomery, J. I.; Brown, M. F.; Reilly, U.; Miller, A. A.; O'Donnell, J. P. (2014) LpxC inhibitors as new antibacterial agents and tools for studying regulation of lipid A biosynthesis in Gram-negative pathogens MBio, 5, e01551-14. DOI: 10.1128/mBio.01551-14.
74. Jiang, X.; Hao, X.; Jing, L.; Wu, G.; Kang, D.; Liu, X.; Zhan, P. (2019) Recent applications of click chemistry in drug discovery Expert Opin Drug Discov, 14, 779-789. DOI: 10.1080/17460441.2019.1614910.
75. Fu, J.; Karur, S.; Madera, A. M.; Pecchi, S.; Sweeney, Z. K.; Tjandra, M.; Yifru, A. (2014) Preparation of hydroxamic acid derivatives as LpxC inhibitors useful for the treatment of bacterial infection, WO2014160649.
76. Basu, R.; Wang, N.; Basak, S.; Daryaee, F.; Babar, M.; Allen, E. K.; Walker, S. G.; Haley, J. D.; Tonge, P. J. (2021) Impact of Target Turnover on the Translation of Drug-Target Residence Time to Time-Dependent Antibacterial Activity ACS Infect Dis, 7, 2755-2763. DOI: 10.1021/acsinfecdis.1c00317.
77. Brown, M. F.; Reilly, U.; Abramite, J. A.; Arcari, J. T.; Oliver, R.; Barham, R. A.; Che, Y.; Chen, J. M.; Collantes, E. M.; Chung, S. W.; Desbonnet, C.; Doty, J.; Doroski, M.; Engtrakul, J. J.; Harris, T. M.; Huband, M.; Knafels, J. D.; Leach, K. L.; Liu, S.; Marfat, A.; Marra, A.; McElroy, E.; Melnick, M.; Menard, C. A.; Montgomery, J. I.; Mullins, L.; Noe, M. C.; O'Donnell, J.; Penzien, J.; Plummer, M. S.; Price, L. M.; Shanmugasundaram, V.; Thoma, C.; Uccello, D. P.; Warmus, J. S.; Wishka, D. G. (2012) Potent inhibitors of LpxC for the treatment of Gram-negative infections J Med Chem, 55, 914-23. DOI: 10.1021/jm2014748.
78. Yu, W.; Neckles, C.; Chang, A.; Bommineni, G. R.; Spagnuolo, L.; Zhang, Z.; Liu, N.; Lai, C.; Truglio, J.; Tonge, P. J. (2015) A [(32)P]NAD(+)-based method to identify and quantitate long residence time enoyl-acyl carrier protein reductase inhibitors Anal Biochem, 474, 40-9. DOI: 10.1016/j.ab.2014.12.022.
79. PyMOL. (2015) The PyMOL Molecular Graphics System, Version 2.5 Schrödinger, LLC.
80. Aeschlimann, J. R.; Hershberger, E.; Rybak, M. J. (1999) Analysis of vancomycin population susceptibility profiles, killing activity, and postantibiotic effect against vancomycin-intermediate Staphylococcus aureus Antimicrob Agents Chemother, 43, 1914-8. DOI: 10.1128/AAC.43.8.1914.
81. Davoodi, S.; Daryaee, F.; Chang, A.; Walker, S. G.; Tonge, P. J. (2020) Correlating Drug-Target Residence Time and Post-antibiotic Effect: Insight into Target Vulnerability ACS Infect Dis, 6, 629-636. DOI: 10.1021/acsinfecdis.9b00484.
82. Si, Y.; Basak, S.; Li, Y.; Merino, J.; Iuliano, J. N.; Walker, S. G.; Tonge, P. J. (2019) Antibacterial Activity and Mode of Action of a Sulfonamide-Based Class of Oxaborole Leucyl-tRNA-Synthetase Inhibitors ACS Infect Dis, 5, 1231-1238. DOI: 10.1021/acsinfecdis.9b00071.
83. Schneider, E. V.; Bottcher, J.; Huber, R.; Maskos, K.; Neumann, L. (2013) Structure-kinetic relationship study of CDK8/CycC specific compounds Proc Natl Acad Sci USA, 110, 8081-6. DOI: 10.1073/pnas.1305378110.
84. Schoop, A.; Dey, F. (2015) On-rate based optimization of structure-kinetic relationship—surfing the kinetic map Drug Discov Today Technol, 17, 9-15. DOI: 10.1016/j.ddtec.2015.08.003.
85. Spagnuolo, L. A.; Eltschkner, S.; Yu, W.; Daryaee, F.; Davoodi, S.; Knudson, S. E.; Allen, E. K.; Merino, J.; Pschibul, A.; Moree, B.; Thivalapill, N.; Truglio, J. J.; Salafsky, J.; Slayden, R. A.; Kisker, C.; Tonge, P. J. (2017) Evaluating the Contribution of Transition-State Destabilization to Changes in the Residence Time of Triazole-Based InhA Inhibitors J Am Chem Soc, 139, 3417-3429. DOI: 10.1021/jacs.6b11148.
86. Xia, L.; Burger, W. A. C.; van Veldhoven, J. P. D.; Kuiper, B. J.; van Duijl, T. T.; Lenselink, E. B.; Paasman, E.; Heitman, L. H.; AP, I. J. (2017) Structure-Affinity Relationships and Structure-Kinetics Relationships of Pyrido[2,1-f]purine-2,4-dione Derivatives as Human Adenosine A3 Receptor Antagonists J Med Chem, 60, 7555-7568. DOI: 10.1021/acs.jmedchem.7b00950.
87. O'Shea, R.; Moser, H. E. (2008) Physicochemical properties of antibacterial compounds: implications for drug discovery J. Med. Chem., 51, 2871-8. DOI: 10.1021/jm700967e.
88. Richter, M. F.; Drown, B. S.; Riley, A. P.; Garcia, A.; Shirai, T.; Svec, R. L.; Hergenrother, P. J. (2017) Predictive compound accumulation rules yield a broad-spectrum antibiotic Nature. DOI: 10.1038/nature22308.
89. Ropponen, H. K.; Richter, R.; Hirsch, A. K. H.; Lehr, C. M. (2021) Mastering the Gram-negative bacterial barrier—Chemical approaches to increase bacterial bioavailability of antibiotics Adv Drug Deliv Rev, 172, 339-360. DOI: 10.1016/j.addr.2021.02.014.
90. Daryaee, F.; Chang, A.; Schiebel, J.; Lu, Y.; Zhang, Z.; Kapilashrami, K.; Walker, S. G.; Kisker, C.; Sotriffer, C. A.; Fisher, S. L.; Tonge, P. J. (2016) Correlating Drug-Target Kinetics and In vivo Pharmacodynamics: Long Residence Time Inhibitors of the FabI Enoyl-ACP Reductase Chem Sci, 7, 5945-5954. DOI: 10.1039/C6SC01000H.
91. Lee, C. J.; Liang, X.; Wu, Q.; Najeeb, J.; Zhao, J.; Gopalaswamy, R.; Titecat, M.; Sebbane, F.; Lemaitre, N.; Toone, E. J.; Zhou, P. (2016) Drug design from the cryptic inhibitor envelope Nat Commun, 7, 10638. DOI: 10.1038/ncomms10638.
92. Clayton, G. M.; Klein, D. J.; Rickert, K. W.; Patel, S. B.; Kornienko, M.; Zugay-Murphy, J.; Reid, J. C.; Tummala, S.; Sharma, S.; Singh, S. B.; Miesel, L.; Lumb, K. J.; Soisson, S. M. (2013) Structure of the bacterial deacetylase LpxC bound to the nucleotide reaction product reveals mechanisms of oxyanion stabilization and proton transfer J Biol Chem, 288, 34073-34080. DOI: 10.1074/jbc.M113.513028.
93. Lee, C. J.; Liang, X.; Gopalaswamy, R.; Najeeb, J.; Ark, E. D.; Toone, E. J.; Zhou, P. (2014) Structural basis of the promiscuous inhibitor susceptibility of Escherichia coli LpxC ACS Chem Biol, 9, 237-46. DOI: 10.1021/cb400067g.
94. Lee, C. J.; Liang, X.; Chen, X.; Zeng, D.; Joo, S. H.; Chung, H. S.; Barb, A. W.; Swanson, S. M.; Nicholas, R. A.; Li, Y.; Toone, E. J.; Raetz, C. R.; Zhou, P. (2011) Species-specific and inhibitor-dependent conformations of LpxC: implications for antibiotic design Chem Biol, 18, 38-47. DOI: 10.1016/j.chembiol.2010.11.011.
95. Barb, A. W.; McClerren, A. L.; Snehelatha, K.; Reynolds, C. M.; Zhou, P.; Raetz, C. R. (2007) Inhibition of lipid A biosynthesis as the primary mechanism of CHIR-090 antibiotic activity in Escherichia coli Biochemistry, 46, 3793-802. DOI: 10.1021/bi6025165.
96. Surivet, J. P.; Panchaud, P.; Specklin, J. L.; Diethelm, S.; Blumstein, A. C.; Gauvin, J. C.; Jacob, L.; Masse, F.; Mathieu, G.; Mirre, A.; Schmitt, C.; Lange, R.; Tidten-Luksch, N.; Gnerre, C.; Seeland, S.; Herrmann, C.; Seiler, P.; Enderlin-Paput, M.; Mac Sweeney, A.; Wicki, M.; Hubschwerlen, C.; Ritz, D.; Rueedi, G. (2020) Discovery of Novel Inhibitors of LpxC Displaying Potent in Vitro Activity against Gram-Negative Bacteria J Med Chem, 63, 66-87. DOI: 10.1021/acs.jmedchem.9b01604.
97. Li, H. J.; Lai, C. T.; Pan, P.; Yu, W.; Liu, N.; Bommineni, G. R.; Garcia-Diaz, M.; Simmerling, C.; Tonge, P. J. (2014) A structural and energetic model for the slow-onset inhibition of the Mycobacterium tuberculosis enoyl-ACP reductase InhA ACS Chem Biol, 9, 986-93. DOI: 10.1021/cb400896g.
98. Lai, C. T.; Li, H. J.; Yu, W.; Shah, S.; Bommineni, G. R.; Perrone, V.; Garcia-Diaz, M.; Tonge, P. J.; Simmerling, C. (2015) Rational Modulation of the Induced-Fit Conformational Change for Slow-Onset Inhibition in Mycobacterium tuberculosis InhA Biochemistry, 54, 4683-91. DOI: 10.1021/acs.biochem.5b00284.
99. Pan, A. C.; Borhani, D. W.; Dror, R. O.; Shaw, D. E. (2013) Molecular determinants of drug-receptor binding kinetics Drug Discov Today, 18, 667-73. DOI: 10.1016/j.drudis.2013.02.007.
100. Miller, D. C.; Lunn, G.; Jones, P.; Sabnis, Y.; Davies, N. L.; Driscoll, P. (2012) Investigation of the effect of molecular properties on the binding kinetics of a ligand to its biological target Medchemcomm, 3, 449-449. DOI: 10.1039/c2md00270a.
101. Lu, H.; Iuliano, J. N.; Tonge, P. J. (2018) Structure-kinetic relationships that control the residence time of drug-target complexes: insights from molecular structure and dynamics Curr Opin Chem Biol, 44, 101-109. DOI: 10.1016/j.cbpa.2018.06.002.
102. Tresadern, G.; Bartolome, J. M.; Macdonald, G. J.; Langlois, X. (2011) Molecular properties affecting fast dissociation from the D2 receptor Bioorg Med Chem, 19, 2231-41. DOI: 10.1016/j.bmc.2011.02.033.
103. Zhao, Y. and Lin, Y. H. (2010) Whole-cell protein identification using the concept of unique peptides. Genomics Proteomics Bioinformatics 8, 33-41. DOI: 10.1016/S1672-0229(10)60004-6.
104. Frank, A. M. (2009) A ranking-based scoring function for peptide-spectrum matches. J Proteome Res 8, 2241-52. DOI: 10.1021/pr800678b.
105. Kall, L., Storey, J. D., MacCoss, M. J. and Noble, W. S. (2008) Posterior error probabilities and false discovery rates: two sides of the same coin. J Proteome Res 7, 40-4. DOI: 10.1021/pr700739d.
106. Andersen, N. H., Bowman, J., Erwin, A. L., Harwood, E. A., Kline, T., Mdluli, K. E., Ng, S., Pfister, K. B., Shawar, R., Wagman, A. S. and Yabannavar, A. Antibacterial Agents. U.S. Pat. No. 8,084,615 B2, 2011.
107. Himo, F., Lovell, T., Hilgraf, R., Rostovtsev, V. V., Noodleman, L., Sharpless, K. B. and Fokin, V. V. (2005) Copper(I)-catalyzed synthesis of azoles. DFT study predicts unprecedented reactivity and intermediates. J Am Chem Soc 127, 210-6. DOI: 10.1021/ja0471525.
108. Brown, M. F., Reilly, U., Abramite, J. A., Arcari, J. T., Oliver, R., Barham, R. A., Che, Y., Chen, J. M., Collantes, E. M., Chung, S. W., Desbonnet, C., Doty, J., Doroski, M., Engtrakul, J. J., Harris, T. M., Huband, M., Knafels, J. D., Leach, K. L., Liu, S., Marfat, A., Marra, A., McElroy, E., Melnick, M., Menard, C. A., Montgomery, J. I., Mullins, L., Noe, M. C., O'Donnell, J., Penzien, J., Plummer, M. S., Price, L. M., Shanmugasundaram, V., Thoma, C., Uccello, D. P., Warmus, J. S. and Wishka, D. G. (2012) Potent inhibitors of LpxC for the treatment of Gram-negative infections. J Med Chem 55, 914-23. DOI: 10.1021/jm2014748.
109. Montgomery, J. I., Brown, M. F., Reilly, U., Price, L. M., Abramite, J. A., Arcari, J., Barham, R., Che, Y., Chen, J. M., Chung, S. W., Collantes, E. M., Desbonnet, C., Doroski, M., Doty, J., Engtrakul, J. J., Harris, T. M., Huband, M., Knafels, J. D., Leach, K. L., Liu, S., Marfat, A., McAllister, L., McElroy, E., Menard, C. A., Mitton-Fry, M., Mullins, L., Noe, M. C., O'Donnell, J., Oliver, R., Penzien, J., Plummer, M., Shanmugasundaram, V., Thoma, C., Tomaras, A. P., Uccello, D. P., Vaz, A. and Wishka, D. G. (2012) Pyridone methylsulfone hydroxamate LpxC inhibitors for the treatment of serious gram-negative infections. J Med Chem 55, 1662-70. DOI: 10.1021/jm2014875.
110. Montgomery, J. I.; Brown, M. F.; Reilly, U.; Price, L. M.; Abramite, J. A.; Arcari, J.; Barham, R.; Che, Y.; Chen, J. M.; Chung, S. W.; Collantes, E. M.; Desbonnet, C.; Doroski, M.; Doty, J.; Engtrakul, J. J.; Harris, T. M.; Huband, M.; Knafels, J. D.; Leach, K. L.; Liu, S.; Marfat, A.; McAllister, L.; McElroy, E.; Menard, C. A.; Mitton-Fry, M.; Mullins, L.; Noe, M. C.; O'Donnell, J.; Oliver, R.; Penzien, J.; Plummer, M.; Shanmugasundaram, V.; Thoma, C.; Tomaras, A. P.; Uccello, D. P.; Vaz, A.; Wishka, D. G. Pyridone methylsulfone hydroxamate LpxC inhibitors for the treatment of serious gram-negative infections. J. Med. Chem. 2012, 55, 1662-70. DOT: 10.1021/jm2014875.
111. Fu, J.; Karur, S.; Madera, A. M.; Pecchi, S.; Sweeney, Z. K.; Tjandra, M.; Yifru, A. (2014) Preparation of hydroxamic acid derivatives as LpxC inhibitors useful for the treatment of bacterial infection, WO2014160649.
112. Masuda, N.; Sakagawa, E.; Ohya, S.; Gotoh, N.; Tsujimoto, H.; Nishino, T. (2000) Substrate specificities of MexAB-OprM, MexCD-OprJ, and MexXY-oprM efflux pumps in Pseudomonas aeruginosa. Antimicrob. Agents Chemother. 2000, 44, 3322-7. DOI: 10.1128/aac.44.12.3322-3327.2000.
113. Mochalkin, I.; Knafels, J. D.; Lightle, S. Crystal structure of LpxC from Pseudomonas aeruginosa complexed with the potent BB-78485 inhibitor. Protein Sci. 2008, 17, 450-7. DOI: 10.1110/ps.073324108.
114. Kabsch, W. Xds Acta Crystallogr. D Biol. Crystallogr. 2010, 66, 125-32. DOI: 10.1107/S0907444909047337.
115. Emsley, P.; Lohkamp, B.; Scott, W. G.; Cowtan, K. Features and development of Coot. Acta Crystallogr. D Biol. Crystallogr. 2010, 66, 486-501. DOI: doi:10.1107/50907444910007493.
116. Adams, P. D.; Afonine, P. V.; Bunkoczi, G.; Chen, V. B.; Davis, I. W.; Echols, N.; Headd, J. J.; Hung, L. W.; Kapral, G. J.; Grosse-Kunstleve, R. W.; McCoy, A. J.; Moriarty, N. W.; Oeffner, R.; Read, R. J.; Richardson, D. C.; Richardson, J. S.; Terwilliger, T. C.; Zwart, P. H. PHENIX: a comprehensive Python-based system for macromolecular structure solution. Acta Crystallogr. D Biol. Crystallogr. 2010, 66, 213-221. DOI: 10.1107/s0907444909052925.
117. Chen, V. B.; Arendall, W. B., 3rd; Headd, J. J.; Keedy, D. A.; Immormino, R. M.; Kapral, G. J.; Murray, L. W.; Richardson, J. S.; Richardson, D. C. (2010) MolProbity: all-atom structure validation for macromolecular crystallography. Acta Crystallogr. D Biol. Crystallogr. 2010, 66, 12-21. DOI: 10.1107/S0907444909042073.
118. Johnson, K. A. In Methods Enzymol 2009; Vol. 467, p 601-626.
119. Walkup, G. K.; You, Z.; Ross, P. L.; Allen, E. K.; Daryaee, F.; Hale, M. R.; O'Donnell, J.; Ehmann, D. E.; Schuck, V. J.; Buurman, E. T.; Choy, A. L.; Hajec, L.; Murphy-Benenato, K.; Marone, V.; Patey, S. A.; Grosser, L. A.; Johnstone, M.; Walker, S. G.; Tonge, P. J.; Fisher, S. L. Translating slow-binding inhibition kinetics into cellular and in vivo effects. Nat. Chem. Biol. 2015, 11, 416-23. DOI: 10.1038/nchembio.1796.
120. Basu, R.; Wang, N.; Basak, S.; Daryaee, F.; Babar, M.; Allen, E. K.; Walker, S. G.; Haley, J. D.; Tonge, P. J. Impact of Target Turnover on the Translation of Drug-Target Residence Time to Time-Dependent Antibacterial Activity. ACS Infect. Dis. 2021, 7, 2755-2763. DOI: 10.1021/acsinfecdis.1c00317.
121. CLSI In CLSI Standard M07; 11 ed.; Clinical and Laboratory Standards Institute: Wayne, PA, 2018.
122. Bundtzen, R. W.; Gerber, A. U.; Cohn, D. L.; Craig, W. A. Postantibiotic suppression of bacterial growth. Rev. Infect. Dis. 1981, 3, 28-37. DOI: 10.1093/clinids/3.1.28.
123. Si, Y.; Basak, S.; Li, Y.; Merino, J.; Iuliano, J. N.; Walker, S. G.; Tonge, P. J. Antibacterial Activity and Mode of Action of a Sulfonamide-Based Class of Oxaborole Leucyl-tRNA-Synthetase Inhibitors. ACS Infect. Dis. 2019, 5, 1231-1238. DOI: 10.1021/acsinfecdis.9b00071.

What is claimed is:

1. A compound having the structure: 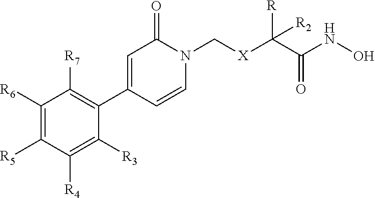
wherein R₁ and R₂ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, -alkyl-OR₁₄, C₁₋₁₀ alkyl, C₂₋₁₀ alkenyl, or C₂₋₁₀ alkynyl; or wherein R₁ and R₂ together form into a cycloalkyl or cycloheteroalkyl;
wherein R₃ and R₇ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl;
wherein R₄ and R₆ are each independently H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl;
wherein R₅ is —NO₂, —NHR₁₄, —NR₁₄R₁₅, —SR₁₄, —SO₂R₁₄, —(C═O)—NH—R₁₄, —CO₂R₁₄, -alkyl-NR₁₄R₁₅, -alkyl-OR₁₄, C₂₋₁₀ alkenyl, C₂₋₁₀ alkynyl, —(C₀₋₁₀ alkyl)-heterocyclyl, 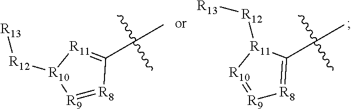
wherein R₈, R₉, R₁₀, and R₁₁, are each independently N or CH;
wherein R₁₂ is C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₃ is halogen, —NO₂, —CN, —NHR₁₄, —NR₁₄R₁₅, —SR₁₄, —SO₂R₁₄, —OR₁₄, —CO₂R₁₄, —CF₃, —NR₁₄R₁₅, —OR₁₄, C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₄ is H, C₂₋₁₀ alkenyl, C₂₋₁₀ alkynyl, —C═O-alkyl-aryl, or alkyl-aryl;
wherein R₁₅ is H, C₁₋₁₀ alkyl, C₂₋₁₀ alkenyl, C₂₋₁₀ alkynyl, —C═O-alkyl-aryl, or alkyl-aryl;
wherein X is CH₂ or NH;
wherein R₃ and R₅ are not both halogen, R₅ and R₇ are not both halogen;
wherein when R₃ or R₇ is halogen, R₄ and R₆ are not alkyl;
wherein when R₅ is —(C₀₋₁₀ alkyl)-heterocyclyl, the heterocyclyl is not tetrahydropyranyl;
wherein when R₃ or R₇ is halogen, R₄ and R₆ are not —OR₁₄;
wherein when R₃ is alkyl or H, R₆ is not —NHR₁₄ and —NR₁₄R₁₅; wherein when R₇ is alkyl or H, R₄ is not —NHR₁₄ and —NR₁₄R₁₅.

2. The compound of claim 1,
a) wherein R₃ is H, R₄ and R₈ are each independently H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl; or
b) wherein R₄ is H, R₃ and R₇ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl; or
c) wherein R₆ is H, R₃ and R₇ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl; or
d) wherein R₇ is H, R₄ and R₈ are each independently H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl; or
e) wherein R₃ is halogen, R₄ and R₈ are each independently H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl; or
f) wherein R₇ halogen, R₄ and R₈ are each independently H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl;
g) wherein R₃ and R₄ are H, R₆ is H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl, and R₇ is H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl; or
h) wherein R₃ and R₈ are H, R₄ and R₇ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, -alkyl-OR₁₄, C₁₋₁₀ alkyl, C₂₋₁₀ alkenyl or C₂₋₁₀ alkynyl; or
i) wherein R₃ and R₇ are H, R₄ and R₈ are each independently H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl; or
j) wherein R₄ and R₈ are H, R₃ and R₇ are each independently H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl; or
k) wherein R₄ and R₇ are H, R₃ is H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl, and R₈ is H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl; or
l) wherein R₆ and R₇ are H, R₃ is H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl, and R₄ is H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl; or
m) wherein R₄ and R₈ are each independently H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl;
n) wherein R₃, R₄ and R₆ are H, R₇ is H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl; or
o) wherein R₃, R₄ and R₇ are H, R₆ is H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl; or
p) wherein R₃, R₈ and R₇ are H, R₄ is H, —NO₂, —SR₁₄, —SO₂R₁₄, —OR₁₄, —CO₂R₁₄, -alkyl-NR₁₄R₁₅, -alkyl-OR₁₄, C₂₋₁₀ alkenyl or C₂₋₁₀ alkynyl; or
q) wherein R₄, R₈ and R₇ are H, R₃ is H, halogen, —NO₂, —CN, —NHR₁₄, —NR₁₄R₁₅, —SR₁₄, —SO₂R₁₄, —OR₁₄, —CO₂R₁₄, —CF₃, -alkyl-NR₁₄R₁₅, C₁₋₁₀ alkyl, C₂₋₁₀ alkenyl or C₂₋₁₀ alkynyl.

3. (canceled)

4. (canceled)

5. The compound of claim 1, wherein (a) the halogen is F, Cl, Br or I (b) R₃ is F, R₄, R₆ and R₇ are H; (c) R₇ is F, R₄, R₆ and R₃ are H; (d) R₃, R₄, R₆ and R₇ are H; or (e) R₁ and R₂ are each independently —SO₂R₁₄, —OR₁₄, —CO₂R₁₄, —CF₃, -alkyl-NR₁₄R₁₅, -alkyl-OR₁₄, C₁₋₁₀ alkyl, C₂₋₁₀ alkenyl, or C₂₋₁₀ alkynyl.

6. (canceled)

7. (canceled)

8. (canceled)

9. The compound of claim 1, wherein R₁ and R₂ are each independently —SO₂R₁₄ or C₁₋₁₀ alkyl; or are each independently H or -alkyl-OR₁₄.

10. (canceled)

11. (canceled)

12. The compound of claim 1, wherein R₁ and R₂ are each independently H or 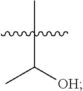 [SMILES: *C(C)O]
or R₁ and R₂ together form into a cycloheteroalkyl.

13. (canceled)

14. The compound of claim 1 having the structure: 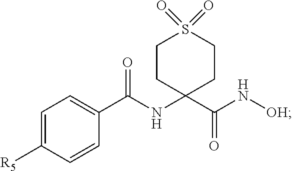
wherein R₅ is NO₂, —NHR₁₄, —NR₁₄R₁₅, —SR₁₄, —SO₂R₁₄, —CO₂R₁₄, -alkyl-NR₁₄R₁₅, -alkyl-OR₁₄, C₂₋₁₀ alkenyl, C₂₋₁₀ alkynyl, —(C₀₋₁₀ alkyl)-heterocyclyl,

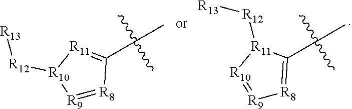

15. (canceled)

16. (canceled)

17. (canceled)

18. (canceled)

19. (canceled)

20. The compound of claim 14, wherein R₅ is 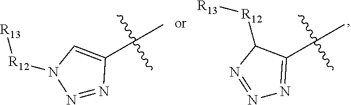
wherein R₁₂ is methyl.

21. (canceled)

22. (canceled)

23. (canceled)

24. The compound of claim 14, wherein (a) R₁₂ is methyl; (b) R₁₃ is phenyl; (c) R₅ is —NHR₁₄, and R₁₄ is —C═O— methyl-phenyl; (c) R₅ is —(C═O)—NH—R₁₄, wherein R₁₄ is methyl-phenyl or (d) R₅ is

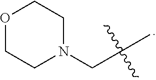 [SMILES: *CN1CCOCC1 ; "."]

25. (canceled)

26. (canceled)

27. (canceled)

28. (canceled)

29. (canceled)

30. (canceled)

31. (canceled)

32. The compound of claim 1, wherein
a) R₈ is N, R₉, R₁₀, and R₁₁ are CH; or
b) R₉ is N, R₈, R₁₀, and R₁₁ are CH; or
c) R₁₀ is N, R₈, R₉, and R₁₁ are CH; or
d) R₁₁ is N, R₈, R₉, and R₁₀ are CH; or
e) R₈ and R₉ is N, R₁₀, and R₁₁ are CH; or
f) R₈ and R₁₀ is N, R₉, and R₁₁ are CH; or
g) R₈ and R₁₁ is N, R₉, and R₁₀ are CH; or
h) R₉ and R₁₀ is N, R₈, and R₁₁ are CH; or
i) R₉ and R₁₁ is N, R₈, and R₁₀ are CH; or
j) R₁₀ and R₁₁ is N, R₈, and R₉ are CH; or
k) R₈, R₉, and R₁₀ is N, R₁₁ is CH; or
l) R₈, R₉, and R₁₁ is N, R₁₀ is CH; or
m) R₉, R₁₀, and R₁₁ is N, R₈ is CH; or
(n) R₈ is CH, R₉, R₁₀, and R₁₁ are N; or
(o) R₉ is CH, R₈, R₁₀, and R₁₁ are N; or
(p) R₁₀ is CH, R₈, R₉, and R₁₁ are N; or
(q) R₁₁ is CH, R₈, R₉, and R₁₀ are N; or
(r) R₈ and R₉ is CH, R₁₀, and R₁₁ are N; or
(s) R₈ and R₁₀ is CH, R₉, and R₁₁ are N; or
(t) R₈ and R₁₁ is CH, R₉, and R₁₀ are N; or
(u) R₉ and R₁₀ is CH, R₈, and R₁₁ are N; or
(v) R₉ and R₁₁ is CH, R₈, and R₁₀ are N; or
(w) R₁₀ and R₁₁ is CH, R₈, and R₉ are N; or
(x) R₈, R₉, and R₁₀ is CH, R₁₁ is N; or
(y) R₈, R₉, and R₁₁ is CH, R₁₀ is N; or
(z) R₉, R₁₀, and R₁₁ is CH, R₈ is N.

33. (canceled)

34. The compound of claim 32, wherein R₈, R₉, and R₁₀ is N, R₁₁ is CH; or wherein R₉, R₁₀, and R₁₁ is N, R₈ is CH; or wherein R₁₂ is C₁₋₁₀ alkyl, C₂₋₁₀ alkenyl or C₂₋₁₀ alkynyl or wherein R₁₂ is methyl.

35. (canceled)

36. (canceled)

37. (canceled)

38. The compound of claim 1, having the structure: 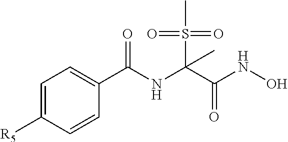
wherein R₅ is 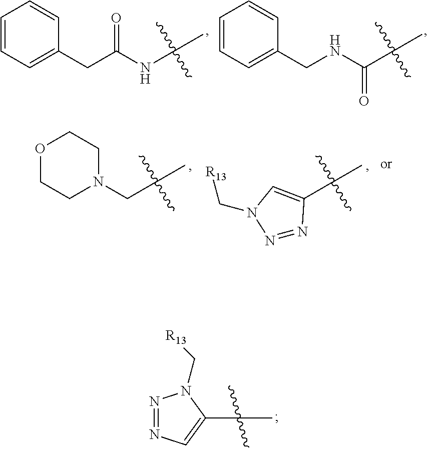
or having the structure 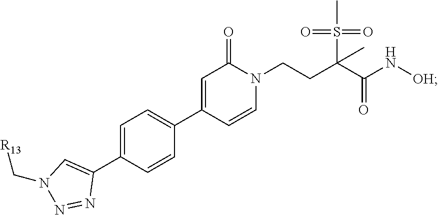
or having the structure: 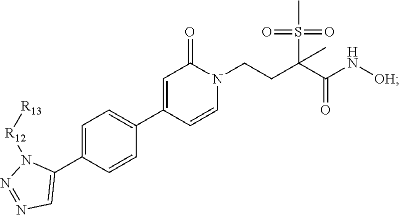
wherein R₁₂ is C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₃ is halogen, —NO₂, —CN, —NHR₁₄, —NR₁₄R₁₅, —SR₁₄, —SO₂R₁₄, —OR₁₄, —CO₂R₁₄, —CF₃, —NR₁₄R₁₅, —OR₁₄, C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₄ is H, C₂₋₁₀ alkenyl, C₂₋₁₀ alkynyl, —C═O-alkyl-aryl, or alkyl-aryl;
wherein R₁₅ is H, C₁₋₁₀ alkyl, C₂₋₁₀ alkenyl, C₂₋₁₀ alkynyl, —C═O-alkyl-aryl, or alkyl-aryl;
or having the structure: 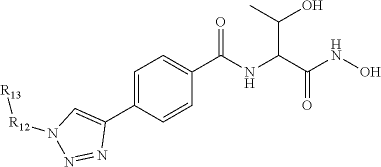
wherein R₁₂ is C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₃ is halogen, —NO₂, —CN, —NHR₁₄, —NR₁₄R₁₅, —SR₁₄, —SO₂R₁₄, —OR₁₄, —CO₂R₁₄, —CF₃, —NR₁₄R₁₅, —OR₁₄, C₁₋₁₀ alkyl, C₂₋₁₀ alkenyl, C₂₋₁₀ alkynyl, aryl, heteroaryl, cycloalkyl, cycloheteroalky, or heterocyclyl;
wherein R₁₄ is H, C₂₋₁₀ alkenyl, C₂₋₁₀ alkynyl, —C═O-alkyl-aryl, or alkyl-aryl;
wherein R₁₅ is H, C₁₋₁₀ alkyl, C₂₋₁₀ alkenyl, C₂₋₁₀ alkynyl, —C═O-alkyl-aryl, or alkyl-aryl.

39. (canceled)

40. The compound of claim 38, wherein R₁₃ is —NHR₁₄, —NR₁₄R₁₅, —SR₁₄, —SO₂R₁₄, —OR₁₄, —CO₂R₁₄, —CF₃, —NR₁₄R₁₅, —OR₁₄, C₁₋₁₀ alkyl, C₂₋₁₀ alkenyl, C₂₋₁₀ alkynyl, aryl, heteroaryl, cycloalkyl, cycloheteroalky, or heterocyclyl.

41. (canceled)

42. The compound of claim 38, wherein R₁₃ is

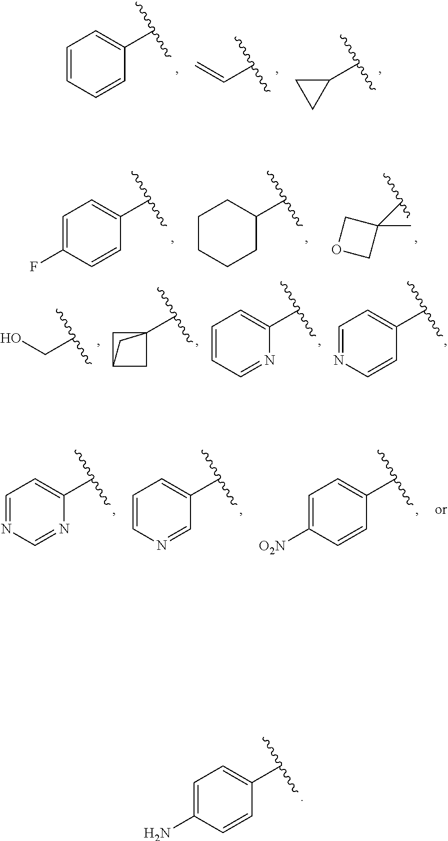

43. (canceled)

44. The compound of claim 1 having the structure:

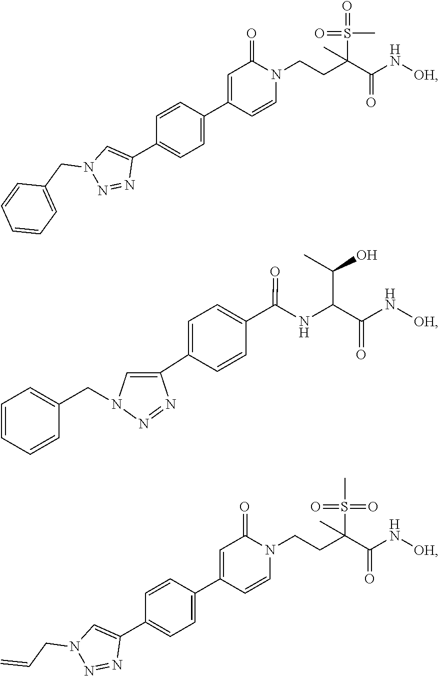

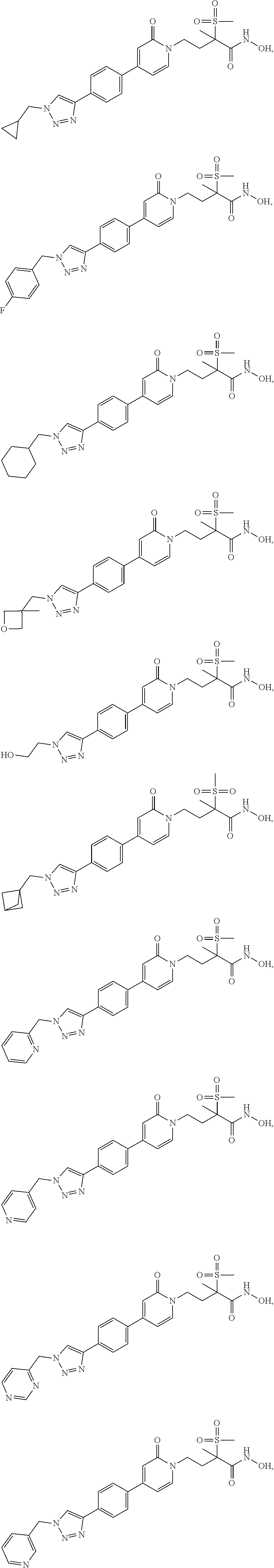

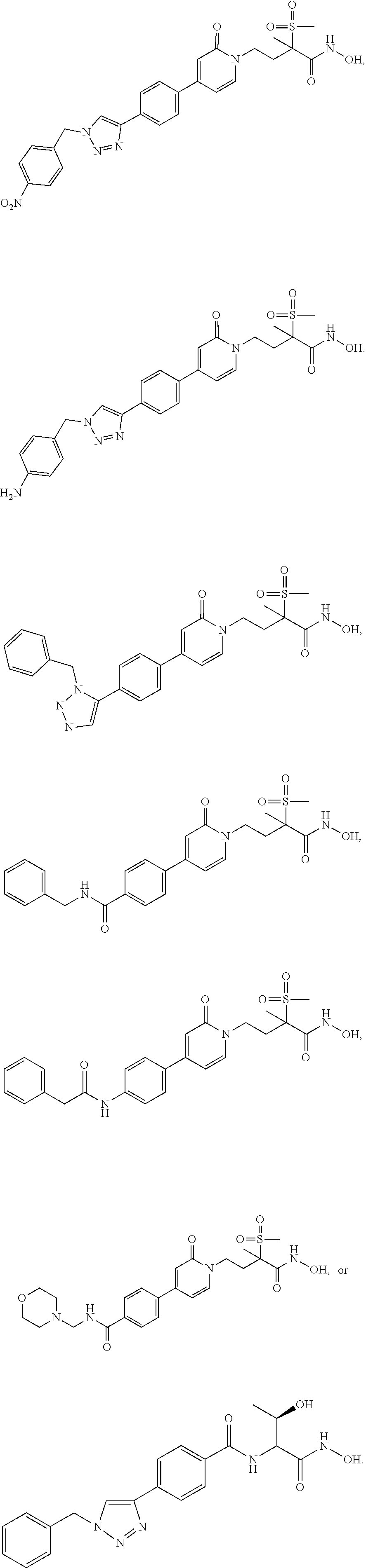

45. (canceled)

46. (canceled)

47. (canceled)

48. (canceled)

49. (canceled)

50. (canceled)

51. (canceled)

52. (canceled)

53. (canceled)

54. A pharmaceutical composition comprising a compound of claim 1 in admixture with at least one pharmaceutically acceptable excipient, diluent or carrier.

55. A method of inhibiting bacteria growth, comprising contacting the bacteria with an amount of compound of claim 1.

56. A method of treating a bacterial infection in a patient, comprising administering to the patient a therapeutically effective amount of the compound of any one of claim 1.

57. (canceled)

58. The method of claim 56, wherein the bacterial infection is caused by a Gram-negative bacteria, wherein the Gram-negative bacteria is Mannheimia haemotytica, Pasteurella multocida, Histophilus somni, Actinobacillus pleuropneumoniae, Salmonella enteritidis, Salmonella gallinahum, Lawsonia intracellularis, Brachyspira hyodysenteriae, Brachyspira pilosicoli, Acinetobacter baumannii, Acinetobacter spp., Citrobacter spp., Enterobacter aerogenes, Enterobacter cloacae, Escherichia coli, Klebsiella oxytoca, Klebsiella pneumoniae, Serratia marcescens, Stenotrophomonas maltophilia, or Pseudomonas aeruginosa.

59. The method of claim 58, wherein the Gram-negative bacterial infection is hospital-acquired pneumonia, urinary tract infections (UTI) intra-abdominal infections (IAI), catheter-associated bloodstream infections, respiratory infection, gastrointestinal infection, nosocomial pneumonia, urinary tract infection, bacteremia, sepsis, skin infection, soft-tissue infection, intraabdominal infection, lung infection, endocarditis, diabetic foot infection, osteomyelitis or central nervous system infection.

60. The method of claim 56, wherein the therapeutically effective amount of the compound is administered orally, topically, or by injection.

61. (canceled)

62. (canceled)
